(19) United States
(12) Patent Application Publication
(10) Pub. No.: US 2026/0274852 A1
(43) Pub. Date: Sep. 17, 2026

(54) SUBSTITUTED PYRIDINE AMIDES

(71) Applicant: Architect Therapeutics, Inc., San Diego, CA (US)

(72) Inventors: Young K. Chen, San Diego, CA (US); You-Chen Lin, San Diego, CA (US); Guangrong Meng, San Diego, CA (US); Robert Borzilleri, San Diego, CA (US)

(21) Appl. No.: 19/563,507

(22) Filed: Mar. 11, 2026

Related U.S. Application Data
(60) Provisional application No. 63/770,770, filed on Mar. 12, 2025.
(60) Provisional application No. 63/877,782, filed on Sep. 8, 2025.

(51) Int. Cl. C07D 487/08; A61K 31/444; A61K 31/497; C07D 403/14; C07D 487/04; C07D 487/10

(52) U.S. Cl. CPC C07D 487/08; A61K 31/444; A61K 31/497; C07D 403/14; C07D 487/04; C07D 487/10

(57) ABSTRACT

The present disclosure describes compounds of Formula I, or a pharmaceutically acceptable salt thereof, pharmaceutical compositions comprising the compounds, methods of preparation, and methods of use. The compounds of the disclosure can inhibit the interaction between SLC15A4 and TASL, and are believed to be useful for treating an autoimmune disease, such as systemic lupus erythematosus, or an inflammatory condition.

### CROSS-REFERENCE TO RELATED APPLICATIONS

This application claims priority to U.S. provisional application No. 63/770,770, filed Mar. 12, 2025, and U.S. provisional application No. 63/877,782, filed Sep. 8, 2025, which are each incorporated herein in their entireties for all purposes.

### BACKGROUND

Dysregulation of pathogen-recognition pathways of the innate immune system is associated with multiple autoimmune disorders. Due to the intricacies of the molecular network involved, the identification of pathway- and disease-specific therapeutics has been challenging.

Endolysosomal toll-like receptors, e.g., TLR7, TLR8, and TLR9, are critical for detecting pathogens and initiating immune responses. However, their aberrant activation is associated with autoimmune diseases, including systemic lupus erythematosus (SLE). Interferon regulatory factor 5 (IRF5) belongs to a family of transcription factors originally implicated in antiviral responses and interferon production. It is believed that IRF5 plays a central role in inflammation by induction of proinflammatory cytokines. This is also supported by genome-wide association studies (GWAS) where genetic variants within or near IRF5 are robustly associated with SLE. Recently, the mechanism of endolysosomal TLR pathway activation of IRF5 has been reported in several studies. The discovery of the endolysosomal solute carrier family 15 member 4 (SLC15A4) was implicated in TLR7/8/9-mediated immune responses, and genetic loss of function mutant (aka ‘feeble’) and haploinsufficiency can completely protect against murine SLE disease models. Indeed, SLC15A4 loss results in a defect in IRF5 activation and dampened proinflammatory cytokine production.

The discovery of a previously uncharacterized protein encoded by the X-linked CXorf21 gene (also known as TASL for TLR Adaptor interacting with SLC15A4 on the Lysosome) was indicated as a key adaptor protein of IRF5 signaling axis. TASL interacts with both SLC15A4 (via its N-terminus) and IRF5 (via its C-terminus), forming a complex that brings IRF5 close to the endolysosome where endolysosomal TLR components can induce its phosphorylation. Cells deficient in TASL can no longer activate IRF5 downstream of endolysosomal TLR stimulation, phenocopying SLC15A4 deficient cells. This unique molecular positioning of the SLC15A4-TASL-IRF5 complex within the endolysosomal TLR pathway may permit the opportunity to selectively inactivate IRF5 without affecting ligand-receptor interaction of upstream TLR signaling (MAPK and NFκB). Therefore, agents which can disrupt the SLC15A4-TASL-IRF5 signaling axis may have benefit in treating human autoimmune disorders, including SLE.

PCT application publication WO 2024/089045 reported synthesis and evaluation of a small number of quinoline amide compounds on disrupting the SLC15A4-TASL interaction. Compound C5 has been evaluated mechanistically with regard to its ability to disrupt the SLC15A4-TASL adapter module. See, Boeszermenyi, A. et al. Nature Communications 2023, 14: 6626, pages 1-12.

There is an urgent need for new treatments and therapeutic modalities against autoimmune diseases, in particular against systemic lupus erythematosus, as well as inflammation conditions. The present invention addresses this problem through provision of new chemical entities useful in the treatment or prevention of autoimmune diseases, in particular against systemic lupus erythematosus, as well as inflammation conditions.

### BRIEF SUMMARY

The present disclosure provides a compound of Formula I:

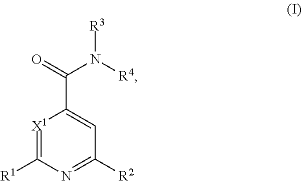

or pharmaceutically acceptable salt thereof,
wherein
R¹ is a C₃-C₈ cycloalkyl, heterocyclyl, C₆-C₁₀ aryl, or heteroaryl, which are each substituted by 0, 1, 2, or 3 R^{1a};
R² is a C₃-C₈ cycloalkyl, heterocyclyl, C₆-C₁₀ aryl, or heteroaryl, which are each substituted by 0, 1, 2, or 3 R^{2a};
X¹ is C—X^{1a} or N;
X^{1a} is H, C₁-C₆ alkyl, C₂-C₆ alkenyl, C₂-C₆ alkynyl, C₁-C₆ alkoxy, C₁-C₆ aminoalkyl, C₁-C₆ sulfonylalkyl, C₁-C₆ haloalkyl, C₁-C₆ haloalkoxy, halogen, or —CN;
R³ is —(C₀-C₃ alkylene)(C₃-C₈ cycloalkyl), —(C₀-C₃ alkylene)(heterocyclyl), —(C₀-C₃ alkylene)(C₆-C₁₀ aryl), or —(C₀-C₃ alkylene)(heteroaryl), which are each substituted by 0, 1, 2, or 3 R^{3a};
R^{3a} is C₁-C₆ alkyl, C₂-C₆ alkenyl, C₂-C₆ alkynyl, C₁-C₆ alkoxy, C₁-C₆ haloalkyl, C₁-C₆ haloalkoxy, C₃-C₆ cycloalkyl, heterocyclyl, C₆-C₁₀ aryl, heteroaryl, halogen, —OR^{3b}, —NR^{3c}R^{3d}, —CN, —C(O)R^{3b}, —C(O)NR^{3c}R^{3d}, —OC(O)NR^{3c}R^{3d}; —N(R^{3b})C(O)NR^{3c}R^{3d}, or —S(O)₂NR^{3c}R^{3d};
R⁴ is C₁-C₆ alkyl, C₂-C₆ alkenyl, C₂-C₆ alkynyl, or C₁-C₆ haloalkyl; or
alternatively, R³ and R⁴ together with the nitrogen to which they are attached form a heterocyclyl, which is substituted by 0, 1, 2, or 3 R^{3a};
each R^{1a} and R^{2a} is independently C₁-C₆ alkyl, C₂-C₆ alkenyl, C₂-C₆ alkynyl, C₁-C₆ alkoxy, C₁-C₆ haloalkyl, C₁-C₆ haloalkoxy, C₃-C₆ cycloalkyl, heterocyclyl, C₆-C₁₀ aryl, heteroaryl, halogen, —OR^{1b}, —NR^{1c}R^{1d}, —CN, —C(O)R^{1b}, —C(O)NR^{1c}R^{1d}, —OC(O)NR^{1c}R^{1d}, —N(R^{1b})C(O)NR^{1c}R^{1d}, or —S(O)₂NR^{1c}R^{1d}; and
each R¹, R^{1c}, R^{1d}, R^{3b}, R^{3c}, and R^{3d} is independently H, C₁-C₆ alkyl, or C₁-C₆ haloalkyl, or
alternatively, R^{3c} and R^{3d} together with the nitrogen to which they are attached form a heterocyclyl.

Also provided herein is a pharmaceutical composition comprising a compound or pharmaceutically acceptable salt thereof of the present disclosure.

Further provided herein is a method of inhibiting binding between SLC15A4 and TASL in an immune cell, comprising administering to the immune cell an effective amount of a compound or pharmaceutically acceptable salt thereof of the present disclosure, or a pharmaceutical composition of the present disclosure.

Further provided herein is a method of treating an autoimmune disorder or an inflammatory condition in a subject in need thereof, comprising administering to the subject a therapeutically effective amount of a compound or pharmaceutically acceptable salt thereof of the present disclosure, or a pharmaceutical composition of the present disclosure.

### DETAILED DESCRIPTION

### I. General

The disclosure relates generally to substituted pyridine and pyrimidine compounds, pharmaceutically acceptable salts thereof, and methods and uses thereof, for interfering with SLC15A4-TASL complex formation. The following description sets forth exemplary methods, parameters and the like. It should be recognized, however, that such description is not intended as a limitation on the scope of the present disclosure but is instead provided as a description of exemplary embodiments.

### II. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CONH₂ is attached through the carbon atom. A dash at the front or end of a chemical group is a matter of convenience; chemical groups can be depicted with or without one or more dashes without losing their ordinary meaning. A wavy line drawn through a line in a structure indicates a point of attachment of a group.

A squiggly line on a chemical group as shown below, for example,

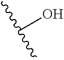 [SMILES: *O]

indicates a point of attachment, i.e., it shows the broken bond by which the group is connected to another described group.

The prefix “Cᵤ-Cᵥ” indicates that the following group has from u to v carbon atoms. For example, “C₁-C₈ alkyl” indicates that the alkyl group has from 1 to 8 carbon atoms.

“Alkyl” refers to a monovalent unbranched or branched saturated hydrocarbon chain. For example, an alkyl group can have 1 to 20 carbon atoms (i.e., C₁-C₂₀ alkyl), 1 to 8 carbon atoms (i.e., C₁-C₅ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃. Other alkyl groups include, but are not limited to, heptyl, octyl, nonyl, decyl, undecyl, dodecyl, pentadcyl, hexadecyl, heptadecyl and octadecyl.

“Alkylene” or “alkylene chain” refers to a straight or branched divalent hydrocarbon (alkyl) chain linking the rest of the molecule to a radical group, consisting solely of carbon and hydrogen, respectively. Alkylenes can have from one to twelve carbon atoms, e.g., methylene, ethylene, propylene, n-butylene, and the like. The alkylene chain is attached to the rest of the molecule through a single or double bond. The points of attachment of the alkylene chain to the rest of the molecule can be through one carbon or any two carbons within the chain. “Optionally substituted alkylene” refers to alkylene or substituted alkylene.

“Alkenyl” refers to an unbranched or branched hydrocarbon chain containing at least two carbon atoms and at least one carbon-carbon double bond. As used herein, alkenyl can have from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Alkenyl can include any number of carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, or any range therein. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to an unbranched or branched hydrocarbon chain containing at least one carbon-carbon triple bond. For example, an alkynyl group can have from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond. Examples of C₂₋₆alkynyl include, but are not limited to, ethynyl, prop-1-ynyl, but-1-ynyl, pent-1-ynyl, pent-4-ynyl and penta-1,4-diynyl.

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), 1 to 8 carbon atoms (i.e., C₁-C₈ alkoxy), 1 to 6 carbon atoms (i.e., C₁-C₆ alkoxy) or 1 to 3 carbon atoms (i.e., C₁-C₃ alkoxy). Examples of suitable alkoxy groups include, but are not limited to, methoxy (—O—CH₃ or —OMe), ethoxy (—OCH₂CH₃ or —OEt), isopropoxy (—O—CH(CH₃)₂), t-butoxy (—O—C(CH₃)₃ or —OtBu) and the like. Other examples of suitable alkoxy groups include, but are not limited to, sec-butoxy, tert-butoxy, pentoxy, hexoxy, and the like.

“Aminoalkyl” is an alkyl group, as defined above, in which one or more hydrogen atoms of the alkyl group is replaced with an amine group (—NR₂, where R═H or alkyl). The alkyl portion of an aminoalkyl group can have 1 to 20 carbon atoms (i.e., C₁-C₂₀ aminoalkyl), 1 to 12 carbon atoms (i.e., C₁-C₁₂ aminoalkyl), 1 to 8 carbon atoms (i.e., C₁-C₅ aminoalkyl), 1 to 6 carbon atoms (i.e., C₁-C₆ aminoalkyl) or 1 to 3 carbon atoms (i.e., C₁-C₃ aminoalkyl). The alkyl groups can be substituted with 1, 2, 3, 4, 5, 6, 7, 8, 9 or more amines. Examples of suitable aminoalkyl groups include, but are not limited to, —CH₂NH₂, —CH₂NHCH₃, —CH₂N(CH₃)₂, —CH₂CH₂NHCH₃, —CH₂CH₂N(CH₃)₂, and —CH₂CH₂CH₂N(CH₃)₂.

“Sulfonylalkyl” is an alkyl group, as defined above, in which one or more hydrogen atoms of the alkyl group is replaced with a sulfonyl group (—SO₂R, where R═H or alkyl). The alkyl portion of an sulfonylalkyl group can have 1 to 20 carbon atoms (i.e., C₁-C₂₀ sulfonylalkyl), 1 to 12 carbon atoms (i.e., C₁-C₁₂ sulfonylalkyl), 1 to 8 carbon atoms (i.e., C₁-C₅ sulfonylalkyl), 1 to 6 carbon atoms (i.e., C₁-C₆ sulfonylalkyl) or 1 to 3 carbon atoms (i.e., C₁-C₃ sulfonylalkyl). The alkyl groups can be substituted with 1, 2, 3, 4, 5, 6, 7, 8, 9 or more sulfonyl groups. Examples of suitable sulfonylalkyl groups include, but are not limited to, —CH₂SO₂CH₃, —CH₂CH₂SO₂CH₃, —CH₂CH₂SO₂CH₂CH₃, and —CH₂CH₂CH₂SO₂CH₃.

“Halo” or “halogen” as used herein refers to fluoro (—F), chloro (—Cl), bromo (—Br) and iodo (—I).

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₅ haloalkyl), 1 to 6 carbon atoms (i.e., C₁-C₆ alkyl) or 1 to 3 carbon atoms (i.e., C₁-C₃ alkyl). The alkyl groups can be substituted with 1, 2, 3, 4, 5, 6, 7, 8, 9 or more halogens. Examples of suitable haloalkyl groups include, but are not limited to, —CF₃, —CHF₂, —CFH₂, —CH₂CF₃, fluorochloromethyl, difluorochloromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, 4, 5, 6, 7, 8, 9 or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a saturated or partially saturated cyclic alkyl group having a single ring or multiple rings, such as 2, 3, 4 or more, wherein the multiple rings can be fused, bridged, spiro, or any combination thereof. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include, but are not limited to, cyclopropyl, cyclobutyl, cyclopentyl, cyclohexyl, cycloheptyl, and cyclooctyl. Cycloalkyl groups also include partially unsaturated ring systems containing one or more double bonds, including fused ring systems with one aromatic ring and one non-aromatic ring, but not fully aromatic ring systems.

“Alkylene-cycloalkyl” refers to a radical having an alkylene component and a cycloalkyl component, where the alkylene component links the cycloalkyl component to the point of attachment. The alkylene component is as defined above to link to the cycloalkyl component and to the point of attachment. In some instances, the alkylene component can be absent. The alkylene component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkylene-cycloalkyl groups include, but are not limited to, —CH₂-cyclopropyl, —CH₂-cyclobutyl, —CH₂-cyclopentyl and —CH₂-cyclohexyl. “Substituted” alkylene-cycloalkyl groups include those having substitutions on the alkylene component and substitutions on the cycloalkyl component.

“Heterocycle” or “heterocyclyl” or “heterocycloalkyl” refer to a saturated or partially unsaturated cyclic alkyl group, with one or more ring heteroatoms independently selected from nitrogen, oxygen, sulfur and silicon. A heterocyclyl can be a single ring or multiple rings, such as 2, 3, 4 or more, wherein the multiple rings can be fused, bridged, spiro, or any combination thereof. As used herein, heterocyclyl has 3 to 20 ring atoms (i.e., 3 to 20 membered heterocyclyl), 3 to 12 ring atoms (i.e., 3 to 12 membered heterocyclyl), 3 to 10 ring atoms (i.e., 3 to 10 membered heterocyclyl), 3 to 8 ring atoms (i.e., 3 to 8 membered heterocyclyl), 4 to 12 ring carbon atoms (i.e., 4 to 12 membered heterocyclyl), 4 to 8 ring atoms (i.e., 4 to 8 membered heterocyclyl), or 4 to 6 ring atoms (i.e., 4 to 6 membered heterocyclyl). Examples of heterocyclyl groups include pyrrolidinyl, piperidinyl, piperazinyl, oxetanyl, dioxolanyl, azetidinyl, and morpholinyl.

“Alkylene-heterocyclyl” refers to a radical having an alkylene component and a heterocyclyl component, where the alkylene component links the heterocyclyl component to the point of attachment. The alkylene component is as defined above to link to the heterocyclyl component and to the point of attachment. The alkylene component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkylene component can be absent. The heterocyclyl component is as defined above. “Substituted” alkylene-heterocyclyl groups include those having substitutions on the alkylene component and substitutions on the heterocyclyl component.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Exemplary aryl groups include, but are not limited to, radicals derived from benzene (e.g., phenyl), naphthalene, anthracene, biphenyl, and the like.

“Alkylene-aryl” refers to a radical having an alkylene component and an aryl component, where the alkylene component links the aryl component to the point of attachment. The alkylene component is as defined above to link to the aryl component and to the point of attachment. The alkylene component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkylene component can be absent. The aryl component is as defined above. Examples of alkylene-aryl groups include, but are not limited to, benzyl and ethylene-benzene. “Substituted” alkylene-aryl groups include those having substitutions on the alkylene component and substitutions on the aryl component.

“Heteroaryl” refers to an aromatic group, including groups having an aromatic tautomer or resonance structure, having a single ring, multiple rings, or multiple fused rings, with at least one heteroatom in the ring, i.e., one or more ring heteroatoms independently selected from nitrogen, oxygen, and sulfur, wherein the nitrogen or sulfur can be oxidized. Thus, the term includes rings having one or more annular O, N, S, S(O), S(O)₂, and N-oxide groups. The term includes rings having one or more annular C(O) groups. As used herein, heteroaryl include 5 to 20 ring atoms (i.e., 5- to 20-membered heteroaryl), 5 to 12 ring atoms (i.e., 5- to 12-membered heteroaryl), or 5 to 10 ring atoms (i.e., 5- to 10-membered heteroaryl), and 1 to 5 heteroatoms independently selected from nitrogen, oxygen, and sulfur, and oxidized forms of the heteroatoms. Examples of heteroaryl groups include, but are not limited to, pyridin-2(1H)-one, pyridazin-3(2H)-one, pyrimidin-4(3H)-one, quinolin-2(1H)-one, pyrimidinyl, purinyl, pyridyl, pyridazinyl, benzothiazolyl, and pyrazolyl. Heteroaryl does not encompass or overlap with aryl as defined above.

“Alkylene-heteroaryl” refers to a radical having an alkylene component and a heteroaryl component, where the alkylene component links the heteroaryl component to the point of attachment. The alkylene component is as defined above to link to the heteroaryl component and to the point of attachment. The alkylene component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkylene component can be absent. The heteroaryl component is as defined within. “Substituted” alkylene-heteroaryl groups include those having substitutions on the alkylene component and substitutions on the heteroaryl component.

“Substituted” as used herein refers to wherein one or more hydrogen atoms of the group are independently replaced by one or more substituents (e.g., 1, 2, 3, or 4 or more) as indicated.

A “compound of the present disclosure” includes compounds disclosed herein, for example a compound of the present disclosure includes compounds of Formula I, including the compounds of the Examples.

“Treatment” or “treating” as used herein is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The term “therapeutically effective amount,” as used herein, is the amount of compound disclosed herein present in a formulation described herein that is needed to provide a desired level of drug in the bloodstream of a subject to be treated to give an anticipated physiological response or desired biological effect when such a formulation is administered by the chosen route of administration. The precise amount will depend upon numerous factors, for example the particular compound disclosed herein, the specific activity of the formulation, the delivery device employed, the physical characteristics of the formulation, its intended use, as well as subject considerations such as severity of the disease state, subject cooperation, etc., and can readily be determined by one skilled in the art based upon the information provided herein.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. The administration can be carried out according to a schedule specifying frequency of administration, dose for administration, and other factors.

A “subject” or “patient” is meant to describe a human or vertebrate animal including a dog, cat, pocket pet, marmoset, horse, cow, pig, sheep, goat, elephant, giraffe, chicken, lion, monkey, owl, rat, squirrel, slender loris, and mouse. A “pocket pet” refers to a group of vertebrate animals capable of fitting into a commodious coat pocket such as, for example, hamsters, chinchillas, ferrets, rats, guinea pigs, gerbils, rabbits and sugar gliders.

### III. Compounds

In one embodiment, the present disclosure provides a compound of Formula I:

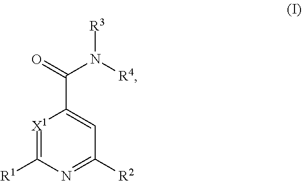

or pharmaceutically acceptable salt thereof,
wherein
R¹ is a C₃-C₈ cycloalkyl, heterocyclyl, C₆-C₁₀ aryl, or heteroaryl, which are each substituted by 0, 1, 2, or 3 R^{1a};
R² is a C₃-C₈ cycloalkyl, heterocyclyl, C₆-C₁₀ aryl, or heteroaryl, which are each substituted by 0, 1, 2, or 3 R^{2a};
X¹ is C—X^{1a} or N;
X^{1a} is H, C₁-C₆ alkyl, C₂-C₆ alkenyl, C₂-C₆ alkynyl, C₁-C₆ alkoxy, C₁-C₆ aminoalkyl, C₁-C₆ sulfonylalkyl, C₁-C₆ haloalkyl, C₁-C₆ haloalkoxy, halogen, or —CN;
R³ is —(C₀-C₃ alkylene)(C₃-C₈ cycloalkyl), —(C₀-C₃ alkylene)(heterocyclyl), —(C₀-C₃ alkylene)(C₆-C₁₀ aryl), or —(C₀-C₃ alkylene)(heteroaryl), which are each substituted by 0, 1, 2, or 3 R^{3a};
R^{3a} is C₁-C₆ alkyl, C₂-C₆ alkenyl, C₂-C₆ alkynyl, C₁-C₆ alkoxy, C₁-C₆ haloalkyl, C₁-C₆ haloalkoxy, C₃-C₆ cycloalkyl, heterocyclyl, C₆-C₁₀ aryl, heteroaryl, halogen, —OR^{3b}, —NR³CR^{3d}—CN, —C(O)R^{3b}, —C(O)NR^{3c}R^{3d}, —OC(O)NR^{3c}R^{3d}, —N(R^{3b})C(O)NR^{3c}R^{3d}, or —S(O)₂NR^{3c}R^{3d};
R⁴ is C₁-C₆ alkyl, C₂-C₆ alkenyl, C₂-C₆ alkynyl, or C₁-C₆ haloalkyl; or
alternatively, R³ and R⁴ together with the nitrogen to which they are attached form a heterocyclyl, which is substituted by 0, 1, 2, or 3 R^{3a};
each R^{1a} and R^{2a} is independently C₁-C₆ alkyl, C₂-C₆ alkenyl, C₂-C₆ alkynyl, C₁-C₆ alkoxy, C₁-C₆ haloalkyl, C₁-C₆ haloalkoxy, C₃-C₆ cycloalkyl, heterocyclyl, C₆-C₁₀ aryl, heteroaryl, halogen, —OR^{1b}, —NR^{c}R^{1d}—CN, —C(O)R^{1b}, —C(O)NR^{1c}R^{1d}, —OC(O)NR^{1c}R^{1d}, —N(R^{1b})C(O)NR^{1c}R^{1d}, or —S(O)₂NR^{1c}R^{1d}; and
each R^{1b}, R^{1c}, R^{1d}, R^{3b}, R^{3c}, and R^{3d} is independently H, C₁-C₆ alkyl, or C₁-C₆ haloalkyl, or
alternatively, R^{3c} and R^{3d} together with the nitrogen to which they are attached form a heterocyclyl.

In some embodiments of a compound of Formula I, or pharmaceutically acceptable salt thereof, X¹ is C—X^{1a}. In some embodiments of the compound of the present disclosure or pharmaceutically acceptable salt thereof, X¹ is C—F. In some embodiments of the compound of the present disclosure or pharmaceutically acceptable salt thereof, X¹ is C—H. In some embodiments of the compound of the present disclosure or pharmaceutically acceptable salt thereof, X¹ is N.

In some embodiments of a compound of Formula I, or pharmaceutically acceptable salt thereof, X^{1a} is H, C₁-C₆ alkyl, C₁-C₆ haloalkyl, halogen, or —CN. In some embodiments of the compound of the present disclosure or pharmaceutically acceptable salt thereof, X^{1a} is H, halogen, or —CN. In some embodiments of the compound of the present disclosure or pharmaceutically acceptable salt thereof, X^{1a} is H, F, or —CN.

In some embodiments of a compound of Formula I, or pharmaceutically acceptable salt thereof, X¹ is C—X^{1a}; X^{1a} is H, halogen, or —CN.

In some embodiments of a compound of Formula I, or pharmaceutically acceptable salt thereof, R¹ is heterocyclyl or heteroaryl, which are each substituted by 0, 1, 2, or 3 R^{1a}. In some embodiments, R¹ is a 4- to 7-membered heterocyclyl or 5- to 6-membered heteroaryl, which are each substituted by 0, 1, 2, or 3 R^{1a}. In some embodiments, R¹ is a 5- to 6-membered heteroaryl, which is substituted by 0, 1, 2, or 3 R^{1a}. In some embodiments, R¹ is a pyrazolyl, pyridyl, pyridazinyl, pyrimidyl, or pyrazinyl, which are each substituted by 0, 1, 2, or 3 R^{1a}. In some embodiments, R¹ is a pyrazolyl, pyridyl, or pyrimidyl, which are each substituted by 0, 1, 2, or 3 R^{1a}.

In some embodiments of a compound of Formula I, or pharmaceutically acceptable salt thereof, R¹ is a 6-membered heteroaryl, which is substituted by 1, 2, or 3 R^{1a}. In some embodiments, R¹ is a pyridyl, pyridazinyl, pyrimidyl, or pyrazinyl, which are each substituted by 1, 2, or 3 R^{1a}. In some embodiments, R¹ is a pyridyl or pyrimidyl, which are each substituted by 1, 2, or 3 R^{1a}

In some embodiments of a compound of Formula I, or pharmaceutically acceptable salt thereof, each R^{1a} is independently C₁-C₆ alkyl, C₁-C₆ alkoxy, C₁-C₆ haloalkyl, C₁-C₆ haloalkoxy, halogen, —OR^{1b}, —NR^{1c}R^{1d} or —CN; and each R^{1b}, R^{1c}, and R^{1d} is independently H and C₁-C₆ alkyl. In some embodiments, each R^{1a} is independently C₁-C₆ alkyl, C₁-C₆ alkoxy, C₁-C₆ haloalkyl, C₁-C₆ haloalkoxy, halogen, —OR^{1b}, —NR^{1c}R^{1d} or —CN; and each R^{1b}, R^{1c}, and R^{1d} is independently H and C₁-C₆ alkyl.

In some embodiments of a compound of Formula I, or pharmaceutically acceptable salt thereof, R¹ is

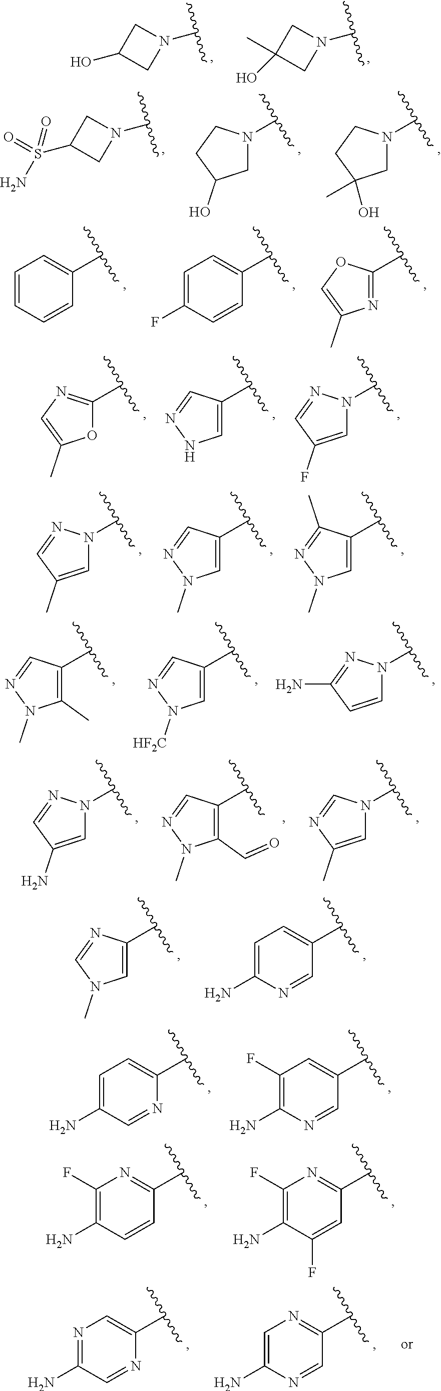

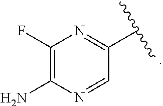 [SMILES: *c1cnc(N)c(F)n1]

In some embodiments of a compound of Formula I, or pharmaceutically acceptable salt thereof, R¹ is

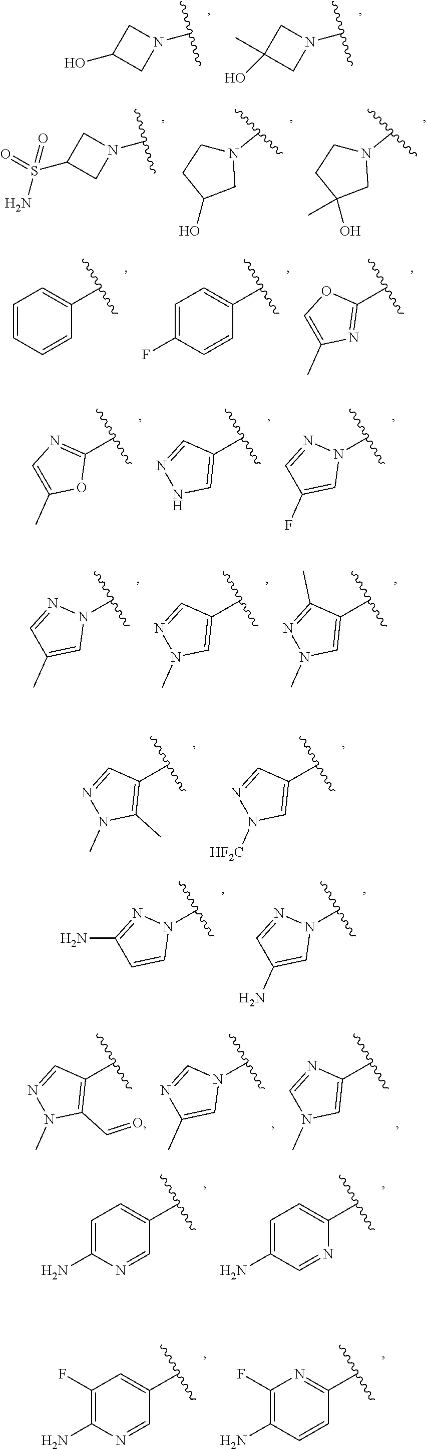

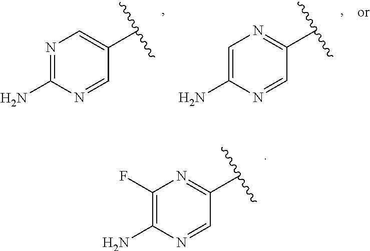 [SMILES: *c1cnc(N)nc1 ; *c1cnc(N)cn1 ; *c1cnc(N)c(F)n1]

In some embodiments of a compound of Formula I, or pharmaceutically acceptable salt thereof, R is

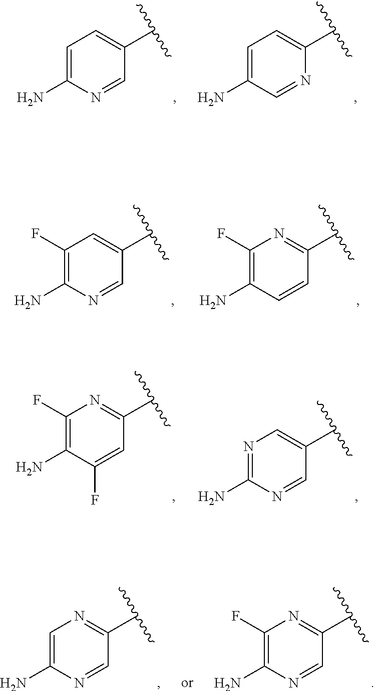

In some embodiments of a compound of Formula I, or pharmaceutically acceptable salt thereof, R is

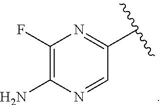 [SMILES: *c1cnc(N)c(F)n1]

In some embodiments of a compound of Formula I, or pharmaceutically acceptable salt thereof, R² is a C₆-C₁₀ aryl or heteroaryl, which are each substituted by 0, 1, 2, or 3 R^{2a}. In some embodiments, R² is a C₆-C₁₀ aryl, which is substituted by 0, 1, 2, or 3 R^{2a}. In some embodiments, R² is a heteroaryl, which is substituted by 0, 1, 2, or 3 R^{2a}. In some embodiments, R² is a 5- to 6-membered heteroaryl, which is substituted by 0, 1, 2, or 3 R^{2a}. In some embodiments, R² is phenyl or pyridyl, which are each substituted by 0, 1, 2, or 3 R^{2a}.

In some embodiments of a compound of Formula I, or pharmaceutically acceptable salt thereof, each R^{2a} is independently C₁-C₆ alkyl, C₁-C₆ alkoxy, C₁-C₆ haloalkyl, C₁-C₆ haloalkoxy, C₃-C₆ cycloalkyl, halogen, —OR^{1b}, —NR^{c}R^{1d} or —CN. In some embodiments, R^{2a} is C₁-C₆ haloalkyl. In some embodiments, R^{2a} is —OCH₂CF₃.

In some embodiments of a compound of Formula I, or pharmaceutically acceptable salt thereof, R² is:

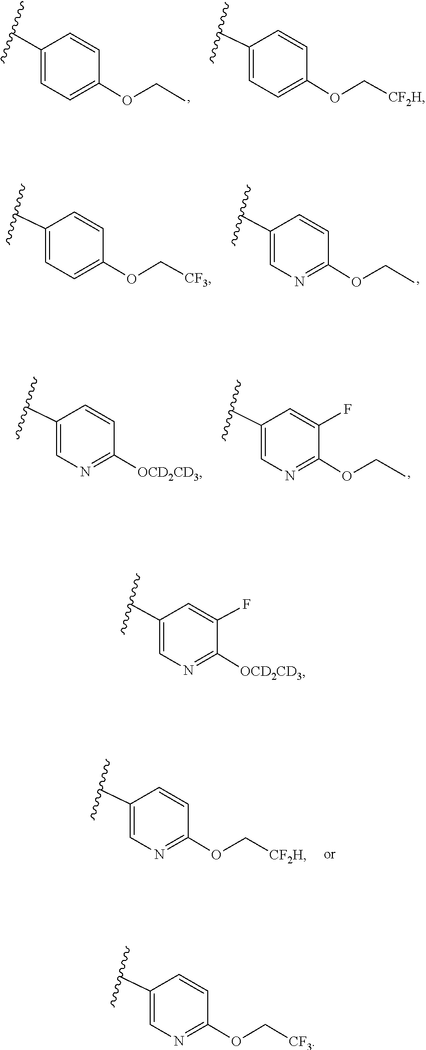

In some embodiments of a compound of Formula I, or pharmaceutically acceptable salt thereof, R² is:

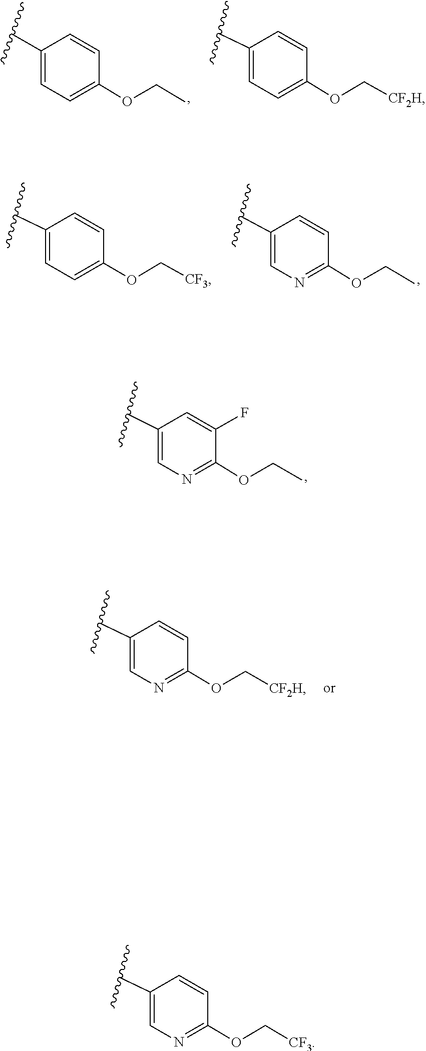

In some embodiments of a compound of Formula I, or pharmaceutically acceptable salt thereof, R² is:

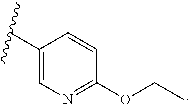 [SMILES: *c1ccc(OCC)nc1]

In some embodiments of a compound of Formula I, or pharmaceutically acceptable salt thereof, R³ is C₃-C₈ cycloalkyl, heterocyclyl, C₆-C₁₀ aryl, or heteroaryl, which are each substituted by 0, 1, 2, or 3 R^{3a}. In some embodiments, R³ is heterocyclyl substituted by 0, 1, 2, or 3 R^{3a}. In some embodiments, R³ is piperazinyl, diazepanyl, diazabicyclo[2.2.1]heptyl, diazabicyclo[2.2.2]octyl, diazabicyclo[3.2.1]octyl, or diazaspiro[3.5]nonyl, which is substituted by 0, 1, 2, or 3 R^{3a}

In some embodiments of a compound of Formula I, or pharmaceutically acceptable salt thereof, R^{3a} is C₁-C₆ alkyl, C₂-C₆ alkenyl, C₂-C₆ alkynyl, C₁-C₆ alkoxy, C₁-C₆ haloalkyl, C₁-C₆ haloalkoxy, C₃-C₆ cycloalkyl, heterocyclyl, C₆-C₁₀ aryl, heteroaryl, or halogen. In some embodiments, R^{3a} is C₁-C₆ alkyl, C₁-C₆ alkoxy, C₁-C₆ haloalkyl, C₁-C₆ haloalkoxy, or halogen. In some embodiments, R^{3a} is C₁-C₆ alkyl, C₁-C₆ haloalkyl, or halogen. In some embodiments, R^{3a} is C₁-C₆ alkyl. In some embodiments, R^{3a} is methyl.

In some embodiments of a compound of Formula I, or pharmaceutically acceptable salt thereof, R^{3a} is C₁-C₆ alkyl, C₁-C₆ haloalkyl, halogen, or —NR^{3c}R^{3d}. In some embodiments, R^{3a} is methyl, CD₃, ethyl, CD₂CD₃, or —NH₂.

In some embodiments of a compound of Formula I, or pharmaceutically acceptable salt thereof, R⁴ is C₁-C₆ alkyl or C₁-C₆ haloalkyl. In some embodiments, R⁴ is C₁-C₆ alkyl. In some embodiments, R⁴ is methyl.

In some embodiments of a compound of Formula I, or pharmaceutically acceptable salt thereof, R³ and R⁴ together with the nitrogen to which they are attached form a heterocyclyl, which is substituted by 0, 1, 2, or 3 R^{3a}. In some embodiments, R³ and R⁴ together with the nitrogen to which they are attached form a 6- to 9-membered heterocyclyl, which is substituted by 0, 1, 2, or 3 R^{3a}. In some embodiments, R³ and R⁴ together with the nitrogen to which they are attached form a piperazinyl, diazepanyl, diazabicyclo[2.2.1]heptyl, diazabicyclo[2.2.2]octyl, diazabicyclo[3.2.1]octyl, or diazaspiro[3.5]nonyl, which is substituted by 0, 1, 2, or 3 R^{3a}

In some embodiments of a compound of Formula I, or pharmaceutically acceptable salt thereof, unless otherwise indicated, a heteroaryl in each instance is 5- to 10-membered ring having 1, 2, 3, or 4 heteroatoms selected from N, O, and S; and a heterocyclyl in each instance is 3- to 12-membered ring having 1, 2, 3, or 4 heteroatoms selected from N, O, and S.

In some embodiments of a compound of Formula I, or pharmaceutically acceptable salt thereof, R³ and R⁴ together with the nitrogen to which they are attached form

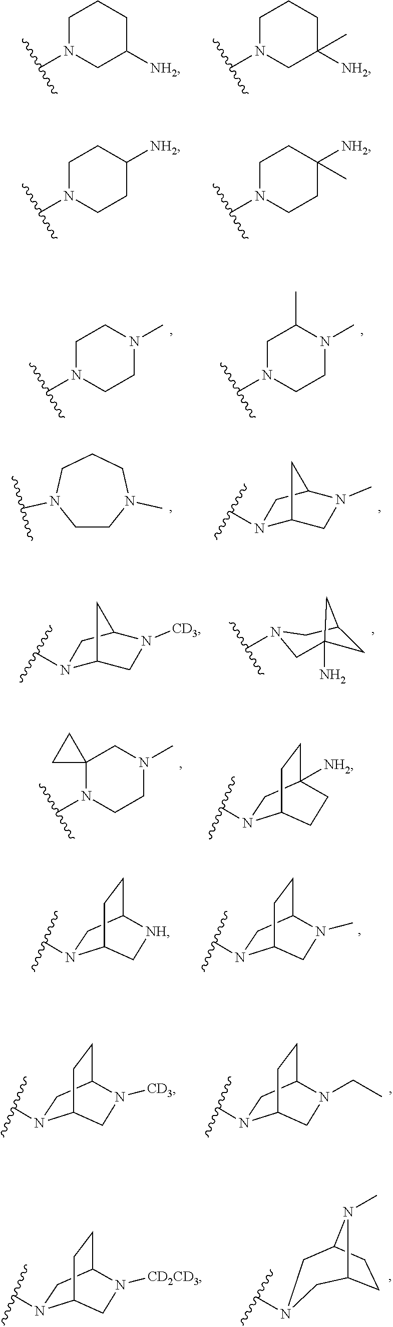

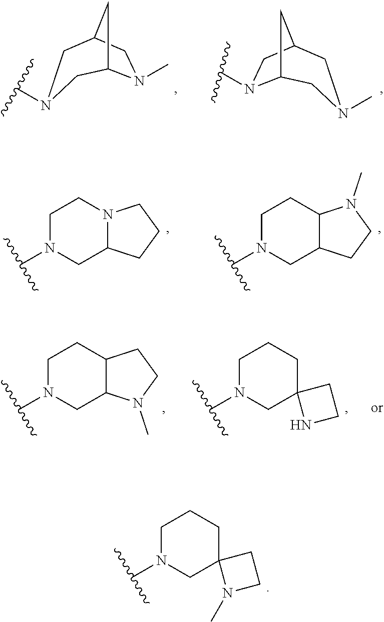

In some embodiments of a compound of Formula I, or pharmaceutically acceptable salt thereof, R³ and R⁴ together with the nitrogen to which they are attached form

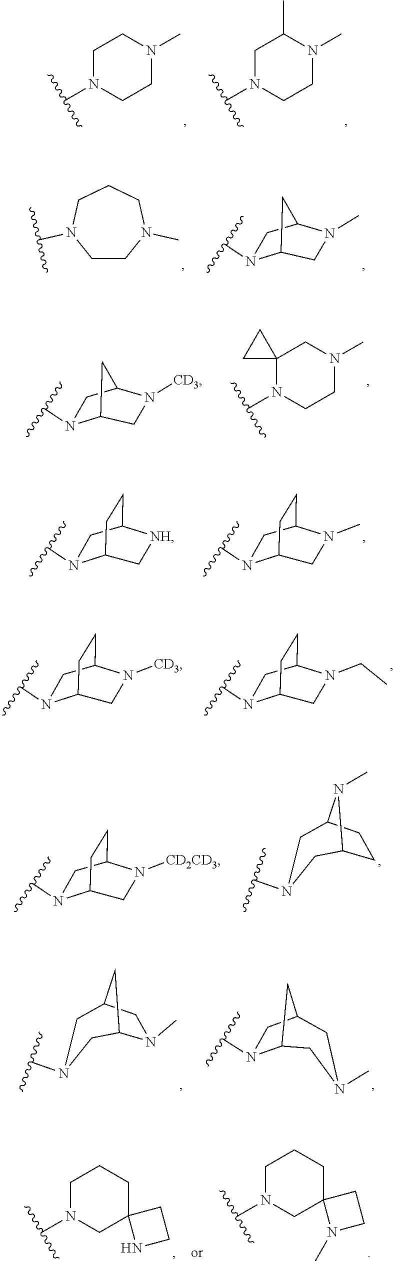

In some embodiments of a compound of Formula I, or pharmaceutically acceptable salt thereof, R³ and R⁴ together with the nitrogen to which they are attached form

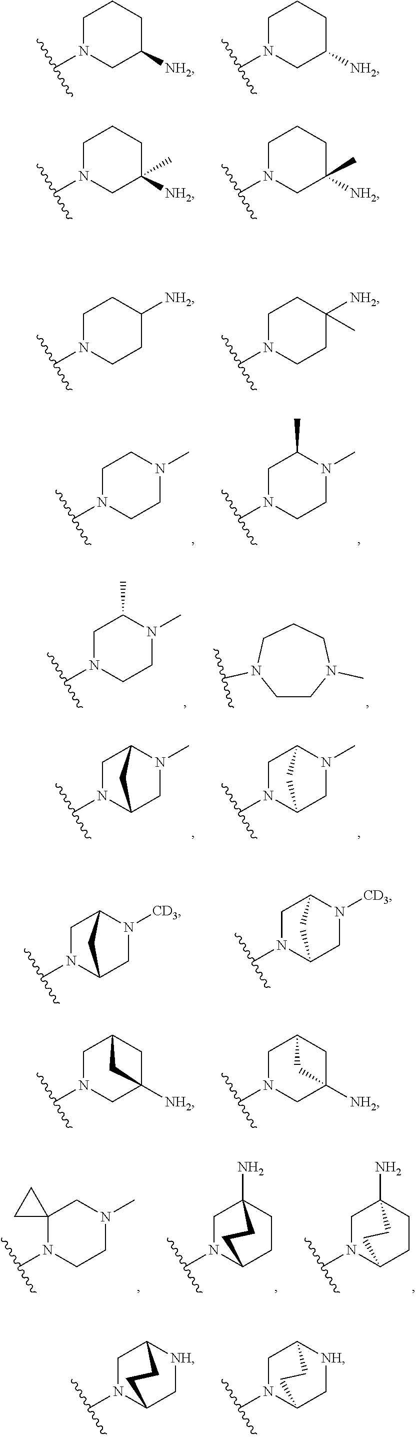

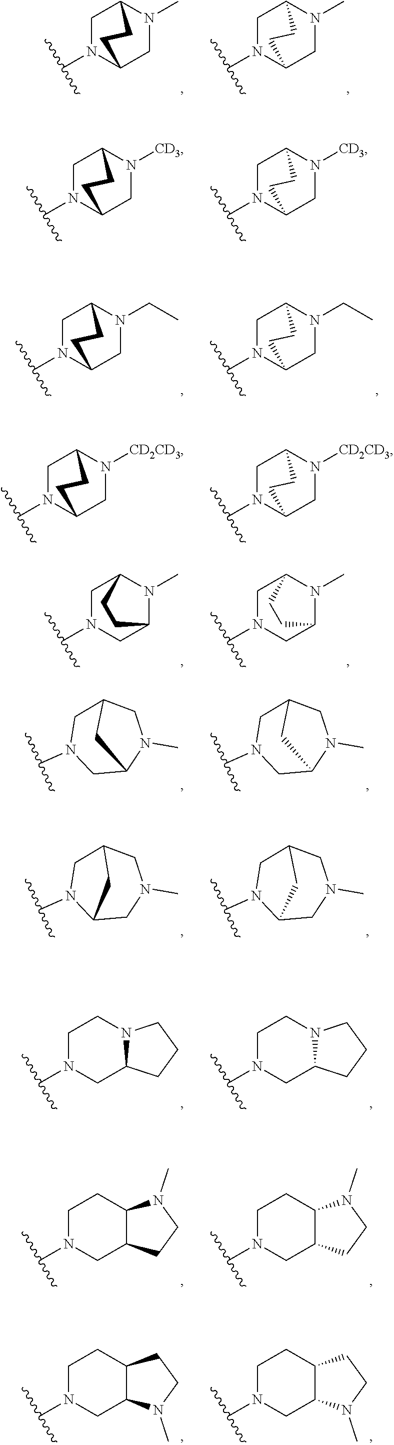

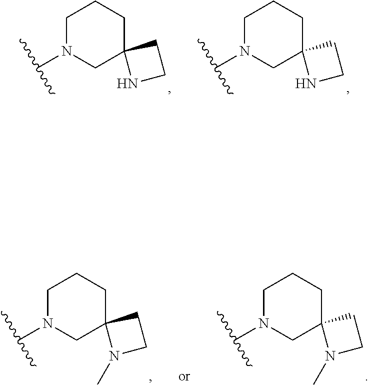 [SMILES: *N1CCC[C@@]2(CCN2)C1 ; *N1CCC[C@]2(CCN2)C1 ; *N1CCC[C@@]2(CCN2C)C1 ; *N1CCC[C@]2(CCN2C)C1]

In some embodiments of a compound of Formula I, or pharmaceutically acceptable salt thereof, R³ and R⁴ together with the nitrogen to which they are attached form

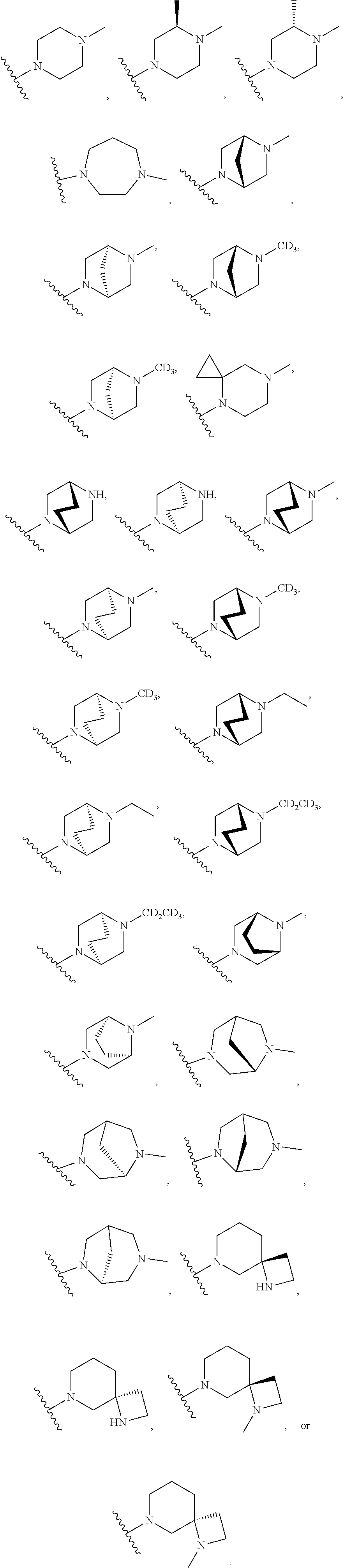

In some embodiments of a compound of Formula I, or pharmaceutically acceptable salt thereof, R³ and R⁴ together with the nitrogen to which they are attached form

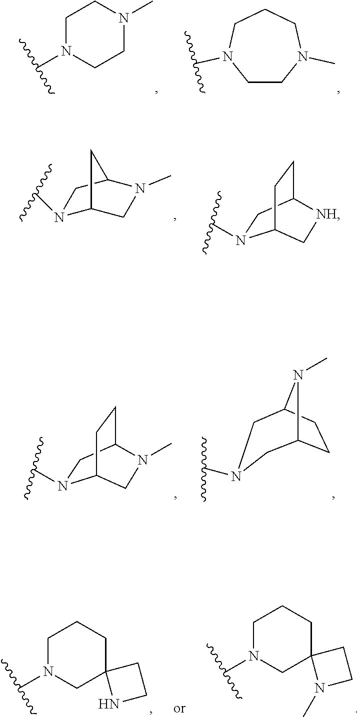

In some embodiments of a compound of Formula I, or pharmaceutically acceptable salt thereof, the compound is any one of the following:

TABLE 1
Compounds

| Example | Name |
|---|---|
| 1 | (6-(5-amino-6-fluoropyrazin-2-yl)-6′-ethoxy-5-fluoro-[2,3′-bipyridin]-4- |
| | yl)((1R,4R)-5-methyl-2,5-diazabicyclo[2.2.2]octan-2-yl)methanone |
| 2 | (6-(5-amino-6-fluoropyrazin-2-yl)-6′-ethoxy-5-fluoro-[2,3′-bipyridin]-4-yl)(8- |
| | methyl-3,8-diazabicyclo[3.2.1]octan-3-yl)methanone |
| 3 | (2-(5-amino-6-fluoropyrazin-2-yl)-6-(4-(2,2-difluoroethoxy)phenyl)-3- |
| | fluoropyridin-4-yl)(5-methyl-2,5-diazabicyclo[2.2.2]octan-2-yl)methanone |
| 4 | [2-(5-amino-6-fluoro-pyrazin-2-yl)-6-[4-(2,2-difluoroethoxy)phenyl]-3- |
| | fluoro-4-pyridyl]-(1,8-diazaspiro[3.5]nonan-8-yl)methanone |
| 5 | (2-(5-amino-6-fluoropyrazin-2-yl)-6-(4-(2,2-difluoroethoxy)phenyl)-3- |
| | fluoropyridin-4-yl)(8-methyl-3,8-diazabicyclo[3.2.1]octan-3-yl)methanone |
| 6 | [2-(5-amino-6-fluoro-pyrazin-2-yl)-6-[4-(2,2-difluoroethoxy)phenyl]-3- |
| | fluoro-4-pyridyl]-(4-methyl-1,4-diazepan-1-yl)methanone |
| 7 | (2-(5-amino-6-fluoropyrazin-2-yl)-6-(4-(2,2-difluoroethoxy)phenyl)-3- |
| | fluoropyridin-4-yl)(2,5-diazabicyclo[2.2.2]octan-2-yl)methanone |
| 8 | (2-(5-amino-6-fluoropyrazin-2-yl)-6-(4-(2,2-difluoroethoxy)phenyl)-3- |
| | fluoropyridin-4-yl)((1S,4S)-5-methyl-2,5-diazabicyclo[2.2.1]heptan-2-yl) |
| | methanone |
| 9 | (2-(5-amino-6-fluoropyrazin-2-yl)-6-(4-(2,2-difluoroethoxy)phenyl)-3- |
| | fluoropyridin-4-yl)((1R,4R)-5-methyl-2,5-diazabicyclo[2.2.1]heptan-2- |
| | yl)methanone |
| 10 | (2-(5-amino-6-fluoropyrazin-2-yl)-6-(4-(2,2-difluoroethoxy)phenyl)-3- |
| | fluoropyridin-4-yl)(1,7-diazaspiro[3.5]nonan-7-yl)methanone |
| 11 | [2-(5-amino-6-fluoro-pyrazin-2-yl)-6-[4-(2,2-difluoroethoxy)phenyl]-3- |
| | fluoro-4-pyridyl]-(4-methylpiperazin-1-yl)methanone |
| 12 | (6-(5-amino-6-fluoropyrazin-2-yl)-6′-ethoxy-5-fluoro-[2,3′-bipyridin]-4- |
| | yl)((1R,4R)-5-methyl-2,5-diazabicyclo[2.2.1]heptan-2-yl)methanone |
| 13 | (6-(5-amino-6-fluoropyrazin-2-yl)-6′-ethoxy-5-fluoro-[2,3′-bipyridin]-4-yl) |
| | (4-methylpiperazin-1-yl) methanone |
| 14 | [2-(5-amino-6-fluoro-pyrazin-2-yl)-6-(6-ethoxy-3-pyridyl)-3-fluoro-4- |
| | pyridyl]-[(1S,4S)-5-methyl-2,5-diazabicyclo[2.2.1]heptan-2-yl]methanone |
| 15 | (2-(5-amino-6-fluoropyrazin-2-yl)-6-(4-(2,2-difluoroethoxy)phenyl)-3- |
| | fluoropyridin-4-yl)((1R,4R)-5-methyl-2,5-diazabicyclo[2.2.2]octan-2- |
| | yl)methanone |
| 16 | (2-(5-amino-6-fluoropyrazin-2-yl)-6-(4-(2,2-difluoroethoxy)phenyl)-3- |
| | fluoropyridin-4-yl)((1S,4S)-5-methyl-2,5-diazabicyclo[2.2.2]octan-2- |
| | yl)methanone |
| 17 | (6-(5-amino-6-fluoropyrazin-2-yl)-6′-ethoxy-5-fluoro-[2,3′-bipyridin]-4-yl)(1- |
| | methyl-1,6-diazaspiro[3.5]nonan-6-yl)methanone |
| 18 | (2-(5-amino-6-fluoropyrazin-2-yl)-6-(4-(2,2-difluoroethoxy)phenyl)-3- |
| | fluoropyridin-4-yl)(1-methyl-1,6-diazaspiro[3.5]nonan-6-yl)methanone |
| 19 | (6-(5-amino-6-fluoropyrazin-2-yl)-6′-ethoxy-5-fluoro-[2,3′-bipyridin]-4- |
| | yl)((1S,4S)-5-methyl-2,5-diazabicyclo[2.2.2]octan-2-yl)methanone |
| 20 | 6-(5-amino-6-fluoropyrazin-2-yl)-6′-(cyclopropylmethoxy)-5-fluoro-N-(2- |
| | methyl-2-azabicyclo[2.2.2]octan-4-yl)-[2,3′-bipyridine]-4-carboxamide |
| 21 | (5-amino-6″-ethoxy-3′,5″,6-trifluoro-[2,2′:6′,3″-terpyridin]-4′-yl)((1S,4S)-5- |
| | (methyl-d3)-2,5-diazabicyclo[2.2.2]octan-2-yl)methanone; formic acid |
| 22 | (5-amino-6″-(ethoxy-d5)-3′,6-difluoro-[2,2′:6′,3″-terpyridin]-4′-yl)((1S,4S)-5- |
| | (methyl-d3)-2,5-diazabicyclo[2.2.2]octan-2-yl)methanone |
| 23 | (5-amino-6″-ethoxy-3′,6-difluoro-[2,2′:6′,3″-terpyridin]-4′-yl)((1S,4S)-5- |
| | (methyl-d3)-2,5-diazabicyclo[2.2.2]octan-2-yl)methanone |
| 24 | (6-(5-amino-6-fluoropyrazin-2-yl)-6′-(ethoxy-d5)-5-fluoro-[2,3′-bipyridin]-4- |
| | yl)((1S,4S)-5-(methyl-d3)-2,5-diazabicyclo[2.2.2]octan-2-yl)methanone |
| 25 | (5-amino-6″-ethoxy-3′,5″,6-trifluoro-[2,2′:6′,3″-terpyridin]-4′-yl)((1S,4S)-5- |
| | methyl-2,5-diazabicyclo[2.2.2]octan-2-yl)methanone |
| 26 | (5-amino-6″-ethoxy-3′,6-difluoro-[2,2′:6′,3″-terpyridin]-4′-yl)((1S,4S)-5- |
| | methyl-2,5-diazabicyclo[2.2.2]octan-2-yl)methanone |
| 27 | (6-(5-amino-6-fluoropyrazin-2-yl)-6′-ethoxy-5-fluoro-[2,3′-bipyridin]-4-yl)(1- |
| | methyl-1,6-diazaspiro[3.5]nonan-6-yl)methanone |
| 28 | (R)-(6-(5-amino-6-fluoropyrazin-2-yl)-6′-ethoxy-5-fluoro-[2,3′-bipyridin]-4- |
| | yl)(1-methyl-1,6-diazaspiro[3.5]nonan-6-yl)methanone |
| 29 | (S)-(6-(5-amino-6-fluoropyrazin-2-yl)-6′-ethoxy-5-fluoro-[2,3′-bipyridin]-4- |
| | yl)(1-methyl-1,6-diazaspiro[3.5]nonan-6-yl)methanone |
| 30 | (2-(5-amino-6-fluoropyrazin-2-yl)-6-(4-(2,2-difluoroethoxy)phenyl)-3- |
| | fluoropyridin-4-yl)(1-methyl-1,6-diazaspiro[3.5]nonan-6-yl)methanone |
| 31 | (6-(5-amino-6-fluoropyrazin-2-yl)-6′-ethoxy-5-fluoro-[2,3′-bipyridin]-4- |
| | yl)((1R,4R)-5-(methyl-d3)-2,5-diazabicyclo[2.2.2]octan-2-yl)methanone |
| 32 | (6-(5-amino-6-fluoropyrazin-2-yl)-6′-ethoxy-5-fluoro-[2,3′-bipyridin]-4- |
| | yl)((1R,4R)-5-ethyl-2,5-diazabicyclo[2.2.2]octan-2-yl)methanone |
| 33 | (S)-(6-(5-amino-6-fluoropyrazin-2-yl)-6′-ethoxy-5-fluoro-[2,3′-bipyridin]-4- |
| | yl)(3,4-dimethylpiperazin-1-yl)methanone |
| 34 | (6-(5-amino-6-fluoropyrazin-2-yl)-6′-ethoxy-5-fluoro-[2,3′-bipyridin]-4-yl)(7- |
| | methyl-4,7-diazaspiro[2.5]octan-4-yl)methanone |
| 35 | (R)-(6-(5-amino-6-fluoropyrazin-2-yl)-6′-ethoxy-5-fluoro-[2,3′-bipyridin]-4- |
| | yl)(3,4-dimethylpiperazin-1-yl)methanone |
| 36 | (6-(5-amino-6-fluoropyrazin-2-yl)-6′-ethoxy-5-fluoro-[2,3′-bipyridin]-4-yl)(3- |
| | methyl-3,6-diazabicyclo[3.2.1]octan-6-yl)methanone |
| 37 | (6-(5-amino-6-fluoropyrazin-2-yl)-6′-ethoxy-5-fluoro-[2,3′-bipyridin]-4- |
| | yl)((1S,4S)-5-(methyl-d3)-2,5-diazabicyclo[2.2.2]octan-2-yl)methanone |
| 38 | (6-(5-amino-6-fluoropyrazin-2-yl)-6′-ethoxy-5-fluoro-[2,3′-bipyridin]-4- |
| | yl)((5S)-6-methyl-3,6-diazabicyclo[3.2.1]octan-3-yl)methanone |
| 39 | (6-(5-amino-6-fluoropyrazin-2-yl)-6′-ethoxy-5-fluoro-[2,3′-bipyridin]-4- |
| | yl)((5R)-6-methyl-3,6-diazabicyclo[3.2.1]octan-3-yl)methanone |
| 40 | (6-(5-amino-6-fluoropyrazin-2-yl)-6′-ethoxy-5,5′-difluoro-[2,3′-bipyridin]-4- |
| | yl)((1S,4S)-5-methyl-2,5-diazabicyclo[2.2.2]octan-2-yl)methanone |
| 41 | [2-(5-amino-6-fluoro-pyrazin-2-yl)-6-(6-ethoxy-3-pyridyl)-3-fluoro-4- |
| | pyridyl]-[(1S,4S)-5-ethyl-2,5-diazabicyclo[2.2.2]octan-2-yl]methanone |
| 42 | (6-(5-amino-6-fluoropyrazin-2-yl)-6′-ethoxy-5,5′-difluoro-[2,3′-bipyridin]-4- |
| | yl)((1S,4S)-5-ethyl-2,5-diazabicyclo[2.2.2]octan-2-yl)methanone |
| 43 | (6-(5-amino-6-fluoropyrazin-2-yl)-6′-ethoxy-5-fluoro-[2,3′-bipyridin]-4- |
| | yl)((1R,4R)-5-(ethyl-d5)-2,5-diazabicyclo[2.2.2]octan-2-yl)methanone |
| 44 | (6-(5-amino-6-fluoropyrazin-2-yl)-6′-ethoxy-5-fluoro-[2,3′-bipyridin]-4- |
| | yl)((1S,4S)-2,5-diazabicyclo[2.2.2]octan-2-yl)methanone |
| 45 | (6-(5-amino-6-fluoropyrazin-2-yl)-6′-ethoxy-5,5′-difluoro-[2,3′-bipyridin]-4- |
| | yl)((1S,4S)-2,5-diazabicyclo[2.2.2]octan-2-yl)methanone |
| 46 | (6-(5-amino-6-fluoropyrazin-2-yl)-6′-ethoxy-5,5′-difluoro-[2,3′-bipyridin]-4- |
| | yl)((1S,4S)-5-(methyl-d3)-2,5-diazabicyclo[2.2.2]octan-2-yl)methanone |
| 47 | (6-(5-amino-6-fluoropyrazin-2-yl)-6′-ethoxy-5-fluoro-[2,3′-bipyridin]-4- |
| | yl)((1R,4R)-2,5-diazabicyclo[2.2.2]octan-2-yl)methanone |
| 48 | (6-(5-amino-6-fluoropyrazin-2-yl)-6′-ethoxy-5,5′-difluoro-[2,3′-bipyridin]-4- |
| | yl)((1R,4R)-5-(methyl-d3)-2,5-diazabicyclo[2.2.2]octan-2-yl)methanone |
| 49 | (6-(5-amino-6-fluoropyrazin-2-yl)-6′-ethoxy-5,5′-difluoro-[2,3′-bipyridin]-4- |
| | yl)((1R,4R)-5-methyl-2,5-diazabicyclo[2.2.2]octan-2-yl)methanone |
| 50 | (6-(5-amino-6-fluoropyrazin-2-yl)-6′-ethoxy-5,5′-difluoro-[2,3′-bipyridin]-4- |
| | yl)((1R,4R)-5-(ethyl-d5)-2,5-diazabicyclo[2.2.2]octan-2-yl)methanone |
| 51 | (5-amino-6″-(ethoxy-d5)-3′,5″,6-trifluoro-[2,2′:6′,3″-terpyridin]-4′- |
| | yl)((1S,4S)-5-(methyl-d3)-2,5-diazabicyclo[2.2.2]octan-2-yl)methanone |
| 52 | (S)-(5-amino-6″-ethoxy-3′,6-difluoro-[2,2′:6′,3″-terpyridin]-4′- |
| | yl)(hexahydropyrrolo[1,2-a]pyrazin-2(1H)-yl)methanone |
| 53 | and (R)-(5-amino-6″-ethoxy-3′,6-difluoro-[2,2′:6′,3″-terpyridin]-4′- |
| | yl)(hexahydropyrrolo[1,2-a]pyrazin-2(1H)-yl)methanone |
| 54 | (S)-(5-amino-6″-ethoxy-3′,5″,6-trifluoro-[2,2′:6′,3″-terpyridin]-4′- |
| | yl)(hexahydropyrrolo[1,2-a]pyrazin-2(1H)-yl)methanone |
| 55 | (R)-(5-amino-6″-ethoxy-3′,5″,6-trifluoro-[2,2′:6′,3″-terpyridin]-4′- |
| | yl)(hexahydropyrrolo[1,2-a]pyrazin-2(1H)-yl)methanone |
| 56 | (3-amino-3-methylpiperidin-1-yl)(5-amino-6″-ethoxy-3′,5″,6-trifluoro- |
| | [2,2′:6′,3″-terpyridin]-4′-yl)methanone |
| 57 | (3-amino-3-methylpiperidin-1-yl)(5-amino-6″-ethoxy-3′,6-difluoro-[2,2′:6′,3″- |
| | terpyridin]-4′-yl)methanone |
| 58 | (5-amino-6″-ethoxy-3′,5″,6-trifluoro-[2,2′:6′,3″-terpyridin]-4′-yl)(3- |
| | aminopiperidin-1-yl)methanone |
| 59 | (R)-(5-amino-6″-ethoxy-3′,5″,6-trifluoro-[2,2′:6′,3″-terpyridin]-4′-yl)(3- |
| | aminopiperidin-1-yl)methanone |
| 60 | (R)-(5-amino-6″-ethoxy-3′,6-difluoro-[2,2′:6′,3″-terpyridin]-4′-yl)(3- |
| | aminopiperidin-1-yl)methanone |
| 61 | (R)-(6-(5-amino-6-fluoropyrazin-2-yl)-6′-ethoxy-5-fluoro-[2,3′-bipyridin]-4- |
| | yl)(3-aminopiperidin-1-yl)methanone |
| 62 | (S)-(5-amino-6″-ethoxy-3′,6-difluoro-[2,2′:6′,3″-terpyridin]-4′-yl)(3- |
| | aminopiperidin-1-yl)methanone |
| 63 | (R)-(6-(5-amino-6-fluoropyrazin-2-yl)-6′-ethoxy-5,5′-difluoro-[2,3′- |
| | bipyridin]-4-yl)(3-aminopiperidin-1-yl)methanone |
| 64 | (R)-(3-amino-3-methylpiperidin-1-yl)(5-amino-6″-ethoxy-3′,5″,6-trifluoro- |
| | [2,2′:6′,3″-terpyridin]-4′-yl)methanone |
| 65 | (S)-(3-amino-3-methylpiperidin-1-yl)(5-amino-6″-ethoxy-3′,5″,6-trifluoro- |
| | [2,2′:6′,3″-terpyridin]-4′-yl)methanone |
| 66 | (5-amino-6″-ethoxy-3′,6-difluoro-[2,2′:6′,3″-terpyridin]-4′-yl)(4- |
| | aminopiperidin-1-yl)methanone |
| 67 | (5-amino-6″-ethoxy-3′,5″,6-trifluoro-[2,2′:6′,3″-terpyridin]-4′-yl)(4- |
| | aminopiperidin-1-yl)methanone |
| 68 | (4-amino-4-methylpiperidin-1-yl)(5-amino-6″-ethoxy-3′,6-difluoro-[2,2′:6′,3″- |
| | terpyridin]-4′-yl)methanone |
| 69 | (4-amino-4-methylpiperidin-1-yl)(5-amino-6″-ethoxy-3′,5″,6-trifluoro- |
| | [2,2′:6′,3″-terpyridin]-4′-yl)methanone |
| 70 | (R)-(5-amino-6″-ethoxy-3′,4,6-trifluoro-[2,2′:6′,3″-terpyridin]-4′-yl)(3- |
| | aminopiperidin-1-yl)methanone |
| 71 | (4-amino-4-methylpiperidin-1-yl)(5-amino-6″-ethoxy-3′,4,6-trifluoro- |
| | [2,2′:6′,3″-terpyridin]-4′-yl)methanone |
| 72 | (S)-(5-amino-6″-ethoxy-3′,4,6-trifluoro-[2,2′:6′,3″-terpyridin]-4′-yl)(3- |
| | aminopiperidin-1-yl)methanone |
| 73 | (5-amino-6″-ethoxy-3′,5″,6-trifluoro-[2,2′:6′,3″-terpyridin]-4′-yl)(1-amino-3- |
| | azabicyclo[3.1.1]heptan-3-yl)methanone |
| 74 | (5-amino-6″-ethoxy-3′,5″,6-trifluoro-[2,2′:6′,3″-terpyridin]-4′-yl)(4-amino-2- |
| | azabicyclo[2.2.2]octan-2-yl)methanone |
| 75 | (5-amino-6″-ethoxy-3′,5″,6-trifluoro-[2,2′:6′,3″-terpyridin]-4′-yl)((3aS,7aR)-1- |
| | methyloctahydro-5H-pyrrolo[3,2-c]pyridin-5-yl)methanone |
| 76 | (5-amino-6″-ethoxy-3′,5″,6-trifluoro-[2,2′:6′,3″-terpyridin]-4′-yl)((3aR,7aS)-1- |
| | methyloctahydro-5H-pyrrolo[3,2-c]pyridin-5-yl)methanone |
| 77 | (5-amino-6″-ethoxy-3′,5″,6-trifluoro-[2,2′:6′,3″-terpyridin]-4′-yl)((3aR,7aR)-1- |
| | methyloctahydro-6H-pyrrolo[2,3-c]pyridin-6-yl)methanone |
| 78 | (5-amino-6″-ethoxy-3′,5″,6-trifluoro-[2,2′:6′,3″-terpyridin]-4′-yl)((3aS,7aS)-1- |
| | methyloctahydro-6H-pyrrolo[2,3-c]pyridin-6-yl)methanone |
| 79 | (5-amino-6″-ethoxy-3′,6-difluoro-[2,2′:6′,3″-terpyridin]-4′-yl)(4-amino-2- |
| | azabicyclo[2.2.2]octan-2-yl)methanone |
| 80 | (6-(5-amino-6-fluoropyrazin-2-yl)-6′-ethoxy-5-fluoro-[2,3′-bipyridin]-4-yl)(4- |
| | amino-2-azabicyclo[2.2.2]octan-2-yl)methanone |
| 81 | (5-amino-6″-ethoxy-3′,6-difluoro-[2,2′:6′,3″-terpyridin]-4′-yl)((3aS,7aR)-1- |
| | methyloctahydro-5H-pyrrolo[3,2-c]pyridin-5-yl)methanone |
| 82 | (5-amino-6″-ethoxy-3′,6-difluoro-[2,2′:6′,3″-terpyridin]-4′-yl)((3aR,7aS)-1- |
| | methyloctahydro-5H-pyrrolo[3,2-c]pyridin-5-yl)methanone |
| 83 | (6-(5-amino-6-fluoropyrazin-2-yl)-6′-ethoxy-5,5′-difluoro-[2,3′-bipyridin]-4- |
| | yl)(4-amino-2-azabicyclo[2.2.2]octan-2-yl)methanone |

Provided are also compounds described herein or pharmaceutically acceptable salts, isomers, or a mixture thereof, in which from 1 to n hydrogen atoms attached to a carbon atom can be replaced by a deuterium atom or D, in which n is the number of hydrogen atoms in the molecule. As known in the art, the deuterium atom is a non-radioactive isotope of the hydrogen atom. Such compounds can increase resistance to metabolism, and thus can be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” TRENDS PHARMACOL. SCI., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I can generally be prepared by conventional techniques known to those skilled in the art or by processes analogous to those described in the Examples as set out below using an appropriate isotopically-labeled reagent in place of the non-labeled reagent previously employed.

Furthermore, the compounds of Formula I or pharmaceutically acceptable salt thereof may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of Formula I are contemplated as being part of the present disclosure, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved (i.e., separated) by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present disclosure further encompasses any tautomers of the compounds of Formula I. It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The compounds of the embodiments disclosed herein, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)- isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds can exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of ADVANCED ORGANIC CHEMISTRY, 4th ed., J. March, John Wiley & Sons, New York, 1992).

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, formulations, dosage forms and other materials which are useful in preparing a pharmaceutical composition that is suitable for veterinary or human pharmaceutical use.

The compounds described herein can be prepared and/or formulated as pharmaceutically acceptable salts or when appropriate as a free base. Pharmaceutically acceptable salts are non-toxic salts of a free base form of a compound that possess the desired pharmacological activity of the free base. These salts can be derived from inorganic or organic acids or bases. For example, a compound that contains a basic nitrogen can be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in REmINGTON: THE SCIENCE AND PRACTICE OF PHARMACY, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

### IV. Compositions

Also provided herein is a pharmaceutical composition comprising a pharmaceutically effective amount of a compound of the present disclosure, or a pharmaceutically acceptable salt thereof, and a pharmaceutically acceptable carrier or excipient.

As used herein, “pharmaceutically acceptable carrier” or “pharmaceutically acceptable excipient” includes, but is not limited to, any and all solvents, dispersion media, coatings, antibacterial and antifungal agents, isotonic and absorption delaying agents, and combinations thereof. The use of pharmaceutically acceptable carriers and pharmaceutically acceptable excipients for pharmaceutically active substances is well known in the art. Except insofar as any conventional media or agent is incompatible with the active ingredient, its use in the therapeutic formulations is contemplated. Supplementary active ingredients can also be incorporated into the formulations. The carrier(s) must be “acceptable” in the sense of being compatible with the other ingredients of the formulation and physiologically innocuous to the recipient thereof.

The compounds disclosed herein can be formulated with conventional carriers and excipients. Tablets can contain, for instance, excipients, glidants, fillers, binders, or a combination thereof. Aqueous formulations are prepared in sterile form, and when intended for delivery by other than oral administration generally will be isotonic. Exemplary excipients include, but are not limited to, those set forth in the “HANDBOOK OF PHARMACEUTICAL EXCIPIENTS” (1986). Excipients can include, for example, ascorbic acid and other antioxidants, chelating agents such as EDTA, carbohydrates such as dextran, hydroxyalkylcellulose, hydroxyalkylmethylcellulose, stearic acid, and combinations thereof. In some embodiments, the formulation is basic. In some embodiments, the formulation is acidic. In some embodiments, the formulation has a neutral pH. In some embodiments, the pH of the formulations is from 2 to 11 (e.g., 2, 3, 4, 5, 6, 7, 8, 9, 10, 11, 2-3, 2-4, 2-5, 2-6, 2-7,2-8,2-9,2-10, 3-4, 3-5, 3-6, 3-7, 3-8, 3-9, 3-10,4-5,4-6,4-7,4-8, 4-9, 4-10, 4-11, 5-6, 5-7, 5-8, 5-9, 5-10, 5-11, 6-7, 6-8, 6-9, 6-10, 6-11, 7-8, 7-9, 7-10, 7-11, 8-9, 8-10, 8-11, 9-10, or 9-11).

In some embodiments, the compounds disclosed herein have pharmacokinetic properties (e.g., oral bioavailability) suitable for oral administration of the compounds. Formulations suitable for oral administration can, for instance, be presented as discrete units such as capsules, cachets or tablets, each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient can also be administered, for instance, as a bolus, electuary, or paste.

A tablet can be made by compression or molding, optionally with at least accessory ingredients. Compressed tablets can be prepared by compressing in a suitable machine the active ingredient in a free-flowing form such as, for instance, a powder or granules, optionally mixed with a binder, lubricant, inert diluent, preservative, surface active, dispersing agent, or a combination thereof. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom.

Formulations for oral use can be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin, or olive oil.

For diseases of the eye or other external tissues (e.g., mouth and skin), the formulations can be applied as a topical ointment or cream containing the active ingredient(s) in an amount of, for example, 0.075% to 20% w/w (including active ingredient(s) in a range from 0.1% to 20% in increments of 0.1% w/w such as 0.6% w/w, 0.7% w/w, etc.), from 0.2% to 15% w/w, or from 0.5% to 10% w/w. When formulated in an ointment, the active ingredients can be employed in some embodiments with either a paraffinic or a water-miscible ointment base. Alternatively, the active ingredients can be formulated in a cream with an oil-in-water cream base.

In some embodiments, the aqueous phase of the cream base can include, for example, from 30% to 90% (e.g., 35%, 40%, 45%, 50%, 55%, 60%, 65%, 70%, 75%, 80%, 85%) w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. In some embodiments, the cream base can include, for instance, a compound that enhances absorption or penetration of the active ingredient through the skin or other affected areas. Examples of such dermal penetration enhancers include, but are not limited to, dimethyl sulfoxide and related analogs. In some embodiments, the cream or emulsion does not include water.

The oily phase of the emulsions can be constituted from known ingredients in a known manner. In some embodiments, the phase comprises merely an emulsifier (otherwise known as an emulgent). In some embodiments, the phase comprises a mixture of at least one emulsifier with a fat, an oil, or a combination thereof. In some embodiments, a hydrophilic emulsifier is included together with a lipophilic emulsifier that acts as a stabilizer. Together, the emulsifier(s) with or without stabilizer(s) can make up the so-called emulsifying wax, and the wax together with the oil and fat make up the so-called emulsifying ointment base that can form the oily dispersed phase of the cream formulations.

Emulgents and emulsion stabilizers suitable for use in the formulation can include, but are not limited to, TWEEN® 60, TWEEN® 80, SPAN® 80, cetostearyl alcohol, benzyl alcohol, myristyl alcohol, glyceryl mono-stearate, sodium lauryl sulfate, captisol (and other beta cyclodextrin vehicles), and combinations thereof.

The choice of suitable oils or fats for the formulation can be based on achieving the desired cosmetic properties. In some embodiments, the cream can be a non-greasy, non-staining, and washable product with suitable consistency to avoid leakage from tubes or other containers. In some embodiments, esters can be included, such as, for example, straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate, a blend of branched chain esters known as CRODAMOL® CAP, or a combination thereof. In some embodiments, high melting point lipids such as white soft paraffin and/or liquid paraffin or other mineral oils can be included.

Effective dose of active ingredient depends at least on the nature of the condition being treated, toxicity, whether the compound is being used prophylactically (lower doses) or against an active viral infection, the method of delivery, and the pharmaceutical composition, and will be determined by the clinician using conventional dose escalation studies. In some embodiments, the effective dose is from 0.0001 to 100 mg/kg body weight per day; for instance, from 10 to 30 mg/kg body weight per day; from 15 to 25 mg/kg body weight per day; from 10 to 15 mg/kg body weight per day; or from 20 to 30 mg/kg body weight per day. For example, the daily candidate dose for an adult human of approximately 70 kg body weight can range from 1 mg to 2000 mg (e.g., from 5 mg to 500 mg, from 500 mg to 1000 mg, from 1000 mg to 1500 mg, from 1500 mg to 2000 mg), and can take the form of single or multiple doses. For example, the daily candidate dose for an adult human of approximately 70 kg body weight can range from 1 mg to 1000 mg (e.g., from 5 mg to 500 mg), and can take the form of single or multiple doses.

### V. Kits

Also provided herein are kits that includes a compound disclosed herein or a pharmaceutically acceptable salt thereof. In some embodiments the kits described herein can comprise a label and/or instructions for use of the compound in the treatment of a disease or condition in a subject (e.g., human) in need thereof.

In some embodiments, the kit can also comprise one or more additional therapeutic agents and/or instructions for use of additional therapeutic agents in combination with the compound disclosed herein in the treatment of the disease or condition in a subject (e.g., human) in need thereof.

In some embodiments, the kits provided herein comprise individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units can include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit can contain a single dosage unit and in others multiple dosage units are present, such as the number of dosage units required for a specified regimen or period.

Also provided are articles of manufacture that include a compound disclosed herein, or a pharmaceutically acceptable salt, stereoisomer, mixture of stereoisomers or tautomer thereof; and a container. In some embodiments, the container of the article of manufacture is a vial, jar, ampoule, preloaded syringe, blister package, tin, can, bottle, box, an intravenous bag, an inhaler, or a nebulizer.

### VI. Administration

One or more of the compounds of Formula I or pharmaceutically acceptable salt thereof (herein referred to as the active ingredients) are administered by any route appropriate to the condition to be treated. Suitable routes include oral, rectal, nasal, pulmonary, topical (including buccal and sublingual), vaginal and parenteral (including subcutaneous, intramuscular, intravenous, intradermal, intrathecal and epidural), and the like. It will be appreciated that the route may vary with for example the condition of the recipient. An advantage of the compounds herein is that they are orally bioavailable and can be dosed orally.

The compounds of the present disclosure can be administered by any route appropriate to the condition to be treated.

Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the route may vary with for example the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The dosage or dosing frequency of a compound of the present disclosure may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A compound of the present disclosure may be combined with one or more additional therapeutic agents in any dosage amount of the compound of the present disclosure (e.g., from about 1 mg to about 1000 mg of compound).

### VII Methods

Without being bound by the theory, as TASL is regulated by proteostatic interaction with SLC15A4, the compound of Formula I or pharmaceutically acceptable salt thereof described herein inhibits interaction of TASL protein with SLC15A4. Accordingly, in some embodiments, the method of the present disclosure is a method of inhibiting binding between SLC15A4 and TASL in an immune cell, comprising administering to the immune cell an effective amount of a compound or pharmaceutically acceptable salt thereof of the present disclosure or a pharmaceutical composition of the present disclosure.

Any immune cell can be used in a method of the present disclosure. In some embodiments, the immune cell is a B-cell, neutrophil, dendritic cell, or monocyte.

It has been reported in the literature that SLC15A4 and/or TASL play a role in autoimmune diseases (including systemic lupus erythematosus) and inflammatory conditions (including inflammatory bowel disease, psoriasiform dermatitis and endosomal TLR-dependent inflammation.)

Based on the role of SLC15A4 and/or TASL in IRF5 activation, the compounds of the present disclosure are expected to be useful in the treatment or prevention of autoimmune disorders and inflammatory conditions, including systemic lupus erythematosus, cutaneous lupus erythematosus, acute cutaneous lupus erythematosus, subacute cutaneous lupus erythematosus, chronic cutaneous lupus erythematosus, discoid lupus erythematosus, lupus nephritis, endosomal TLR-dependent hyperinflammation, macrophage activation syndrome, rheumatoid arthritis, scleroderma/systemic sclerosis, sarcoidosis, Sjogren's syndrome, inflammatory myopathy, polymyositis, dermatomyositis, necrotizing autoimmune myositis, inclusion body myositis, juvenile dermatomyositis, primary biliary cirrhosis, multiple sclerosis, ankylosing spondylitis, oral ulcers, periodontitis, Behget's disease (syndrome), psoriasis, psoriatic arthritis, Raynaud's disease, myasthenia gravis, irritable bowel syndrome, inflammatory bowel disease, Crohn's disease, ulcerative colitis, psoriasiform dermatitis, allergic airway inflammation, allergic blepharitis, atopic dermatitis, bullous pemphigoid, epidermolysis bullosa, prurigo nodularis, severe stress, thyroid disease, tinea cruris, rosacea, cutaneous amyloidosis, autoimmune atrophic gastritis, chronic pancreatitis, erythromelalgia, fibromyalgia, gout, headaches, herniated disc, interstitial cystitis, polymyalgia rheumatica, sacroiliac joint dysfunction, transverse myelitis, Steven-Johnson syndrome, toxic epidermal necrolysis, appendicitis, bursitis, phlebitis psoriasis, graft-versus-host disease, reactive airway disorder, asthma, airway infection, autoinflammatory disease, celiac disease, chronic prostatitis, diverticulitis, glomerulonephritis, hidradenitis suppurativa, hypersensitivities, intestinal disorder, epithelial intestinal disorder, otitis, pelvic inflammatory disease, and vasculitis.

Accordingly, in some embodiments, a method of the present disclosure is a method of treating an autoimmune disorder or an inflammatory condition in a subject in need thereof, comprising administering to the subject a therapeutically effective amount of a compound or pharmaceutically acceptable salt thereof of the present disclosure, or a pharmaceutical composition of the present disclosure. In some embodiments, the autoimmune disorder comprises systemic lupus erythematosus (SLE).

Genetic studies of systemic lupus erythematosus (SLE) in human cells suggest SLC15A4 has a causative role in this disorder. The genetic association of SLC15A4 and TASL with SLE was identified in a cohort of European ancestry and separately in a cohort of a Chinese Han population. He, C.-F. and coworkers identified through GWAS the genetic association of SLC15A4 with SLE-related discoid rash in a Chinese Han population. Langefeld and coworkers identified through GWAS the genetic association of SLC15A4 with SLE in a cohort of European ancestry.

Further, there is a body of evidence based on genetic studies of systemic lupus erythematosus (SLE) in human cells indicating the role TASL in this disorder. Odhams et al. proposed that genetic variants of TASL (CXorf21) associated with SLE lead to increased TASL expression in an interferon- and sex specific manner.

Mouse models have showed the importance of TASL/SLC15A4 in systemic lupus erythematosus. A protective role was suggested for SLC15A4 deficiency in the development of SLE in a mouse model using the C57BL/6-Fas(lpr) strain. SLC15A4-deficiency was protective in two models of SLE (pristane-induced and C57BL/61pr7lpr mice) and that SLC15A4 was required in B cells for endosomal TLR function. SLC15A4-deficiency was protective in a mercury-induced model of SLE.

Accordingly, the compounds of the present disclosure are believed to be useful in the treatment or prevention of autoimmune disorders, in particular systemic lupus erythematosus.

Based on the role of SLC15A4 and/or TASL in IRF5 activation, the compounds of the present disclosure are also expected to be useful in the treatment of prevention of autoimmune disorders, preferably selected from systemic lupus erythematosus, rheumatoid arthritis, scleroderma, Sjogren's syndrome, polymyositis/dermatomyositis, primary biliary cirrhosis, multiple sclerosis, oral ulcers, periodontitis, Behget's disease, myasthenia gravis, and ankylosing spondylitis.

Further genetic studies demonstrate the role of SLC15A4 and/or TASL in inflammatory conditions. Heinz and coworkers have shown essential role of SLC15A4-TASL and their relationship in human cell lines and primary cells for endosomal TLR function.

There is further a body of evidence based on the studies in mouse models indicating the role of SLC15A4 and/or TASL in inflammatory conditions. SLC15A4 was identified as an essential component in endosomal TLR function in plasmacytoid dendritic cells. SLC15A4 deficiency has been found to impair CpG-induced production of inflammatory cytokines from dendritic cells. Furthermore, SLC15A4 deficiency was found to be protective in a mouse model of inflammatory bowel disease. SLC15A4-deficient mice also showed defective cytokine production upon activation of the NOD-like receptor NOD1. An important role was suggested for SLC15A4 in pDCs in controlling viral persistence, as evidenced in a model of LCMV infection.

Based on the role of SLC15A4 and/or TASL in IRF5 activation, the compounds of the present disclosure are also expected to be useful in the treatment of prevention of inflammatory condition, preferably selected from inflammatory bowel disease, psoriasiform dermatitis, endosomal TLR-dependent inflammation, ulcerative colitis, Crohn's disease, endosomal TLR-induced hyperinflammation, macrophage activation syndrome, allergic airway inflammation, and sarcoidosis, preferably wherein the inflammatory condition is selected from inflammatory bowel disease, psoriasiform dermatitis, and endosomal TLR-dependent inflammation. In some embodiments, the inflammatory condition comprises inflammatory bowel disease.

Accordingly, the compounds of the present disclosure are believed to be useful in treatment or prevention of an inflammatory condition.

Thus, in one embodiment, the present disclosure relates to the compound of Formula I or its pharmaceutically acceptable salt, or the pharmaceutical composition of the present disclosure, for use in treatment or prevention of an autoimmune disorder or an inflammatory condition. In one embodiment, the present disclosure relates to the compound of Formula I or its pharmaceutically acceptable salt, or the pharmaceutical composition of the present disclosure, for use in treatment or prevention of an autoimmune disorder. In one embodiment, the present disclosure relates to the compound of Formula I or its pharmaceutically acceptable salt, or the pharmaceutical composition of the present disclosure, for use in treatment or prevention of an inflammatory condition.

In some embodiments, the autoimmune disorder is selected from systemic lupus erythematosus, rheumatoid arthritis, scleroderma, Sjogren's syndrome, polymyositis/dermatomyositis, primary biliary cirrhosis, multiple sclerosis, oral ulcers, periodontitis, Behget's disease, myasthenia gravis, and ankylosing spondylitis. In some embodiments, the autoimmune disorder is systemic lupus erythematosus.

In some embodiments, the inflammatory condition is selected from inflammatory bowel disease, psoriasiform dermatitis, endosomal TLR-dependent inflammation, ulcerative colitis, Crohn's disease, endosomal TLR-induced hyperinflammation, macrophage activation syndrome, allergic airway inflammation, and sarcoidosis. In some embodiments, the inflammatory condition is selected from inflammatory bowel disease, psoriasiform dermatitis, and endosomal TLR-dependent inflammation.

In some embodiments, the present disclosure relates to use of the compound of Formula I or its pharmaceutically acceptable salt, or the pharmaceutical composition of the present disclosure, in the manufacture of a medicament for use in the treatment or prevention of an autoimmune disorder or an inflammatory condition. In one embodiment, the present disclosure relates to use of the compound of Formula I or its pharmaceutically acceptable salt, or the pharmaceutical composition of the present disclosure, in the manufacture of a medicament for use in the treatment or prevention of an autoimmune disorder. In one embodiment, the present disclosure relates to use of the compound of Formula I or its pharmaceutically acceptable salt, or the pharmaceutical composition of the present disclosure, in the manufacture of a medicament for use in the treatment or prevention of an inflammatory condition.

In some embodiments, the present disclosure relates to the method of treating an autoimmune disorder, the method comprising administering the compound of Formula I or its pharmaceutically acceptable salt, or the pharmaceutical composition of the present disclosure, to a subject in need thereof. It is to be understood that said compound or its salt or said pharmaceutical composition is to be administered in a therapeutically effective amount as described herein.

In one embodiment, the present disclosure relates to the method of treating an inflammatory condition, the method comprising administering the compound of Formula I or its pharmaceutically acceptable salt, or the pharmaceutical composition of the present disclosure, to a subject in need thereof. It is to be understood that said compound or its salt or said pharmaceutical composition is to be administered in a therapeutically effective amount, preferably as described herein.

It is to be noted that the compound of Formula I or its salt has been shown by the present inventors to inhibit SLC15 peptide transporter (i.e., SLC15A4). Accordingly and preferably, it is expected that the therapeutic effect of the compound of Formula I or its salt is based on the inhibition of SLC15 peptide transporter (i.e., SLC15A4). Accordingly, in one embodiment the present disclosure relates to the compound of Formula I or its salt, or the pharmaceutical composition of the present disclosure for use in the treatment or prevention of an autoimmune disorder, wherein said compound or said pharmaceutical composition inhibits SLC15 peptide transporter. Given the ability of the compound of the present disclosure, or its salt, to inhibit the SLC15 peptide transporter, said compound is particularly useful in the treatment of the autoimmune disorder, wherein the autoimmune disorder is a disorder associated with SLC15 peptide transporter. Accordingly, the present disclosure in one embodiment relates to the compound of Formula I or its salt, or the pharmaceutical composition of the present disclosure wherein said compound or said pharmaceutical composition for use in the treatment or prevention of an autoimmune disorder, wherein the autoimmune disorder is a disorder associated with SLC15 peptide transporter.

The compounds provided in the present disclosure are also useful in treatment or prevention of other lupus diseases such as cutaneous or neonatal lupus erythematosus.

### VIIL EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

Many general references providing commonly known chemical synthetic schemes and conditions useful for synthesizing the disclosed compounds are available (see, e.g., Smith, March's Advanced Organic Chemistry: Reactions, Mechanisms, and Structure, 7^{th} edition, Wiley-Interscience, 2013.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. For example, disclosed compounds can be purified via silica gel chromatography. See, e.g., Introduction to Modern Liquid Chromatography, 3^{rd} ed., ed. L. R. Snyder, J. J. Kirkland, J. W. Dolan. John Wiley and Sons, 2011; and Thin Layer Chromatography, E. Stahl (ed.), Springer-Verlag, New York, 1969.

Compounds were characterized using standard instrumentation methods. Identification of the compound was carried out by hydrogen nuclear magnetic resonance spectrum (¹H-NMR) and mass spectrum (MS). ¹H-NMR was measured at 400 MHz, unless otherwise specified. In some cases, exchangeable hydrogen could not be clearly observed depending on the compound and measurement conditions. The designation br. or broad, used herein, refers to a broad signal. LCMS were performed according to the following. HPLC preparative chromatography was carried out according to the following conditions, unless otherwise specified.

### General LCMS Conditions:

LCMS (Shimadzu), column: YMC-Triart C18, 50×4.6 mm, 5 μm, mobile phase: Solvent A: Solvent A: H₂O/MeCN/NH₄OH=90/10/0.05; Solvent B: MeCN, Flow rate: 3 mL/min, temperature: 40° C.; gradient: 0.01 min @20% B, 1.79 min gradient (20-95% B), then 0.7 min @95% B.

LCMS (Shimadzu), column: YMC-Triart C18, 50×4.6 mm, 5 μm, RP-18e, 50×4.6 mm, mobile phase: Solvent A: H₂O (10 mmol/L NH₄HCO₃)/MeCN=90/10 Solvent B: H2O/MeCN=10/90, Flow rate: 2.5 mL/min, temperature: 40° C.; gradient: 0.01 min @20% B, 1.79 min gradient (20-95% B), then 0.7 min @95% B.

LCMS (Shimadzu), column: YMC-Triart C18, 50×4.6 mm, 5 μm, RP-18e, 50×4.6 mm, mobile phase: Solvent Solvent A: H₂O/MeCN/FA=90/10/0.05 Solvent B: MeCN, Flow rate: 2.5 mL/min, temperature: 40° C.; gradient: 0.01 min @20% B, 1.79 min gradient (20-95% B), then 0.7 min @95% B.

General preparatory HPLC conditions: HPLC purifications were performed on a SHIMADZU LC-8A, Shimadzu LH-40 or Shimadzu LC-8A; Column: YMC-Triart C18, 250*20 mm, 5 μm, YMC-Triart C₈, 250*20 mm, 5 μm, YMC-Pack ODS-AQ, 250*20 mm, 5 m or Xbridge Prep C18 5 m OBD etc., 250*20 mm, 5 m with UV detection which were controlled by LC solution Chemstation software. H₂O (FA/TFA) or H₂O NH₄OH/NH₄HCO₃) and MeOH (MeCN) as mobile phase at the indicated flow rate (15-20 mL/min) at room temperature.

The Examples provided herein describe the synthesis of compounds disclosed herein as well as intermediates used to prepare the compounds. It is to be understood that individual steps described herein may be combined. It is also to be understood that separate batches of a compound may be combined and then carried forth in the next synthetic step.

Representative syntheses of compounds of the present disclosure are described in schemes below, and the particular examples that follow.

Abbreviations. Certain abbreviations and acronyms are used in describing the experimental details. Although most of these would be understood by one skilled in the art, the following table contains a list of many of these abbreviations and acronyms.

TABLE 2
List of abbreviations and acronyms.

| Abbreviation | Meaning |
|---|---|
| ACN | acetonitrile |
| Bn | benzyl |
| Boc | tert-butoxycarbonyl |
| (Bpin)₂ | bis(pinacolato)diboron |
| Bu | butyl |
| dba | dibenzylideneacetone |
| DCE | 1,2-dichloroethane |
| DCM | dichloromethane |
| DIEA | N,N-diisopropylethylamine |
| DMB | 2,4-dimethoxybenzyl |
| DMSO | dimethylsulfoxide |
| DMF | dimethylformamide |
| DPPA | diphenyl phosphoryl azide |
| dppf | 1,1′-bis(diphenylphosphino)ferrocene |
| dtbpf | 1,1′-bis(di-tert-butylphosphino)ferrocene |
| Et | ethyl |
| EtOH | ethanol |
| EtOAc | ethyl acetate |
| FA | formic acid |
| HATU | N-[(dimethylamino)-1H-1,2,3-triazolo-[4,5-b]pyridin-1-ylmethylene]-N- |
| | methylmethanaminium hexafluorophosphate N-oxide |
| HPLC | high performance liquid chromatography |
| HPLCMS | high performance liquid chromatography-mass spectrometry |
| IPA | isopropanol |
| ISRE | interferon stimulated response element |
| KOPiv | potassium pivalate or potassium trimethylacetate |
| LC | liquid chromatography |
| LCMS | liquid chromatography-mass spectrometry |
| Me | methyl |
| MeOH | methanol |
| m/z | mass to charge ratio |
| MS or ms | mass spectrum |
| NFKB | nuclear factor kappa-light-chain-enhancer of activated B cells |
| NMI | N-methylimidazole |
| Pd₂dba₃ | tris(dibenzylideneacetone)dipalladium |
| PE | petroleum ether |
| PMB | para-methoxybenzyl |
| RT | retention time |
| TCFH | N,N,N′,N′-tetramethylchloroformamidinium hexafluorophosphate |
| TEA | triethylamine |
| TFA | trifluoroacetic acid |
| THF | tetrahydrofuran |
| TMSCl | trimethylsilyl chloride |
| TsOH | para-toluenesulfonic acid |
| UV | ultraviolet |
| XantPhos | 4,5-bis(diphenylphosphino)-9,9-dimethylxanthene |
| δ | parts per million referenced to residual non-deuterated solvent peak |
| s | singlet |
| m | multiplet |
| d | doublet |
| dd | doublet of doublet |
| t | triplet |

### Example 1. (6-(5-amino-6-fluoropyrazin-2-yl)-6′-ethoxy-5-fluoro-[2,3′-bipyridin]-4-yl)((1R,4R)-5-methyl-2,5-diazabicyclo[2.2.2]octan-2-yl)methanone

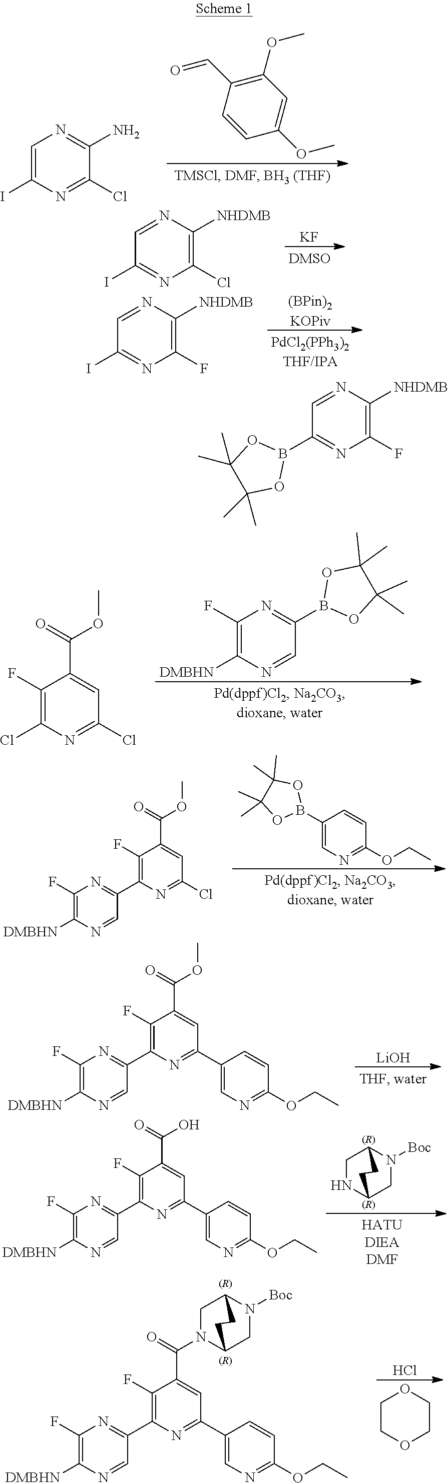

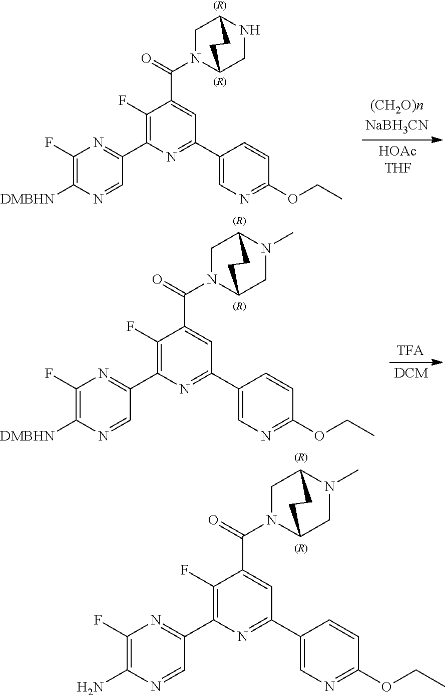

Step 1: To a solution of 3-chloro-5-iodopyrazin-2-amine (1.00 eq, 50 g, 195.77 mmol) and 2,4-dimethoxybenzaldehyde (1.30 eq, 42.3 g, 254.51 mmol) in DMF (500 mL) was added TMSCl (3.00 eq, 63.8 g, 587.26 mmol) at 0° C. in one portion and the reaction was stirred at 60° C. for 2 h under N₂ atmosphere. Upon completion, the yellow mixture was cooled to −5° C. and BH₃ (2.04 eq, 400 mmol, 400 mL, 1 M in THF) was added dropwise at this temperature (Caution: H2 gas was released during the addition. Please handle with care). After addition (~20 min), the yellow solution was warmed and stirred at ambient temperature for 2 h. The solution was concentrated in vacuo and the crude product was purified by column chromatography on silica gel (PE in EtOAc=5%) to afford 3-chloro-N-(2,4-dimethoxybenzyl)-5-iodopyrazin-2-amine (64 g, 157.79 mmol, 80.6% yield) as a white solid. LC purity: 95% (UV at 254 nm); Mass calculated for C₁₃H₁₃CIN₃O₂[M+1]⁺, 406.0, found 405.9; Retention time: 0.965 min. ¹H NMR (400 MHz, DMSO-d₆) δ 8.19 (s, 1H), 7.49 (t, J=6.0 Hz, 1H), 6.97-6.95 (d, J=8.4 Hz, 1H), 6.55-6.45 (d, J=2.4 Hz, 1H), 6.43-6.41 (d, J=8.4 Hz, 1H), 4.44-4.42 (d, J=6 Hz, 1H), 3.80 (s, 3H), 3.74 (s, 3H).

Step 2: To a solution of 3-chloro-N-(2,4-dimethoxybenzyl)-5-iodopyrazin-2-amine (1.00 eq, 50 g, 123.27 mmol), 18-crown-6 (2.00 eq, 65.2 g, 246.67 mmol), and KF (3.00 eq, 21.5 g, 370.05 mmol) in DMSO (500 mL) was added DIEA (2.99 eq, 47.7 g, 369.08 mmol) at ambient temperature and the reaction was stirred at 120° C. for 8 h under N₂ atmosphere. Upon completion, the reaction mixture was diluted with water and extracted with EtOAc. The organic layers were washed with brine over 4 times, dried over anhydrous sodium sulfate, filtered and concentrated in vacuo. The residue was purified by column chromatography on silica gel (PE in EtOAc=10%) to afford N-(2,4-dimethoxybenzyl)-3-fluoro-5-iodopyrazin-2-amine (33.6 g, 86.33 mmol, 70.0% yield) as a white solid. LC purity: 90% (UV at 254 nm); Mass calculated for C₁₃H₁₃FIN₃O₂[M+1]⁺, 390.0, found 390.0; Retention time: 0.938 min.

Step 3: To a stirred solution of N-(2,4-dimethoxybenzyl)-3-fluoro-5-iodopyrazin-2-amine (1.00 eq, 5 g, 12.85 mmol) and bis(pinacolato)diboron (1.50 eq, 4.9 g, 19.30 mmol) in THF (36 mL)/IPA (12 mL) were added potassium pivalate (1.26 eq, 2.3 g, 16.17 mmol) and PdCl₂(PPh₃)₂(0.05 eq, 451 mg, 0.64 mmol) under N₂ atmosphere. The reaction was stirred at 40° C. for 20 h. The resulting mixture was evaporated carefully at ambient temperature. The residue was diluted with dichloromethane, filtered to remove undissolved salts and the filtrate was evaporated to give a semi-solid crude (The crude material is stable in both semi-solid form and in dioxane solution at ambient temperature) which was used in the next step without purification. LC purity: 83% (UV at 254 nm); Mass calculated for C₁₉H₂₅BFN₃O₄[M+1]⁺, 390.2, found 390.3; Retention time: 1.948 & 0.691 min.

Step 4: To a mixture of methyl 2,6-dichloro-3-fluoroisonicotinate (1.00 eq, 800 mg, 3.57 mmol), N-(2,4-dimethoxybenzyl)-3-fluoro-5-(4,4,5,5-tetramethyl-1,3,2-dioxaborolan-2-yl)pyrazin-2-amine (0.80 eq, 1113 mg, 2.86 mmol) and Na₂CO₃ (2.00 eq, 757 mg, 7.14 mmol) in 1,4-dioxane (10 mL) and water (1 mL) was added Pd(dtbpf)Cl₂ (0.10 eq, 232 mg, 0.357 mmol). The reaction was stirred at 40° C. for 2 h under N₂ atmosphere. The reaction was then diluted with water and extracted with ethyl acetate. The combined organic layers were washed with brine, dried over sodium sulfate, filtered, and concentrated in vacuo. The residue was purified by column chromatography on silica gel with petroleum ether/ethyl acetate (3/2) to give methyl 6-chloro-2-(5-((2,4-dimethoxybenzyl)amino)-6-fluoropyrazin-2-yl)-3-fluoroisonicotinate (480 mg, 1.06 mmol, 30.0% yield) as a yellow solid. LC purity: 83% (UV at 254 nm); Mass calculated for C₂₀H₁₇ClF₂N₄O₄ [M+1]⁺ 451.1, found 451.1; Retention time: 0.766 min.

Step 5: To a mixture of methyl 6-chloro-2-(5-((2,4-dimethoxybenzyl)amino)-6-fluoropyrazin-2-yl)-3-fluoroisonicotinate (1.00 eq, 500 mg, 1.11 mmol), 2-ethoxy-5-(4,4,5,5-tetramethyl-1,3,2-dioxaborolan-2-yl)pyridine (1.20 eq, 334 mg, 1.34 mmol) and Na₂CO₃ (2.00 eq, 235 mg, 2.22 mmol) in 1,4-dioxane (10 mL) and water (1 mL) was added Pd(dppf)Cl₂ (0.12 eq, 97 mg, 0.13 mmol). The reaction was stirred at 80° C. for 2 h under N₂ atmosphere. The reaction was then diluted with water and extracted with ethyl acetate. The combined organic layers were washed with brine, dried over sodium sulfate, filtered, and concentrated in vacuo. The residue was purified by column chromatography on silica gel with petroleum ether/ethyl acetate (1/1) to give methyl 6-(5-((2,4-dimethoxybenzyl)amino)-6-fluoropyrazin-2-yl)-6′-ethoxy-5-fluoro-[2,3′-bipyridine]-4-carboxylate (400 mg, 0.74 mmol, 66.3% yield) as a yellow solid. LC purity: 80% (UV at 254 nm); Mass calculated for Chemical Formula: C₂₇H₂₅F₂N₅O₅[M+1]⁺, 538.2, found 538.2; Retention time: 0.994 min.

Step 6: To a solution of methyl 6-(5-((2,4-dimethoxybenzyl)amino)-6-fluoropyrazin-2-yl)-6′-ethoxy-5-fluoro-[2,3′-bipyridine]-4-carboxylate (1.00 eq, 400 mg, 0.74 mmol) in THF (3 mL) and water (1 mL) was added LiGH (10.04 eq, 178 mg, 7.43 mmol). The reaction was stirred for 1 h at ambient temperature. The mixture was evaporated and the residue was added DMSO, filtered and purified via phase flash chromatography (C18 silica gel) using a gradient of 20% to 50% ACN in water (with 10 mmol/L NH₄HCO₃.) to afford 6-(5-((2,4-dimethoxybenzyl)amino)-6-fluoropyrazin-2-yl)-6′-ethoxy-5-fluoro-[2,3′-bipyridine]-4-carboxylic acid (310 mg, 0.59 mmol, 79.8% yield) as a yellow solid. LC purity: 90% (UV at 254 nm); Mass calculated for Chemical Formula: C₂₆H₂₃F₂N₅O₅[M+1]⁺, 524.2, found 524.2; Retention time: 0530 min.

Step 7: To a solution of 6-(5-((2,4-dimethoxybenzyl)amino)-6-fluoropyrazin-2-yl)-6′-ethoxy-5-fluoro-[2,3′-bipyridine]-4-carboxylic acid (1.00 eq, 60 mg, 0.11 mmol), tert-butyl (1R,4R)-2,5-diazabicyclo[2.2.2]octane-2-carboxylate (1.54 eq, 36 mg, 0.17 mmol) in DMF (3 mL) were added HATU (1.55 eq, 65 mg, 0.17 mmol) and DIEA (3.00 eq, 43 mg, 0.33 mmol) at 0° C. sequentially. The reaction was stirred for 2 h at ambient temperature. The crude product was diluted with DMSO, filtered and purified via phase flash chromatography (C18 silica gel) using a gradient of 20% to 80% ACN in water (with 10 mmol/L NH₄HCO₃) to afford tert-butyl (1R,4R)-5-(6-(5-((2,4-dimethoxybenzyl)amino)-6-fluoropyrazin-2-yl)-6′-ethoxy-5-fluoro-[2,3′-bipyridine]-4-carbonyl)-2,5-diazabicyclo[2.2.2]octane-2-carboxylate (60 mg, 0.083 mmol, 75.4% yield) as a yellow solid. LC purity: 85% (UV at 254 nm); Mass calculated for C₃₇H₄₁F₂N₇O₆[M+1]⁺, 718.3, found 718.1; Retention time: 0852 min..

Step 8: To a solution of tert-butyl (1R,4R)-5-(6-(5-((2,4-dimethoxybenzyl)amino)-6-fluoropyrazin-2-yl)-6′-ethoxy-5-fluoro-[2,3′-bipyridine]-4-carbonyl)-2,5-diazabicyclo-[2.2.2]octane-2-carboxylate (1.00 eq, 60 mg, 0.083 mmol) in 1,4-dioxane (3 mL) was added HCl (3.0 mL, 4.0 M in dioxane) at 0° C. The reaction was stirred at ambient temperature for 1 h under N₂ atmosphere. The resulting solution was concentrated in vacuo to afford the crude ((1R,4R)-2,5-diazabicyclo[2.2.2]octan-2-yl)(6-(5-((2,4-dimethoxybenzyl)amino)-6-fluoropyrazin-2-yl)-6′-ethoxy-5-fluoro-[2,3′-bipyridin]-4-yl)methanone hydrochloride as a yellow solid which was used directly in the next step without further purification. LC purity: 92% (UV at 254 nm); Mass calculated for C₃₂H₃₃F₂N₇O₄[M+1]⁺, 618.3, found 618.0; Retention time: 0.625 min.

Step 9: To a solution of ((1R,4R)-2,5-diazabicyclo[2.2.2]octan-2-yl)(6-(5-((2,4-dimethoxybenzyl)amino)-6-fluoropyrazin-2-yl)-6′-ethoxy-5-fluoro-[2,3′-bipyridin]-4-yl)methanone hydrochloride (1.00 eq, 40 mg, 0.061 mmol) in THF (1 mL) was added DIEA (2.54 eq, 20 mg, 0.15 mmol), and the mixture was stirred at ambient temperature for 10 mins. Then to the above mixture, a mixture of (CH₂O)n (1.52 eq, 3 mg, 0.099 mmol) and HOAc (2.72 eq, 10 mg, 0.166 mmol) in THF (0.5 mL) was added. The reaction was stirred for 0.5 h. Then NaBH₃CN (4.92 eq, 20 mg, 0.32 mmol) was added in several portions and the mixture was stirred at 50° C. overnight. Upon completion, the mixture was diluted with water and extracted with EtOAc. The combined organic layers were washed with brine, dried over anhydrous Na₂SO₄, filtered, and concentrated in vacuo. The residue was added DMSO, filtered and purified via phase flash chromatography (C18 silica gel) using a gradient of 20% to 60% ACN in water (with 0.1% FA) to afford (6-(5-((2,4-dimethoxybenzyl)amino)-6-fluoropyrazin-2-yl)-6′-ethoxy-5-fluoro-[2,3′-bipyridin]-4-yl)((1R,4R)-5-methyl-2,5-diazabicyclo[2.2.2]octan-2-yl)methanone (20 mg, 0.032 mmol, 50% yield) as a light yellow solid. LC purity: 90% (UV at 254 nm); Mass calculated for C₃₃H₃₅F₂N₇O₄[M+1]⁺, 632.3, found 632.3; Retention time: 0.704 min.

Step 10: To a solution of (6-(5-((2,4-dimethoxybenzyl)amino)-6-fluoropyrazin-2-yl)-6′-ethoxy-5-fluoro-[2,3′-bipyridin]-4-yl)((1R,4R)-5-methyl-2,5-diazabicyclo[2.2.2]-octan-2-yl)methanone (1.00 eq, 20 mg, 0.032 mmol) in DCM (0.5 mL) was added TFA (1 mL) dropwise. The reaction was stirred for 1 h at 50° C. The resulting mixture was concentrated in vacuo. The residue was dissolved into MeCN and treated with 1 mL ammonium hydroxide at 0° C., stirred for 30 min and concentrated in vacuo. The residue was purified by Prep-HPLC using the following conditions: Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient (B %): 23% B to 45% B in 8 min; Wavelength: 254 nm/220 nm; RT1 (min): 6.87. LC purity: 97% (UV at 254 nm). The fraction was collected and concentrated under vacuum, and the residue was re-dissolved in CH₃CN and H₂O, and then was lyophilized to afford the title compound (6.98 mg, 0.0144 mmol, 45% yield) as a white solid. LC purity: 97% (UV at 254 nm); Mass calculated for Chemical Formula: C₂₋₄H₂₅F₂N₇O₂ [M+1]⁺, 482.2, found 482.1; Retention time: 0.612 min. ¹H NMR (400 MHz, DMSO) δ 8.96-8.93 (m, 1H), 8.73 (dd, J=8.8, 4.6 Hz, 1H), 8.52-8.44 (m, 1H), 8.04-8.01 (m, 1H), 7.38 (s, 2H), 6.93 (m, 1H), 4.47-4.34 (m, 2H), 3.71-3.69 (m, 1H), 3.52-3.42 (m, 2H), 2.94-2.84 (m, 2H), 2.68 (m, 1H), 2.32-2.30 (m, 3H), 2.00 (m, 1H), 1.82 (m, 1H), 1.70-1.58 (m, 2H), 1.40-1.32 (m, 3H).

### Example 2. (6-(5-amino-6-fluoropyrazin-2-yl)-6′-ethoxy-5-fluoro-[2,3′-bipyridin]-4-yl)(8-methyl-3,8-diazabicyclo[3.2.1]octan-3-yl)methanone

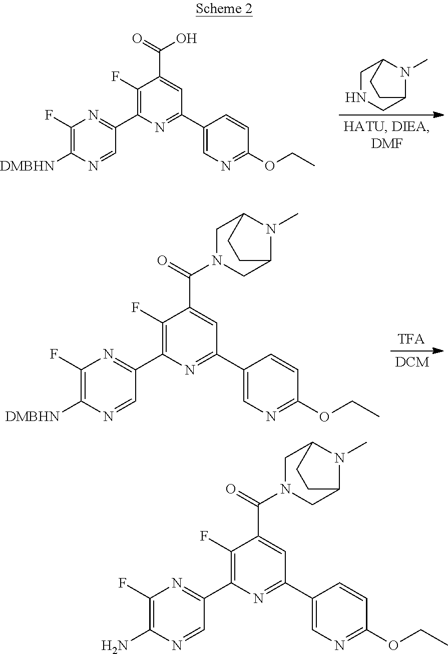

Step 1: To a solution of 2-[5-[(2,4-dimethoxyphenyl)methylamino]-6-fluoro-pyrazin-2-yl]-6-(6-ethoxy-3-pyridyl)-3-fluoro-pyridine-4-carboxylic acid (preparation in Example 1, 1.00 eq, 40 mg, 0.07 mmol) and 8-methyl-3,8-diazabicyclo[3.2.1]octane (1.50 eq, 14 mg, 0.11 mmol) in DMF (5 mL) were treated with HATU (1.50 eq, 44 mg, 0.11 mmol) and DIEA (2.00 eq, 20 mg, 0.15 mmol), and the reaction mixture was stirred for 1 h at room temperature. The resulting mixture was directly purified via reversed phase flash chromatography (C18 silica gel) using a gradient of 50% to 80% ACN in water (with 10 mmol/L NH₄HCO₃) to afford [2-[5-[(2,4-dimethoxyphenyl)methylamino]-6-fluoro-pyrazin-2-yl]-6-(6-ethoxy-3-pyridyl)-3-fluoro-4-pyridyl]-(8-methyl-3, 8-diazabicyclo[3.2.1]octan-3-yl)methanone (40 mg, 0.06 mmol, 82% yield) as a white solid. LC purity: 85% (UV at 254 nm); Mass calculated for C₃₃H₃₅F₂N₇O₄[M+H]⁺ 632, found [M+H]⁺ 632; Retention time: 0.831 min.

Step 2: To a solution of [2-[5-[(2,4-dimethoxyphenyl)methylamino]-6-fluoro-pyrazin-2-yl]-6-(6-ethoxy-3-pyridyl)-3-fluoro-4-pyridyl]-(8-methyl-3,8-diazabicyclo[3.2.1]octan-3-yl)methanone (1.00 eq, 50 mg, 0.07 mmol) in DCM (3 mL) was added TFA (1 mL), and the reaction mixture was stirred at room temperature for 2 h. The resulting mixture was concentrated in vacuo. The residue was dissolved in MeCN and treated with 1 mL ammonium hydroxide at 0° C., stirred for 30 min and concentrated in vacuo. The residue was purified by Prep-HPLC using the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 24% B to 54% B in 10 min; Wavelength: 254 nm/220 nm; RT1(min): 6.72). The fraction was collected and concentrated under vacuum, and the residue was re-dissolved in CH₃CN and H₂O, and then the mixture was lyophilized to afford [2-(5-amino-6-fluoro-pyrazin-2-yl)-6-(6-ethoxy-3-pyridyl)-3-fluoro-4-pyridyl]-(8-methyl-3,8-diazabicyclo[3.2.1]octan-3-yl)methanone (14 mg, 0.02 mmol, 35% yield) as a white solid. LC purity: 99.4% (UV at 254 nm); Mass calculated for C₂₋₄H₂₅F₂N₇O₂[M+1]⁺, 482.2, found 482.3; Retention time: 1.256 min. ¹H NMR (400 MHz, DMSO-d₆) δ 8.95 (m, 1H), 8.74 (d, J=4.6 Hz, 1H), 8.47 (m, 1H), 7.99 (m, 1H), 7.39 (s, 2H), 6.93 (d, J=8.7 Hz, 1H), 4.39-4.36 (m, 2H), 4.20-4.12 (m, 1H), 3.30-3.10 (m, 3H), 2.99 (m, 2H), 2.19 (s, 3H), 1.93 (m, 2H), 1.52 (m, 2H), 1.36 (t, J=7.0 Hz, 3H).

### Example 3. (2-(5-amino-6-fluoropyrazin-2-yl)-6-(4-(2,2-difluoroethoxy)phenyl)-3-fluoropyridin-4-yl)(5-methyl-2,5-diazabicyclo[2.2.2]octan-2-yl)methanone

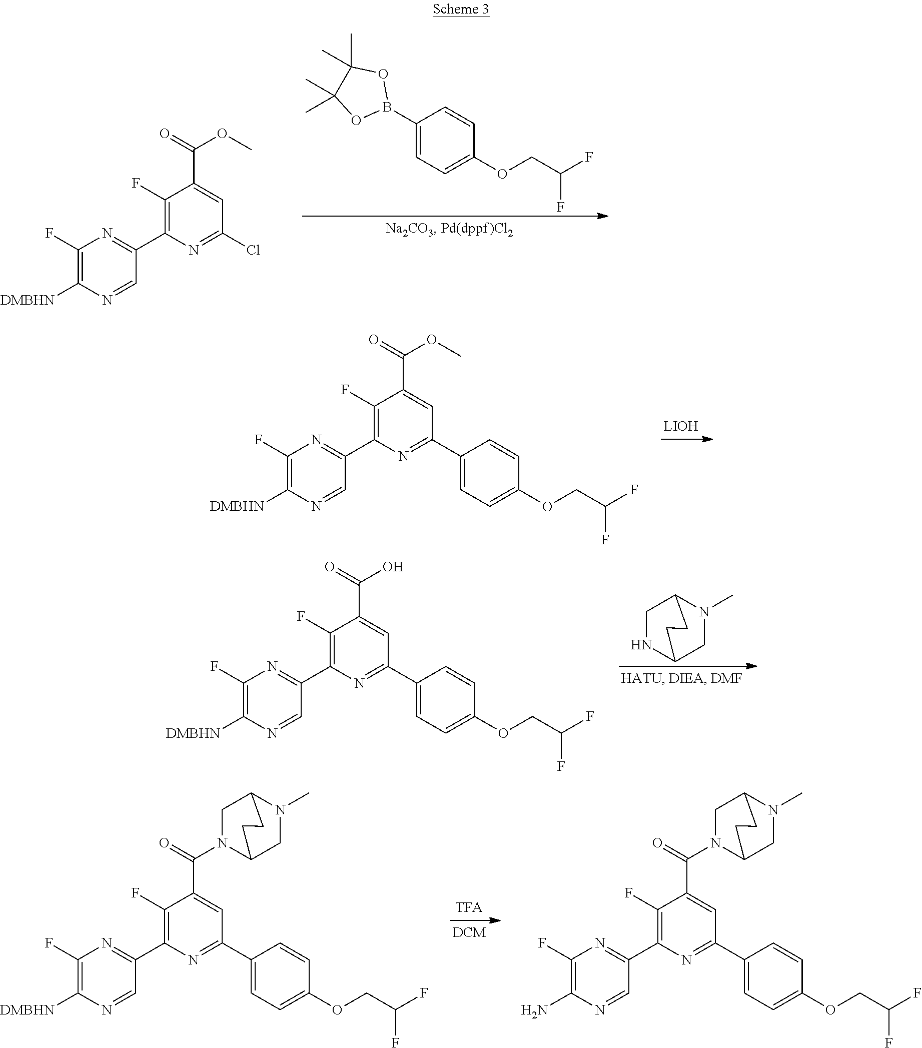

Step 1: To a mixture of methyl 6-chloro-2-(5-((2,4-dimethoxybenzyl)amino)-6-fluoropyrazin-2-yl)-3-fluoroisonicotinate (preparation in Example 1, 1.00 eq, 500 mg, 1.11 mmol), 2-(4-(2,2-difluoroethoxy)phenyl)-4,4,5,5-tetramethyl-1,3,2-dioxaborolane (1.20 eq, 381 mg, 1.34 mmol) and Na₂CO₃ (3.00 eq, 352 mg, 3.33 mmol) in 1,4-dioxane (10 mL) and water (1 mL) was added Pd(dppf)Cl₂ (0.12 eq, 97 mg, 0.13 mmol). The reaction was stirred at 80° C. for 2 h under N₂ atmosphere. The reaction was then diluted with water and extracted with ethyl acetate. The combined organic layers were washed with brine, dried over sodium sulfate, filtered, and concentrated in vacuo. The residue was purified by column chromatography on silica gel with petroleum ether/ethyl acetate (1/1) to give methyl 6-(4-(2,2-difluoroethoxy)phenyl)-2-(5-((2,4-dimethoxybenzyl)amino)-6-fluoropyrazin-2-yl)-3-fluoroisonicotinate (400 mg, 0.70 mmol, 62.9% yield) as a yellow solid. LC purity: 91% (UV at 254 nm); Mass calculated for Chemical Formula: C₂₈H₂₆F₂N₄O₅[M+1]⁺, 573.2, found 573.2; Retention time: 0.978 min.

Step 2: To a solution of methyl 6-(4-(2,2-difluoroethoxy)phenyl)-2-(5-((2,4-dimethoxybenzyl)amino)-6-fluoropyrazin-2-yl)-3-fluoroisonicotinate (1.00 eq, 400 mg, 0.70 mmol) in THF (3 mL) and water (1 mL) was added LiGH (5.01 eq, 84 mg, 3.51 mmol). The reaction was stirred for 1 h at ambient temperature. The mixture was evaporated and the residue was added DMSO, filtered and purified via phase flash chromatography (C18 silica gel) using a gradient of 20% to 50% ACN in water (with 10 mmol/L NH₄HCO₃) to afford 6-(4-(2,2-difluoroethoxy)phenyl)-2-(5-((2,4-dimethoxybenzyl)amino)-6-fluoropyrazin-2-yl)-3-fluoroisonicotinic acid (360 mg, 0.64 mmol, 91.4% yield) as a yellow solid. LC purity: 95% (UV at 254 nm); Mass calculated for Chemical Formula: C₂₇H₂₂F₄N₄O₅[M+1]⁺, 559.2, found 559.2; Retention time: 0.598 min.

Step 3& 4: The title compound (10 mg) was prepared in a 21.5% overall yield as an off-white solid, following a procedure analogous to that described in Example 2 using 6-(4-(2,2-difluoroethoxy)phenyl)-2-(5-((2,4-dimethoxybenzyl)amino)-6-fluoropyrazin-2-yl)-3-fluoroisonicotinic acid and 2-methyl-2,5-diazabicyclo[2.2.2]octane in Step 3. prep-HPLC conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 39% B to 64% B in 10 min; Wavelength: 254 nm/220 nm; RT1 (min): 8.82. LC purity: 97.5% (UV at 254 nm); Mass calculated for C₂₅H₂₄F₄N₆O₂[M+1]⁺, 517.2, found 517.2; Retention time: 0.626 min. ¹H NMR (400 MHz, DMSO-d₆) δ 8.67 (d, J=4.7 Hz, 1H), 8.25 (m, 2H), 7.86 (m, 1H), 7.35 (s, 2H), 7.13 (dd, J=8.8, 4.1 Hz, 2H), 6.61-6.10 (m, 1H), 4.37 (m, 2H), 3.80 (m, 1H), 3.63 (m, 2H), 2.90-2.60 (m, 3H), 2.30 (m, 3H), 2.00 (m, 1H), 1.90-1.70 (m, 1H), 1.68-1.50 (m, 2H).

### Example 4. (2-(5-amino-6-fluoropyrazin-2-yl)-6-(4-(2,2-difluoroethoxy)phenyl)-3-fluoropyridin-4-yl)(1,6-diazaspiro[3.5]nonan-6-yl)methanone

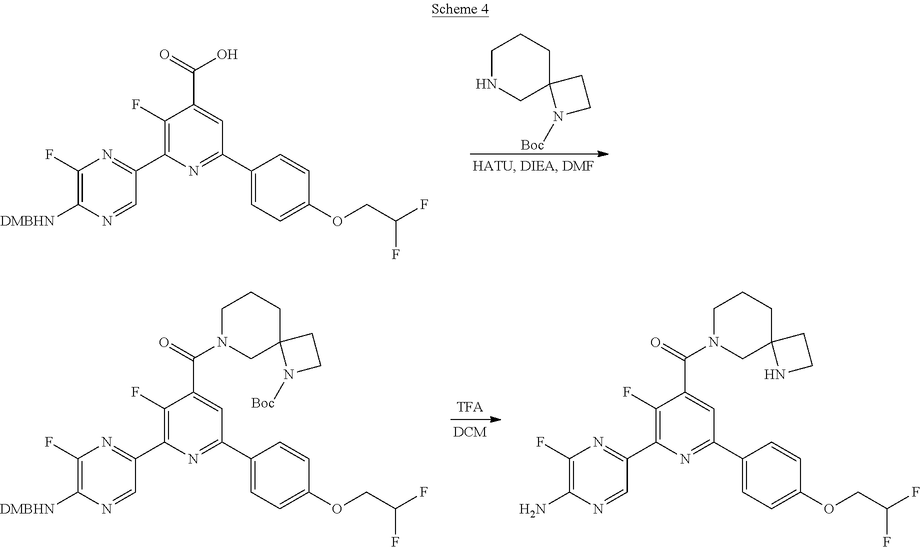

The title compound (9.6 mg) was prepared in a 24.8% overall yield as an off-white solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and tert-butyl 1,6-diazaspiro[3.5]nonane-1-carboxylate in Step 1. prep-HPLC conditions: Column: XBridge Prep OBD C18 Column, 30*150 mm, 5 m; Mobile Phase A: Water (10 nmol/L NH₄HCO₃), Mobile Phase B: ACN; Flow rate: 60 mL/min; Gradient (B %): 23% B to 48% B in 8 min; Wavelength: 254 nm/220 nm; RT1 (min): 7.73. LC purity: 97.3% (UV at 254 nm); Mass calculated for C₂₅H₂₄F₄N₆O₂[M+1]⁺, 517.2, found 517.2; Retention time: 1.377 min. ¹H NMR (400 MHz, DMSO-d₆) δ 8.67 (d, J=4.7 Hz, 1H), 8.25 (m, 2H), 7.86 (m, 1H), 7.13 (dd, J=8.8, 4.1 Hz, 2H), 6.61-6.10 (m, 1H), 4.37 (m, 2H), 4.20-3.98 (m, 1H), 3.63 (m, 2H), 3.40-3.26 (m, 3H), 2.02 (m, 1H), 1.87-1.78 (m, 2H), 1.59-1.25 (m, 3H).

### Example 5. (2-(5-amino-6-fluoropyrazin-2-yl)-6-(4-(2,2-difluoroethoxy)phenyl)-3-fluoropyridin-4-yl)(8-methyl-3,8-diazabicyclo[3.2.1]octan-3-yl)methanone

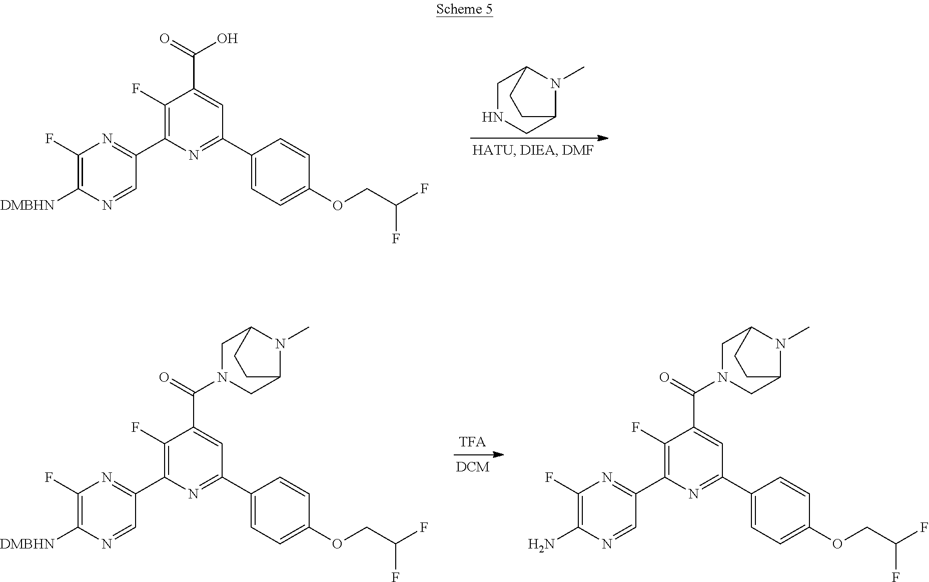

The title compound (15 mg) was prepared in a 34.9% overall yield as a white solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and 8-methyl-3,8-diazabicyclo[3.2.1]octane in Step 1. prep-HPLC conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 28% B to 53% B in 10 min; Wavelength: 254 nm/220 nm; RT1 (min): 8.52. LC purity: 99.9% (UV at 254 nm); Mass calculated for C₂₋₄H₂₅F₃N₆O₂[M+1]⁺, 517.2, found 517.2; Retention time: 0.928 min. ¹H NMR (400 MHz, DMSO-d₆) δ 8.71 (d, J=4.7 Hz, 1H), 8.16 (s, 2H), 7.94 (s, 1H), 7.37 (s, 2H), 7.18-7.11 (m, 2H), 6.50 (m, 1H), 4.41 (m, 2H), 4.21-4.12 (m, 1H), 3.31 (m, 1H), 3.23-3.17 (m, 1H), 3.11 (m, 1H), 2.99 (m, 2H), 2.19 (s, 3H), 1.93 (m, 2H), 1.50 (m, 2H).

### Example 6. (2-(5-amino-6-fluoropyrazin-2-yl)-6-(4-(2,2-difluoroethoxy)phenyl)-3-fluoropyridin-4-yl)(4-methyl-1,4-diazepan-1-yl)methanone

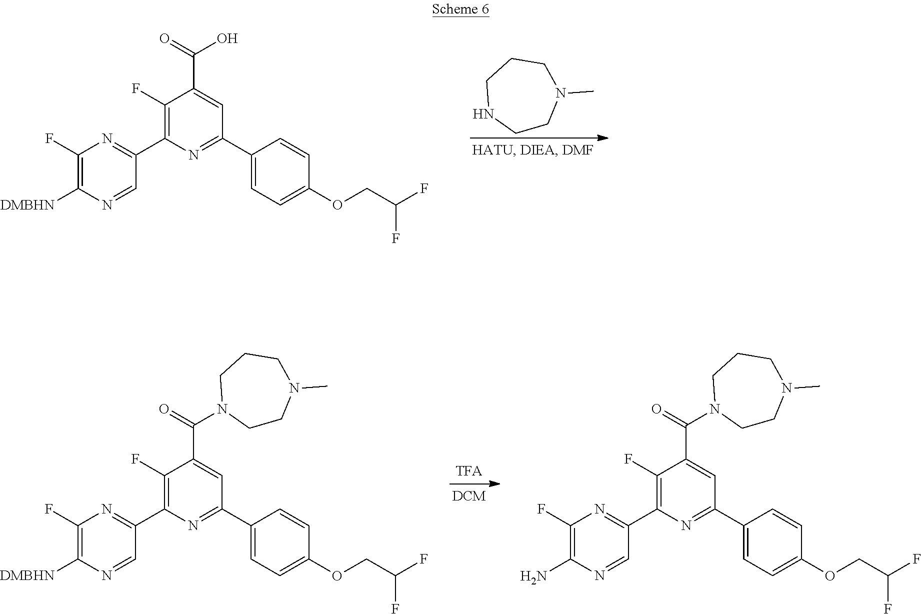

The title compound (8.4 mg) was prepared in a 26.3% overall yield as a white solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and 1-methyl-1,4-diazepane in Step 1. prep-HPLC conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 23% B to 53% B in 10 min; Wavelength: 254 nm/220 nm nm; RT1 (min): 7.8. LC purity: 99.3% (UV at 254 nm); Mass calculated for C₂₋₄H₂₄F₄N₆O₂[M+1]⁺, 505.2, found 505.1; Retention time: 1.513 min. H NMR (400 MHz, DMSO-d₆) δ 8.71 (dd, J=4.7, 2.0 Hz, 1H), 8.21-8.12 (m, 2H), 7.95 (dd, J=3.7, 1.1 Hz, 1H), 7.37 (s, 2H), 7.19-7.10 (m, 2H), 6.43 (t, J=3.5 Hz, 1H), 4.41 (m, 2H), 3.70 (m, 2H), 3.48-3.35 (m, 4H), 2.66-2.50 (m, 2H), 2.30-2.24 (d, 3H), 1.93-1.86 (m, 1H), 1.80-1.63 (m, 1H).

### Example 7. (2-(5-amino-6-fluoropyrazin-2-yl)-6-(4-(2,2-difluoroethoxy)phenyl)-3-fluoropyridin-4-yl)(2,5-diazabicyclo[2.2.2]octan-2-yl)methanone

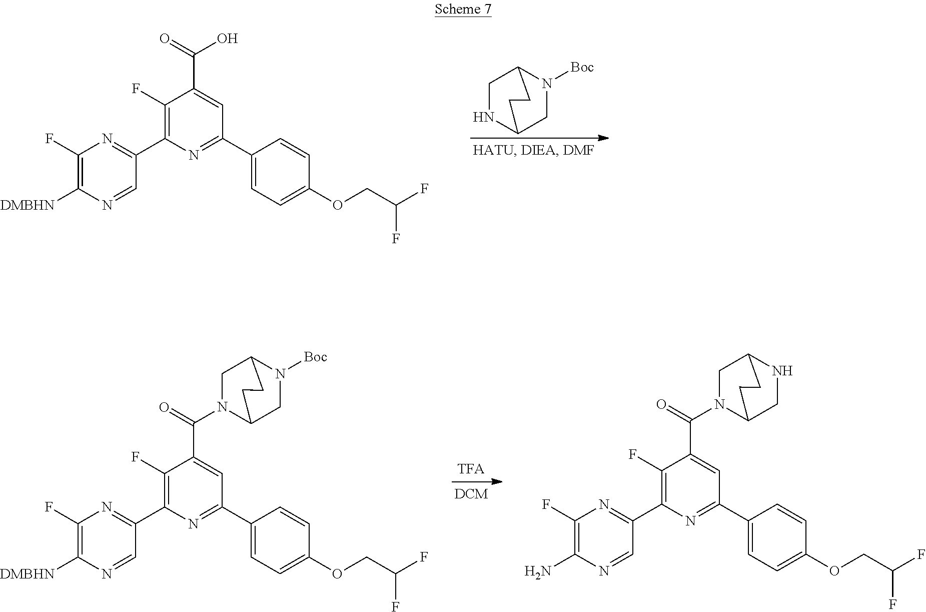

The title compound (12 mg) was prepared in a 25.9% overall yield as a white solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and tert-butyl 2,5-diazabicyclo[2.2.2]octane-2-carboxylate in Step 1. prep-HPLC conditions: Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient (B %): 18% B to 48% B in 9 min; Wavelength: 254 nm/220 nm; RT 1 (min): 8.03. LC purity: 97.1% (UV at 254 nm); Mass calculated for C₂₋₄H₂₂F₄N₆O₂[M+1]⁺ 503.2, found 503.1; Retention time: 0.601 min. ¹H NMR (400 MHz, DMSO-d₆) δ 8.75-8.67 (m, 1H), 8.17 (dd, J=8.7, 3.4 Hz, 2H), 8.00 (m, 1H), 7.37 (s, 2H), 7.15 (dd, J=8.9, 2.6 Hz, 2H), 6.43 (m, 1H), 4.50-4.22 (m, 3H), 3.60-3.56 (m, 3H), 3.09 (m, 1H), 2.88 (m, 1H), 1.80 (m, 4H).

### Example 8. (2-(5-amino-6-fluoropyrazin-2-yl)-6-(4-(2,2-difluoroethoxy)phenyl)-3-fluoropyridin-4-yl) ((1S,4S)-5-methyl-2,5-diazabicyclo [2.2.1]heptan-2-Yl) methanone

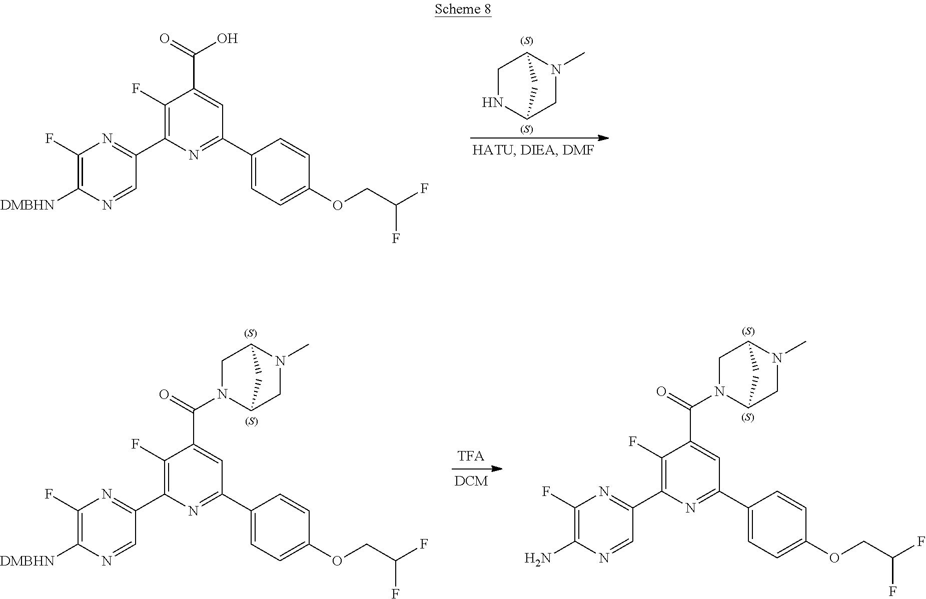

The title compound (15 mg) was prepared in a 34% overall yield as an off-white solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and (1S,4S)-2-methyl-2,5-diazabicyclo[2.2.1]heptane in Step 1. prep-HPLC conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 31% B to 61% B in 10 min; Wavelength: 254 nm/220 nm; RT1 (min): 8.82. LC purity: 99.4% (UV at 254 nm); Mass calculated for C₂₋₄H₂₂F₄N₆O₂[M+1]⁺, 503.2, found 503.1; Retention time: 1.398 min. ¹H NMR (400 MHz, DMSO-d₆) δ 8.71 (d, J=4.6 Hz, 1H), 8.23-8.11 (m, 2H), 7.99 (dd, J=11.9, 3.6 Hz, 1H), 7.37 (s, 2H), 7.25-7.04 (m, 2H), 6.43 (m, 1H), 4.76-4.02 (m, 3H), 3.63-3.47 (m, 1H), 3.42-3.36 (m, 1H), 3.03 (m, 1H), 2.71-2.59 (m, 2H), 2.33 (m, 3H), 1.93-1.69 (m, 2H).

### Example 9. (2-(5-amino-6-fluoropyrazin-2-yl)-6-(4-(2,2-difluoroethoxy)phenyl)-3-fluoropyridin-4-yl)((1R,4R)-5-methyl-2,5-diazabicyclo[2.2.1]heptan-2-yl)methanone

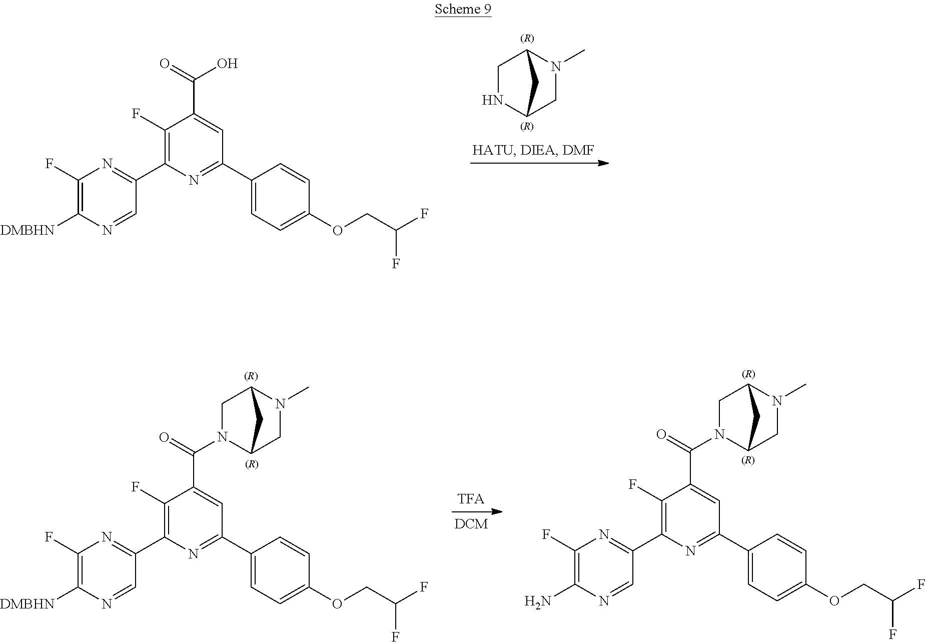

The title compound (16 mg) was prepared in a 34% overall yield as a light-yellow solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and (1R,4R)-2-methyl-2,5-diazabicyclo[2.2.1]heptane in Step 1. prep-HPLC conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 21% B to 51% B in 8 min; Wavelength: 254 nm/220 nm; RT1 (min): 7.08. LC purity: 99% (UV at 254 nm); Mass calculated for C₂₋₄H₂₂F₄N₆O₂[M+1]⁺, 503.2, found 503.2; Retention time: 0.873 min. ¹H NMR (400 MHz, DMSO-d₆) δ 8.71 (d, J=4.6 Hz, 1H), 8.17 (dd, J=8.9, 2.6 Hz, 2H), 7.98 (dd, J=11.9, 3.6 Hz, 1H), 7.37 (s, 2H), 7.19-7.11 (m, 2H), 6.59-6.27 (m, 1H), 4.76-4.00 (m, 3H), 3.63-3.43 (m, 1H), 3.42-3.34 (m, 1H), 3.24-2.83 (m, 1H), 2.72-2.57 (m, 2H), 2.33 (d, J=12.0 Hz, 3H), 1.85 (m, 1H), 1.78-1.70 (m, 1H).

### Example 10. (2-(5-amino-6-fluoropyrazin-2-yl)-6-(4-(2,2-difluoroethoxy)phenyl)-3-fluoropyridin-4-yl)(1,7-diazaspiro[3.5]nonan-7-yl)methanone

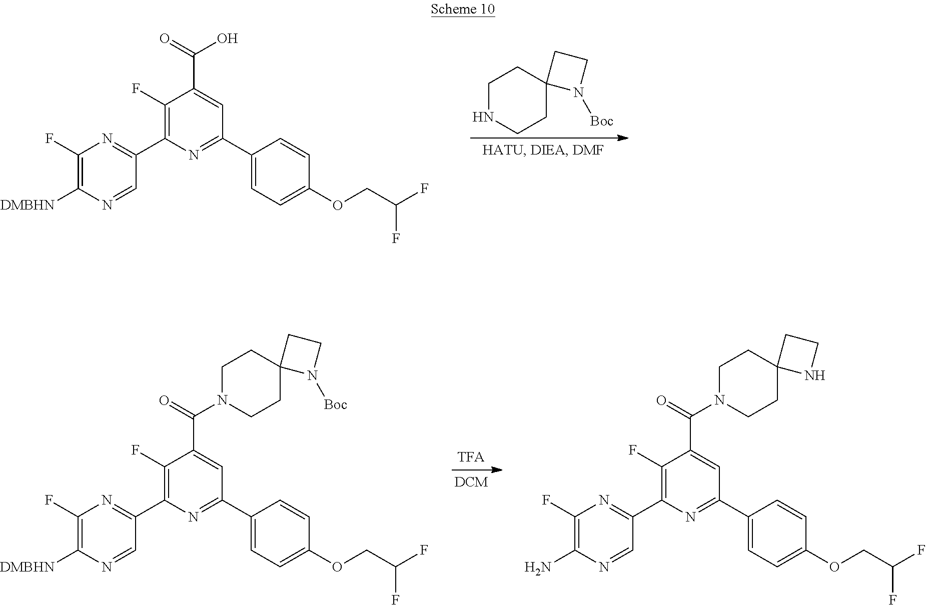

The title compound (16 mg) was prepared in a 40.3% overall yield as a white solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and tert-butyl 1,7-diazaspiro[3.5]nonane-1-carboxylate in Step 1. prep-HPLC conditions: Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient (B %): 20% B to 50% B in 10 min; Wavelength: 254 nm/220 nm; RT1 (min): 6.92. LC purity: 97.2% (UV at 254 nm); Mass calculated for C₂₅H₂₄F₄N₆O₂[M+1]⁺, 517.2, found 517.2; Retention time: 1.396. ¹H NMR (400 MHz, DMSO-d₆) δ 8.71 (d, J=4.6 Hz, 1H), 8.20-8.13 (m, 2H), 7.97 (m, 1H), 7.37 (s, 2H), 7.19-7.11 (m, 2H), 6.43 (m, 1H), 4.41-4.38 (m, 2H), 3.63 (m, 2H), 3.50-3.10 (m, 4H), 2.03 (m, 2H), 1.90-1.50 (m, 4H).

### Example 11. (2-(5-amino-6-fluoropyrazin-2-yl)-6-(4-(2,2-difluoroethoxy)phenyl)-3-fluoropyridin-4-yl)(4-methylpiperazin-1-yl)methanone

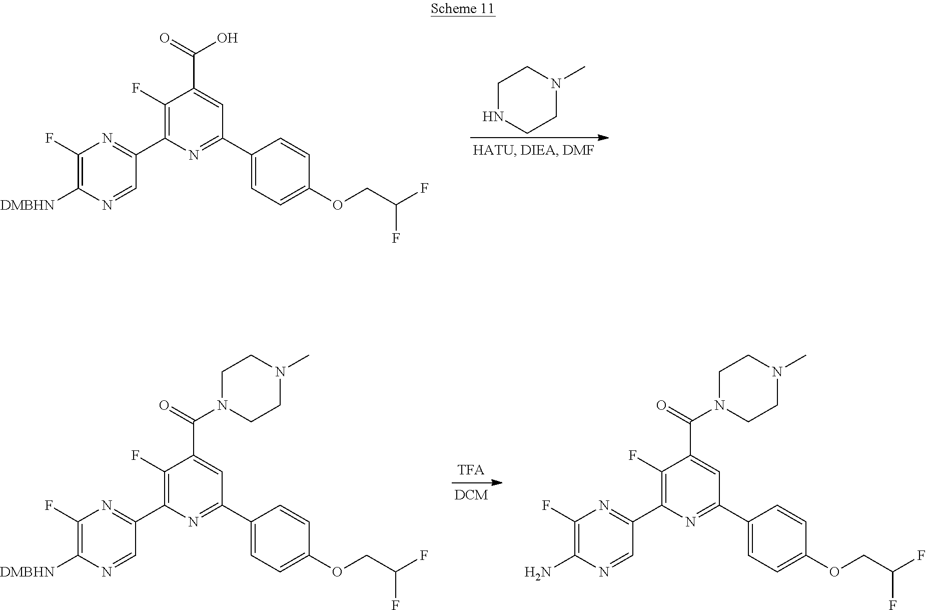

The title compound (14 mg) was prepared in a 38.4% overall yield as a white solid, following a procedure analogous to that described in Example 2, using 6-(4-(2,2-difluoroethoxy)phenyl)-2-(5-((2,4-dimethoxybenzyl)amino)-6-fluoropyrazin-2-yl)-3-fluoroisonicotinic acid and 1-methylpiperazine in Step 1. prep-HPLC conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 23% B to 48% B in 10 min; Wavelength: 254 nm/220 nm; RT 1 (min): 8.22. LC purity: 99.6% (UV at 254 nm); Mass calculated for C₂₃H₂₂F₄N₆O₂[M+1]⁺, 491.2, found 491.1; Retention time: 1.228 min. ¹H NMR (400 MHz, DMSO-d₆) δ 8.71 (d, J=4.6 Hz, 1H), 8.20-8.12 (m, 2H), 7.96 (d, J=3.6 Hz, 1H), 7.37 (s, 2H), 7.19-7.10 (m, 2H), 6.43 (m, 1H), 4.41 (m, 2H), 3.73 (m, 2H), 3.32-2.26 (m, 2H), 2.51-2.23 (m, 4H), 2.21 (s, 3H).

### Example 12. (6-(5-amino-6-fluoropyrazin-2-yl)-6′-ethoxy-5-fluoro-[2,3′-bipyridin]-4-yl)((1R,4R)-5-methyl-2,5-diazabicyclo[2.2.1]heptan-2-yl)methanone

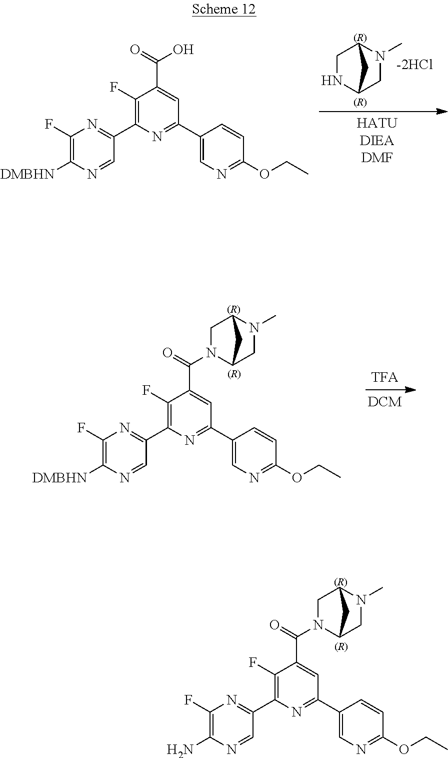

The title compound (12 mg) was prepared in a 31.9% overall yield as a white solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and (1R,4R)-2-methyl-2,5-diazabicyclo[2.2.1]heptane;dihydrochlo-ride in Step 1. prep-HPLC conditions: Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient (B %): 21% B to 51% B in 10 min; Wavelength: 254 nm/220 nm; RT1 (min): 6.27. LC purity: 99.1% (UV at 254 nm); Mass calculated for C₂₃H₂₃F₂N₇O₂[M+1]⁺, 468.2, found 468.2; Retention time: 0.567 min. ¹H NMR (400 MHz, DMSO-d₆) δ 8.95 (dd, J=2.4, 1.3 Hz, 1H), 8.74 (d, J=4.6 Hz, 1H), 8.48-8.42 (m, 1H), 8.04 (dd, J=11.7, 3.7 Hz, 1H), 7.39 (s, 2H), 6.93 (dd, J=8.7, 0.7 Hz, 1H), 4.39-4.35 (m, 3H), 3.58 (m, 1H), 3.45 (m, 1H), 3.23-2.87 (m, 1H), 2.72-2.57 (m, 2H), 2.33 (d, J=11.4 Hz, 3H), 1.90-1.69 (m, 2H), 1.36 (t, J=7.0 Hz, 3H).

### Example 13. (6-(5-amino-6-fluoropyrazin-2-yl)-6′-ethoxy-5-fluoro-[2,3′-bipyridin]-4-yl) (4-methylpiperazin-1-yl) methanone

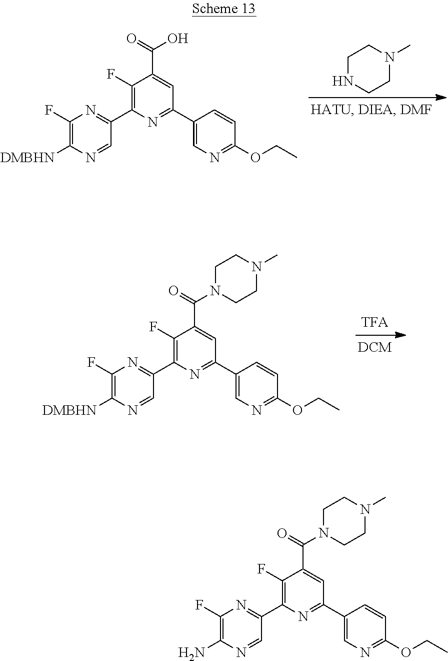

The title compound (12 mg) was prepared in a 25.8% overall yield as a white solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and 1-methylpiperazine in Step 1. prep-HPLC conditions: Column: Xbridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 16% B to 46% B in 10 min; Wavelength: 254 nm/220 nm; RT1 (min): 8.78. LC purity: 98.8% (UV at 254 nm); Mass calculated for C₂₂H₂₃F₂N₇O₂[M+1]⁺, 456.2, found 456.1; Retention time: 1.500 min. ¹H NMR (400 MHz, DMSO-d₆) δ 8.95 (d, J=2.5 Hz, 1H), 8.73 (d, J=4.7 Hz, 1H), 8.47 (dd, J=8.7, 2.6 Hz, 1H), 8.01 (d, J=3.7 Hz, 1H), 7.38 (s, 2H), 6.92 (d, J=8.7 Hz, 1H), 4.38 (q, J=7.0 Hz, 2H), 3.69 (m, 2H), 3.30 (d, J=5.6 Hz, 2H), 2.44-2.32 (m, 4H), 2.21 (s, 3H), 1.35 (t, J=7.0 Hz, 3H).

### Example 14. (6-(5-amino-6-fluoropyrazin-2-yl)-6′-ethoxy-5-fluoro-[2,3′-bipyridin]-4-yl)((1S,4S)-5-methyl-2,5-diazabicyclo[2.2.1]heptan-2-yl)methanone

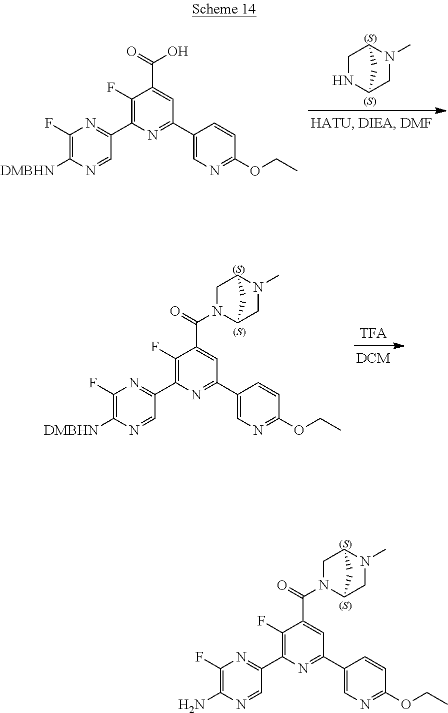

The title compound (13 mg) was prepared in a 25.4% overall yield as a white solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and (1S,4S)-2-methyl-2,5-diazabicyclo[2.2.1]heptane in Step 1. prep-HPLC conditions: Column: Xbridge Prep OBD C18, 30*150 mm, 5 μm; Mobile Phase A: Water (10 mmol/L NH₄HCO₃), Mobile Phase B: ACN; Flow rate: 60 mL/min; Gradient (B %): 20% B to 40% B in 7 min; Wavelength: 254 nm/220 nm; RT1 (min): 6.28. LC purity: 99.7% (UV at 254 nm); Mass calculated for C₂₃H₂₃F₂N₇O₂[M+1]⁺, 468.1, found 468.1; Retention time: 1.264 min. ¹H NMR (400 MHz, DMSO-d₆) δ 8.95 (d, J=2.5 Hz, 1H), 8.73 (d, J=4.6 Hz, 1H), 8.48 (m, 1H), 8.03 (dd, J=11.6, 3.7 Hz, 1H), 7.38 (s, 2H), 6.92 (dd, J=8.8, 0.7 Hz, 1H), 4.71-4.04 (m, 3H), 3.58-3.38 (m, 2H), 3.20-2.86 (m, 1H), 2.61-2.58 (m, 2H), 2.32 (m, 4H), 1.89-1.74 (m, 2H), 1.35 (m, 3H).

### Example 15. (2-(5-amino-6-fluoropyrazin-2-yl)-6-(4-(2,2-difluoroethoxy)phenyl)-3-fluoropyridin-4-yl)((1R,4R)-5-methyl-2,5-diazabicyclo[2.2.2]octan-2-yl)methanone

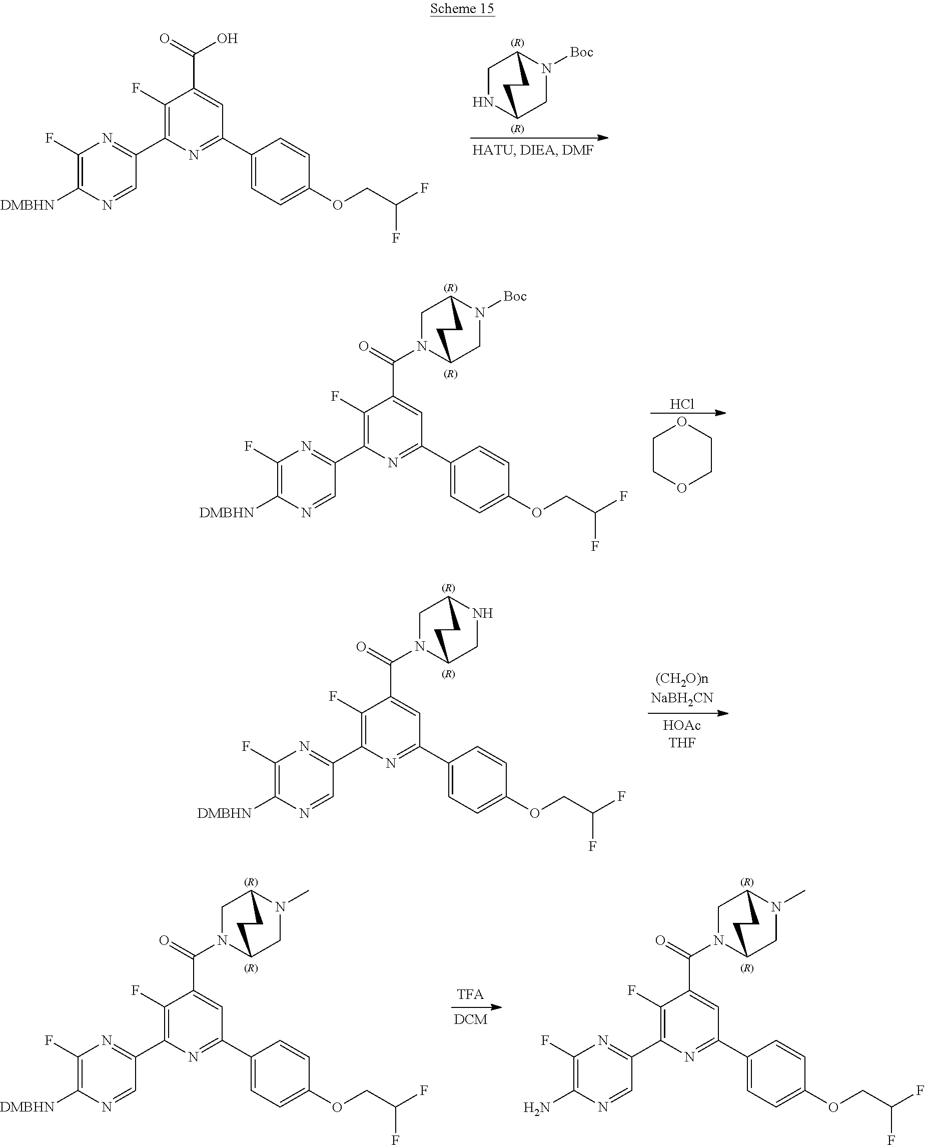

The title compound (9.2 mg) was prepared in a 13.1% overall yield as a white solid, following a procedure analogous to that described in Example 1 (Step 7 to Step 10), using tert-butyl (1R,4R)-2,5-diazabicyclo[2.2.2]octane-2-carboxylate in Step 1. prep-HPLC conditions: Column: Waters Xbridge C18 OBD Column 30*150 mm 5 μm; Mobile Phase A: water (10 mM NH₄HCO₃), Mobile Phase B: ACN; Flow rate: 60 mL/min; Gradient (B %): 22% B to 50% B in 7 min; Wavelength: 254 nm/220 nm; RT1 (min): 6.82. LC purity: 98.9% (UV at 254 nm); Mass calculated for C₂₅H₂₄F₄N₆O₂[M+1]⁺, 517.2, found 517.2; Retention time: 1.223 min. H NMR (400 MHz, DMSO) δ 8.83-8.58 (m, 1H), 8.34-8.15 (m, 2H), 8.10-7.79 (m, 1H), 7.37 (s, 2H), 7.23-6.97 (m, 2H), 6.60-6.30 (m, 1H), 4.50-4.30 (m, 2H), 4.00-3.40 (m, 3H), 2.98-2.69 (m, 3H), 2.35-2.30 (m, 3H), 2.07-1.92 (m, 1H), 1.90-1.72 (m, 1H), 1.64 (m, 2H).

### Example 16. (2-(5-amino-6-fluoropyrazin-2-yl)-6-(4-(2,2-difluoroethoxy)phenyl)-3-fluoropyridin-4-yl)((1S,4S)-5-methyl-2,5-diazabicyclo[2.2.2]octan-2-yl)methanone

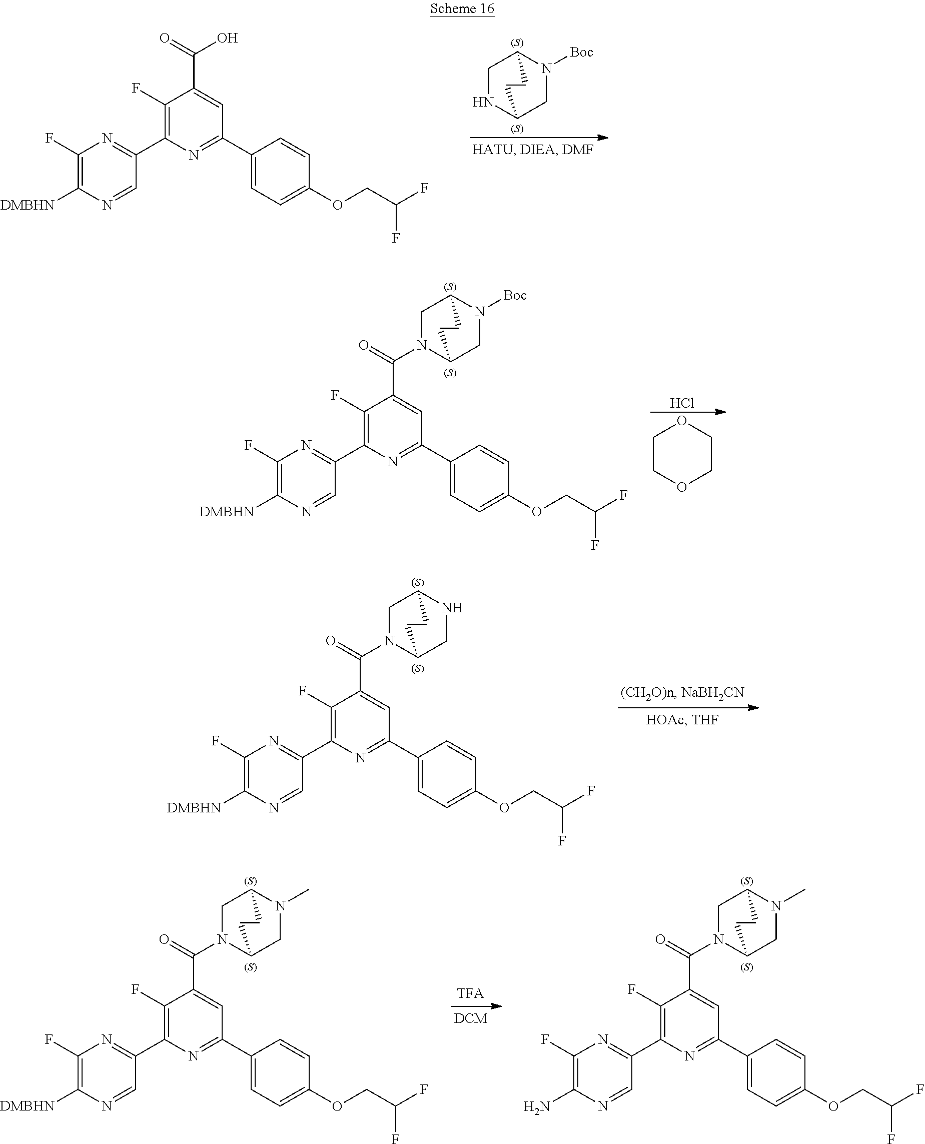

The title compound (4.73 mg) was prepared in a 12.1% overall yield as a white solid, following a procedure analogous to that described in Example 1 (Step 7 to Step 10), using tert-butyl (1S,4S)-2,5-diazabicyclo[2.2.2]octane-2-carboxylate in Step 1. prep-HPLC conditions: Column: XBridge Prep OBD C18 Column, 30*150 mm, 5 μm; Mobile Phase A: Water (10 nmol/L NH₄HCO₃), Mobile Phase B: ACN; Flow rate: 60 mL/min; Gradient (B %): 27% B to 45% B in 9 min; Wavelength: 254 nm/220 nm; RT1 (min): 7.88. LC purity: 98.9% (UV at 254 nm); C₂₅H₂₄F₄N₆O₂[M+1]⁺, 517.2, found 517.2; Retention time: 0.625 min.)H NMR (400 MHz, DMSO) δ 8.71-8.69 (m, 1H), 8.17 (dd, J=8.8, 6.2 Hz, 2H), 7.99-7.96 (m, 1H), 7.37 (s, 2H), 7.21-7.09 (m, 2H), 6.60-6.24 (m, 1H), 4.50-4.34 (m, 2H), 3.82-3.78 (m, 1H), 3.58-3.39 (m, 2H), 2.92-2.66 (m, 3H), 2.33 (s, 3H), 2.00 (m, 1H), 1.82 (m, 1H), 1.64-1.61 (m, 2H).

### Example 17. (6-(5-amino-6-fluoropyrazin-2-yl)-6′-ethoxy-5-fluoro-[2,3′-bipyridin]-4-yl)(1-methyl-1,6-diazaspiro[3.5]nonan-6-yl)methanone

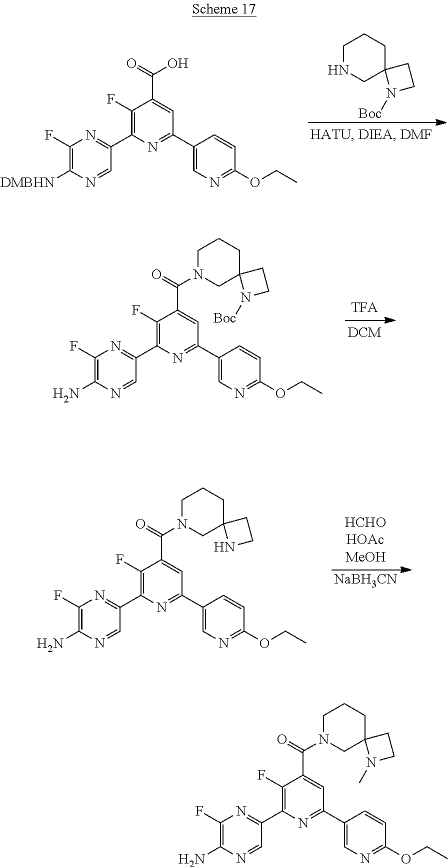

The title compound (21 mg) was prepared in a 23.6% overall yield as a solid, following a procedure analogous to that described in Example 1 (Step 7 to Step 10), using tert-butyl 1,6-diazaspiro[3.5]nonane-1-carboxylate in Step 1. Prep-HPLC conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 20% B to 50% B in 10 min; Wavelength: 254 nm/220 nm; RT1 (min): 7.27. The fractions collected were concentrated under vacuum, and the residue was re-dissolved in CH₃CN and H₂O and then was lyophilized to afford the title compound (21 mg, 0.042 mmol, 40% yield) as a white solid. LC purity: 99% (UV at 254 nm); Mass calculated for C₂₅H₂₇F₂N₇O₂[M+1]⁺ 496.2, found 496.2; Retention time: 0.892 min. ¹H NMR (400 MHz, DMSO) δ 8.94 (s, 1H), 8.74 (t, J=5.2 Hz, 1H), 8.47 (d, J=8.7 Hz, 1H), 7.94 (m, 1H), 7.39 (s, 2H), 6.92 (dd, J=8.8, 4.6 Hz, 1H), 4.61-4.00 (m, 3H), 3.31 (m, 1H), 3.27-3.08 (m, 2H), 3.04-2.79 (m, 2H), 2.23 (s, 2H), 2.08 (m, 1H), 1.99-1.40 (m, 6H), 1.35 (t, J=7.0 Hz, 3H).

### Example 18. (2-(5-amino-6-fluoropyrazin-2-yl)-6-(4-(2,2-difluoroethoxy)phenyl)-3-fluoropyridin-4-yl)(1-methyl-1,6-diazaspiro[3.5]nonan-6-yl)methanone

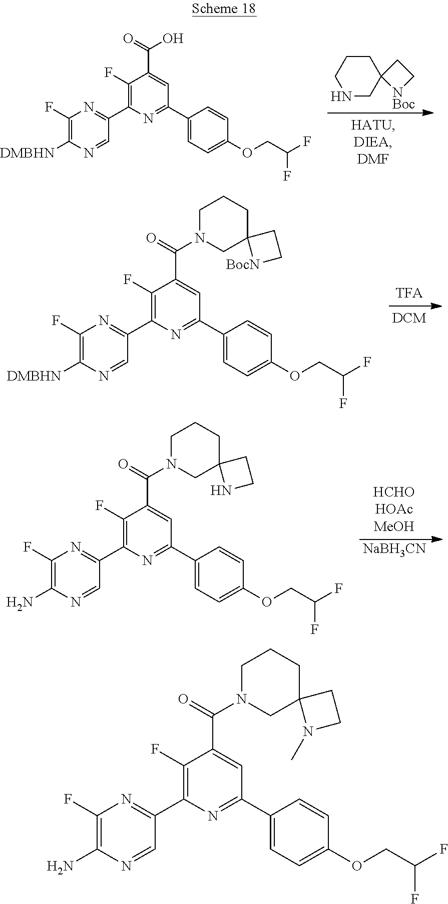

The title compound (15.15 mg) was prepared in a 25.1% overall yield as a white solid, following a procedure analogous to that described in Example 1 (Step 7 to Step 10), using tert-butyl 1,6-diazaspiro[3.5]nonane-1-carboxylate in Step 1. prep-HPLC conditions: Column: XBridge Prep OBD C18 Column, 30*150 mm, 5 μm; Mobile Phase A: water (10 nmol/L NH₄HCO₃), Mobile Phase B: ACN; Flow rate: 60 mL/min; Gradient (B %): 28% B to 45% B in 10 min; Wavelength: 254 nm/220 nm; RT1 (min): 8.99. LC purity: 99.3% (UV at 254 nm); Mass calculated for C₂₆H₂₆F₄N₆O₂[M+1]⁺, 531.2, found 531.1; Retention time: 1.258 min. ¹H NMR (400 MHz, DMSO) δ 8.71 (s, 1H), 8.16 (d, J=8.2 Hz, 2H), 8.06-7.75 (m, 1H), 7.37 (s, 2H), 7.28-7.00 (m, 2H), 6.43 (m, 1H), 4.53-4.04 (m, 3H), 3.42-3.33 (m, 1H), 3.26-3.08 (m, 2H), 3.08-2.90 (m, 1H), 2.90-2.76 (m, 1H), 2.16 (s, 3H), 1.90 (m, 2H), 1.77 (m, 1H), 1.73-1.55 (m, 2H), 1.47 (m, 1H).

### Example 19. (6-(5-amino-6-fluoropyrazin-2-yl)-6′-ethoxy-5-fluoro-[2,3′-bipyridin]-4-yl)((1S,4S)-5-methyl-2,5-diazabicyclo[2.2.2]octan-2-yl)methanone

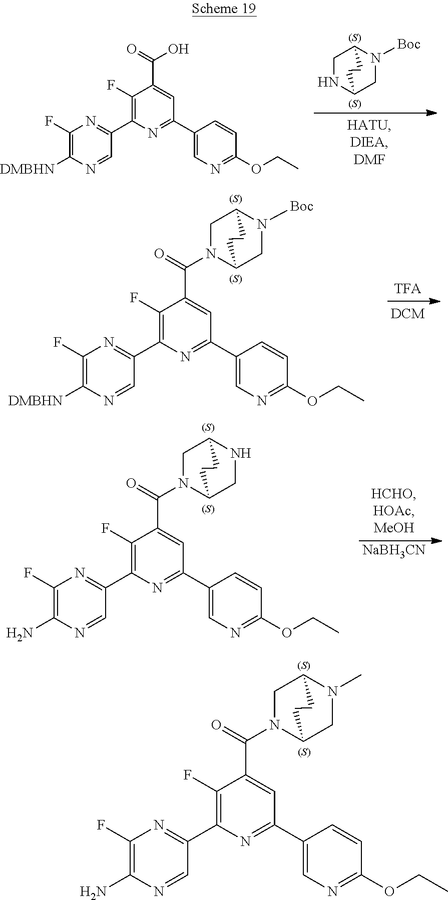

The title compound (15 mg) was prepared in a 17.8% overall yield as a white solid, following a procedure analogous to that described in in Example 1 (Step 7 to Step 10), using tert-butyl (1S,4S)-2,5-diazabicyclo[2.2.2]octane-2-carboxylate in Step 1. prep-HPLC conditions: Column: XBridge Prep OBD C18 Column, 30*150 mm, 5 μm; Mobile Phase A: Water(10 nmol/L NH₄HCO₃), Mobile Phase B: ACN; Flow rate: 60 mL/min; Gradient (B %): 31% B to 56% B in 8 min; Wavelength: 254 nm/220 nm; RT1 (min): 7.08. LC purity: 99.2% (UV at 254 nm); Mass calculated for C₂₋₄H₂₅F₂N₇O₂[M+1]⁺, 482.2, found 482.2; Retention time: 0.607 min. H NMR (400 MHz, DMSO) δ 8.95 (dd, J=9.2, 2.5 Hz, 1H), 8.73 (dd, J=8.8, 4.6 Hz, 1H), 8.52-8.43 (m, 1H), 8.03 (dd, J=6.6, 3.6 Hz, 1H), 7.38 (s, 2H), 6.92 (dd, J=8.7, 4.0 Hz, 1H), 4.46-4.33 (m, 2H), 3.86-3.77 (m, 1H), 3.51-3.41 (m, 1H), 2.87 (s, 3H), 2.31 (m, 3H), 1.99 (m, 1H), 1.82 (m, 1H), 1.64 (m, 2H), 1.40-1.31 (m, 3H).

### Example 20. 6-(5-amino-6-fluoropyrazin-2-yl)-6′-(cyclopropylmethoxy)-5-fluoro-N-(2-methyl-2-azabicyclo[2.2.2]octan-4-yl)-[2,3′-bipyridine]-4-carboxamide

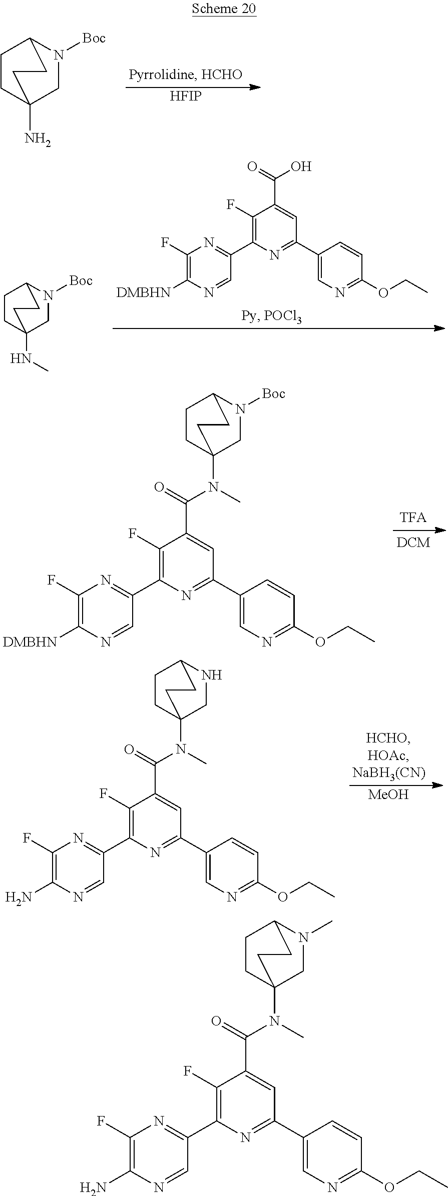

Step 1: To a solution of tert-butyl 4-amino-2-azabicyclo[2.2.2]octane-2-carboxylate (100 mg, 0.442 mmol, 1.0 equiv) and formaldehyde solution (331.7 mg, 4.420 mmol, 10.0 equiv) in HFIP (10 mL) was added pyrrolidine (314.2 mg, 4.420 mmol, 10.0 equiv) at 0° C. The resulting mixture was stirred at 50° C. for 72 h. The mixture was extracted with EA (100 mL×3), dried over anhydrous Na₂SO₄ and filtered. The filtrate was concentrated in vacuo and the residue was purified by Prep-HPLC (Column: YMC-Actus Triart C18 ExRS 250*20 mm; Mobile Phase A: Water(10 mmol/L NH₄HCO₃), Mobile Phase B: ACN′; Flow rate: 20 mL/min; Gradient (B %): isocratic 10-40B IN 10 MIN; Wavelength: 220/254 nm; RT1(min): 6.73; detector, UV 254 nm.) to afford tert-butyl 4-(methylamino)-2-azabicyclo[2.2.2]octane-2-carboxylate (70 mg, 0.292 mmol, 66% yield) as a light-yellow solid. Mass calculated for C₁₃H₂₄N₂O₂ [M+H]⁺ 241, found 241.

Step 2: To a solution of tert-butyl 4-(methylamino)-2-azabicyclo[2.2.2]octane-2-carboxylate (1.0 eq, 70 mg, 0.292 mmol) and 6-(5-((2,4-dimethoxybenzyl)amino)-6-fluoropyrazin-2-yl)-6′-ethoxy-5-fluoro-[2,3′-bipyridine]-4-carboxylic acid (preparation in Example 1, 1.0 eq, 152 mg, 0.292 mmol) in pyridine (10 mL) was treated with POCl₃ (5.0 eq, 224 mg, 1.46 mmol) at 0° C. The resulting mixture was stirred at room temperature for 24 h. The residue was purified by reversed-phase flash chromatography (column, C18 silica gel; mobile phase, MeCN in Water (0.1% FA), 0% to 80% gradient in 20 min) to afford tert-butyl 4-(6-(5-((2,4-dimethoxybenzyl)amino)-6-fluoropyrazin-2-yl)-6′-ethoxy-5-fluoro-N-methyl-[2,3′-bipyridine]-4-carboxamido)-2-azabicyclo[2.2.2]octane-2-carboxylate (45 mg, 0.06 mmol, 21% yield) as a white solid.LC purity: 70% (UV at 254 nm). Mass calculated for C₃₉H₄₅F₂N₇O₆[M+H]⁺ 746.3, found 746.3. Retention time: 1.165 min.

Step 3: To a solution of tert-butyl 4-(6-(5-((2,4-dimethoxybenzyl)amino)-6-fluoropyrazin-2-yl)-6′-ethoxy-5-fluoro-N-methyl-[2,3′-bipyridine]-4-carboxamido)-2-azabicyclo[2.2.2]octane-2-carboxylate (1.0 eq, 45 mg, 0.06 mmol) in DCM (3 mL) was treated with TFA (1 mL) at room temperature. The mixture was stirred at room temperature for 1 h. The resulting mixture was concentrated in vacuo and —residue was purified by reversed-phase flash chromatography with the following conditions: column, C18 silica gel; mobile phase, MeCN in Water (0.1% FA), 0o % to 80% gradient in 18 min; detector, UV 254 nm. The resulting mixture was concentrated under vacuum. This resulted in 6-(5-amino-6-fluoropyrazin-2-yl)-N-(2-azabicyclo[2.2.2]octan-4-yl)-6′-ethoxy-5-fluoro-N-methyl-[2,3′-bipyridine]-4-carboxamide (15 mg, 0.03 mmol, 50% yield) as a white solid. LC purity: 81% (UV at 254 nm). Mass calculated for C₂₅H₂₇F₂N₇O₂[M+H]⁺ 496, found 496. Retention time: 0.546 min.

Step 4: To a stirred solution of 6-(5-amino-6-fluoropyrazin-2-yl)-N-(2-azabicyclo[2.2.2]octan-4-yl)-6′-ethoxy-5-fluoro-N-methyl-[2,3′-bipyridine]-4-carboxamide (1.0 eq, 15 mg, 0.03 mmol) in Methanol (2 mL) were added HOAc (5.0 eq, 9.1 mg, 0.15 mmol) and formaldehyde solution (1.5 eq, 3.7 mg, 0.045 mmol). NaBH₃CN (3.0 eq, 5.6 mg, 0.091 mmol) was added in the reaction mixture and the resulting mixture was stirred for 1 h at room temperature. Upon completion the reaction mixture was diluted with water (10 mL) and extracted with EA (3*50 mL). The combined organic layers were washed with saturated NaCl solution (40 mL) and dried over anhydrous Na₂SO₄ and filtered. The filtrate was concentrated in vacuo. The residue was purified by prep-HPLC using the following conditions: Column: Xselect CSH Prep C18 OBD Column, 30*150 mm, 5 μm; Mobile Phase A: Water (0.1% FA), Mobile Phase B: ACN; Flow rate: 60 mL/min mL/min; Gradient (B %): 3% B to 33% B in 10 min; Wavelength: 254 nm/220 nm nm; RT1(min): 9.1. Pure fractions were evaporated to afford the title compound (1.3 mg, 0.002 mmol, 7.40% yield) as an off-white solid. LC purity: 99.9% (UV at 254 nm); Mass calculated for C₂₆H₂₉F₂N₇O₂[M+H]⁺ 510.2, found 510.2. Retention time: 1.368 min. H NMR (400 MHz, DMSO) δ 9.81 (s, 1H), 8.94 (d, J=2.5 Hz, 1H), 8.75 (d, J=4.7 Hz, 1H), 8.46 (dd, J=8.7, 2.5 Hz, 1H), 7.97 (d, J=3.6 Hz, 1H), 7.40 (s, 2H), 6.94 (d, J=8.7 Hz, 1H), 4.44-4.34 (m, 2H), 4.11 (s, 1H), 3.40 (s, 3H), 2.93-2.90 (m, 3H), 2.90-2.84 (m, 2H), 2.42-2.31 (m, 1H), 2.29 (s, 1H), 2.05 (s, 4H), 1.98-1.82 (m, 2H), 1.36 (t, J=7.0 Hz, 3H), 0.54 (s, 1H).

### Example 21. (5-amino-6″-ethoxy-3′,5″,6-trifluoro-[2,2′:6′,3″-terpyridin]-4′-yl)((1S,4S)-5-(methyl-d3)-2,5-diazabicyclo[2.2.2]octan-2-yl)methanone

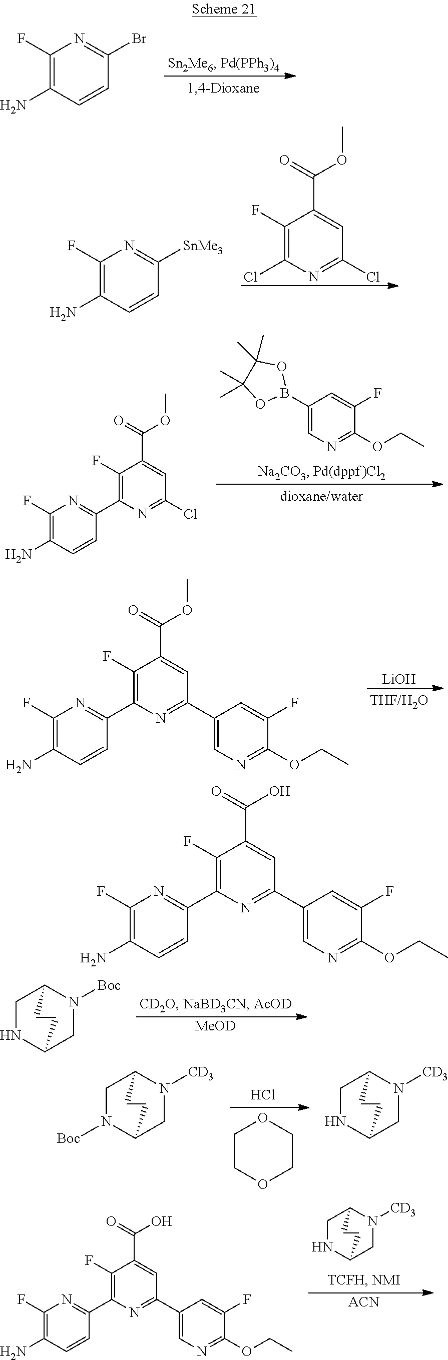

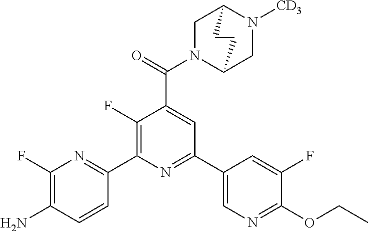 [SMILES: [2H]C([2H])([2H])N1C[C@@H]2CC[C@H]1CN2C(=O)c1cc(-c2cnc(OCC)c(F)c2)nc(-c2ccc(N)c(F)n2)c1F]

Step 1: To a solution of 6-bromo-2-fluoropyridin-3-amine (1.0 eq, 4.0 g, 20.9 mmol) and Pd(PPh₃)₄(0.1 eq, 2.4 g, 2.0 mmol) in 1,4-dioxane (40 mL) were added Sn₂Me₆ (1.5 eq, 10.3 g, 31.4 mmol). The resulting mixture was stirred at 100° C. for 2 h under nitrogen atmosphere and the resulting mixture was concentrated in vacuo. The resulting mixture was extracted with EtOAc (2×100 mL) and the combined organic layers were washed with saturated aqueous KF (3×50 mL), dried over anhydrous Na₂SO₄ and filtered. The filtrate was concentrated in vacuo. The residue was purified via silica gel chromatography using a gradient of 0 to 30% EtOAc in PE to afford 2-fluoro-6-(trimethylstannyl)pyridin-3-amine (3.80 g, 13.8 mmol, 66% yield) as a yellow solid. LC purity: 57.0% (UV at 254 nm); Mass calculated for C₈H₁₃FN₂Sn[M+1]⁺, 277, found 277; Retention time: 0.838 min.

Step 2: To a solution of methyl 2,6-dichloro-3-fluoroisonicotinate (1.0 eq, 500 mg, 2.2 mmol) and methyl 2-fluoro-6-(trimethylstannyl)pyridin-3-amine (0.8 eq, 491 mg, 1.7 mmol) in 1,4-dioxane (6 mL) were added PCy₃ (0.3 eq, 18 mg, 0.6 mmol) and Pd(OAc)₂ (0.2 eq, 100 mg, 0.4 mmol). The resulting mixture was stirred at 70° C. under nitrogen atmosphere for overnight. The resulting mixture was concentrated in vacuo. The residue was purified via reversed phase flash chromatography (C18 silica gel) using a gradient of 50% to 70% ACN in water (with 10 mmol/L NH₄HCO₃) to afford methyl 5′-amino-6-chloro-3,6′-difluoro-[2,2′-bipyridine]-4-carboxylate (540 mg, 1.8 mmol, 80% yield) as a yellow solid. LC purity: 40.1% (UV at 254 nm); Mass calculated for C₁₂H₈F₂N₃O₂[M+1]⁺, 300.1, found 300.1; Retention time: 0.743 min.

Step 3: To a solution of methyl 5′-amino-6-chloro-3,6′-difluoro-[2,2′-bipyridine]-4-carboxylate (1.0 eq, 446 mg, 1.6 mmol) and 2-ethoxy-3-fluoro-5-(4,4,5,5-tetramethyl-1,3,2-dioxaborolan-2-yl)pyridine (1.0 eq, 500 mg, 1.6 mmol) in 1,4-dioxane (6 mL) and Water (0.6 mL) were added Na₂CO₃ (3.0 eq, 531 mg, 5.0 mmol) and Pd(dppf)Cl₂ (0.1 eq, 122 mg, 0.1 mmol). The resulting mixture was stirred at 80° C. for 2 h under nitrogen atmosphere. The resulting mixture was extracted with EtOAc (2×100 mL) and the combined organic layers were washed with brine (1×30 mL), dried over anhydrous Na₂SO₄ and filtered. The filtrate was concentrated in vacuo to afford crude product methyl 5-amino-6″-ethoxy-3′,5″,6-trifluoro-[2,2′:6′,3″-terpyridine]-4′-carboxylate (600 mg, 1.4 mmol, 89% yield) as a light yellow solid which was used directly in the next step without further purification. LC purity: 82.1% (UV at 254 nm); Mass calculated for C₁₉H₁₅F₃N₄O₃[M+1]⁺, 405.1, found 405.1; Retention time: 0.935 min.

Step 4: To a stirred solution of methyl 5-amino-6″-ethoxy-3′,5″,6-trifluoro-[2,2′:6′,3″-terpyridine]-4′-carboxylate (1.0 eq, 600 mg, 1.4 mmol) in THF (6 mL) and Water (2 mL) was added LiGH (3.00 eq, 107 mg, 4.45 mmol). The resulting mixture was stirred at 20° C. for 2 h. The resulting mixture was extracted with EtOAc (2×100 mL) and the combined organic layers were washed with brine (1×30 mL), dried over anhydrous Na₂SO₄ and filtered. The filtrate was concentrated in vacuo to afford 5-amino-6″-ethoxy-3′,5″,6-trifluoro-[2,2′:6′,3″-terpyridine]-4′-carboxylic acid (400 mg, 1.0 mmol, 69% yield) as a light yellow solid which was used directly in the next step without further purification. LC purity: 76.0% (UV at 254 nm); Mass calculated for C₁₈H₁₃F₃N₄O₃[M+1]⁺, 391.1, found 391.1; Retention time: 0.855 min.

Step 5: To a solution of tert-butyl (1S,4S)-2,5-diazabicyclo[2.2.2]octane-2-carboxylate (1.0 eq, 300 mg, 1.4 mmol) in MeOD (5 mL) were added CD₂0 (1.2 eq, 219 mg, 1.7 mmol) and AcOD (3.0 eq, 271 mg, 4.2 mmol), the resulting mixture was stirred at room temperature for 20 min. NaBD₃CN (2.0 eq, 186 mg, 2.8 mmol) was added to above mixture at 0° C. and the reaction was stirred at room temperature for 1 h. The reaction was quenched with water (50 mL) and extracted with ethyl acetate (2×50 mL). The organic layers were washed with brine (50 mL), dried over anhydrous sodium sulfate, filtered and concentrated in vacuo. The residue was purified by reversed phase flash chromatography using a gradient of 20% to 50% ACN in water (with 10 mmol/L NH₄HCO₃) to afford tert-butyl (1S,4S)-5-(methyl-d3)-2,5-diazabicyclo[2.2.2]octane-2-carboxylate (220 mg, 1.0 mmol, 67% yield) as a colorless oily liquid. LC purity: 80% (UV at 254 nm); Mass calculated for C₁₂H₁₉D₃N₂O₂[M+1]⁺ 230.3, found 230.3; Retention time: 0.707 min.

Step 6: To a solution of tert-butyl (1S,4S)-5-(methyl-d3)-2,5-diazabicyclo[2.2.2]octane-2-carboxylate (1.0 eq, 230 mg, 1.0 mmol) in 1,4-dioxane (4 mL) was added HCl (4 mL). The resulting mixture was stirred at room temperature for 1 h, and concentrated in vacuo. The resulting crude product was used directly in the next step without further purification. LC purity: NA (UV at 254 nm); Mass calculated for C₇H₁₁D₃N₂ [M+1]⁺, 130.2, found 130.1; Retention: 0.157.

Step 7: To a solution of 5-amino-6″-ethoxy-3′, 5″,6-trifluoro-[2,2′:6′,3″-terpyridine]-4′-carboxylic acid (1.0 eq, 60.0 mg, 0.1 mmol), (1S,4S)-2-(methyl-d3)-2,5-diazabicyclo[2.2.2]octane (1.1 eq, 21.8 mg, 0.1 mmol) in DMF (1 mL) were added HATU (1.5 eq, 87.6 mg, 0.2 mmol) and DIEA (3.0 eq, 59.5 mg, 0.5 mmol). The resulting solution was stirred at room temperature for 1 h. The resulting solution was purified by Prep-HPLC using the following conditions: Column: X-Select Prep OBD C18 Column, 30*150 mm, 5 m; Mobile Phase A: Water(0.1% FA), Mobile Phase B: ACN; Flow rate: 60 mL/min; Gradient (B %): 13% B to 40% B in 10 min; Wavelength: 254 nm/220 nm; RT1(min): 7.58. Pure fractions were evaporated to afford the title compound (43.3 mg, 0.1 mmol, 51% yield) as an off-white solid.LC purity: 99% (UV at 254 nm); Mass calculated for C₂₅H₂₂D₃F₃N₆O₂ [M+1]⁺ 502.2, found 502.2; Retention time: 1.346 min. ¹H NMR (400 MHz, DMSO-d₆) δ 8.79 (dd, J=10.2, 2.0 Hz, 1H), 8.46-8.36 (m, 1H), 8.10-8.01 (m, 2H), 7.35-7.25 (m, 1H), 6.00 (s, 2H), 4.54-4.43 (m, 2H), 3.92-3.90 (m, 1H), 3.84-3.44 (m, 2H), 3.32-3.03 (m, 3H), 2.09-2.07 (m, 1H), 1.99-1.59 (m, 3H), 1.44-1.35 (m, 3H).

### Example 22. (5-amino-6″-(ethoxy-d5)-3′,6-difluoro-[2,2′:6′,3″-terpyridin]-4′-yl)((1S,4S)-5-(methyl-d3)-2,5-diazabicyclo[2.2.2]octan-2-yl)methanone

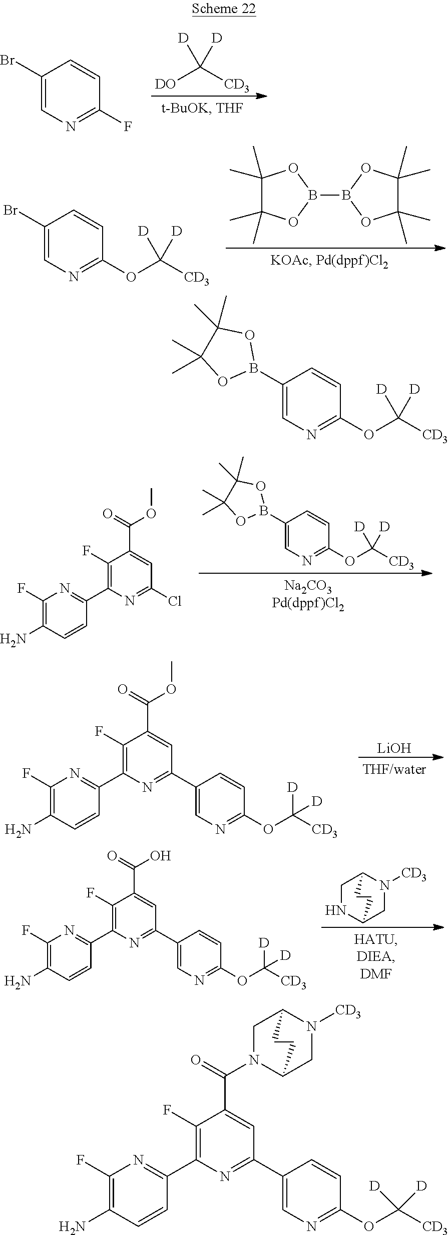

Step 1: To a solution of ethan-1,1,2,2,2-d₅-1-ol-d (1.0 eq, 280 mg, 5.4 mmol) and 5-bromo-2-fluoropyridine (1.5 eq, 1418 mg, 8.1 mmol) in THF (30 mL) was added t-BuOK (2.0 eq, 1204 mg, 10.7 mmol) at 0° C. The reaction was stirred at room temperature for 12 h. The resulting mixture was diluted with iced-water and extracted with EtOAc. The combined organic layers were washed with brine, dried over anhydrous Na₂SO₄, filtered, and the filtrate was concentrated in vacuo. The residue was purified via silica gel chromatography using a gradient of 0 to 15% EtOAc in PE to afford 5-bromo-2-(ethoxy-d₅)pyridine (370 mg, 1.8 mmol, 33% yield) as a colorless oil. LC purity: 70% (UV at 254 nm); Mass calculated for C₇H3D₅BrNO [M+1]⁺ 207.0, found 207.0; Retention time: 0.819 min.

Step 2: To a solution of 4,4,4′,4′,5,5,5′,5′-octamethyl-2,2′-bi(1,3,2-dioxaborolane) (2.0 eq, 907.0 mg, 3.6 mmol) and 5-bromo-2-(ethoxy-d5)pyridine (1.0 eq, 370.0 mg, 1.8 mmol) in 1,4-dioxane (20 mL) were added KOAc (2.0 eq, 350.0 mg, 3.6 mmol) and Pd(dppf)Cl₂ (0.1 eq, 146.0 mg, 0.2 mmol) under N₂ at room temperature. The reaction was stirred at 80° C. for 2 h. The resulting mixture was filtered, and the filtrate was concentrated to afford crude 2-(ethoxy-d5)-5-(4,4,5,5-tetramethyl-1,3,2-dioxaborolan-2-yl)pyridine (370.0 mg, 1.0 mmol, 57% yield) which was used directly in the next step without further purification. LC purity: 83% (UV at 254 nm); Mass calculated for C₁₃H₁₅D₅BNO₃ [M+1]⁺ 255.2, found 255.1; Retention time: 1.124 min.

Step 3: To a solution of methyl 5′-amino-6-chloro-3,6′-difluoro-[2,2′-bipyridine]-4-carboxylate (1.0 eq, 150.0 mg, 0.5 mmol) and 2-(ethoxy-d5)-5-(4,4,5,5-tetramethyl-1,3,2-dioxaborolan-2-yl)pyridine (1.3 eq, 165.0 mg, 0.6 mmol) in 1,4-dioxane (10 mL) and water (1 mL) were added Na₂CO₃ (3.0 eq, 159.0 mg, 1.5 mmol) and Pd(dppf)Cl₂ (0.1 eq, 41.0 mg, 0.1 mmol) under N₂ at room temperature. The reaction was stirred at 80° C. for 1 h. The resulting mixture was purified via reversed phase flash chromatography using a gradient of 40% to 70% ACN in water (with 10 mmol/L NH₄HCO₃) to afford methyl 5-amino-6″-(ethoxy-d5)-3′,6-difluoro-[2,2′:6′,3″-terpyridine]-4′-carboxylate (100.0 mg, 0.2 mmol, 48% yield) as a yellow solid. LC purity:93% (UV at 254 nm); Mass calculated for C₁₃H₁₅D₅BNO3 [M+1]⁺ 392.2, found 392.0; Retention time: 0.665 min.

Step 4: To a solution of methyl 5-amino-6″-(ethoxy-d5)-3′,6-difluoro-[2,2′:6′,3″-terpyridine]-4′-carboxylate (1.0 eq, 100.0 mg, 0.2 mmol) in THF (2 mL) and water (2 mL) was added LiGH H₂O (3.0 eq, 32.0 mg, 0.7 mmol) at room temperature. The reaction was stirred at room temperature for 1 h. The resulting mixture was purified via reverse phase flash chromatography using a gradient of 20% to 50% ACN in water (with 0.1% FA) to afford 5-amino-6″-(ethoxy-d5)-3′,6-difluoro-[2,2′:6′,3″-terpyridine]-4′-carboxylic acid (100.0 mg, 0.2 mmol, 96% yield) as a yellow solid. LC purity: 92% (UV at 254 nm); Mass calculated for C₁₈H9D₅F₂N₄O₃[M+1]⁺ 378.1, found 378.0; Retention time: 0.609 min.

Step 5: To a solution of 5-amino-6″-(ethoxy-d5)-3′,6-difluoro-[2,2′:6′,3″-terpyridine]-4′-carboxylic acid (1.0 eq, 60.0 mg, 0.1 mmol), (1S,4S)-2-(methyl-d3)-2,5-diazabicyclo[2.2.2]octane (1.1 eq, 22.6 mg, 0.2 mmol) in DMF (1 mL) were added HATU (1.5 eq, 90.6 mg, 0.2 mmol) and DIEA (3.0 eq, 61.5 mg, 0.5 mmol). The resulting solution was stirred at room temperature for 1 h. The resulting mixture was purified by Prep-HPLC using the following conditions: Column: XBridge Prep Shield RP18 OBD C18 Column, 30*150 mm, 5 μm; Mobile Phase A: Water (10 nmol/LNH₄HCO₃+0.05% NH₃·H₂O), Mobile Phase B: ACN; Flow rate: 60 mL/min; Gradient (B %): 20% B to 50% B in 10 min; Wavelength: 254 nm/220 nm; RT1 (min): 9.27. Pure fractions were evaporated to afford the title compound (29.5 mg, 0.1 mmol, 37% yield) as an off-white solid. LC purity: 99.3% (UV at 254 nm); Mass calculated for C₂₅H₁₈D₈F₂N₆O₂ [M+1]⁺ 489.3, found 489.2; Retention time: 1.162 min. ¹H NMR (400 MHz, DMSO) δ 8.99-8.91 (m, 1H), 8.46-8.43 (m, 1H), 8.05-7.93 (m, 2H), 7.35-7.25 (m, 1H), 6.92 (dd, J=8.7, 4.0 Hz, 1H), 5.97 (s, 2H), 3.86-3.77 (m, 1H), 3.60 (d, J=10.7 Hz, 1H), 3.51-3.40 (m, 1H), 2.87-2.85 (m, 2H), 2.79-2.60 (m, 1H), 2.15-1.72 (m, 2H), 1.64-1.62 (m, 2H).

### Example 23. (5-amino-6″-ethoxy-3′,6-difluoro-[2,2′:6′,3″-terpyridin]-4′-yl)((1S,4S)-5-(methyl-d3)-2,5-diazabicyclo[2.2.2]octan-2-yl)methanone

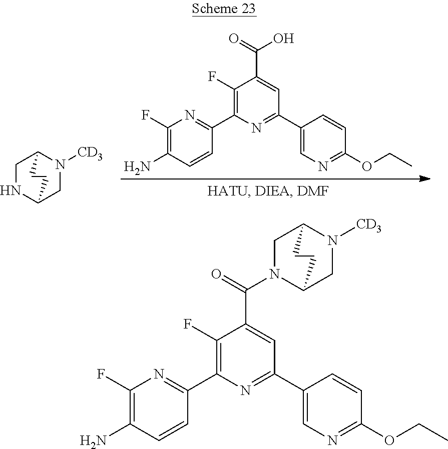 [SMILES: [2H]C([2H])([2H])N1C[C@@H]2CC[C@H]1CN2 ; CCOc1ccc(-c2cc(C(=O)O)c(F)c(-c3ccc(N)c(F)n3)n2)cn1 ; [2H]C([2H])([2H])N1C[C@@H]2CC[C@H]1CN2C(=O)c1cc(-c2ccc(OCC)nc2)nc(-c2ccc(N)c(F)n2)c1F]

The title compound (38.8 mg) was prepared in a 50% yield as an off-white solid, following a procedure analogous to that described in Example 22 (Step 5). Prep-HPLC conditions: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 21% B to 46% B in 8 min; Wavelength: 254 nm/220 nm; RT1 (min): 7.26. LC purity: 98% (UV at 254 nm); Mass calculated for C₂₅H₂₃D₃F₂N₆O₂ [M+1]⁺ 484.5, found 484.2; Retention time: 1.179 min. ¹H NMR (400 MHz, DMSO-d₆) δ 8.95 (dd, J=8.9, 2.5 Hz, 1H), 8.51-8.42 (m, 1H), 8.05-7.95 (m, 2H), 7.35-7.25 (m, 1H), 6.96-6.88 (m, 1H), 5.97 (s, 2H), 4.46-4.33 (m, 2H), 3.86-3.77 (m, 1H), 3.60-3.58 (m, 1H), 3.49-3.41 (m, 1H), 2.87-2.84 (m, 2H), 2.74-2.63 (m, 1H), 1.99-1.95 (m, 1H), 1.93-1.74 (m, 1H), 1.63-1.60 (m, 2H), 1.35 (t, J=6.4 Hz, 3H).

### Example 24. (6-(5-amino-6-fluoropyrazin-2-yl)-6′-(ethoxy-d5)-5-fluoro-[2,3′-bipyridin]-4-yl)((1S,4S)-5-(methyl-d3)-2,5-diazabicyclo[2.2.2]octan-2-yl)methanone

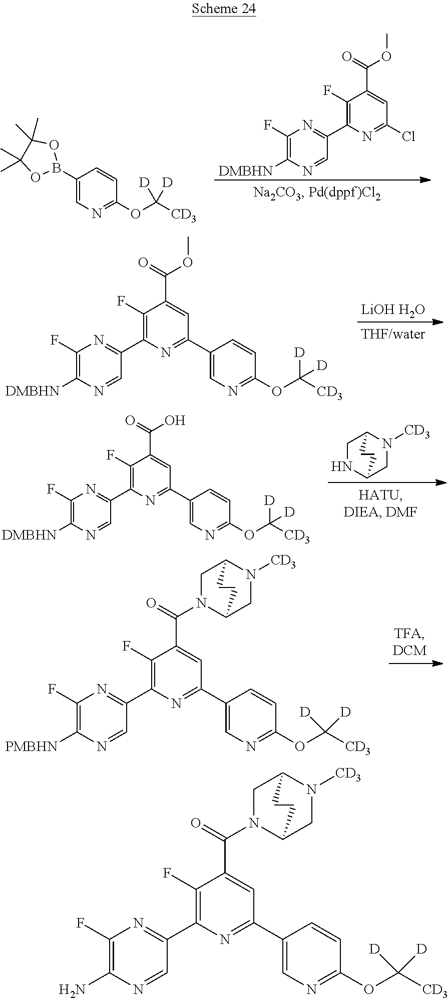

Step 1: To a solution of methyl 6-chloro-2-(5-((2,4-dimethoxybenzyl)amino)-6-fluoropyrazin-2-yl)-3-fluoroisonicotinate (1.0 eq, 200 mg, 0.4 mmol) and 2-(ethoxy-d5)-5-(4,4,5,5-tetramethyl-1,3,2-dioxaborolan-2-yl)pyridine (1.5 eq, 169 mg, 0.6 mmol) in 1,4-dioxane (10 mL) and water (1 mL) were added Na₂CO₃ (3.0 eq, 141 mg, 1.3 mmol) and Pd(dppf)Cl₂ (0.1 eq, 36 mg, 0.04 mmol). The reaction was stirred at 80° C. for 1 h under N₂. The resulting mixture was purified via reversed phase flash chromatography (C18 silica gel) using a gradient of 50% to 80% ACN in water (with 10 mmol/L NH₄HCO₃) to afford methyl 6-(5-((2,4-dimethoxybenzyl)amino)-6-fluoropyrazin-2-yl)-6′-(ethoxy-d5)-5-fluoro-[2,3′-bipyridine]-4-carboxylate (110 mg, 0.1 mmol, 34% yield) as a yellow solid. LC purity:75.6% (UV at 254 nm); Mass calculated for C₂₇H₂₀D₅F₂N₅O₅ [M+1]⁺, 543.2, found 543.2; Retention time: 1.045 min.

Step 2: To a solution of methyl 6-(5-((2,4-dimethoxybenzyl)amino)-6-fluoropyrazin-2-yl)-6′-(ethoxy-d5)-5-fluoro-[2,3′-bipyridine]-4-carboxylate (1.0 eq, 110 mg, 0.1 mmol) in THF (5 mL) and water (0.5 mL) was added LiOH·H₂O (3.0 eq, 56 mg, 1.3 mmol). The reaction was stirred at room temperature for 1 h. The resulting mixture was purified via reverse phase flash chromatography (C18 silica gel) using a gradient of 10% to 40% ACN in water (with 0.1% FA) to afford 6-(5-((2,4-dimethoxybenzyl)amino)-6-fluoropyrazin-2-yl)-6′-(ethoxy-d5)-5-fluoro-[2,3′-bipyridine]-4-carboxylic acid (100 mg, 0.1 mmol, 88% yield) as a yellow solid. LC purity:84.9% (UV at 254 nm); Mass calculated for C₂₆H₁₈D₅F₂N₅O₅ [M+1]⁺, 529.2, found 529.2; Retention time: 0.965 min.

Step 3: To a solution of (1S,4S)-2-(methyl-d3)-2,5-diazabicyclo[2.2.2]octane (2.0 eq, 52 mg, 0.4 mmol) and 6-(5-((2,4-dimethoxybenzyl)amino)-6-fluoropyrazin-2-yl)-6′-(ethoxy-d5)-5-fluoro-[2,3′-bipyridine]-4-carboxylic acid (1.0 eq, 100 mg, 0.2 mmol) in DMF (3 mL) were added HATU (1.5 eq, 115 mg, 0.3 mmol) and DIEA (3.0 eq, 78 mg, 0.6 mmol), and the reaction was stirred at room temperature for overnight. The reaction mixture was quenched with water, and extracted with EtOAc (2×100 mL). The combined organic layers were washed with brine (1×30 mL), dried over anhydrous Na₂SO₄ and filtered. The filtrate was concentrated in vacuo to afford crude product (6-(5-((2,4-dimethoxybenzyl)amino)-6-fluoropyrazin-2-yl)-6′-(ethoxy-d5)-5-fluoro-[2,3′-bipyridin]-4-yl)((1S,4S)-5-(methyl-d3)-2,5-diazabicyclo[2.2.2]octan-2-yl)methanone (95 mg, 0.1 mmol, 77% yield) as a light yellow oil which was used directly in the next step without further purification. LC purity: 69.8% (UV at 254 nm); Mass calculated for C₃₃H₂₇D₈F₂N₇O₄ [M+1]⁺, 640.3, found 640.3; Retention time: 0.884 min.

Step 4: To a solution of (6-(5-((2,4-dimethoxybenzyl)amino)-6-fluoropyrazin-2-yl)-6′-(ethoxy-d5)-5-fluoro-[2,3′-bipyridin]-4-yl)((1S,4S)-5-(methyl-d3)-2,5-diazabicyclo[2.2.2]octan-2-yl)methanone (1.0 eq, 95 mg, 0.1 mmol) in DCM (2.5 mL) was added TFA (2.5 mL) at 0° C. The resulting mixture was stirred at room temperature for 1 h. The mixture was concentrated in vacuo, and was treated with 1 mL NH₄OH at 0° C. and concentrated in vacuo. The residue was purified by Prep-HPLC with following condition: (Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 27% B to 53% B in 10 min; Wavelength: 254 nm/220 nm; RT1 (min): 8.28). The pure fractions were concentrated and lyophilized to afford title compound (20 mg, 0.04 mmol, 27% yield) as a white solid. LC purity: 99.0% (UV at 254 nm); Mass calculated for C₂₋₄H₁₇D₈F₂N₇O₂ [M+1]⁺, 490.3, found 490.3; Retention time: 0.878 min. ¹H NMR (400 MHz, MeOD) δ 8.85 (dd, J=9.2, 2.5 Hz, 1H), 8.67 (dd, J=6.0, 4.3 Hz, 1H), 8.41-8.39 (m, 1H), 7.88 (dd, J=13.7, 3.7 Hz, 1H), 6.93-6.85 (m, 1H), 4.02-4.00 (m, 1H), 3.80-3.57 (m, 2H), 3.20-3.17 (m, 2H), 3.04-2.96 (m, 1H), 2.26-2.23 (m, 1H), 2.00-1.97 (m, 1H), 1.94-1.73 (m, 2H).

### Example 25. (5-amino-6″-ethoxy-3′,5″,6-trifluoro-[2,2′:6′,3″-terpyridin]-4′-yl)((1S,4S)-5-methyl-2,5-diazabicyclo[2.2.2]octan-2-yl)methanone

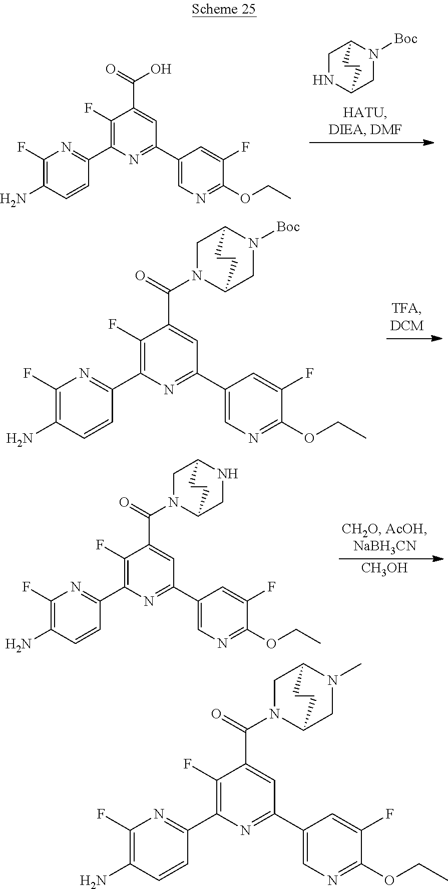

Step 1: To a solution of 5-amino-6″-ethoxy-3′, 5″,6-trifluoro-[2,2′: 6′,3″-terpyridine]-4′-carboxylic acid (preparation in Example 21, 1.0 eq, 70.0 mg, 0.2 mmol), tert-butyl (1S,4S)-2,5-diazabicyclo[2.2.2]octane-2-carboxylate (1.1 eq, 41.9 mg, 0.2 mmol) in DMF (3 mL) were added HATU (1.5 eq, 102.2 mg, 0.3 mmol) and DIEA (3.0 eq, 69.4 mg, 0.5 mmol). The resulting solution was stirred at room temperature for 1 h. The reaction was then quenched with water and extracted with EtOAc. The organic layers were washed with brine, dried over anhydrous sodium sulfate, filtered and concentrated in vacuo. The product was used in the next step directly without further purification. LC purity: 80% (UV at 254 nm); Mass calculated for C₂₉H₃₁F₃N₆O₄[M+1]⁺ 585.6, found 585.3; Retention time: 0.743 min.

Step 2: To a solution of tert-butyl (1S,4S)-5-(5-amino-6″-ethoxy-3′,5″,6-trifluoro-[2,2′:6′,3″-terpyridine]-4′-carbonyl)-2,5-diazabicyclo[2.2.2]octane-2-carboxylate (1.0 eq, 80.0 mg, 0.1 mmol) in DCM (5 mL) was added TFA (0.5 mL). The mixture was stirred at room temperature for 1 h. The resulting solution was concentrated in vacuo. The residue was added DMSO, filtered and purified via reversed phase flash chromatography using a gradient of 40% to 70% ACN in water (with 5 mM NH₄HCO₃) to afford (5-amino-6″-ethoxy-3′,5″,6-trifluoro-[2,2′:6′,3″-terpyridin]-4′-yl)((1S,4S)-2,5-diazabicyclo[2.2.2]octan-2-yl)methanone (60.0 mg, 0.1 mmol, 103% yield) as a light yellow solid. LC purity: 87% (UV at 254 nm); Mass calculated for C₂₋₄H₂₃F₃N₆O₂[M+1]⁺ 485.5, found 485.1; Retention time: 0.472 min.

Step 3: To a solution of (5-amino-6″-ethoxy-3′,5″,6-trifluoro-[2,2′:6′,3″-terpyridin]-4′-yl)((1S,4S)-2,5-diazabicyclo[2.2.2]octan-2-yl)methanone (1.0 eq, 70.0 mg, 0.1 mmol) in methanol (7 mL) were added AcOH (3.0 eq, 26.0 mg, 0.4 mmol) and formaldehyde solution (1.1 eq, 4.8 mg, 0.1 mmol). Then NaBH₃CN (2.0 eq, 18.2 mg, 0.3 mmol) was added in the reaction mixture at 0° C. The resulting mixture was stirred for 1 h at room temperature, then diluted with water (10 mL) and extracted with EA (3*50 mL). The combined organic layers were washed with saturated NaCl solution (40 mL), dried over anhydrous Na₂SO₄, filtered and concentrated in vacuo. The residue was purified by prep-HPLC using the following conditions: Column: XSelect CSH Prep C18 OBD Column, 30*150 mm, 5 μm; Mobile Phase A: Water (0.1% FA), Mobile Phase B: ACN; Flow rate: 60 mL/min; Gradient (B %): 11% B to 41% B in 8 min; Wavelength: 254 nm/220 nm; RT1 (min): 6.56. The pure fractions were concentrated and lyophilized to afford title compound (22.8 mg, 0.1 mmol, 28.7% yield) as an off-white solid. LC purity: 99% (UV at 254 nm); Mass calculated for C₂₅H₂₅F₃N₆O₂ [M+1]⁺ 499.2, found 499.2; Retention time: 1.354 min. ¹H NMR (400 MHz, DMSO) δ 8.80 (dd, J=7.9, 2.0 Hz, 1H), 8.47-8.37 (m, 1H), 8.09-8.00 (m, 2H), 7.35-7.25 (m, 1H), 5.99 (s, 2H), 4.53-4.43 (m, 2H), 3.82-3.80 (m, 1H), 3.74-3.44 (m, 2H), 2.90-2.88 (m, 2H), 2.77-2.63 (m, 1H), 2.34-2.31 (m, 3H), 2.01-1.98 (m, 1H), 1.82-1.80 (m, 1H), 1.65-1.62 (m, 2H), 1.43-1.35 (m, 3H).

### Example 26. (5-amino-6″-ethoxy-3′,6-difluoro-[2,2′:6′,3″-terpyridin]-4′-yl)((1S,4S)-5-methyl-2,5-diazabicyclo[2.2.2]octan-2-yl)methanone

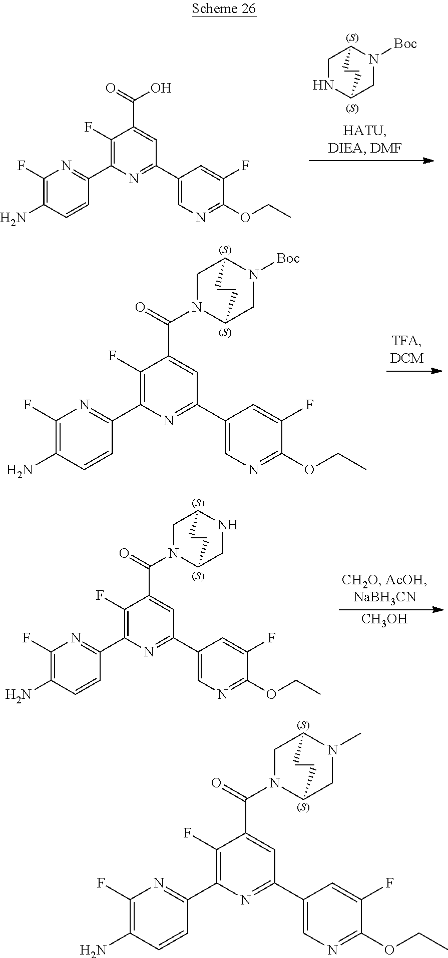

Step 1: To a solution of 5-amino-6″-ethoxy-3′,6-difluoro-[2,2′:6′,3″-terpyridine]-4′-carboxylic acid (1.0 eq, 70 mg, 0.2 mmol), tert-butyl (1S,4S)-2,5-diazabicyclo[2.2.2]octane-2-carboxylate (1.1 eq, 44 mg, 0.2 mmol) in DMF (3 mL) were added HATU (1.5 eq, 107 mg, 0.3 mmol) and DIEA (3.0 eq, 73 mg, 0.5 mmol). The resulting solution was stirred at room temperature for 1 h. The reaction was then quenched with water and extracted with EtOAc (2×100 mL). The organic layers were washed with brine (1×30 mL), dried over anhydrous sodium sulfate, filtered and concentrated in vacuo. The product was used in the next step directly without further purification. LC purity: 80% (UV at 254 nm); Mass calculated for C₂₉H₃₂F₂N₆O₄[M+1]⁺ 567.6, found 567.3; Retention time: 0.822 min.

Step 2: To a solution of tert-butyl (1S,4S)-5-(5-amino-6″-ethoxy-3′,6-difluoro-[2,2′:6′,3″-terpyridine]-4′-carbonyl)-2,5-diazabicyclo[2.2.2]octane-2-carboxylate (1.0 eq, 80 mg, 0.1 mmol) in DCM (5 mL) was added TFA (0.5 mL). The mixture was stirred at room temperature for 1 h. The resulting solution was concentrated in vacuo and the residue was added DMSO, filtered and purified via reversed phase flash chromatography using a gradient of 40% to 70% ACN in water (with 5 mM NH₄HCO₃) to afford (5-amino-6″-ethoxy-3′,6-difluoro-[2,2′:6′,3″-terpyridin]-4′-yl)((1S,4S)-2,5-diazabicyclo[2.2.2]octan-2-yl)methanone (50 mg, 0.1 mmol, 86% yield) as a light yellow solid. LC purity: 87% (UV at 254 nm); Mass calculated for C₂₋₄H₂₄F₂N₆O₂[M+1]⁺ 467.5, found 466.9; Retention time: 0.499 min.

Step 3: To a solution of (5-amino-6″-ethoxy-3′,6-difluoro-[2,2′:6′,3″-terpyridin]-4′-yl)((1S,4S)-2,5-diazabicyclo[2.2.2]octan-2-yl)methanone (1.0 eq, 70 mg, 0.2 mmol) in methanol (7 mL) were added formaldehyde solution (1.1 eq, 5 mg, 0.2 mmol) and AcOH (3.0 eq, 27 mg, 0.5 mmol). Then NaBH₃CN (2.0 eq, 19 mg, 0.3 mmol) was added to above mixture at 0° C. and the reaction was stirred at room temperature for 1 h. The reaction mixture was quenched with water and extracted with EtOAc (2×100 mL). The combined organic layers were washed with brine over 4 times, dried over anhydrous sodium sulfate, filtered and concentrated in vacuo. The residue was purified by Prep-HPLC using the following conditions: Column: Sunfire Prep C18 OBD Column, 30*150 mm, 5 μm; Mobile Phase A: Water (0.1% FA), Mobile Phase B: MEOH; Flow rate: 60 mL/min; Gradient (B %): 24% B to 54% B in 8 min; Wavelength: 254 nm/220 nm; RT1(min): 6.9. The pure fractions were concentrated and lyophilized to afford title compound (47 mg, 0.0965 mmol, 64.34% yield) as an off-white solid. LC purity: 99.0% (UV at 254 nm); Mass calculated for C₂₅H₂₆F₂N₆O₂ [M+1]⁺ 481.5, found 481.2; Retention time: 1.187 min. 1H NMR (400 MHz, DMSO) δ 8.99-8.91 (m, 1H), 8.51-8.42 (m, 1H), 8.05-7.95 (m, 2H), 7.35-7.25 (m, 1H), 6.92 (dd, J=8.7, 4.0 Hz, 1H), 5.97 (s, 2H), 4.47-4.37 (m, 2H), 3.60 (d, J=10.6 Hz, 2H), 3.49-3.42 (m, 1H), 2.88 (d, J=6.6 Hz, 3H), 2.31 (d, J=14.2 Hz, 3H), 1.99 (s, 1H), 1.90-1.79 (m, 1H), 1.64 (t, J=11.5 Hz, 2H), 1.39-1.31 (m, 3H).

### Example 27. (6-(5-amino-6-fluoropyrazin-2-yl)-6′-ethoxy-5-fluoro-[2,3′-bipyridin]-4-yl)(1-methyl-1,6-diazaspiro[3.5]nonan-6-yl)methanone

### Example 28. (R)-(6-(5-amino-6-fluoropyrazin-2-yl)-6′-ethoxy-5-fluoro-[2,3′-bipyridin]-4-yl)(1-methyl-1,6-diazaspiro[3.5]nonan-6-yl)methanone

### Example 29. (S)-(6-(5-amino-6-fluoropyrazin-2-yl)-6′-ethoxy-5-fluoro-[2,3′-bipyridin]-4-yl)(1-methyl-1,6-diazaspiro[3.5]nonan-6-yl)methanone

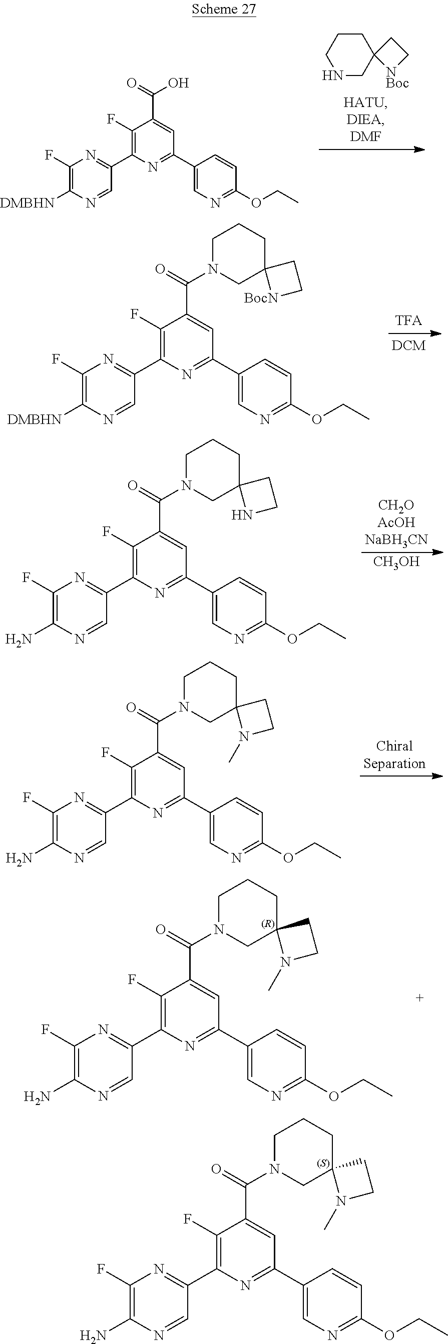

Step 1 & 2: (6-(5-amino-6-fluoropyrazin-2-yl)-6′-ethoxy-5-fluoro-[2,3′-bipyridin]-4-yl)(1,6-diazaspiro[3.5]nonan-6-yl)methanone (52 mg) was prepared in a 48% overall yield as a light yellow solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and tert-butyl 1,6-diazaspiro[3.5]nonane-1-carboxylate in Step 1. LC purity: 75% (UV at 254 nm); Mass calculated for C₂₋₄H₂₅F₂N₇O₂[M+1]⁺ 482.2; found 482.2; Retention time: 0.423 min.

Step 3: To a solution of (6-(5-amino-6-fluoropyrazin-2-yl)-6′-ethoxy-5-fluoro-[2,3′-bipyridin]-4-yl)(1,6-diazaspiro[3.5]nonan-6-yl)methanone (1.00 eq, 50 mg, 0.104 mmol) in methanol (1 mL) were added HOAc (3.0 eq, 16 mg, 0.3 mmol) and formaldehyde (1.2 eq, 4 mg, 0.1 mmol). NaBH₃CN (3.0 eq, 17 mg, 0.3 mmol) was added to the reaction mixture and the resulting mixture was stirred for 1 h at room temperature. Upon completion, the reaction mixture was diluted with water (10 mL) and extracted with EA (3*50 mL). The combined organic layers were washed with saturated NaCl solution (40 mL) and dried over anhydrous Na₂SO₄ and filtered. The filtrate was concentrated in vacuo and the residue was purified by Prep-HPLC to give Example 27 (Column: XBridge Prep OBD C18 Column, 30*150 mm, 5 μm; Mobile Phase A: Water (10 mmol/LNH₄HCO₃), Mobile Phase B: ACN; Flow rate: 60 mL/min; Gradient (B %): 20% B to 50% B in 10 min; Wavelength: 254 nm/220 nm; RT1(min): 7.27). LC purity: 99% (UV at 254 nm); Mass calculated for C₂₅H₂₇F₂N₇O₂[M+1]⁺, 496.2, found 496.2; Retention time: 0.890 min. ¹H NMR (400 MHz, DMSO) δ 8.94 (s, 1H), 8.74 (t, J=5.2 Hz, 1H), 8.47 (d, J=8.7 Hz, 1H), 7.94 (s, 1H), 7.39 (s, 2H), 6.92 (dd, J=8.8, 4.6 Hz, 1H), 4.38-4.35 (m, 3H), 3.37-3.31 (m, 1H), 3.26-3.09 (m, 2H), 3.04-2.76 (m, 2H), 2.21 (s, 2H), 2.09 (s, 1H), 1.99-1.41 (m, 6H), 1.35 (t, J=7.0 Hz, 3H).

Step 4: (6-(5-amino-6-fluoropyrazin-2-yl)-6′-ethoxy-5-fluoro-[2,3′-bipyridin]-4-yl)(1-methyl-1,6-diazaspiro[3.5]nonan-6-yl)methanone (1.00 eq, 16 mg, 0.03 mmol) was purified by Prep-chiral-HPLC (Column: JW-CHIRALPAK IM, 20*250 mm, Sum; Mobile Phase A: EtOH: DCM=1: 1-HPLC, Mobile Phase B: Hex (0.1% 2M NH3-MeOH)-HPLC; Flow rate: 20 mL/min; Gradient (B %): 70% B to 70% B in 21 min; Wavelength: 220/254 nm; RT1(min): 10.43; RT2(min): 17.37; Sample Solvent: EtOH: DCM=1: 1-HPLC; Injection Volume: 2.0 mL). Pure fractions (The first peak compound and the second peak compound were arbitrarily assigned as the S and R isomers, respectively.) were evaporated to afford the title compound (R)-(6-(5-amino-6-fluoropyrazin-2-yl)-6′-ethoxy-5-fluoro-[2,3′-bipyridin]-4-yl)(1-methyl-1,6-diazaspiro[3.5]nonan-6-yl)methanone (Example 28, 5.7 mg, 0.01 mmol, 35% yield) as a white solid and (S)-(6-(5-amino-6-fluoropyrazin-2-yl)-6′-ethoxy-5-fluoro-[2,3′-bipyridin]-4-yl)(1-methyl-1,6-diazaspiro[3.5]nonan-6-yl)methanone (Example 29, 5.6 mg, 0.01 mmol, 34% yield) as a white solid.

Example 28: LC purity: 99% (UV at 254 nm); Mass calculated for C₂₅H₂₇F₂N₇O₂ [M+1]⁺, 496.2, found 496.2; Retention time: 1.335 min. ¹H NMR (400 MHz, DMSO-d₆) δ 8.95 (s, 1H), 8.75 (t, J=5.3 Hz, 1H), 8.48 (d, J=8.8 Hz, 1H), 7.95 (s, 1H), 7.39 (s, 2H), 6.93 (dd, J=8.7, 5.0 Hz, 1H), 4.53-4.03 (m, 3H), 3.27-3.08 (m, 2H), 3.01 (s, 1H), 2.87 (d, J=7.6 Hz, 1H), 2.24 (s, 2H), 2.08 (s, 1H), 2.00-1.44 (m, 7H), 1.36 (t, J=7.0 Hz, 3H).

Example 29: LC purity: 99% (UV at 254 nm); Mass calculated for C₂₅H₂₇F₂N₇O₂ [M+1]⁺, 496.2, found 496.2; Retention time: 1.342 min. ¹H NMR (400 MHz, DMSO-d₆) δ 8.95 (s, 1H), 8.75 (t, J=5.3 Hz, 1H), 8.48 (d, J=8.8 Hz, 1H), 7.95 (s, 1H), 7.39 (s, 2H), 6.93 (dd, J=8.7, 5.0 Hz, 1H), 4.53-4.03 (m, 3H), 3.27-3.08 (m, 2H), 3.01 (s, 1H), 2.87 (d, J=7.6 Hz, 1H), 2.24 (s, 2H), 2.08 (s, 1H), 2.00-1.44 (m, 7H), 1.36 (t, J=7.0 Hz, 3H).

### Example 30. (2-(5-amino-6-fluoropyrazin-2-yl)-6-(4-(2,2-difluoroethoxy)phenyl)-3-fluoropyridin-4-yl)(1-methyl-1,6-diazaspiro[3.5]nonan-6-yl)methanone

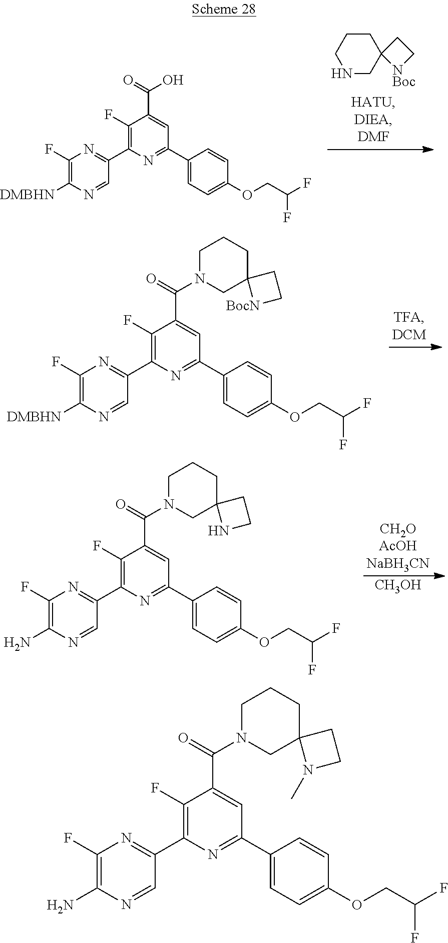

The title compound (15 mg) was prepared in a 25% overall yield as a solid, following a procedure analogous to that described in Example 26, using 6-(4-(2,2-difluoroethoxy)phenyl)-2-(5-((2,4-dimethoxybenzyl)amino)-6-fluoropyrazin-2-yl)-3-fluoroisonicotinic acid and tert-butyl 1,6-diazaspiro[3.5]nonane-1-carboxylate in Step 1. Prep-HPLC conditions: Column: XBridge Prep OBD C18 Column, 30*150 mm, 5 m; Mobile Phase A: water (10 nmol/LNH₄HCO₃), Mobile Phase B: ACN; Flow rate: 60 mL/min; Gradient (B %): 28% B to 45% B in 10 min; Wavelength: 254 nm/220 nm; RT1 (min): 8.99. LC purity: 99% (UV at 254 nm); Mass calculated for C₂₆H₂₆F₄N₆O₂[M+1]⁺, 531.1, found 531.1; Retention time: 1.258 min. ¹H NMR (400 MHz, DMSO) δ 8.71 (s, 1H), 8.16 (d, J=8.2 Hz, 2H), 8.06-7.75 (m, 1H), 7.37 (s, 2H), 7.28-7.00 (m, 2H), 6.43-6.41 (m, 1H), 4.53-4.04 (m, 3H), 3.42-3.33 (m, 1H), 3.26-3.08 (m, 2H), 3.08-2.90 (m, 1H), 2.90-2.76 (m, 1H), 2.16-2.13 (m, 3H), 1.90-1.88 (m, 2H), 1.77 (s, 1H), 1.73-1.65 (m, 2H), 1.47 (d, J=10.0 Hz, 1H).

### Example 31. (6-(5-amino-6-fluoropyrazin-2-yl)-6′-ethoxy-5-fluoro-[2,3′-bipyridin]-4-yl)((1R,4R)-5-(methyl-d3)-2,5-diazabicyclo[2.2.2]octan-2-yl)methanone

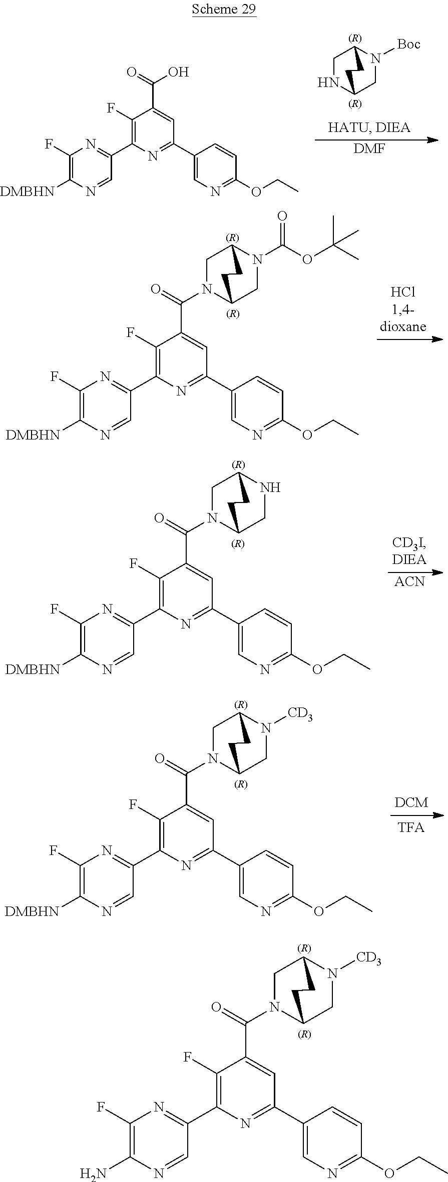

Step 1 & 2: ((1R,4R)-2,5-diazabicyclo[2.2.2]octan-2-yl)(6-(5-((2,4-dimethoxybenzyl)amino)-6-fluoropyrazin-2-yl)-6′-ethoxy-5-fluoro-[2,3′-bipyridin]-4-yl)methanone (70 mg) was prepared in a 30% overall yield as a solid, following a procedure analogous to that described in Example 1, using 6-(5-((2,4-dimethoxybenzyl)amino)-6-fluoropyrazin-2-yl)-6′-ethoxy-5-fluoro-[2,3′-bipyridine]-4-carboxylic acid and tert-butyl (1R,4R)-2,5-diazabicyclo[2.2.2]octane-2-carboxylate in Step 1. LC purity: 75% (UV at 254 nm); Mass calculated for C₃₂H₃₃F₂N₇O₄[M+1]⁺ 618.3; found 618.3; Retention time: 0.439 min.

Step 3: To a solution of ((1R,4R)-2,5-diazabicyclo[2.2.2]octan-2-yl)(6-(5-((2,4-dimethoxybenzyl)amino)-6-fluoropyrazin-2-yl)-6′-ethoxy-5-fluoro-[2,3′-bipyridin]-4-yl)methanone (1.0 eq, 70 mg, 0.1 mmol) and iodomethane-d3 (1.5 eq, 25 mg, 0.2 mmol) in MeCN (5 mL) was added with DIEA (3.0 eq, 44 mg, 0.3 mmol). The resulting mixture was stirred at 80° C. for 6 h. The mixture was concentrated, and the residue was purified by reversed-phase flash chromatography using a gradient of 20% to 80% ACN in water (with 10 mmol/L NH₄HCO₃) to afford (6-(5-((2,4-dimethoxybenzyl)amino)-6-fluoropyrazin-2-yl)-6′-ethoxy-5-fluoro-[2,3′-bipyridin]-4-yl)((1R,4R)-5-(methyl-d3)-2,5-diazabicyclo[2.2.2]octan-2-yl)methanone (50 mg, 0.1 mmol, 70% yield) as a yellow solid. LC purity: 72% (UV at 254 nm); Mass calculated for C₃₃H₃₂D₃F₂N₇O₄ [M+1]⁺ 635.3; found 635.3. Retention time: 0.936 min.

Step 4: To a solution of (6-(5-((2,4-dimethoxybenzyl)amino)-6-fluoropyrazin-2-yl)-6′-ethoxy-5-fluoro-[2,3′-bipyridin]-4-yl)((1R,4R)-5-(methyl-d3)-2,5-diazabicyclo[2.2.2]octan-2-yl)methanone (1.0 eq, 50 mg, 0.1 mmol) in DCM (1 mL) and added TFA (1 mL) and the mixture was stirred at room temperature for 2 h. The resulting solution was concentrated in vacuo and the residue was purified by Prep-HPLC (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 22% B to 42% B in 10 min; Wavelength: 254 nm/220 nm; RT1(min): 8.08). Pure fractions were evaporated to afford (6-(5-amino-6-fluoropyrazin-2-yl)-6′-ethoxy-5-fluoro-[2,3′-bipyridin]-4-yl)((1R,4R)-5-(methyl-d3)-2,5-diazabicyclo[2.2.2]octan-2-yl)methanone (5.2 mg, 0.01 mmol, 13% yield) was a white solid. LC purity: 97.5% (UV at 254 nm); Mass calculated for C₂₋₄H₂₂D₃F₂N₇O₂ [M+H]⁺ 485.2, found 485.2. Retention time: 1.127 min. ¹H NMR (400 MHz, DMSO) δ 8.99-8.92 (m, 1H), 8.73 (dd, J=8.8, 4.5 Hz, 1H), 8.48 (dd, J=8.6, 2.7 Hz, 1H), 8.04 (t, J=5.1 Hz, 1H), 7.39 (s, 2H), 6.93 (dd, J=8.6, 4.0 Hz, 1H), 4.46-4.34 (m, 2H), 3.82 (d, J=12.4 Hz, 1H), 3.51-3.42 (m, 2H), 2.87 (s, 2H), 2.00 (s, 1H), 1.83 (s, 1H), 1.64 (s, 2H), 1.36 (dd, J=7.7, 6.4 Hz, 3H).

### Example 32. (6-(5-amino-6-fluoropyrazin-2-yl)-6′-ethoxy-5-fluoro-[2,3′-bipyridin]-4-yl)((1R,4R)-5-ethyl-2,5-diazabicyclo[2.2.2]octan-2-yl)methanone

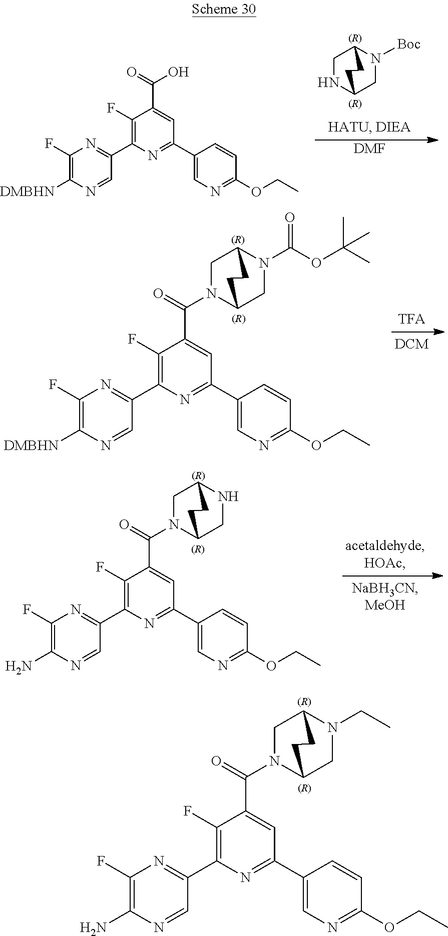

Step 1 & 2: (6-(5-amino-6-fluoropyrazin-2-yl)-6′-ethoxy-5-fluoro-[2,3′-bipyridin]-4-yl)((1R,4R)-2,5-diazabicyclo[2.2.2]octan-2-yl)methanone (40 mg) was prepared in a 69% overall yield as a with solid, following a procedure analogous to that described in Example 26, using 6-(5-((2,4-dimethoxybenzyl)amino)-6-fluoropyrazin-2-yl)-6′-ethoxy-5-fluoro-[2,3′-bipyridine]-4-carboxylic acid and tert-butyl (1R,4R)-2,5-diazabicyclo[2.2.2]octane-2-carboxylate in Step 1. LC purity: 80% (UV at 254 nm); Mass calculated for C₂₃H₂₃F₂N₇O₂[M+H]⁺ 468.2, found 468.2. Retention time: 0.549 min.

Step 3: To a solution of 6-(5-amino-6-fluoropyrazin-2-yl)-6′-ethoxy-5-fluoro-[2,3′-bipyridin]-4-yl)((1R,4R)-2,5-diazabicyclo[2.2.2]octan-2-yl)methanone (1.0 eq, 40 mg, 0.1 mmol) in methanol (0.5 mL) were added HOAc (3.0 eq, 16 mg, 0.3 mmol) and acetaldehyde (1.2 eq, 4 mg, 0.1 mmol), Then NaBH₃CN (3.0 eq, 17 mg, 0.3 mmol) was added in the reaction system after the mixture was stirred for 30 min. The resulting mixture was stirred for 1 h at room temperature, then diluted with water (10 mL) and extracted with EA (3*50 mL). The combined organic layers were washed with saturated NaCl solution (40 mL) and dried over anhydrous Na₂SO₄ and filtered. The filtrate was concentrated in vacuo and the residue was purified by Prep-HPLC (Column: XBridge Prep OBD C18 Column, 30*150 mm, 5 m; Mobile Phase A: Water (10 mmol/L NH₄HCO₃), Mobile Phase B: ACN; Flow rate: 60 mL/min mL/min; Gradient (B %): 36% B to 66% B in 8 min; Wavelength: 254 nm/220 nm; RT1(min): 7.45). Pure fractions were evaporated to afford the title compound (4.1 mg, 0.01 mmol, 9% yield) was an off-white solid. LC purity: 99.1% (UV at 254 nm); Mass calculated for C₂₅H₂₇F₂N₇O₂[M+H]⁺ 496.2, found 496.2. Retention time: 1.156 min. ¹H NMR (400 MHz, DMSO) δ 8.95 (s, 1H), 8.73 (d, J=4.7 Hz, 1H), 8.47 (s, 1H), 8.22 (s, 1H), 8.04 (d, J=4.7 Hz, 1H), 7.38 (s, 2H), 6.93 (d, J=8.6 Hz, 1H), 4.39 (d, J=7.0 Hz, 2H), 3.50 (d, J=8.4 Hz, 2H), 3.00 (s, 1H), 2.31 (s, 1H), 1.64 (s, 2H), 1.36 (t, J=7.0 Hz, 3H), 1.00 (t, J=7.0 Hz, 3H).

### Example 33. (S)-(6-(5-amino-6-fluoropyrazin-2-yl)-6′-ethoxy-5-fluoro-[2,3′-bipyridin]-4-yl)(3,4-dimethylpiperazin-1-yl)methanone

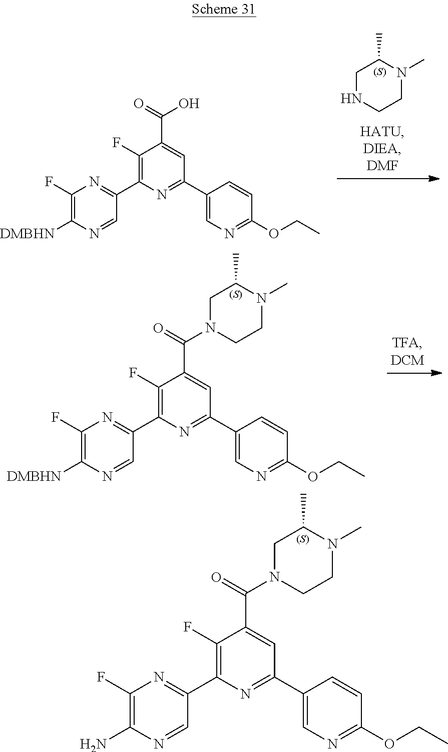

The title compound (9 mg) was prepared in a 12% overall yield as a white solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and (S)-1,2-dimethylpiperazine in Step 1. Prep-HPLC conditions: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 25% B to 45% B in 10 min; Wavelength: 254 nm/220 nm; RT1 (min): 9.17. LC purity: 99.0% (UV at 254 nm); Mass calculated for C₂₃H₂₅F₂N₇O₂[M+1]⁺, 470.2 found 470.1; Retention time: 1.197 min. ¹H NMR (400 MHz, MeOD) δ 8.86 (d, J=2.5 Hz, 1H), 8.71 (d, J=4.2 Hz, 1H), 8.43 (dd, J=8.7, 2.5 Hz, 1H), 7.88 (dd, J=3.8, 2.2 Hz, 1H), 6.92 (d, J=8.7 Hz, 1H), 4.61-4.53 (m, 1H), 4.42 (q, J=7.1 Hz, 2H), 3.70-3.57 (m, 2H), 3.19 (d, J=12.6 Hz, 2H), 2.85 (d, J=12.4 Hz, 2H), 2.68 (d, J=4.2 Hz, 3H), 1.47-1.34 (m, 5H), 1.19 (d, J=6.4 Hz, 1H).

### Example 34. (6-(5-amino-6-fluoropyrazin-2-yl)-6′-ethoxy-5-fluoro-[2,3′-bipyridin]-4-yl)(7-methyl-4,7-diazaspiro[2.5]octan-4-yl)methanone

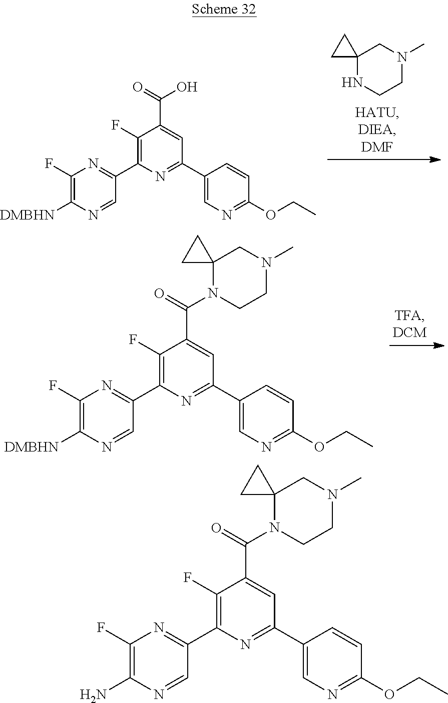

The title compound (19 mg) was prepared in a 22% overall yield as a white solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and 7-methyl-4,7-diazaspiro[2.5]octane in Step 1. Prep-HPLC conditions: Column: X-Select Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 35% B to 45% B in 10 min; Wavelength: 254 nm/220 nm; RT1(min): 7.99. LC purity: 99.4% (UV at 254 nm); Mass calculated for C₂₋₄H₂₅F₂N₇O₂[M+1]⁺, 482.2, found 482.1; Retention time: 1.214 min. ¹H NMR (400 MHz, MeOD) δ 8.85 (s, 1H), 8.70 (d, J=8.6 Hz, 1H), 8.43 (d, J=8.8 Hz, 1H), 7.89 (s, 1H), 6.92 (dd, J=9.0, 2.2 Hz, 1H), 4.90 (s, 1H), 4.42 (m, 2H), 3.73 (s, 1H), 3.04 (s, 4H), 2.74-2.69 (m, 3H), 1.43 (t, J=7.0 Hz, 4H), 1.17 (s, 1H), 0.93 (s, 2H).

### Example 35. (R)-(6-(5-amino-6-fluoropyrazin-2-yl)-6′-ethoxy-5-fluoro-[2,3′-bipyridin]-4-yl)(3,4-dimethylpiperazin-1-yl)methanone

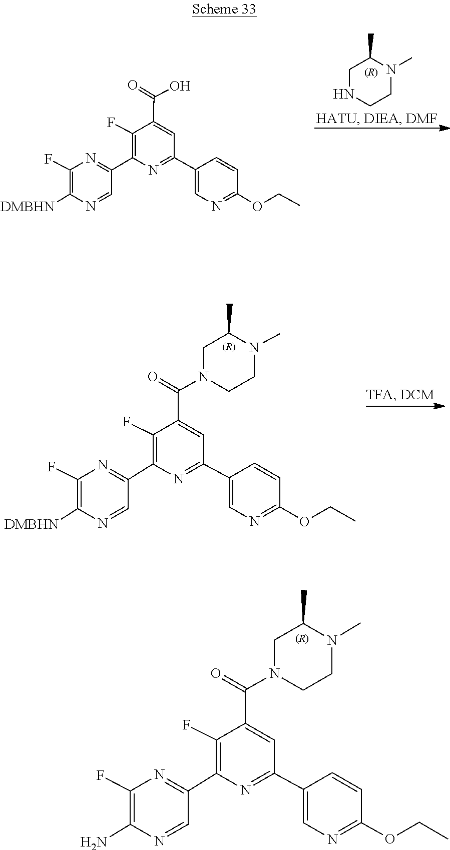

The title compound (13 mg) was prepared in a 9% overall yield as a solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and (R)-1,2-dimethylpiperazine in Step 1. Prep-HPLC conditions: Column: XBridge Prep OBD C18 Column, 3 0*150 mm, 5 μm; Mobile Phase A: water (10 mmol/L NH₄HCO₃+0.05% NH₃H₂O), Mobile Phase B: ACN; Flow rate: 60 mL/min; Gradient (B %): 25% B to 45% B in 10 min; Wavelength: 254 nm/220 nm; RT1(min): 9.05. LC purity: 97.1% (UV at 254 nm); Mass calculated for C₂₃H₂₅F₂N₇O₂[M+1]⁺ 470.1, found 470.1; Retention time: 1.197 min. ¹H NMR (400 MHz, DMSO) δ 8.95 (s, 1H), 8.74 (d, J=3.8 Hz, 1H), 8.58-8.34 (m, 1H), 8.01 (s, 1H), 7.39 (s, 2H), 6.93 (d, J=8.8 Hz, 1H), 4.60-4.00 (m, 3H), 3.43-3.33 (m, 1H), 3.29-3.01 (m, 1H), 2.95-2.77 (m, 1H), 2.67 (s, 1H), 2.21 (s, 3H), 2.19-1.95 (m, 2H), 1.36 (t, J=7.0 Hz, 3H), 1.12-0.81 (m, 3H).

### Example 36. (6-(5-amino-6-fluoropyrazin-2-yl)-6′-ethoxy-5-fluoro-[2,3′-bipyridin]-4-yl)(3-methyl-3,6-diazabicyclo[3.2.1]octan-6-yl)methanone

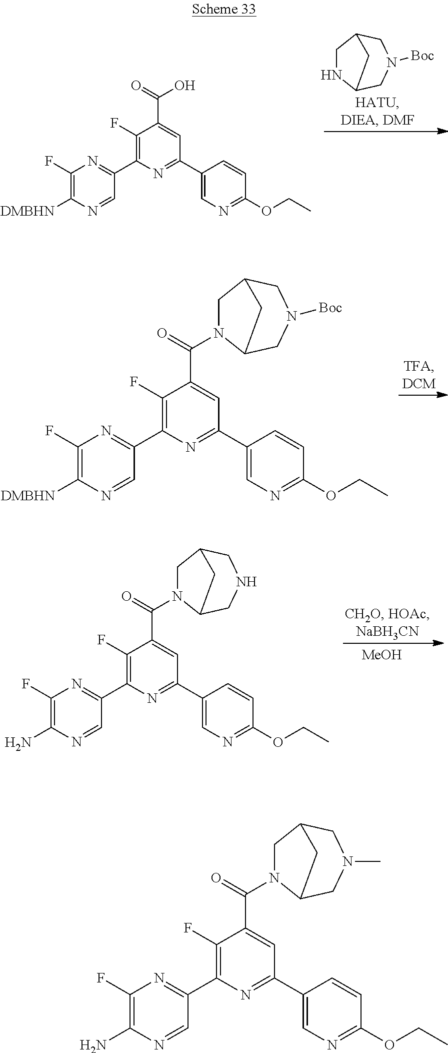

The title compound (30 mg) was prepared in a 17% overall yield as a with solid, following a procedure analogous to that described in Example 26, using 6-(5-((2,4-dimethoxybenzyl)amino)-6-fluoropyrazin-2-yl)-6′-ethoxy-5-fluoro-[2,3′-bipyridine]-4-carboxylic acid and tert-butyl 3,6-diazabicyclo[3.2.1]octane-3-carboxylate in Step 1. Prep-HPLC conditions: Column: xBridge Prep Shield RP18 5 m OBD 30*150 mm; Mobile Phase A: Water (10 mmol/L NH₄HCO₃), Mobile Phase B: ACN′; Flow rate: 60 mL/min; Gradient (B %): isocratic 34-64B IN 10 MIN; Wavelength: 220/254 nm; RT1(min): 6.37. LC purity: 99.6% (UV at 254 nm); Mass calculated for C₂₋₄H₂₅F₂N₇O₂[M+H]⁺ 482.2, found 482.2. Retention time: 0.755 min. ¹H NMR (400 MHz, DMSO) δ 9.01 (d, J=4.7 Hz, 1H), 8.82 (d, J=2.0 Hz, 1H), 8.47 (dd, J=10.9, 2.0 Hz, 1H), 7.89 (d, J=7.1 Hz, 2H), 7.28 (s, 2H), 4.64 (dd, J=10.3, 2.4 Hz, 1H), 4.49-4.41 (m, 2H), 4.06-3.98 (m, 1H), 3.73-3.69 (m, 1H), 3.08 (d, J=11.2 Hz, 1H), 2.73 (d, J=11.4 Hz, 1H), 2.25 (s, 3H), 2.13-2.08 (m, 1H), 1.87 (t, J=10.8 Hz, 1H), 1.40 (t, J=7.0 Hz, 3H).

### Example 37. (6-(5-amino-6-fluoropyrazin-2-yl)-6′-ethoxy-5-fluoro-[2,3′-bipyridin]-4-yl)((1S,4S)-5-(methyl-d3)-2,5-diazabicyclo[2.2.2]octan-2-yl)methanone

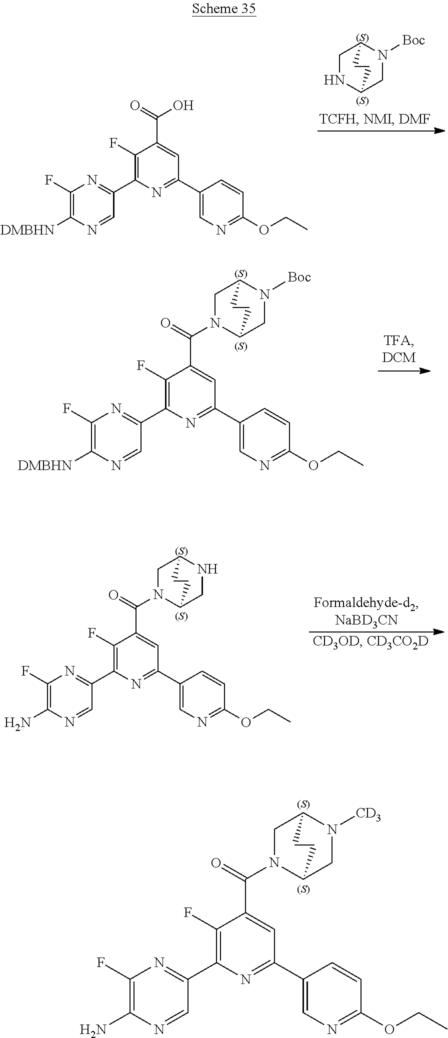

Step 1: The solution of 6-(5-((2,4-dimethoxybenzyl)amino)-6-fluoropyrazin-2-yl)-6′-ethoxy-5-fluoro-[2,3′-bipyridine]-4-carboxylic acid (preparation in Example 1, 1.0 eq, 390 mg, 0.7 mmol) and tert-butyl (1S,4S)-2,5-diazabicyclo[2.2.2]octane-2-carboxylate (1.2 eq, 190 mg, 0.9 mmol) in DMF (10 mL) were added TCFH (2.0 eq, 417 mg, 1.5 mmol) and NMI (3.0 eq, 183 mg, 2.2 mmol) at 0° C. The reaction was stirred at room temperature for 1 h. The resulting mixture was diluted with water and extracted with EtOAc. The organic layers were washed with brine, dried and concentrated to afford crude tert-butyl (1S,4S)-5-(6-(5-((2,4-dimethoxybenzyl)amino)-6-fluoropyrazin-2-yl)-6′-ethoxy-5-fluoro-[2,3′-bipyridine]-4-carbonyl)-2,5-diazabicyclo[2.2.2]octane-2-carboxylate (620 mg, 0.7 mmol, 100% yield) as a black oil. LC purity: 86.8% (UV at 254 nm); Mass calculated for C₃₇H₄₁F₂N₇O₆[M+1]⁺ 718.3, found 718.1; Retention time: 1.020 min.

Step 2: The solution of tert-butyl (1S,4S)-5-(6-(5-((2,4-dimethoxybenzyl)amino)-6-fluoropyrazin-2-yl)-6′-ethoxy-5-fluoro-[2,3′-bipyridine]-4-carbonyl)-2,5-diazabicyclo[2.2.2]octane-2-carboxylate (1.0 eq, 620 mg, 0.7 mmol) in DCM (10 mL) was added TFA (1.0 eq, 1.5 mL, 2.2 mmol) at room temperature. The reaction was stirred for 1 h at room temperature. The resulting mixture was treated with 2 mL NH₄OH at 0° C. and concentrated in vacuo. The residue was purified by reverse phase flash chromatography using a gradient of 40% to 70% ACN in water (with 10 mmol/L NH₄HCO₃) to afford (6-(5-amino-6-fluoropyrazin-2-yl)-6′-ethoxy-5-fluoro-[2,3′-bipyridin]-4-yl)((1S,4S)-2,5-diazabicyclo[2.2.2]octan-2-yl)methanone (230 mg, 0.4 mmol, 63% yield) as a yellow solid. LC purity: 95.4% (UV at 254 nm); Mass calculated for C₂₃H₂₃F₂N₇O₂[M+1]⁺, 468.2, found 486.1; Retention time: 0.460 min.

Step 3: To a solution of (6-(5-amino-6-fluoropyrazin-2-yl)-6′-ethoxy-5-fluoro-[2,3′-bipyridin]-4-yl)((1S,4S)-2,5-diazabicyclo[2.2.2]octan-2-yl)methanone (1.0 eq, 225 mg, 0.5 mmol) and formaldehyde-d₂ (1.2 eq, 92 mg, 0.6 mmol) in CD₃0D (10 mL) was added CD₃CO₂D (5.0 eq, 120 mg, 2.4 mmol) and the solution was stirred for 10 min. NaBD₃CN (3.0 eq, 94 mg, 1.4 mmol) was then added into the above solution at 0° C. and stirred at ambient temperature for 1 h. The resulting mixture was diluted with ice-water and extracted with EtOAc. The organic layers were washed with brine, dried and concentrated in vacuo. The residue was purified with following condition: Column: XBridge Prep OBD C18 Column, 30*150 mm, 5 μm; Mobile Phase A: Water (10 nmol/LNH₄HCO₃+0.05% NH₃·H₂O), Mobile Phase B: ACN; Flow rate: 60 mL/min; Gradient (B %): 23% B to 40% B in 10 min; Wavelength: 254 nm/220 nm; RT1(min): 9.26. The pure fractions were concentrated and lyophilized to afford title compound (148 mg, 0.3 mmol, 63% yield) as an off-white solid. LC purity: 99.6% (UV at 254 nm); Mass calculated for C₂₋₄H₂₂D₃F₂N₇O₂ [M+1]⁺, 485.2, found 485.2; Retention time: 0.944 min. ¹H NMR (400 MHz, CD₃0D) 6 8.91-8.83 (m, 1H), 8.73-8.65 (m, 1H), 8.43-8.41 (m, 1H), 7.90-7.88 (m, 1.7 Hz, 1H), 6.95-6.87 (m, 1H), 4.42-4.40 (m, 2H), 4.04-4.02 (m, 1H), 3.82 (d, J=11.4 Hz, 1H), 3.63 (dd, J=13.3, 2.1 Hz, 1H), 3.02 (d, J=12.7 Hz, 2H), 2.89-2.85 (m, 1H), 2.20-2.03 (s, 2H), 1.86-1.76 (m, 2H), 1.43-1.41 (m, 3H).

### Example 38. (6-(5-amino-6-fluoropyrazin-2-yl)-6′-ethoxy-5-fluoro-[2,3′-bipyridin]-4-yl)((5S)-6-methyl-3,6-diazabicyclo[3.2.1]octan-3-yl)methanone

### Example 39. (6-(5-amino-6-fluoropyrazin-2-yl)-6′-ethoxy-5-fluoro-[2,3′-bipyridin]-4-yl)((5R)-6-methyl-3,6-diazabicyclo[3.2.1]octan-3-yl)methanone

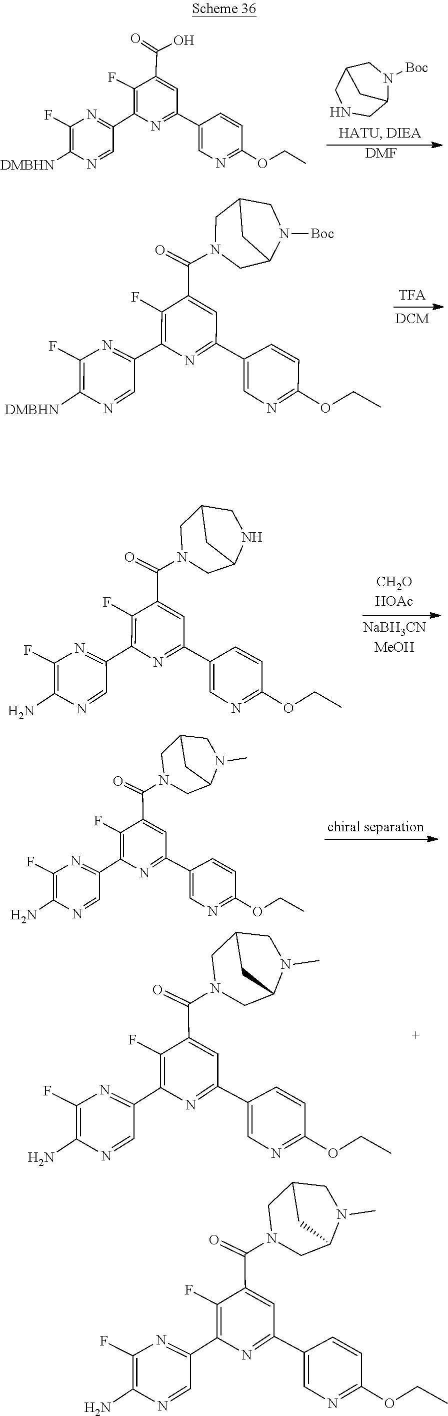

Step 1 & 2: (6-(5-amino-6-fluoropyrazin-2-yl)-6′-ethoxy-5-fluoro-[2,3′-bipyridin]-4-yl)(3,6-diazabicyclo[3.2.1]octan-3-yl)methanone (130 mg) was prepared in a 49% overall yield as a light yellow solid, following a procedure analogous to that described in Example 2, using 6-(5-((2,4-dimethoxybenzyl)amino)-6-fluoropyrazin-2-yl)-6′-ethoxy-5-fluoro-[2,3′-bipyridine]-4-carboxylic acid and tert-butyl 3,6-diazabicyclo[3.2.1]octane-6-carboxylate in Step 1. LC purity: 99.3% (UV at 254 nm); Mass calculated for C₂₃H₂₃F₂N₇O₂[M+H]⁺ 468.2, found 468.2. Retention time: 0.636 min.

Step 3: To a solution of 6-(5-amino-6-fluoropyrazin-2-yl)-6′-ethoxy-5-fluoro-[2,3′-bipyridin]-4-yl)(3,6-diazabicyclo[3.2.1]octan-3-yl)methanone (1.0 eq, 130 mg, 0.3 mmol) in methanol (2 mL) were added HOAc (3.0 eq, 48 mg, 0.9 mmol) and formaldehyde (1.2 eq, 16 mg, 0.4 mmol), Then NaBH₃CN (3.0 eq, 51 mg, 0.9 mmol) was added in the reaction mixture. The resulting mixture was stirred for 1 h at room temperature, then diluted with water (10 mL) and extracted with EA (3*50 mL). The combined organic layers were washed with saturated NaCl solution (40 mL) and dried over anhydrous Na₂SO₄ and filtered. The filtrate was concentrated in vacuo and the residue was purified by reversed column chromatography to afford (6-(5-amino-6-fluoropyrazin-2-yl)-6′-ethoxy-5-fluoro-[2,3′-bipyridin]-4-yl)(6-methyl-3,6-diazabicyclo[3.2.1]octan-3-yl)methanone (65 mg, 0.13 mmol, 43% yield) as a white solid. LC purity: 96% (UV at 254 nm); Mass calculated for C₂₋₄H₂₅F₂N₇O₂[M+H]⁺ 482.2, found 482.2. Retention time: 1.118 min.

Step 4: (6-(5-amino-6-fluoropyrazin-2-yl)-6′-ethoxy-5-fluoro-[2,3′-bipyridin]-4-yl)(6-methyl-3,6-diazabicyclo[3.2.1]octan-3-yl)methanone (1.00 eq, 65 mg, 0.13 mmol) was purified by Prep-chiral-HPLC (Column: JW-CHIRALPAK IH, 20*250 mm, Sum; Mobile Phase A: EtOH: DCM=1: 1--HPLC, Mobile Phase B: Hex (0.1% 2M NH3-MeOH)--HPLC; Flow rate: 20 mL/min; Gradient (B %): 85% B to 85% B in 20 min % B; Wavelength: 220/254 nm; RT1(min): 15.74; RT2(min): 18.04; Sample Solvent: EtOH: DCM=1: 1--HPLC; Injection Volume: 0.5 mL; Number Of Runs: 9). Pure fractions (The first peak compound and the second peak compound were arbitrarily assigned as the S and R isomers, respectively.) were evaporated to afford the title compound (6-(5-amino-6-fluoropyrazin-2-yl)-6′-ethoxy-5-fluoro-[2,3′-bipyridin]-4-yl)((5S)-6-methyl-3,6-diazabicyclo[3.2.1]octan-3-yl)methanone (Example 38, 22 mg, 0.1 mmol, 16% yield) as a white solid and (6-(5-amino-6-fluoropyrazin-2-yl)-6′-ethoxy-5-fluoro-[2,3′-bipyridin]-4-yl)((5R)-6-methyl-3,6-diazabicyclo[3.2.1]octan-3-yl)methanone (Example 39, 23 mg, 0.1 mmol, 17% yield) as a white solid.

Example 38: LC purity: 97.5% (UV at 254 nm); Mass calculated for C₂₋₄H₂₅F₂N₇O₂ [M+H]⁺ 482.2, found 482.2. Retention time: 1.118 min. ¹H NMR (400 MHz, DMSO) δ 8.93 (d, J=2.6 Hz, 1H), 8.73 (dd, J=4.7, 3.0 Hz, 1H), 8.51-8.42 (m, 1H), 7.99-7.96 (m, 1H), 7.39 (s, 2H), 6.93 (dd, J=8.7, 2.3 Hz, 1H), 4.39-4.31 (m, 3H), 3.30 (s, 2H), 3.08 (s, 1H), 3.01-2.87 (m, 2H), 2.78 (d, J=13.2 Hz, 1H), 2.39 (s, 2H), 2.28 (s, 2H), 1.87 (s, 1H), 1.69 (s, 1H), 1.36 (t, J=7.0 Hz, 3H).

Example 39: LC purity: 99.5% (UV at 254 nm); Mass calculated for C₂₋₄H₂₅F₂N₇O₂ [M+H]⁺ 482.2, found 482.2. Retention time: 1.117 min. ¹H NMR (400 MHz, DMSO) δ 8.93 (d, J=2.5 Hz, 1H), 8.73 (t, J=3.9 Hz, 1H), 8.46 (dd, J=8.9, 2.8 Hz, 1H), 7.99-7.95 (m, 1H), 7.39 (s, 2H), 6.93 (dd, J=8.7, 2.4 Hz, 1H), 4.39-4.31 (m, 3H), 3.30 (s, 2H), 3.08 (s, 1H), 3.03-2.86 (m, 2H), 2.78 (d, J=13.5 Hz, 1H), 2.39 (s, 2H), 2.28 (s, 1H), 1.87 (s, 1H), 1.69 (s, 1H), 1.36 (t, J=7.0 Hz, 3H).

### Example 40. (6-(5-amino-6-fluoropyrazin-2-yl)-6′-ethoxy-5,5′-difluoro-[2,3′-bipyridin]-4-yl)((1S,4S)-5-methyl-2,5-diazabicyclo[2.2.2]octan-2-yl)methanone

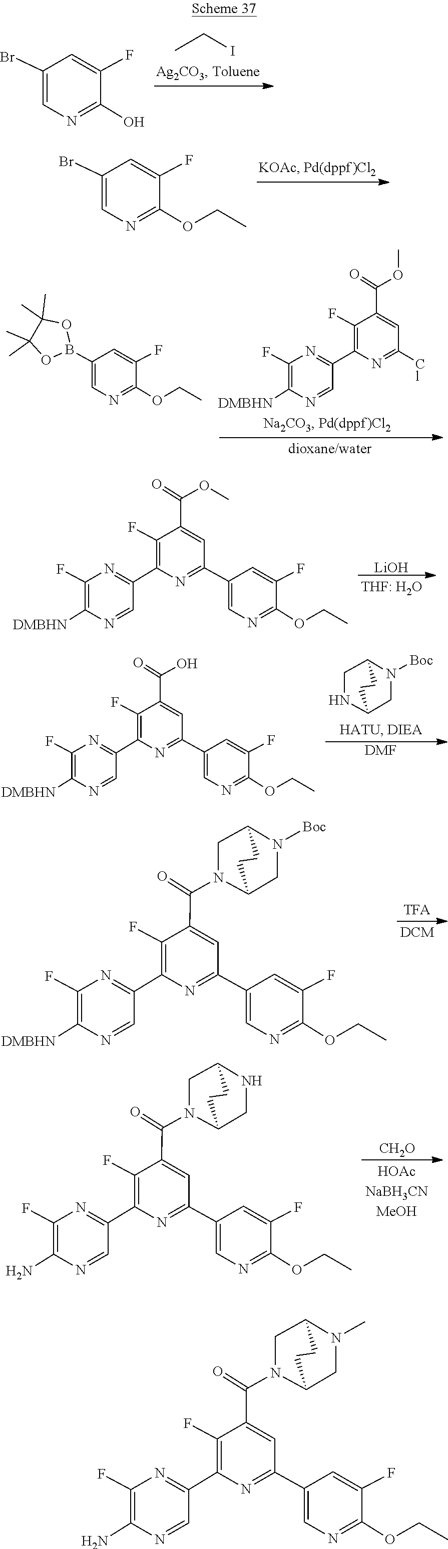

Step 1: To a stirred solution of 5-bromo-3-fluoropyridin-2-ol and iodoethane (1.0 eq, 1.6 g, 10.4 mmol) in Toluene (30 mL) was added Ag₂CO₃ (1.0 eq, 2.9 g, 10.4 mmol) at room temperature. The resulting mixture was heated to 150° C. and stirred for 30 min. The resulting mixture was diluted with water (100 mL) and extracted with EtOAc (3*500 mL). The combined organic layers were washed with saturated NaCl solution (300 mL) and dried over anhydrous Na₂SO₄ and filtered. The filtrate was concentrated in vacuo and the residue was purified by column chromatography on silica gel (PE: EtOAc=0~20%) to afford 5-bromo-2-ethoxy-3-fluoropyridine (1.5 g, 6.8 mmol, 65% yield) as a white solid. LC purity: 97% (UV at 254 nm); Mass calculated for C₇H7BrFNO [M+1]⁺ 220.0, found 220.0; Retention time: 1.097 min.

Step 2: To a solution of 5-bromo-2-ethoxy-3-fluoropyridine (1.0 eq, 700 mg, 3.2 mmol) and 4,4,4′,4′,5,5,5′,5′-octamethyl-2,2′-bi(1,3,2-dioxaborolane) (1.5 eq, 1.2 g, 4.7 mmol) in 1,4-dioxane (15 mL) were added KOAc (2.0 eq, 624 mg, 6.4 mmol) and Pd(dppf)Cl₂ (0.1 eq, 260 mg, 0.3 mmol) at room temperature. The reaction was stirred for 6 h at 90° C. under N₂ atmosphere. Upon completion, the mixture was quenched with water and extracted with EA. The combined organic layers were washed with brine, dried over anhydrous Na₂SO₄, filtered, and concentrated in vacuo to afford 2-ethoxy-3-fluoro-5-(4,4,5,5-tetramethyl-1,3,2-dioxaborolan-2-yl)pyridine (1.8 g, 5.6 mmol, crude) as a brown oil. LC purity: 80% (UV at 254 nm); Mass calculated for C₁₃H₁₉BFNO₃ [M+1]⁺ 268.0, found 268.0; Retention time:1.328 min.

Step 3: To a stirred solution of methyl 6-chloro-2-(5-((2,4-dimethoxybenzyl)amino)-6-fluoropyrazin-2-yl)-3-fluoroisonicotinate (1.0 eq, 300 mg, 0.7 mmol) and 2-ethoxy-3-fluoro-5-(4,4,5,5-tetramethyl-1,3,2-dioxaborolan-2-yl)pyridine (1.0 eq, 178 mg, 0.7 mmol) in 1,4-dioxane (12 mL) and water (1.2 mL) was added Na₂CO₃ (1.0 eq, 71 mg, 0.7 mmol) and Pd(dtbpf)Cl₂ (1.0 eq, 433 mg, 0.7 mmol) at room temperature under nitrogen atmosphere. The resulting mixture was heated to 60° C. and stirred for 2 h. The resulting mixture was diluted with water (30 mL) and extracted with EA (3 * 200 mL). The combined organic layers were washed with saturated NaCl solution (100 mL) and dried over anhydrous Na₂SO₄ and filtered. The filtrate was concentrated in vacuo and the residue was purified by reversed phase flash chromatography (C18 silica gel) using a gradient of 50% to 95% ACN in water (with 10 mmol/L NH₄HCO₃) to afford methyl 6-(5-((2,4-dimethoxybenzyl)amino)-6-fluoropyrazin-2-yl)-6′-ethoxy-5,5′-difluoro-[2,3′-bipyridine]-4-carboxylate (195 mg, 0.3 mmol, 52% yield) as a yellow solid. LC purity: 93% (UV at 254 nm); Mass calculated for C₂₇H₂₄F₃N₅O₅[M+1]⁺ 556.2, found 556.2; Retention time: 1.076 min.

Step 4: To a stirred solution of methyl 6-(5-((2,4-dimethoxybenzyl)amino)-6-fluoropyrazin-2-yl)-6′-ethoxy-5,5′-difluoro-[2,3′-bipyridine]-4-carboxylate (1.0 eq, 185 mg, 0.3 mmol) in water (2 mL) and THF (6 mL) was added Li—OH (8.0 eq, 66 mg, 2.74 mmol) at room temperature. The mixture was concentrated in vacuo. The resulting mixture was diluted with water (10 mL) and the pH was adjusted to 3 by the addition of 1M HCl. The resulting mixture was extracted with EA (3 * 80 mL). The combined organic layer were washed with saturated NaCl solution (100 mL) and dried over anhydrous Na₂SO₄ and filtered. The filtrate was concentrated in vacuo and the residue purified by reversed phase flash chromatography (C18 silica gel) using a gradient of 50% to 95% ACN in water (with 0.1% FA) to afford 6-(5-((2,4-dimethoxybenzyl)amino)-6-fluoropyrazin-2-yl)-6′-ethoxy-5,5′-difluoro-[2,3′-bipyridine]-4-carboxylic acid (155 mg, 0.3 mmol, 80% yield) as a light yellow solid. LC purity: 96% (UV at 254 nm); Mass calculated for C₂₆H₂₂F₃N₅O₅[M+1]⁺ 542.1, found 542.1; Retention time: 0.955 min.

Step 5 & 6: (6-(5-amino-6-fluoropyrazin-2-yl)-6′-ethoxy-5,5′-difluoro-[2,3′-bipyridin]-4-yl)((1S,4S)-2,5-diazabicyclo[2.2.2]octan-2-yl)methanone (76 mg) was prepared in a 28% overall yield as a with solid, following a procedure analogous to that described in Example 2, using 6-(5-((2,4-dimethoxybenzyl)amino)-6-fluoropyrazin-2-yl)-6′-ethoxy-5,5′-difluoro-[2,3′-bipyridine]-4-carboxylic acid and tert-butyl (1S,4S)-2,5-diazabicyclo[2.2.2]octane-2-carboxylate in Step 1. LC purity: 85% (UV at 254 nm); Mass calculated for C₂₃H₂₂F₃N₇O₂[M+H]⁺ 486.2, found 486.2. Retention time: 0.680 min.

Step 7: To a solution of (6-(5-amino-6-fluoropyrazin-2-yl)-6′-ethoxy-5,5′-difluoro-[2,3′-bipyridin]-4-yl)((1S,4S)-2,5-diazabicyclo[2.2.2]octan-2-yl)methanone (1.0 eq, 60 mg, 0.1 mmol) in methanol (2 mL) were added formaldehyde (1.1 eq, 4.1 mg, 0.1 mmol) and acetic acid (3.0 eq, 22 mg, 0.4 mmol), the resulting mixture was stirred at room temperature for 20 min. NaBH₃CN (3.0 eq, 25 mg, 0.4 mmol) was added to above mixture at 0° C. and the reaction was stirred at room temperature for 1 h. The reaction mixture was diluted with water and extracted with EtOAc. The organic layers were washed with brine over 3 times, dried over anhydrous sodium sulfate, filtered and concentrated in vacuo. The residue was purified by Prep-HPLC with the following conditions: Column: xBridge Prep Shield RP18 5 m OBD 30*150 mm; Mobile Phase A: Water (10 mmol/L NH₄HCO₃+0.1% NH₃·H₂O), Mobile Phase B: ACN; Flow rate: 60 mL/min; Wavelength: 220/254 nm; RT1(min): 9.2. The fraction was collected and concentrated under vacuum, and the residue was re-dissolved in CH₃CN/H₂O, and then was lyophilized to afford the title compound (20 mg, 0.1 mmol, 33% yield) as a white solid. LC purity: 99.6% (UV at 254 nm); Mass calculated for C₂₋₄H₂₄F₃N₇O₂ [M+H]⁺ 500.2, found 500.2. Retention time: 1.272 min. H NMR (400 MHz, DMSO) δ 8.85-8.75 (m, 2H), 8.45-8.39 (m, 1H), 8.10 (t, J=3.7 Hz, 1H), 7.40 (s, 2H), 4.49-4.41 (m, 2H), 3.82 (d, J=12.7 Hz, 1H), 3.51-3.42 (m, 2H), 2.88 (s, 3H), 2.32 (d, J=13.9 Hz, 3H), 2.00 (s, 1H), 1.81 (d, J=16.4 Hz, 1H), 1.64 (t, J=10.8 Hz, 2H), 1.40-1.31 (m, 3H).

### Example 41. [2-(5-amino-6-fluoro-pyrazin-2-yl)-6-(6-ethoxy-3-pyridyl)-3-fluoro-4-pyridyl]-[(1S,4S)-5-ethyl-2,5-diazabicyclo[2.2.2]octan-2-yl]methanone

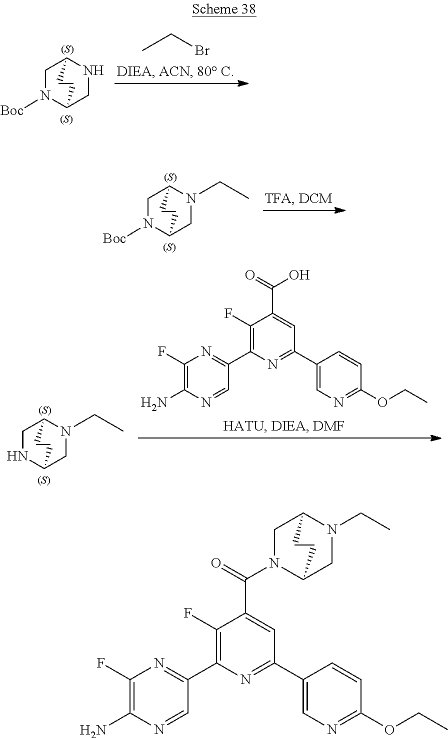

Step 1: To a solution of tert-butyl (1S,4S)-2,5-diazabicyclo[2.2.2]octane-2-carboxylate (1.0 eq, 300 mg, 1.4 mmol) in MeCN (3 mL) was added Bromoethane (1.5 eq, 0.2 mL, 2.1 mmol) and DIEA (2.0 eq, 365 mg, 2.8 mmol). The mixture was stirred at 80° C. for 2 h. The reaction was cooled to room temperature. The mixture was purified by reversed phase column to afford tert-butyl (1S,4S)-5-ethyl-2,5-diazabicyclo[2.2.2]octane-2-carboxylate (110 mg, 0.5 mmol, 32% yield) as a yellow oil. LC purity: N/A; Mass calculated for C₁₃H₂₄N₂O₂ [M+1]⁺ 241.2, found 241.2; Retention time: 0.40 min.

Step 2: To a solution of tert-butyl (1S,4S)-5-ethyl-2,5-diazabicyclo[2.2.2]octane-2-carboxylate (1.0 eq, 110 mg, 0.5 mmol) in DCM (3 mL) was added TFA (3 mL). The reaction was stirred at room temperature for 2 h. The solvent was removed under vacuum to afford crude product as a yellow oil which was used directly in the next step without further purification. LC purity: N/A; Mass calculated for C₈H₁₆N₂[M+1]⁺ 141.1, found 141.2; Retention time: 0.26 min.

Step 3: To a solution of 2-(5-amino-6-fluoro-pyrazin-2-yl)-6-(6-ethoxy-3-pyridyl)-3-fluoro-pyridine-4-carboxylic acid (1.0 eq, 80 mg, 0.2 mmol) and (1S,4S)-2-ethyl-2,5-diazabicyclo[2.2.2]octane (1.0 eq, 30 mg, 0.2 mmol) in DMF (3 mL) were added DIEA (3.0 eq, 83 mg, 0.6 mmol) and HATU (1.5 eq, 122 mg, 0.3 mmol). The mixture was stirred at room temperature for 2 h. The mixture was purified by Prep-HPLC using the following conditions: Column: X-Select Prep OBD C18 Column, 30*150 mm, 5 μm; Mobile Phase A: Water (0.1% FA), Mobile Phase B: ACN; Flow rate: 60 mL/min; Gradient (B %): 7% B to 36% B in 10 min; Wavelength: 254 nm/220 nm; RT1(min): 8.11. The fraction was collected and concentrated under vacuum, and the residue was re-dissolved in CH₃CN/H₂O, and then was lyophilized to afford the title compound (20 mg, 0.1 mmol, 16% yield) as an off-white solid. HPLC purity: 96.7% (UV at 254 nm); Mass calculated for C₂₅H₂₇F₂N₇O₂[M+1]⁺ 496.2, found 496.2; Retention time: 6.51 min. ¹H NMR (400 MHz, DMSO) δ 8.96 (dd, J=7.8, 2.5 Hz, 1H), 8.74 (dd, J=7.7, 4.6 Hz, 1H), 8.52-8.43 (m, 1H), 8.04 (t, J=4.0 Hz, 1H), 7.39 (s, 2H), 6.93 (dd, J=8.7, 3.7 Hz, 1H), 4.44-4.34 (m, 2H), 3.84-3.71 (m, 1H), 3.62-3.58 (m, 2H), 3.03-2.65 (m, 3H), 2.62-2.54 (m, 2H), 2.05-1.75 (m, 2H), 1.66 (d, J=11.7 Hz, 2H), 1.36 (t, J=7.0 Hz, 3H), 1.04-0.94 (m, 3H).

### Example 42. (6-(5-amino-6-fluoropyrazin-2-yl)-6′-ethoxy-5,5′-difluoro-[2,3′-bipyridin]-4-yl)((1S,4S)-5-ethyl-2,5-diazabicyclo[2.2.2]octan-2-yl)methanone

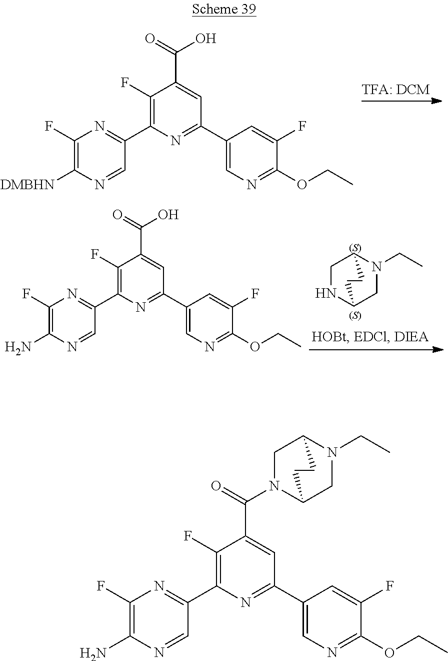

Step 1: To a solution of 6-(5-((2,4-dimethoxybenzyl)amino)-6-fluoropyrazin-2-yl)-6′-ethoxy-5,5′-difluoro-[2,3′-bipyridine]-4-carboxylic acid (preparation in Example 40, 1.0 eq, 360 mg, 0.6 mmol) in DCM (4 mL) was added TFA (4 mL). The mixture was stirred at room temperature for 1 h. The resulting mixture was concentrated under reduced pressure. The crude product was purified by reversed phase flash (FA/H₂O) to afford 5-amino-N-(2-azabicyclo[2.2.2]octan-4-yl)-6″-ethoxy-3′, 5″,6-trifluoro-[2,2′:6′,3″-terpyridine]-4′-carboxamide (240 mg, 0.4 mmol, 80% yield) as a yellow solid. LC purity: 87.1% (UV at 254 nm); Mass calculated for C₁₇H₁₂F₃N₅O₃[M+H]⁺, 392.1, found 392.1; Retention time: 0.601 min.

Step 2: To a solution of 6-(5-amino-6-fluoropyrazin-2-yl)-6′-ethoxy-5,5′-difluoro-[2,3′-bipyridine]-4-carboxylic acid (1.0 eq, 50 mg, 0.1 mmol) and (1S,4S)-2-ethyl-2,5-diazabicyclo[2.2.2]octane (1.0 eq, 18 mg, 0.1 mmol) in methanol (10 mL) were added HOBT (2.2 eq, 38 mg, 0.2 mmol), EDCI (2.2 eq, 55 mg, 0.2 mmol) and DIEA (3.3 eq, 55 mg, 0.4 mmol). The mixture was stirred at room temperature for overnight. The resulting mixture was quenched with water and extracted with EtOAc (2×100 mL). The combined organic layers were washed with brine (1×30 mL), dried over anhydrous Na₂SO₄ and filtered. The filtrate was concentrated in vacuo and the residue was purified by Prep-HPLC with following conditions: Column: XSelect CSH Fluoro Phenyl 30*150 mm, 5 μm; Mobile Phase A: Water (0.1% FA), Mobile Phase B: ACN; Flow rate: 60 mL/min; Gradient (B %): 4% B to 29% B in 10 min; Wavelength: 254 nm/220 nm; RT1(min): 7.85. The pure fractions were concentrated and lyophilized to afford title compound (15 mg, 0.02 mmol, 22% yield) as a white solid. LC purity: 98.8% (UV at 254 nm); Mass calculated for Chemical Formula: C₂₅H₂₆F₃N₇O₂[M+H]⁺, 514.2, found 514.2; Retention time:1.030 min. ¹H NMR (400 MHz, DMSO) δ 8.84-8.74 (m, 2H), 8.49-8.38 (m, 1H), 8.17-8.05 (m, 1H), 7.40 (s, 2H), 4.55-4.44 (m, 2H), 3.94 (s, 1H), 3.79 (d, J=12.7 Hz, 1H), 3.53 (d, J=11.8 Hz, 2H), 3.10 (s, 1H), 2.92 (d, J=10.2 Hz, 1H), 2.69-2.57 (m, 2H), 1.92 (d, J=58.4 Hz, 2H), 1.67 (t, J=11.5 Hz, 2H), 1.39 (t, J=7.0 Hz, 3H), 1.02 (t, J=6.9 Hz, 3H).

### Example 43. (6-(5-amino-6-fluoropyrazin-2-yl)-6′-ethoxy-5-fluoro-[2,3′-bipyridin]-4-yl)((1R,4R)-5-(ethyl-d5)-2,5-diazabicyclo[2.2.2]octan-2-yl)methanone

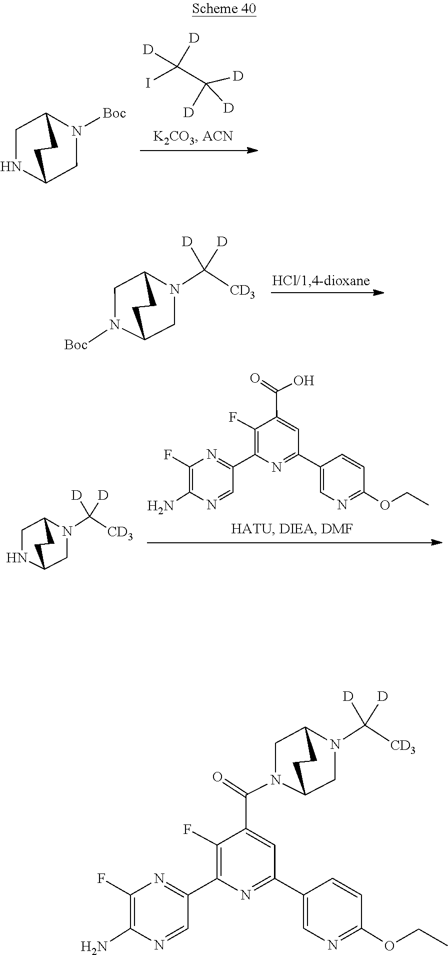 [SMILES: [2H]C([2H])([2H])C([2H])([2H])I ; CC(C)(C)OC(=O)N1C[C@H]2CC[C@@H]1CN2 ; [2H]C([2H])([2H])C([2H])([2H])N1C[C@H]2CC[C@@H]1CN2C(=O)OC(C)(C)C ; [2H]C([2H])([2H])C([2H])([2H])N1C[C@H]2CC[C@@H]1CN2 ; CCOc1ccc(-c2cc(C(=O)O)c(F)c(-c3cnc(N)c(F)n3)n2)cn1 ; [2H]C([2H])([2H])C([2H])([2H])N1C[C@H]2CC[C@@H]1CN2C(=O)c1cc(-c2ccc(OCC)nc2)nc(-c2cnc(N)c(F)n2)c1F]

Step 1: To a solution of 1-iodoethane-1,1,2,2,2-d5 (1.2 eq, 455 mg, 2.8 mmol) and tert-butyl (1R,4R)-2,5-diazabicyclo[2.2.2]octane-2-carboxylate (1.0 eq, 500 mg, 2.3 mmol) in MeCN (8 mL) was added K₂CO₃ (2.0 eq, 650 mg, 4.7 mmol). The mixture was stirred at 50° C. for 2 h. The solid was filtered out and washed with DCM. The filtrate was concentrated in vacuo and the residue was purified by silica gel column chromatography to afford tert-butyl (1R,4R)-5-(ethyl-d5)-2,5-diazabicyclo[2.2.2]octane-2-carboxylate(380 mg, 1.6 mmol, 65% yield) as a colorless oil. LC purity: N/A; Mass calculated for C₁₃H₁₉D₅N₂O₂[M+1]⁺ 246.2, found 246.2; Retention time: 0.40 min.

Step 2: To a solution of tert-butyl (1R,4R)-5-(ethyl-d5)-2,5-diazabicyclo[2.2.2]octane-2-carboxylate (1.0 eq, 150 mg, 0.6 mmol) in 1,4-dioxane (2.0 mL) was added (4M) HCl-dioxane (1.0 mL). The mixture was stirred at room temperature for 1 h. The solvent was removed under vacuum. The crude product (1R,4R)-2-(ethyl-d5)-2,5-diazabicyclo[2.2.2]octane (86 mg, 0.5 mmol, 96% yield) was obtained as a yellow oil without further purification. LC purity: N/A; Mass calculated for C₈H₁₁DsN₂ [M+1]⁺ 146.2, found 146.2; Retention time: 0.23 min.

Step 3: To a solution of (1R,4R)-2-(ethyl-d5)-2,5-diazabicyclo[2.2.2]octane (1.0 eq, 78 mg, 0.5 mmol) and 6-(5-amino-6-fluoropyrazin-2-yl)-6′-ethoxy-5-fluoro-[2,3′-bipyridine]-4-carboxylic acid (1.0 eq, 200 mg, 0.5 mmol) in DMF (8 mL) were added DIEA (3.0 eq, 207 mg, 1.6 mmol) and HATU (1.5 eq, 305 mg, 0.8 mmol). The mixture was stirred at room temperature for 3 h. The mixture was purified by reversed phase column to obtain title compound (117 mg, 0.2 mmol, 42% yield) as a light-yellow solid. LC purity: 97% (UV at 254 nm); Mass calculated for C₂₅H₂₂D₅F₂N₇O₂ [M+1]⁺ 501.2, found 501.2; Retention time: 1.324 min. ¹H NMR (400 MHz, DMSO) δ 8.96 (dd, J=7.9, 2.5 Hz, 1H), 8.73 (dd, J=7.7, 4.6 Hz, 1H), 8.52-8.43 (m, 1H), 8.04 (t, J=4.1 Hz, 1H), 7.39 (s, 2H), 6.93 (dd, J=8.7, 3.7 Hz, 1H), 4.42-4.33 (m, 2H), 3.84-3.73 (m, 1H), 3.63-3.47 (m, 2H), 2.97-2.95 (m, 1H), 2.96-2.68 (m, 2H), 1.81-1.78 (m, 4H), 1.36 (t, J=7.0 Hz, 3H).

### Example 44. (6-(5-amino-6-fluoropyrazin-2-yl)-6′-ethoxy-5-fluoro-[2,3′-bipyridin]-4-yl)((1S,4S)-2,5-diazabicyclo[2.2.2]octan-2-yl)methanone

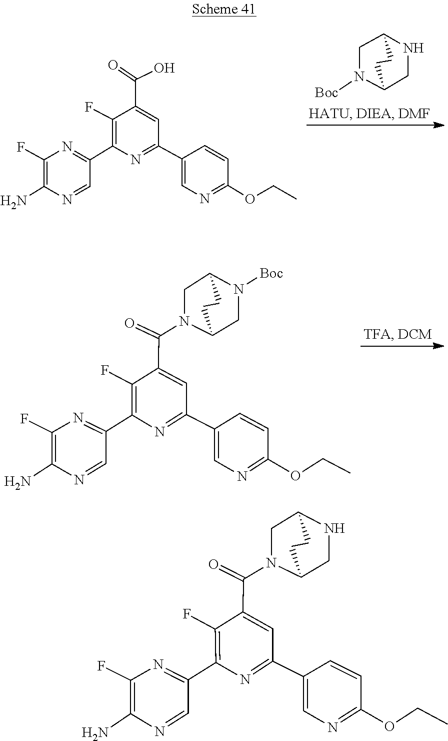

Step 1: To a solution of 6-(5-amino-6-fluoropyrazin-2-yl)-6′-ethoxy-5-fluoro-[2,3′-bipyridine]-4-carboxylic acid (1.0 eq, 80 mg, 0.8 mmol) and tert-butyl (1S,4S)-2,5-diazabicyclo[2.2.2]octane-2-carboxylate (1.0 eq, 50 mg, 0.8 mmol) in DMF (5 mL) were added DIEA (2.0 eq, 60 mg, 1.6 mmol) and HATU (2.0 eq, 120 mg, 1.6 mmol). The mixture was stirred at room temperature for overnight. The resulting mixture was quenched with water and extracted with EtOAc (2×100 mL). The combined organic layers were washed with brine (1×30 mL), dried over anhydrous Na₂SO₄ and filtered. The filtrate was concentrated in vacuo and the residue was purified via reversed phase flash chromatography using a gradient of 70% to 100% ACN in water (with 10 mmol/L NH₄HCO₃) to afford tert-butyl (1S,4S)-5-(6-(5-amino-6-fluoropyrazin-2-yl)-6′-ethoxy-5-fluoro-[2,3′-bipyridine]-4-carbonyl)-2,5-diazabicyclo[2.2.2]octane-2-carboxylate (60 mg, 0.9 mmol, 41% yield) as a yellow solid. LC purity: 90.2% (UV at 254 nm); Mass calculated for C₂₈H₃₁F₂N₇O₄[M+H]⁺, 568.2, found 568.2; Retention time: 0.992 min.

Step 2: To a solution of tert-butyl (1S,4S)-5-(6-(5-amino-6-fluoropyrazin-2-yl)-6′-ethoxy-5-fluoro-[2,3′-bipyridine]-4-carbonyl)-2,5-diazabicyclo[2.2.2]octane-2-carboxylate (1.0 eq, 60 mg, 0.9 mmol) in DCM (4 mL) was added TFA (4 mL). The mixture was stirred at room temperature for 1 h. The resulting mixture was concentrated under reduced pressure. The crude product was purified by Prep-HPLC with following conditions: Column: Waters Xselcet C18 OBD Column 30*150 mm 5 μm; Mobile Phase A: Water (0.1% FA), Mobile Phase B: ACN; Flow rate: 60 mL/min; Gradient (B %): 5% B to 25% B in 7 min; Wavelength: 254/220 nm; RT1(min): 6.52. The pure fractions were concentrated and lyophilized to afford title compound (24 mg, 0.02 mmol, 22% yield) as a white solid. LC purity: 99.4% (UV at 254 nm); Mass calculated for Chemical Formula: C₂₃H₂₃F₂N₇O₂ [M+H]⁺, 468.2, found 468.2; Retention time:0.839 min. ¹H NMR (400 MHz, DMSO) δ 8.92-8.79 (m, 1H), 8.76-8.59 (m, 1H), 8.45-8.36 (m, 1H), 8.34 (s, 1H), 8.01-7.85 (m, 1H), 6.99-6.85 (m, 1H), 4.39-4.27 (m, 2H), 3.76-3.50 (m, 3H), 3.47-3.14 (m, 2H), 3.06 (d, J=11.9 Hz, 1H), 2.05-1.66 (m, 4H), 1.37-1.22 (m, 3H).

### Example 45. (6-(5-amino-6-fluoropyrazin-2-yl)-6′-ethoxy-5,5′-difluoro-[2,3′-bipyridin]-4-yl)((1S,4S)-2,5-diazabicyclo[2.2.2]octan-2-yl)methanone

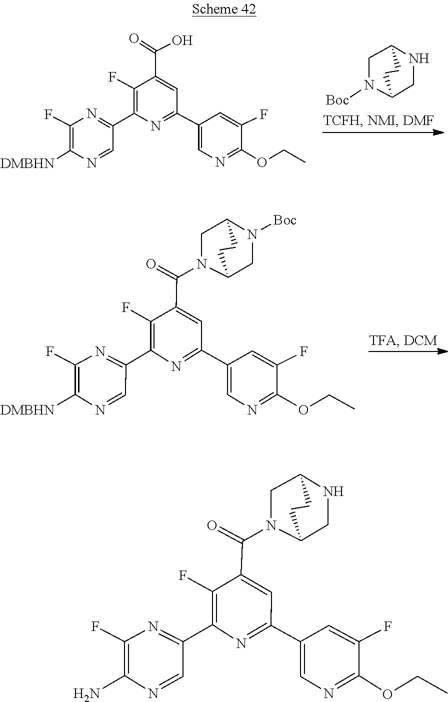

Step 1: To a solution of 6-(5-((2,4-dimethoxybenzyl)amino)-6-fluoropyrazin-2-yl)-6′-ethoxy-5,5′-difluoro-[2,3′-bipyridine]-4-carboxylic acid (preparation in Example 40, 1.0 eq, 50 mg, 0.1 mmol) and tert-butyl (1S,4S)-2,5-diazabicyclo[2.2.2]octane-2-carboxylate (1.2 eq, 24 mg, 0.1 mmol) in DMF (2 mL) were added TCFH (2.0 eq, 52 mg, 0.2 mmol) and NMI (3.0 eq, 23 mg, 0.3 mmol) at 0° C. The reaction was stirred for 1 h at room temperature. The resulting mixture was diluted with water and extracted with EtOAc. The organic layers were washed with brine, dried and concentrated to afford crude tert-butyl (1S,4S)-5-(6-(5-((2,4-dimethoxybenzyl)amino)-6-fluoropyrazin-2-yl)-6′-ethoxy-5,5′-difluoro-[2,3′-bipyridine]-4-carbonyl)-2,5-diazabicyclo[2.2.2]octane-2-carboxylate (80 mg, 0.1 mmol, 82% yield) as a black oil. LC purity: 69.4% (UV at 254 nm); Mass calculated for C₃₇H₄₀F₃N₇O₆[M+1]⁺ 736.3, found 736.2; Retention time: 1.038 min.

Step 2: To a solution of tert-butyl (1S,4S)-5-[2-[5-[(2,4-dimethoxyphenyl)methylamino]-6-fluoro-pyrazin-2-yl]-6-(6-ethoxy-5-fluoro-3-pyridyl)-3-fluoro-pyridine-4-carbonyl]-2,5-diazabicyclo[2.2.2]octane-2-carboxylate (1.0 eq, 87 mg, 0.1 mmol) in DCM (2 mL) was added TFA (0.2 mL) and stirred at room temperature for 1 h. The resulting mixture was treated with 1 mL NH₄OH at 0° C. and concentrated in vacuo. The residue was purified via prep-HPLC with following condition: Column: XBridge Prep OBD C18 Column, 30*150 mm, 5 μm; Mobile Phase A: Water (10 mmol/L NH₄HCO₃+0.05% NH₁₃H₂O), Mobile Phase B: ACN; Flow rate: 60 mL/min; Gradient (B %): 22% B to 42% B in 8 min; Wavelength: 254 nm/220 nm; RT1(min): 7.26. The pure fractions were concentrated and lyophilized to afford title compound (21 mg, 0.04 mmol, 50% yield) as a light yellow solid. LC purity: 97.1% (UV at 254 nm); Mass calculated for C₂₃H₂₂F₃N₇O₂[M+1]⁺ 486.2, found 486.1; Retention time: 2.635 min. ¹H NMR (400 MHz, DMSO) δ 8.84-8.75 (m, 2H), 8.45-8.42 (m, 1H), 8.09 (d, J=3.6 Hz, 1H), 7.40 (s, 2H), 4.49-4.45 (m, 2H), 3.66 (d, J=12.4 Hz, 1H), 3.57 (dd, J=12.3, 2.0 Hz, 1H), 3.45 (d, J=14.7 Hz, 1H), 3.17-3.09 (m, 2H), 2.88 (d, J=12.2 Hz, 1H), 1.87-1.85 (m, 2H), 1.72 (d, J=11.0 Hz, 2H), 1.40-1.38 (m, 3H).

### Example 46. (6-(5-amino-6-fluoropyrazin-2-yl)-6′-ethoxy-5,5′-difluoro-[2,3′-bipyridin]-4-yl)((1S,4S)-5-(methyl-d3)-2,5-diazabicyclo[2.2.2]octan-2-yl)methanone

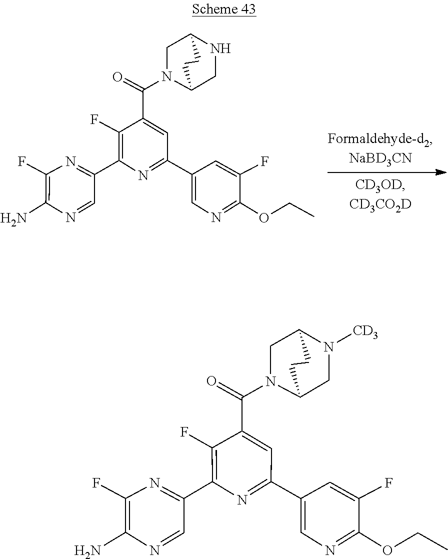 [SMILES: CCOc1ncc(-c2cc(C(=O)N3C[C@@H]4CC[C@H]3CN4)c(F)c(-c3cnc(N)c(F)n3)n2)cc1F ; [2H]C([2H])([2H])N1C[C@@H]2CC[C@H]1CN2C(=O)c1cc(-c2cnc(OCC)c(F)c2)nc(-c2cnc(N)c(F)n2)c1F]

Step 1: To a solution of (6-(5-amino-6-fluoropyrazin-2-yl)-6′-ethoxy-5,5′-difluoro-[2,3′-bipyridin]-4-yl)((1S,4S)-2,5-diazabicyclo[2.2.2]octan-2-yl)methanone (1.0 eq, 200 mg, 0.4 mmol) and formaldehyde-d₂ (1.5 eq, 99 mg, 0.6 mmol) in CD₃0D (5.0 mL) was added CD₃CO₂D (10.0 eq, 206 mg, 4.12 mmol) and the solution was stirred for 10 min and then NaBD₃CN (5.0 eq, 134 mg, 2.1 mmol) was added into the above mixture at 0° C. and then stirred at room temperature for 1 h. The resulting mixture was diluted with ice-water and extracted with EtOAc. The organic layers were washed with brine, dried and concentrated in vacuo. The residue was purified with following condition: Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 27% B to 52% B in 10 min; Wavelength: 254 nm/220 nm; RT1(min): 7.96. The pure fractions were concentrated and lyophilized to afford title compound (117 mg, 0.2 mmol, 56% yield) as a light-yellow solid. LC purity: 99.0% (UV at 254 nm); Mass calculated for C₂₋₄H₂₁D₃F₃N₇O₂ [M+1]⁺, 503.2, found 503.2; Retention time: 0.944 min. ¹H NMR (400 MHz, DMSO) δ 8.85-8.75 (m, 2H), 8.45 (m, 1H), 8.10 (t, J=3.7 Hz, 1H), 7.40 (s, 2H), 4.49 (m, 2H), 3.82 (d, J=12.7 Hz, 1H), 3.60-3.42 (m, 2H), 2.87 (s, 2H), 2.67 (m, 1H), 2.00 (s, 1H), 1.83 (s, 1H), 1.64 (t, J=11.3 Hz, 2H), 1.40 (m, 3H).

### Example 47. (6-(5-amino-6-fluoropyrazin-2-yl)-6′-ethoxy-5-fluoro-[2,3′-bipyridin]-4-yl)((1R,4R)-2,5-diazabicyclo[2.2.2]octan-2-yl)methanone

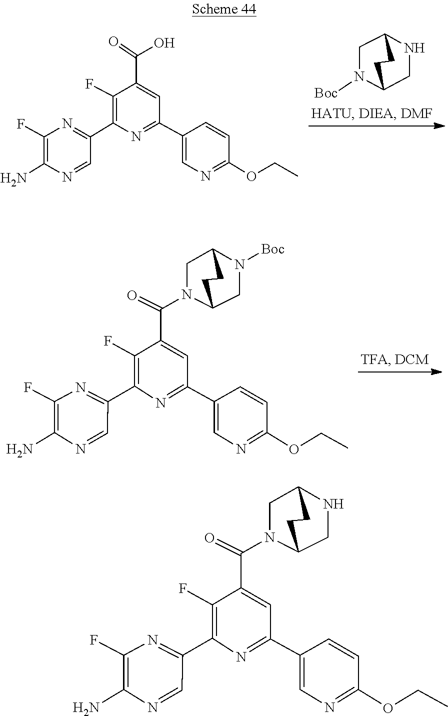

Step 1: To a solution of 6-(5-amino-6-fluoropyrazin-2-yl)-6′-ethoxy-5-fluoro-[2,3′-bipyridine]-4-carboxylic acid (1.0 eq, 80 mg, 0.2 mmol) and tert-butyl (1R,4R)-2,5-diazabicyclo[2.2.2]octane-2-carboxylate (2.0 eq, 91 mg, 0.4 mmol) in DMF (3 mL) were added DIEA (3.0 eq, 83 mg, 0.6 mmol) and HATU (1.5 eq, 122 mg, 0.3 mmol). The mixture was stirred at room temperature overnight. The resulting mixture was quenched with water and extracted with EtOAc (2×100 mL The combined organic layers were washed with brine (1×30 mL), dried over anhydrous Na₂SO₄ and filtered. The filtrate was concentrated in vacuo and the residue was purified via reversed phase flash chromatography using a gradient of 70% to 100% ACN in water (with 10 mmol/L FA) to afford tert-butyl (1R,4R)-5-(6-(5-amino-6-fluoropyrazin-2-yl)-6′-ethoxy-5-fluoro-[2,3′-bipyridine]-4-carbonyl)-2,5-diazabicyclo[2.2.2]octane-2-carboxylate (50 mg, 0.1 mmol, 41% yield) as a yellow solid. LC purity: 81.1% (UV at 254 nm); Mass calculated for C₂₈H₃₁F₂N₇O₄[M+H]⁺, 568.2, found 568.2; Retention time: 0.642 min.

Step 2: To a solution of tert-butyl (1R,4R)-5-(6-(5-amino-6-fluoropyrazin-2-yl)-6′-ethoxy-5-fluoro-[2,3′-bipyridine]-4-carbonyl)-2,5-diazabicyclo[2.2.2]octane-2-carboxylate (1.0 eq, 50 mg, 0.1 mmol) in DCM (2 mL) was added TFA (2 mL) and the mixture was stirred at room temperature for 1 h. The resulting mixture was treated with 1 mL NH₄OH at 0° C. and concentrated in vacuo. The residue was purified with following condition: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 19% B to 39% B in 8 min; Wavelength: 254 nm/220 nm nm; RT1(min): 7.34. The pure fractions were concentrated and lyophilized to afford title compound (21 mg, 0.1 mmol, 50% yield) as a white solid. LC purity: 98.6% (UV at 254 nm); Mass calculated for C₂₃H₂₃F₂N₇O₂ [M+H]⁺ 468.2, found 468.2; Retention time: 1.096 min. ¹H NMR (400 MHz, MeOD) δ 8.86 (dd, J=8.9, 2.5 Hz, 1H), 8.69 (t, J=4.8 Hz, 1H), 8.43-8.40 (m, 1H), 7.89 (dd, J=8.7, 3.7 Hz, 1H), 6.91 (dd, J=8.7, 3.3 Hz, 1H), 4.42-4.40 (m, 2H), 3.88-3.85 (m, 1H), 3.76 (dd, J=12.9, 1.9 Hz, 1H), 3.66 (d, J=11.6 Hz, 1H), 3.27 (t, J=2.9 Hz, 2H), 3.10-3.00 (m, 1H), 2.11-2.00 (m, 2H), 1.98-1.86 (m, 2H), 1.43-1.40 (m, 3H).

### Example 48. (6-(5-amino-6-fluoropyrazin-2-yl)-6′-ethoxy-5,5′-difluoro-[2,3′-bipyridin]-4-yl)((1R,4R)-5-(methyl-d3)-2,5-diazabicyclo[2.2.2]octan-2-yl)methanone

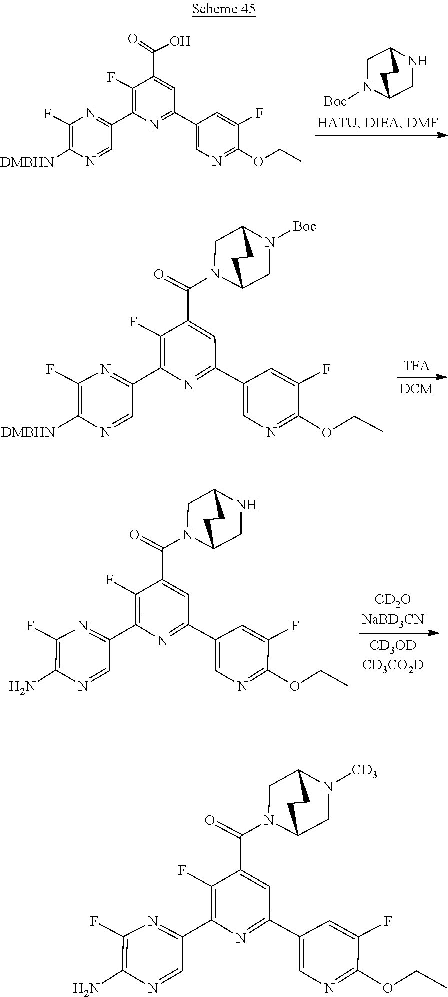

Step 1 & 2: (6-(5-amino-6-fluoropyrazin-2-yl)-6′-ethoxy-5,5′-difluoro-[2,3′-bipyridin]-4-yl)((1R,4R)-2,5-diazabicyclo[2.2.2]octan-2-yl)methanone (420 mg) was prepared in a 52% overall yield as an off-white solid, following a procedure analogous to that described in Example 2, using 6-(5-((2,4-dimethoxybenzyl)amino)-6-fluoropyrazin-2-yl)-6′-ethoxy-5,5′-difluoro-[2,3′-bipyridine]-4-carboxylic acid and tert-butyl (1R,4R)-2,5-diazabicyclo[2.2.2]octane-2-carboxylate in Step 1. LC purity: 74% (UV at 254 nm); Mass calculated for C₂₃H₂₂F₃N₇O₂[M+1]⁺, 486.1, found 486.1; Retention time: 0.726 min.

Step 3: To a solution of (6-(5-amino-6-fluoropyrazin-2-yl)-6′-ethoxy-5,5′-difluoro-[2,3′-bipyridin]-4-yl)((1R,4R)-2,5-diazabicyclo[2.2.2]octan-2-yl)methanone (1.0 eq, 200 mg, 0.5 mmol) and formaldehyde-d₂ (1.2 eq, 79 mg, 0.5 mmol) in CD₃0D (5 mL) was added CD₃COOD (1.5 eq, 40 mg, 0.6 mmol) and the solution was stirred for 10 min and then NaBD₃CN (2.0 eq, 54 mg, 0.8 mmol) was added into the above mixture at 0° C. and stirred at room temperature for 1 h. The resulting mixture was diluted with ice-water and extracted with EtOAc. The organic layers were washed with brine, dried and concentrated in vacuo. The residue was purified by Prep-HPLC with following condition: Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 27% B to 52% B in 10 min; Wavelength: 254 nm/220 nm; RT1(min): 7.96. The pure fractions were concentrated and lyophilized to afford title compound (122 mg, 0.3 mmol, 58% yield) as a white solid. LC purity: 99% (UV at 254 nm); Mass calculated for C₂₋₄H₂₁D₃F₃N₇O₂ [M+1]⁺, 503.2, found 503.2; Retention time: 1.262 min. ¹H NMR (400 MHz, DMSO) δ 8.84-8.74 (m, 2H), 8.51-8.38 (m, 1H), 8.09 (t, J=3.8 Hz, 1H), 7.40 (s, 2H), 4.54-4.37 (m, 2H), 3.86-3.75 (m, 1H), 3.56-3.37 (m, 3H), 2.96-2.71 (m, 2H), 2.00 (s, 1H), 1.90-1.54 (m, 3H), 1.46-1.33 (m, 3H).

### Example 49. (6-(5-amino-6-fluoropyrazin-2-yl)-6′-ethoxy-5,5′-difluoro-[2,3′-bipyridin]-4-yl)((1R,4R)-5-methyl-2,5-diazabicyclo[2.2.2]octan-2-yl)methanone

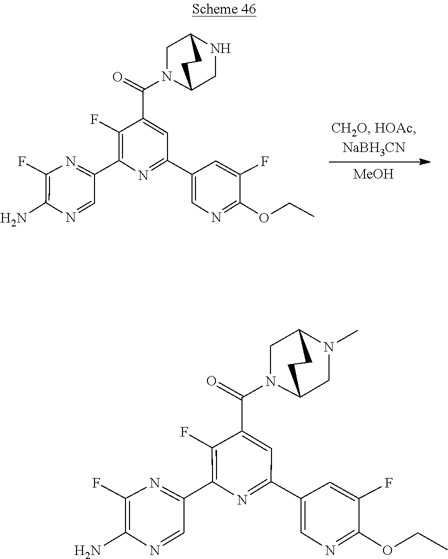 [SMILES: "MeOH" ; CCOc1ncc(-c2cc(C(=O)N3C[C@H]4CC[C@@H]3CN4)c(F)c(-c3cnc(N)c(F)n3)n2)cc1F ; CCOc1ncc(-c2cc(C(=O)N3C[C@H]4CC[C@@H]3CN4C)c(F)c(-c3cnc(N)c(F)n3)n2)cc1F]

Step 1: To a solution of (6-(5-amino-6-fluoropyrazin-2-yl)-6′-ethoxy-5,5′-difluoro-[2,3′-bipyridin]-4-yl)((1R,4R)-2,5-diazabicyclo[2.2.2]octan-2-yl)methanone (preparation in Example 48, 1.0 eq, 200 mg, 0.4 mmol) in methanol (5 mL) were added formaldehyde (1.2 eq, 3708 mg, 0.5 mmol) and acetic acid (1.5 eq, 37 mg, 0.6 mmol), the resulting mixture was stirred at room temperature for 20 min. NaBH₃CN (2.0 eq, 52 mg, 0.8 mmol) was added to above mixture at 0° C. and the reaction was stirred at room temperature for 1 h. The reaction mixture was diluted with water and extracted with EtOAc. The organic layers were washed with brine over 3 times, dried over anhydrous sodium sulfate, filtered and concentrated in vacuo. The residue was purified by Prep-HPLC with the following conditions: Column: X-Select Prep OBD C18 Column, 30*150 mm, 5 μm; Mobile Phase A: Water (0.1% FA), Mobile Phase B: ACN; Flow rate: 60 mL/min; Gradient (B %): 11% B to 40% B in 10 min; Wavelength: 254 nm/220 nm; RT1(min): 7.93. The fraction was collected and concentrated under vacuum, and the residue was re-dissolved in CH₃CN/H₂O, and then was lyophilized to afford the title compound (71 mg, 0.1 mmol, 31% yield) as a white solid. LCMS purity: 98% (UV at 254 nm); Mass calculated for C₂₋₄H₂₄F₃N₇O₂[M+1]⁺, 500.2, found 500.2; Retention time: 1.264 min. ¹H NMR (400 MHz, DMSO) δ 8.86-8.71 (m, 2H), 8.51-8.40 (m, 1H), 8.11-8.06 (m, 1H), 7.40 (s, 2H), 4.54-4.40 (m, 2H), 3.87-3.78 (m, 1H), 3.66-3.40 (m, 3H), 2.97-2.76 (m, 2H), 2.35 (d, J=13.9 Hz, 3H), 2.01 (s, 1H), 1.84 (s, 1H), 1.66 (t, J=10.8 Hz, 2H), 1.40 (t, J=7.0 Hz, 3H).

### Example 50. (6-(5-amino-6-fluoropyrazin-2-yl)-6′-ethoxy-5,5′-difluoro-[2,3′-bipyridin]-4-yl)((1R,4R)-5-(ethyl-d5)-2,5-diazabicyclo[2.2.2]octan-2-yl)methanone

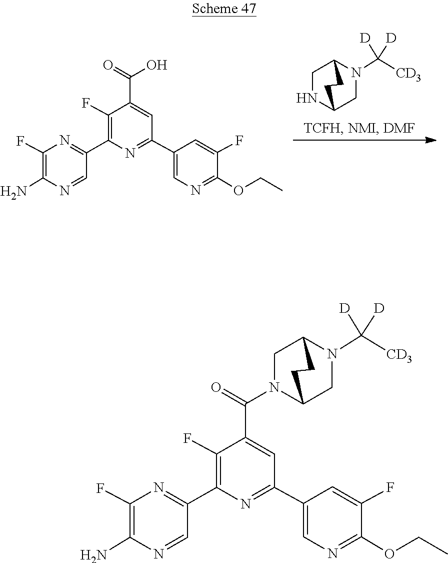 [SMILES: CCOc1ncc(-c2cc(C(=O)O)c(F)c(-c3cnc(N)c(F)n3)n2)cc1F ; [2H]C([2H])([2H])C([2H])([2H])N1C[C@H]2CC[C@@H]1CN2 ; [2H]C([2H])([2H])C([2H])([2H])N1C[C@H]2CC[C@@H]1CN2C(=O)c1cc(-c2cnc(OCC)c(F)c2)nc(-c2cnc(N)c(F)n2)c1F]

Step 1: To a solution of (1R,4R)-2-(ethyl-d5)-2,5-diazabicyclo[2.2.2]octane (preparation in Example 43, step 2, 1.1 eq, 100 mg, 0.7 mmol), 6-(5-amino-6-fluoropyrazin-2-yl)-6′-ethoxy-5,5′-difluoro-[2,3′-bipyridine]-4-carboxylic acid (1.0 eq, 245 mg, 0.6 mmol) and NMI (3.0 eq, 154 mg, 1.9 mmol) in DMF (10 mL) was added TCFH (1.5 eq, 264 mg, 0.9 mmol). The resulting mixture was stirred at room temperature for 1 h. The mixture was purified by reversed phase chromatography using a gradient of 40% to 70% ACN in water (with 5 mmol NH₄HCO₃) to afford the title compound (112 mg, 0.2 mmol, 34% yield) as light-yellow solid. LC purity: 97% (UV at 254 nm); Mass calculated for C₂₅H₂₁D₅F₃N₇O₂ [M+1]⁺, 519.6, found 519.2; Retention: 1.013. ¹H NMR (400 MHz, DMSO) δ 8.83-8.74 (m, 2H), 8.43 (d, J=11.9 Hz, 1H), 8.09 (d, J=3.5 Hz, 1H), 7.40 (s, 2H), 4.48 (d, J=7.2 Hz, 2H), 3.77 (d, J=12.4 Hz, 1H), 3.53-3.44 (m, 2H), 3.01 (s, 1H), 2.97-2.62 (m, 2H), 1.97 (s, 1H), 1.83 (s, 1H), 1.64 (s, 2H), 1.39 (t, J=7.0 Hz, 3H).

### Example 51. (5-amino-6″-(ethoxy-d5)-3′,5″,6-trifluoro-[2,2′:6′,3″-terpyridin]-4′-yl)((1S,4S)-5-(methyl-d3)-2,5-diazabicyclo[2.2.2]octan-2-yl)methanone

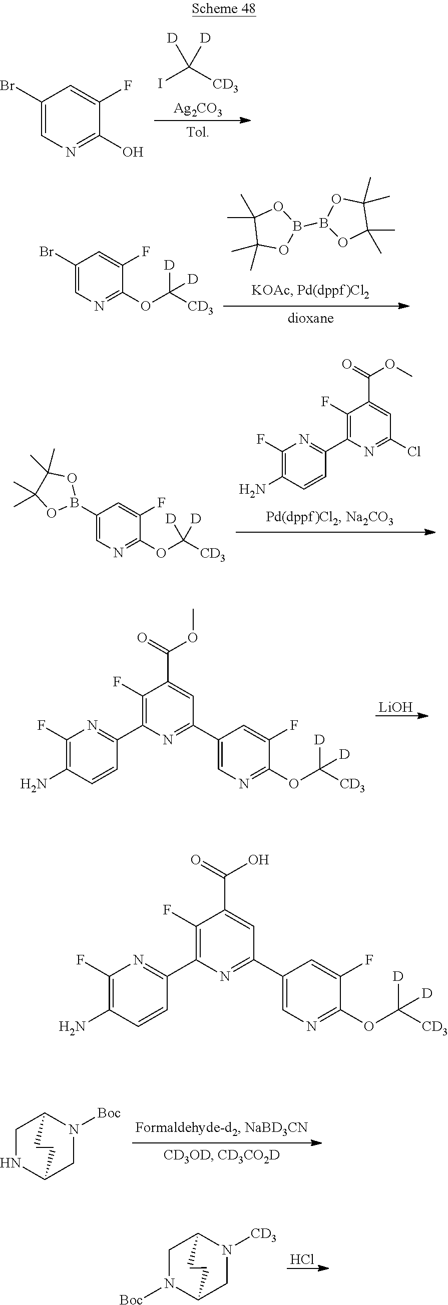

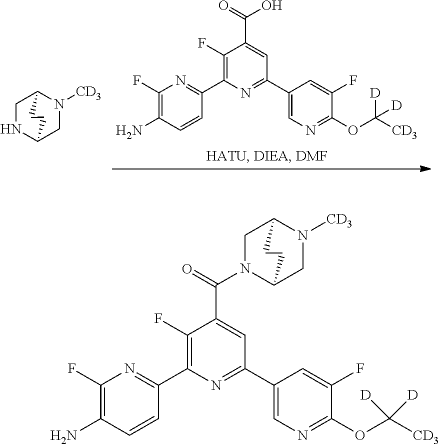 [SMILES: [2H]C([2H])([2H])N1C[C@@H]2CC[C@H]1CN2 ; [2H]C([2H])([2H])C([2H])([2H])Oc1ncc(-c2cc(C(=O)O)c(F)c(-c3ccc(N)c(F)n3)n2)cc1F ; [2H]C([2H])([2H])N1C[C@@H]2CC[C@H]1CN2C(=O)c1cc(-c2cnc(OC([2H])([2H])C([2H])([2H])[2H])c(F)c2)nc(-c2ccc(N)c(F)n2)c1F]

Step 1: To a stirred solution of 5-bromo-3-fluoropyridin-2-ol (1.0 eq, 5.0 g, 26 mmol) and 1-iodoethane-1,1,2,2,2-d₅(1.0 eq, 4.2 g, 26 mmol) in Toluene (30 mL) was added Ag₂CO₃ (1.0 eq, 7.2 g, 26 mmol) at room temperature. The resulting mixture was heated to 150° C. and stirred for 30 min. The resulting mixture was diluted with water (100 mL) and extracted with EtOAc (3*500 mL). The combined organic layer were washed with saturated NaCl solution (300 mL) and dried over anhydrous Na₂SO₄ and filtered. The filtrate was concentrated in vacuo and the residue was purified by column chromatography on silica gel (PE: EtOAc=0~20%) to afford 5-bromo-2-(ethoxy-d₅)-3-fluoropyridine (4.1 g, 18.2 mmol, 70% yield) as a yellow solid. LC purity: 78% (UV at 254 nm); Mass calculated for C₇H2D₅BrFNO [M+1]⁺ 225.0, found 225.0; Retention time: 0.556 min.

Step 2 & 3 & 4: The compound 5-amino-6″-(ethoxy-d5)-3′,5″,6-trifluoro-[2,2′:6′,3″-terpyridine]-4′-carboxylic acid (500 mg) was prepared in a 42% overall yield as a light yellow solid, following a procedure analogous to that described in Example 40 (Step 2 to Step 4). LC purity: 85.7% (UV at 254 nm); Mass calculated for C₁₈H8D₅F₃N₄O₃[M+1]⁺, 396.1, found 396.1; Retention time: 0.955 min.

Step 5: To a solution tert-butyl (1S,4S)-2,5-diazabicyclo[2.2.2]octane-2-carboxylate (1.0 eq, 300 mg, 1.4 mmol) in CD₃0D (5 mL) were added formaldehyde-d₂ (1.2 eq, 219 mg, 1.7 mmol) and CD₃COOD (3.0 eq, 271 mg, 4.2 mmol), the resulting mixture was stirred at room temperature for 20 min. NaBD₃CN (2.0 eq, 186 mg, 2.8 mmol) was added to above mixture at 0° C. and the reaction was stirred at room temperature for 1 h. The reaction mixture was diluted with water and extracted with EtOAc. The combined organic layers were washed with brine, dried over anhydrous sodium sulfate, filtered and concentrated in vacuo. The residue was purified via reversed phase flash chromatography using a gradient of 20% to 50% ACN in water (with 10 mmol/L NH₄HCO₃) to afford tert-butyl (1S,4S)-5-(methyl-d3)-2,5-diazabicyclo[2.2.2]octane-2-carboxylate (220 mg, 1.0 mmol, 68% yield) as a colorless oily liquid. LC purity: NA (UV at 254 nm); Mass calculated for C₁₂H₁₉D₃N₂O₂[M+1]⁺ 230.3, found 230.2; Retention time: 0.697 min.

Step 6: To a solution of tert-butyl (1S,4S)-5-(methyl-d3)-2,5-diazabicyclo[2.2.2]octane-2-carboxylate (1.0 eq, 230 mg, 1.0 mmol) in 1,4-dioxane (4 mL) was added 4 M HCl in dioxane (4 mL). The resulting mixture was stirred at room temperature for 1 h. The solvent was removed under vacuum to afford the crude (1S,4S)-2-(methyl-d3)-2,5-diazabicyclo[2.2.2]octane which was used directly in the next step without further purification. LC purity: NA (UV at 254 nm); Mass calculated for C₇H₁₁D₃N₂ [M+1]⁺, 130.2, found 130.1; Retention: 0.157.

Step 7: To a solution of (1S,4S)-2-(methyl-d3)-2,5-diazabicyclo[2.2.2]octane (1.0 eq, 33 mg, 0.3 mmol), 5-amino-6″-(ethoxy-d5)-3′,5″,6-trifluoro-[2,2′:6′,3″-terpyridine]-4′-carboxylic acid (1.0 eq, 100 mg, 0.3 mmol) in DMF (1 mL) was added DIEA (3.0 eq, 98 mg, 0.8 mmol) and HATU (1.5 eq, 144 mg, 0.4 mmol). The resulting solution was stirred at room temperature for 1 h. The resulting mixture was quenched with water and extracted with EtOAc (2×100 mL). The combined organic layers were washed with brine (1×30 mL), dried over anhydrous Na₂SO₄ and filtered. The filtrate was concentrated in vacuo and the residue was purified by Prep-HPLC with the following conditions: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 25% B to 50% B in 8 min; Wavelength: 254 nm/220 nm; RT1(min): 7.36. The pure fractions were concentrated and lyophilized to afford the title compound (29 mg, 0.1 mmol, 23% yield) as an off-white solid. LC purity: 98.9% (UV at 254 nm); Mass calculated for C₂₅H₁₇D₈F₃N₆O₂ [M+1]⁺, 507.6, found 507.2; Retention time: 1.346 min. ¹H NMR (400 MHz, DMSO) δ 8.84-8.77 (m, 1H), 8.47-8.37 (m, 1H), 8.09-8.01 (m, 2H), 7.35-7.25 (m, 1H), 5.99 (s, 2H), 3.86-3.78 (m, 1H), 3.60 (d, J=10.5 Hz, 2H), 2.87 (s, 2H), 2.75-2.63 (m, 1H), 2.00 (s, 1H), 1.82 (s, 1H), 1.64 (t, J=11.2 Hz, 2H).

### Example 52. (S)-(5-amino-6″-ethoxy-3′,6-difluoro-[2,2′:6′,3″-terpyridin]-4′-yl)(hexahydropyrrolo[1,2-alpyrazin-2(1H)-yl)methanone

### Example 53. (R)-(5-amino-6″-ethoxy-3′,6-difluoro-[2,2′:6′,3″-terpyridin]-4′-yl)(hexahydropyrrolo[1,2-alpyrazin-2(1H)-yl)methanone

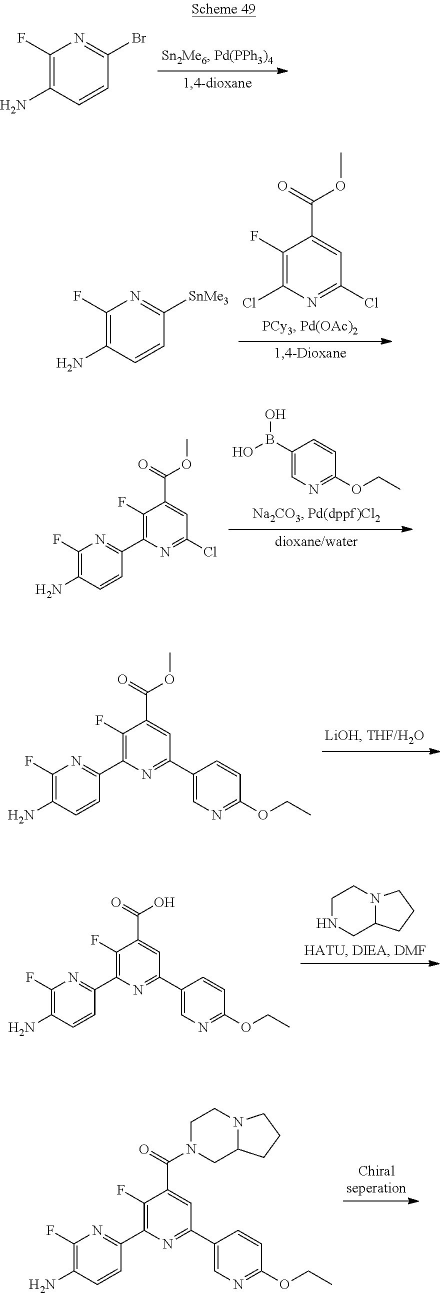

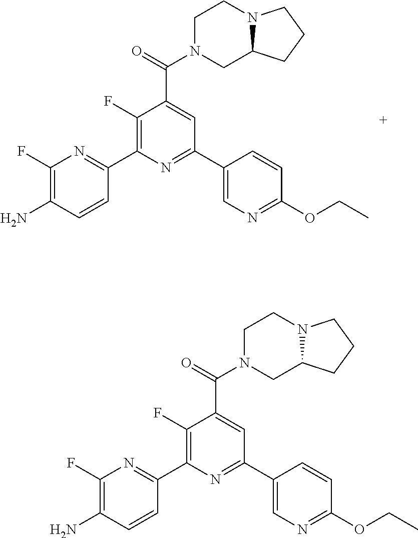 [SMILES: CCOc1ccc(-c2cc(C(=O)N3CCN4CCC[C@H]4C3)c(F)c(-c3ccc(N)c(F)n3)n2)cn1 ; CCOc1ccc(-c2cc(C(=O)N3CCN4CCC[C@@H]4C3)c(F)c(-c3ccc(N)c(F)n3)n2)cn1]

Step 1: To a solution of 6-bromo-2-fluoropyridin-3-amine (1.0 equiv. 4.0 g, 20.9 mmol) and Pd(PPh₃)₄(0.1 equiv. 2.4 g, 2.0 mmol) in 1,4-dioxane (40 mL) was added Sn₂Me₆ (1.5 equiv. 10.3 g, 31.4 mmol). The reaction mixture was stirred at 100° C. for 2 h under a nitrogen atmosphere. After concentration under reduced pressure, the residue was dissolved in EtOAc (200 mL) and washed with saturated aqueous KF (3×50 mL). The organic layer was dried over anhydrous Na₂SO₄, filtered, and concentrated under reduced pressure. The residue was purified via silica gel chromatography using a gradient of 0 to 30% EtOAc in PE to afford 2-fluoro-6-(trimethylstannyl)pyridin-3-amine (3.80 g, 13.8 mmol, 66% yield) as a yellow solid. LC purity: 57.0% (UV at 254 nm); Mass calculated for C₈H₁₃FN₂Sn[M+1]⁺, 277, found 277; Retention time: 0.838 min.

Step 2: To a solution of methyl 2,6-dichloro-3-fluoroisonicotinate (1.0 equiv. 500 mg, 2.23 mmol) and 2-fluoro-6-(trimethylstannyl)pyridin-3-amine (0.8 equiv. 491 mg, 1.79 mmol) in 1,4-dioxane (6 mL) were added PCy₃ (0.3 equiv. 18 mg, 0.67 mmol) and Pd(OAc)₂ (0.2 equiv. 100 mg, 0.4 mmol). The mixture was stirred at 70° C. under nitrogen atmosphere for overnight. The resulting mixture was concentrated under reduced pressure. The resulting residue was purified via reversed phase flash chromatography (C18 silica gel) using a gradient of 50% to 70% ACN in water (with 10 mmol/L NH₄HCO₃) to afford methyl 5′-amino-6-chloro-3,6′-difluoro-[2,2′-bipyridine]-4-carboxylate (540 mg, 1.8 mmol, 80% yield) as a yellow solid. LC purity: 40.1% (UV at 254 nm); Mass calculated for C₁₂H8ClF₂N₃O₂[M+1]⁺, 300.1, found 300.1; Retention time: 0.743 min.

Step 3: To a solution of methyl 5′-amino-6-chloro-3,6′-difluoro-[2,2′-bipyridine]-4-carboxylate (1.0 equiv. 479 mg, 1.6 mmol) and (6-ethoxypyridin-3-yl)boronic acid (1.0 equiv. 267 mg, 1.6 mmol) in 1,4-dioxane (6 mL) and Water (0.6 mL) were added Na₂CO₃ (3.0 equiv. 531 mg, 4.8 mmol) and Pd(dppf)Cl₂ (0.1 equiv. 122 mg, 0.16 mmol). The mixture was stirred at 80° C. for 2 h under nitrogen atmosphere. The resulting mixture was diluted with water and extracted with EtOAc (2×100 mL). The combined organic layers were washed with brine (1×30 mL), dried over anhydrous Na₂SO₄. After filtration, the filtrate was concentrated under reduced pressure to afford crude product methyl 5-amino-6″-ethoxy-3′,6-difluoro-[2,2′:6′,3″-terpyridine]-4′-carboxylate (540 mg, 1.4 mmol, 89% yield) as a light yellow solid, which was used directly in the next step without further purification. LC purity: 82.1% (UV at 254 nm); Mass calculated for C₁₉H₁₆F₂N₄O₃[M+1]⁺, 387.1, found 387.1; Retention time: 0.935 min.

Step 4: To a stirred solution of methyl 5-amino-6″-ethoxy-3′,6-difluoro-[2,2′:6′,3″-terpyridine]-4′-carboxylate (1.0 equiv. 540 mg, 1.4 mmol) in THF (6 mL) and water (2 mL) was added LiGH (3.0 equiv. 107 mg, 4.2 mmol). The mixture was stirred at room temperature for 2 h. The resulting mixture was acidified to pH 5~6 with 1N HCl, causing precipitation of a solid. This solid was collected by filtration, washed with water (2 mL), and dried under vacuum to afford crude 5-amino-6″-ethoxy-3′,6-difluoro-[2,2′:6′,3″-terpyridine]-4′-carboxylic acid (373 mg, 1.0 mmol, 69% yield) as a light yellow solid, which was used directly in the next step without further purification. LC purity: 76.0% (UV at 254 nm); Mass calculated for C₁₈H₁₄F₂N₄O₃[M+1]⁺, 373.1, found 373.1; Retention time: 0.855 min.

Step 5: To a solution of octahydropyrrolo[1,2-a]pyrazine (1.5 equiv. 51 mg, 0.4 mmol) and 5-amino-6″-ethoxy-3′,6-difluoro-[2,2′:6′,3″-terpyridine]-4′-carboxylic acid (1.0 equiv. 100 mg, 0.27 mmol) in DMF (3 mL) were added DIEA (3.0 equiv. 104 mg, 0.81 mmol) and HATU (2.0 equiv. 204 mg, 0.5 mmol). The mixture was stirred at room temperature for 2 h. After filtration, the filtrate was evaporated and the resulting mixture was purified by Prep-HPLC under the following conditions: Column: X-Bridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 31% B to 56% B in 10 min; Wavelength: 254 nm/220 nm; RT1(min): 7.85. The pure fractions were concentrated and lyophilized to afford (5-amino-6″-ethoxy-3′,6-difluoro-[2,2′:6′,3″-terpyridin]-4′-yl)(hexahydropyrrolo[1,2-a]pyrazin-2(1H)-yl)methanone (73 mg, 0.152 mmol, 56.7% yield) as an off-white solid. LC purity: 96.7% (UV at 254 nm); Mass calculated for C₂₅H₂₆F₂N₆O₂[M+1]⁺, 481.3, found 481.3; Retention time: 0.914 min.

Step 6: The racemate (5-amino-6″-ethoxy-3′,6-difluoro-[2,2′:6′,3″-terpyridin]-4′-yl)(hexahydropyrrolo[1,2-a]pyrazin-2(1H)-yl)methanone (73 mg, 0.15 mmol) was purified by prep-chiral-HPLC with following conditions: Column: CHIRALART Cellulose-SB, 5 μm, 250 mm×20 mm; Mobile Phase A: MtBE: Hex=1: 1(with 0.5% NH₃ (2 M in MeOH)), Mobile Phase B: EtOH: DCM=1: 1; Flow rate: 20 mL/min; Gradient (B %)10% B; Wavelength: 254/220 nm; RT1(min): 13.07; RT2(min): 16.51; Sample Solvent: EtOH; Injection Volume: 0.2 mL; Number Of Runs: 8; Single injection run time(min): 20. The pure fractions of first peak RT1 (min): 13.07 and second peak RT2 (min): 16.51 (stereochemistry arbitrarily assigned) were concentrated and the residues were re-dissolved in CH₃CN and H₂O, and then was lyophilized to afford the title compound as white solids.

### Example 52: RT1 (25 mg, 0.051 mmol, 34.2% yield): LC purity: 98.3% (UV at

254 nm); Mass calculated for C₂₅H₂₆F₂N₆O₂[M+1]⁺, 481.3, found 481.3; Retention time: 1.118 min. ¹H NMR (400 MHz, MeOD) δ 8.86 (d, J=2.7 Hz, 1H), 8.43 (dd, J=8.7, 2.8 Hz, 1H), 8.12-7.92 (m, 2H), 7.35 (t, J=9.4 Hz, 1H), 6.91 (d, J=8.8 Hz, 1H), 6.01 (s, 2H), 4.34-4.31 (m, 2H), 3.74-3.72 (m, 1H), 3.33-2.87 (m, 4H), 2.54-2.52 (m, 1H), 2.17-2.13 (m, 2H), 2.01-1.83 (m, 2H), 1.78-1.52 (m, 3H), 1.43-1.39 (m, 3H);

Example 53: RT2 (27 mg, 0.055 mmol, 37.0% yield): LC purity: 98.1% (UV at 254 nm); Mass calculated for C₂₅H₂₆F₂N₆O₂[M+1]⁺, 481.1, found 481.1; Retention time: 1.138 min. 6 8.85 (d, J=2.7 Hz, 1H), 8.45 (dd, J=8.7, 2.8 Hz, 1H), 8.14-7.93 (m, 2H), 7.35 (t, J=9.4 Hz, 1H), 6.92 (d, J=8.8 Hz, 1H), 6.01 (s, 2H), 4.35-4.32 (m, 2H), 3.74-3.72 (m, 1H), 3.33-2.87 (m, 4H), 2.54-2.52 (m, 1H), 2.17-2.13 (m, 2H), 2.01-1.83 (m, 2H), 1.78-1.52 (m, 3H), 1.43-1.39 (m, 3H).

### Example 54. (S)-(5-amino-6″-ethoxy-3′,5″,6-trifluoro-[2,2′:6′,3″-terpyridin]-4′-yl)(hexahydropyrrolo[1,2-alpyrazin-2(1H)-yl)methanone

### Example 55. (R)-(5-amino-6″-ethoxy-3′,5″,6-trifluoro-[2,2′:6′,3″-terpyridin]-4′-yl)(hexahydropyrrolo[1,2-alpyrazin-2(1H)-yl)methanone

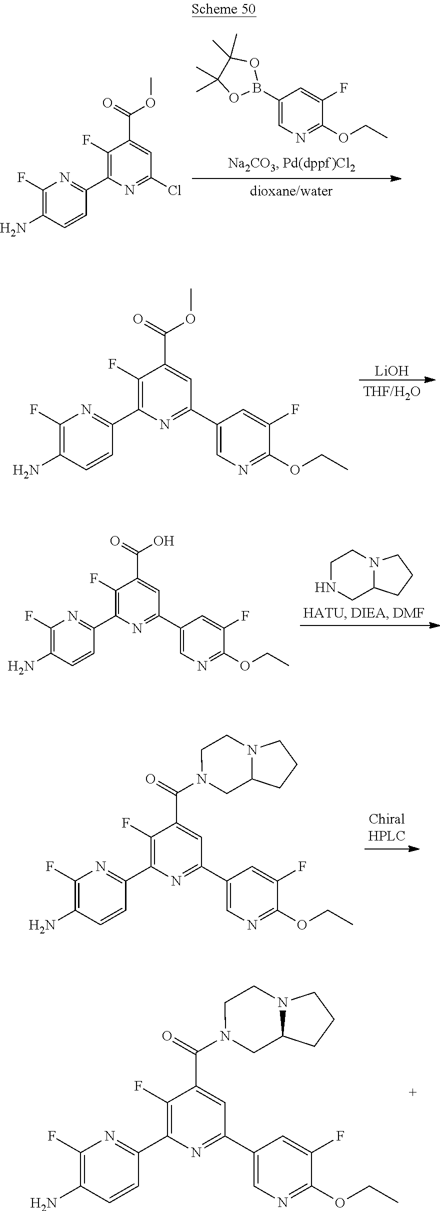

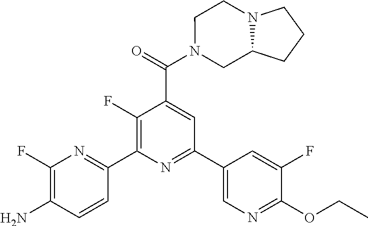 [SMILES: CCOc1ncc(-c2cc(C(=O)N3CCN4CCC[C@@H]4C3)c(F)c(-c3ccc(N)c(F)n3)n2)cc1F]

Step 1: To a solution of methyl 5′-amino-6-chloro-3,6′-difluoro-[2,2′-bipyridine]-4-carboxylate (1.0 equiv. 446 mg, 1.49 mmol) and 2-ethoxy-3-fluoro-5-(4,4,5,5-tetramethyl-1,3,2-dioxaborolan-2-yl)pyridine (1.25 equiv. 500 mg, 1.87 mmol) in 1,4-dioxane (6 mL) and Water (0.6 mL) were added Na₂CO₃ (3.0 equiv. 531 mg, 4.5 mmol) and Pd(dppf)Cl₂ (0.1 equiv. 122 mg, 0.15 mmol). The mixture was stirred at 80° C. for 2 h under nitrogen atmosphere. The resulting mixture was diluted with water and extracted with EtOAc (2×100 mL). The combined organic layers were washed with brine (1×30 mL), dried over anhydrous Na₂SO₄. After filtration, the filtrate was concentrated under reduced pressure to afford crude product methyl 5-amino-6″-ethoxy-3′, 5″,6-trifluoro-[2,2′: 6′,3″-terpyridine]-4′-carboxylate (600 mg, 1.48 mmol, 98% yield) as a light yellow solid, which was used directly in the next step without further purification. LC purity: 82.1% (UV at 254 nm); Mass calculated for C₁₉H₁₅F₃N₄O₃[M+1]⁺, 405.1, found 405.1; Retention time: 0.935 min.

Step 2: To a stirred solution of methyl 5-amino-6″-ethoxy-3′,5″,6-trifluoro-[2,2′:6′,3″-terpyridine]-4′-carboxylate (1.0 equiv. 600 mg, 1.49 mmol) in THF (6 mL) and Water (2 mL) was added LiGH (3.0 equiv. 107 mg, 4.5 mmol). The resulting mixture was stirred at room temperature for 2 h, then acidified to pH 5-6 using 1 N HCl. The precipitated solid was collected by filtration, washed with water (2 mL), and dried to afford crude 5-amino-6″-ethoxy-3′,5″,6-trifluoro-[2,2′:6′,3″-terpyridine]-4′-carboxylic acid (400 mg, 1.0 mmol, 69% yield) as a light yellow solid, which was used directly in the next step without further purification. LC purity: 76.0% (UV at 254 nm); Mass calculated for C₁₈H₁₃F₃N₄O₃ [M+1]⁺, 391.1, found 391.1; Retention time: 0.855 min.

Step 3: To a solution of 5-amino-6″-ethoxy-3′,5″,6-trifluoro-[2,2′:6′,3″-terpyridine]-4′-carboxylic acid (1.0 equiv. 100 mg, 0.26 mmol) in DMF (3 mL) were added DIEA (3.0 equiv. 104 mg, 0.78 mmol) and HATU (2.0 equiv. 204 mg, 0.52 mmol). The mixture was stirred at room temperature for 2 h. The resulting solution was purified by Prep-HPLC with following condition: Column: Kinetex EVO C18 Column, 30*150 mm, 5 μm; Mobile Phase A: Water(10 nmol/LNH₄HCO₃+0.05% NH₃H₂O), Mobile Phase B: ACN; Flow rate: 25 mL/min; Gradient (B %): 34% B to 50% B in 10 min; Wavelength: 254 nm/220 nm; RT1(min): 8.26. The pure fractions were concentrated and lyophilized to afford (5-amino-6″-ethoxy-3′,5″,6-trifluoro-[2,2′:6′,3″-terpyridin]-4′-yl)(hexahydropyrrolo[1,2-a]pyrazin-2(1H)-yl)methanone (71 mg, 0.14 mmol, 53.2% yield) as an off-white solid. LC purity: 96.2% (UV at 254 nm); Mass calculated for C₂₅H₂₅F₃N₆O₂[M+1]⁺, 498.2, found 498.2; Retention time: 0.965 min.

Step 4: The racemate (5-amino-6″-ethoxy-3′, 5″,6-trifluoro-[2,2′:6′,3″-terpyridin]-4′-yl)(hexahydropyrrolo[1,2-a]pyrazin-2(1H)-yl)methanone (71 mg, 0.1 mmol) was purified by prep-chiral-HPLC with following conditions: Column: CHIRAL ART Cellulose-SB, 5 μm, 20 mm 250 mm; Mobile Phase A: EtOH: DCM=1: 1, Mobile Phase B: (MTBE: Hex=45: 55) (with 0.5% NH₃ (2 M in MeOH)); Flow rate: 20 mL/min; Gradient (B %) 40% B; Wavelength: 220/254 nm; RT1(min): 28.11; RT2(min): 34.48; Sample Solvent: EtOH: DCM=1: 1; Injection Volume: 0.4 mL; Number Of Runs: 5; Single injection run time 38 mn. The pure fractions of first peak (RT1: 28.11 min) and second peak (RT2: 34.48 min) were concentrated and the residues were re-dissolved in CH₃CN and H₂O, and lyophilized to afford the title compounds (stereochemistry arbitrarily assigned) as white solids.

Example 54: RT1 (15 mg, 0.03 mmol, 21.2% yield): LC purity: 98.3% (UV at 254 nm); Mass calculated for C₂₅H₂₅F₃N₆O₂, 498.2, found 498.2; Retention time: 0.965 min. ¹H NMR (400 MHz, DMSO) δ 8.79 (s, 1H), 8.41 (d, J=11.7 Hz, 1H), 8.04 (dd, J=11.4, 5.9 Hz, 2H), 7.31 (dd, J=11.1, 8.1 Hz, 1H), 6.00 (s, 2H), 4.48-4.44 (m, 2H), 3.57-3.55 (m, 1H), 3.33-2.82 (m, 4H), 2.59 (s, 1H), 2.18-1.78 (m, 4H), 1.72-1.53 (m, 3H), 1.45-1.42 (m, 3H).

Example 55: RT2 (19 mg, 0.03 mmol, 26.7% yield): LC purity: 97.6% (UV at 254 nm); Mass calculated for C₂₅H₂₅F₃N₆O₂, 498.2, found 498.2; Retention time: 0.965 min. ¹H NMR (400 MHz, DMSO) δ 8.78 (s, 1H), 8.40 (d, J=11.8 Hz, 1H), 8.09-7.96 (m, 2H), 7.31 (dd, J=11.2, 8.1 Hz, 1H), 5.99 (s, 2H), 4.53-4.41 (m, 2H), 3.57 (s, 1H), 3.27-2.83 (m, 4H), 2.65-2.63 (m, 1H), 2.24-1.57 (m, 6H), 1.46-1.33 (m, 4H).

### Example 56. (3-amino-3-methylpiperidin-1-yl)(5-amino-6″-ethoxy-3′,5″,6-trifluoro-[2,2′:6′,3″-terpyridin]-4′-yl)methanone

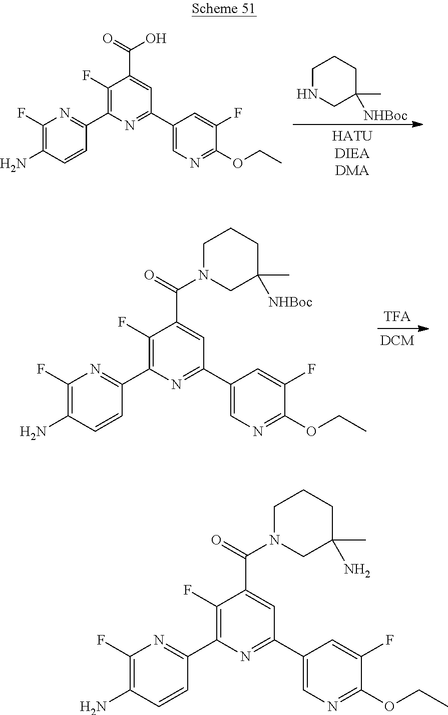 [SMILES: CCOc1ncc(-c2cc(C(=O)O)c(F)c(-c3ccc(N)c(F)n3)n2)cc1F ; CC1(NC(=O)OC(C)(C)C)CCCNC1 ; CCOc1ncc(-c2cc(C(=O)N3CCCC(C)(NC(=O)OC(C)(C)C)C3)c(F)c(-c3ccc(N)c(F)n3)n2)cc1F ; CCOc1ncc(-c2cc(C(=O)N3CCCC(C)(N)C3)c(F)c(-c3ccc(N)c(F)n3)n2)cc1F]

Step 1: To a stirred mixture of 5-amino-6″-ethoxy-3′,5″,6-trifluoro-[2,2′:6′,3″-terpyridine]-4′-carboxylic acid (1.0 equiv. 100 mg, 0.26 mmol) and tert-butyl (3-methylpiperidin-3-yl)carbamate (1.5 equiv. 82 mg, 0.38 mmol) in DMA (5 mL) were added HATU (1.5 equiv. 146 mg, 0.38 mmol) and DIEA (2.0 equiv. 66 mg, 0.52 mmol) at room temperature. The reaction was stirred for 1 h at room temperature. The resulting mixture was diluted with water and extracted with EtOAc. The organic layers were washed with brine, dried and concentrated to afford crude tert-butyl (1-(5-amino-6″-ethoxy-3′,5″,6-trifluoro-[2,2′:6′,3″-terpyridine]-4′-carbonyl)-3-methylpiperidin-3-yl)carbamate (99 mg, 0.17 mmol, 65.8% yield) as a yellow solid. LC purity: 83.1% (UV at 254 nm); Mass calculated for C₂₉H₃₃F₃N₆O₄[M+1]⁺ 587.3, found 587.3; Retention time: 1.091 min.

Step 2: To a stirred mixture of tert-butyl (1-(5-amino-6″-ethoxy-3′,5″,6-trifluoro-[2,2′:6′,3″-terpyridine]-4′-carbonyl)-3-methylpiperidin-3-yl)carbamate (1.0 equiv. 60 mg, 0.1 mmol) in DCM (3 mL) was added TFA (1 mL), and the resulting mixture was stirred at room temperature for 1 h. The resulting mixture was purified with following condition: Column: XBridge Prep OBD C18 Column, 30*75 mm, 3.5 μm; Mobile Phase A: Water (10 mmol/L NH₄HCO₃+0.05% NH₃H₂O), Mobile Phase B: ACN; Flow rate: 60 mL/min; Gradient (B %): 20% B to 40% B in 6 min; Wavelength: 254 nm/220 nm; RT1(min): 5.62. The pure fractions were concentrated and lyophilized to afford title compound (17.4 mg, 0.0358 mmol, 34.96% yield) as a white solid.

Example 56: LC purity: 98.6% (UV at 254 nm); Mass calculated for C₂₋₄H₂₅F₃N₆O₂ [M+1]⁺ 487.3, found 487.3; Retention time: 2.786 min. ¹H NMR (400 MHz, DMSO) δ 8.98-8.69 (m, 1H), 8.51-8.28 (m, 1H), 8.19-7.93 (m, 2H), 7.31 (dd, J=11.2, 8.1 Hz, 1H), 5.99 (d, J=4.2 Hz, 2H), 4.54-4.50 (m, 2H), 4.01-3.57 (m, 1H), 3.31-3.27 (m, 1H), 3.08-2.97 (m, 1H), 1.78-1.45 (m, 5H), 1.40 (t, J=7.0 Hz, 3H), 1.00-0.96 (m, 3H).

### Example 57. (3-amino-3-methylpiperidin-1-yl)(5-amino-6″-ethoxy-3′,6-difluoro-[2,2′:6′,3″-terpyridin]-4′-yl)methanone

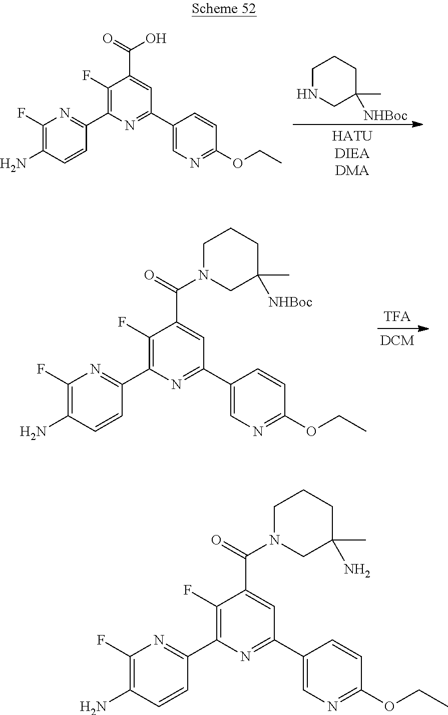 [SMILES: CCOc1ccc(-c2cc(C(=O)O)c(F)c(-c3ccc(N)c(F)n3)n2)cn1 ; CC1(NC(=O)OC(C)(C)C)CCCNC1 ; CCOc1ccc(-c2cc(C(=O)N3CCCC(C)(NC(=O)OC(C)(C)C)C3)c(F)c(-c3ccc(N)c(F)n3)n2)cn1 ; CCOc1ccc(-c2cc(C(=O)N3CCCC(C)(N)C3)c(F)c(-c3ccc(N)c(F)n3)n2)cn1]

Step 1: To a solution of 5-amino-6″-ethoxy-3′,6-difluoro-[2,2′:6′,3″-terpyridine]-4′-carboxylic acid (1.0 equiv. 80 mg, 0.21 mmol) and tert-butyl (3-methylpiperidin-3-yl)carbamate (1.5 equiv. 69 mg, 0.32 mmol) in DMA (2 mL) were added HATU (1.5 equiv. 121 mg, 0.32 mmol) and DIEA (5.0 equiv. 135 mg, 1.05 mmol). The mixture was stirred at room temperature for overnight. The resulting mixture was quenched with water and extracted with EtOAc (2×100 mL). The combined organic layers were washed with brine (1×30 mL), dried over anhydrous Na₂SO₄. After filtration, the filtrate was concentrated under reduced pressure to afford crude tert-butyl (1-(5-amino-6″-ethoxy-3′,6-difluoro-[2,2′:6′,3″-terpyridine]-4′-carbonyl)-3-methylpiperidin-3-yl)carbamate (83.5 mg, 0.15 mmol, 69.5% yield) as a yellow solid. LC purity: 90.0% (UV at 254 nm); Mass calculated for C₂₉H₃₄F₂N₆O₄[M+1]⁺, 569.3, found 569.3; Retention time: 0.958 min.

Step 2: To a solution of tert-butyl (1-(5-amino-6″-ethoxy-3′,6-difluoro-[2,2′:6′,3″-terpyridine]-4′-carbonyl)-3-methylpiperidin-3-yl)carbamate (1.0 equiv. 83.5 mg, 0.15 mmol) in DCM (1.8 mL) was added TFA (0.60 mL). The mixture was stirred at 25° C. for 1 h. After filtration, the filtrate was purified by Prep-HPLC (UV 254 nm/220 nm XBridge Prep OBD C18 Column, 30*150 mm, 5 m Water (10 mmol/L NH₄HCO₃+0.05% NH₃H₂O) ACN 60 mL/min 33% B to 53% B in 8 min 7.59); The pure fractions were concentrated and lyophilized to afford title compound (42 mg, 0.0892 mmol, 50.3% yield) as an off-white solid.

Example 57: LC purity: 99.0% (UV at 254 nm); Mass calculated for C₂₋₄H₂₆F₂N₆O₂ [M+1]⁺, 469.2, found 469.2; Retention time: 1.211 min. ¹H NMR (400 MHz, DMSO) δ 8.98-8.91 (m, 1H), 8.47 (dd, J=8.7, 6.2 Hz, 1H), 8.06-7.92 (m, 2H), 7.34-7.27 (m, 1H), 6.93 (dd, J=8.9, 2.3 Hz, 1H), 5.97 (d, J=4.4 Hz, 2H), 4.45-4.35 (m, 2H), 3.61 (d, J=12.1 Hz, 1H), 3.27-3.07 (m, 2H), 3.00 (s, 1H), 1.57-1.55 (m, 6H), 1.39-1.31 (m, 3H), 1.00-0.97 (m, 3H).

### Example 58. (5-amino-6″-ethoxy-3′,5″,6-trifluoro-[2,2′:6′,3″-terpyridin]-4′-yl)(3-aminopiperidin-1-yl)methanone

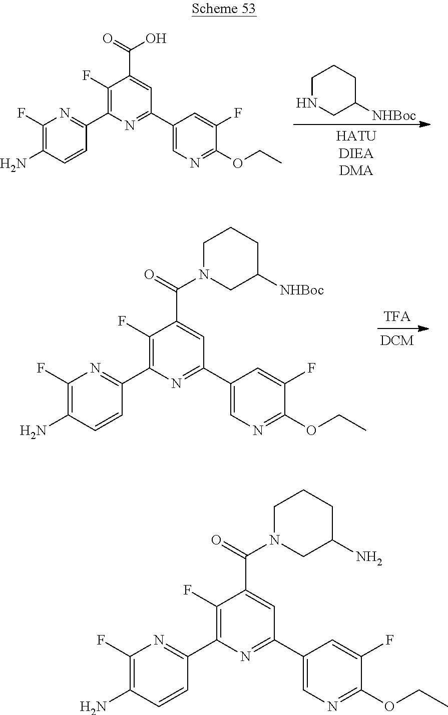 [SMILES: CCOc1ncc(-c2cc(C(=O)O)c(F)c(-c3ccc(N)c(F)n3)n2)cc1F ; CC(C)(C)OC(=O)NC1CCCNC1 ; CCOc1ncc(-c2cc(C(=O)N3CCCC(NC(=O)OC(C)(C)C)C3)c(F)c(-c3ccc(N)c(F)n3)n2)cc1F ; CCOc1ncc(-c2cc(C(=O)N3CCCC(N)C3)c(F)c(-c3ccc(N)c(F)n3)n2)cc1F]

Step 1: To a stirred mixture of 5-amino-6″-ethoxy-3′,5″,6-trifluoro-[2,2′:6′,3″-terpyridine]-4′-carboxylic acid (1.0 equiv. 100 mg, 0.26 mmol) and tert-butyl piperidin-3-ylcarbamate (1.5 equiv. 77 mg, 0.38 mmol) in DMA (5 mL) were added HATU (1.5 equiv. 146 mg, 0.38 mmol) and DIEA (2.0 equiv. 66 mg, 0.52 mmol) at room temperature. The reaction was stirred at room temperature for 1 h. The resulting mixture was diluted with water and extracted with EtOAc. The organic layers were washed with brine, dried and concentrated to afford crude tert-butyl (1-(5-amino-6″-ethoxy-3′,5″,6-trifluoro-[2,2′:6′,3″-terpyridine]-4′-carbonyl)piperidin-3-yl)carbamate (99 mg, 0.17 mmol, 67.4% yield) as a yellow solid. LC purity: 85.5% (UV at 254 nm); Mass calculated for C₂₈H₃₁F₃N₆O₄[M+1]⁺ 573.2, found 573.2; Retention time: 0.989 min.

Step 2: To a stirred mixture of tert-butyl (1-(5-amino-6″-ethoxy-3′,5″,6-trifluoro-[2,2′:6′,3″-terpyridine]-4′-carbonyl)piperidin-3-yl)carbamate (1.0 equiv. 80 mg, 0.14 mmol) and TFA (1 mL) in DCM (3 mL) at room temperature. The resulting mixture was stirred at room temperature for 1 h. The resulting mixture was concentrated under reduced pressure and the residue was purified by prep HPLC with following condition: Column: XBridge Prep OBD C18 Column, 30*75 mm, 3.5 μm; Mobile Phase A: Water (10 mmol/L NH₄HCO₃+0.05% NH₃H₂O), Mobile Phase B: ACN; Flow rate: 60 mL/min; Gradient (B %): 29% B to 49% B in 8 min; Wavelength: 254 nm/220 nm; RT1(min): 7.01. The pure fractions were concentrated and lyophilized to afford title compound (31.6 mg, 0.0669 mmol, 47.5% yield) as a white solid.

Example 58: ¹H NMR (400 MHz, DMSO) δ 8.80 (s, 1H), 8.42 (d, J=11.7 Hz, 1H), 8.16-7.97 (m, 2H), 7.34-7.26 (m, 1H), 5.99 (s, 2H), 4.55-4.43 (m, 2H), 4.35 (d, J=10.1 Hz, 1H), 3.50-3.38 (m, 1H), 3.12-2.96 (m, 1H), 2.89-2.70 (m, 1H), 2.69-2.59 (m, 1H), 1.87 (d, J=12.2 Hz, 1H), 1.76 (s, 1H), 1.64 (s, 2H), 1.48 (s, 1H), 1.40 (t, J=7.0 Hz, 3H), 1.33-1.21 (m, 1H).

### Example 59. (R)-(5-amino-6″-ethoxy-3′,5″,6-trifluoro-[2,2′:6′,3″-terpyridin]-4′-yl)(3-aminopiperidin-1-yl)methanone

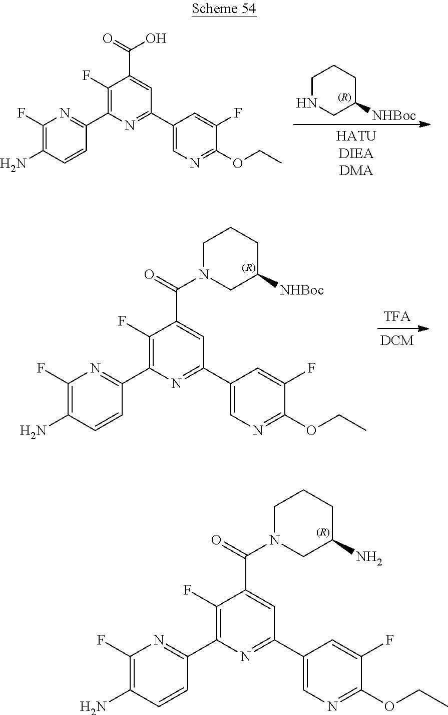

Step 1: To a stirred mixture of 5-amino-6″-ethoxy-3′,5″,6-trifluoro-[2,2′:6′,3″-terpyridine]-4′-carboxylic acid (1.0 equiv. 100 mg, 0.26 mmol) and tert-butyl (R)-piperidin-3-ylcarbamate (1.5 equiv. 77 mg, 0.38 mmol) in DMA (5 mL) were added HATU (1.5 equiv. 146 mg, 0.38 mmol) and DIEA (2.0 equiv. 66 mg, 0.52 mmol). The reaction was stirred at room temperature for 1 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The product was used in the next step directly without further purification. LC purity: 87.1% (UV at 254 nm); Mass calculated for C₂₈H₃₁F₃N₆O₄[M+1]⁺ 573.2, found 573.2; Retention time: 0.716 min.

Step 2: To a stirred mixture of tert-butyl (R)-(1-(5-amino-6″-ethoxy-3′,5″,6-trifluoro-[2,2′:6′,3″-terpyridine]-4′-carbonyl)piperidin-3-yl)carbamate (1.0 equiv. 50 mg, 0.087 mmol) and TFA (1 mL) in DCM (3 mL) at room temperature. The resulting mixture was stirred at room temperature for 1 h. The resulting mixture was concentrated under reduced pressure and the residue was purified with following condition: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 30% B to 42% B in 8 min; Wavelength: 254 nm/220 nm nm; RT1(min): 7.71. The pure fractions were concentrated and lyophilized to afford title compound (28.5 mg, 0.0604 mmol, 69.09% yield) as a white solid.

Example 59: LC purity: 98.8% (UV at 254 nm); Mass calculated for C₂₃H₂₃F₃N₆O₂ [M+1]⁺ 473.2, found 473.2; Retention time: 1.368 min. ¹H NMR (400 MHz, DMSO) δ 8.79 (s, 1H), 8.41 (d, J=11.8 Hz, 1H), 8.19-7.96 (m, 2H), 7.34-7.24 (m, 1H), 5.98 (s, 2H), 4.54-4.42 (m, 2H), 4.40-4.28 (m, 1H), 3.42 (d, J=13.1 Hz, 1H), 3.12-2.95 (m, 1H), 2.86-2.59 (m, 2H), 1.93-1.67 (m, 4H), 1.47 (s, 1H), 1.39 (t, J=7.0 Hz, 3H), 1.31-1.18 (m, 1H).

### Example 60. (R)-(5-amino-6″-ethoxy-3′,6-difluoro-[2,2′:6′,3″-terpyridin]-4′-yl)(3-aminopiperidin-1-yl)methanone

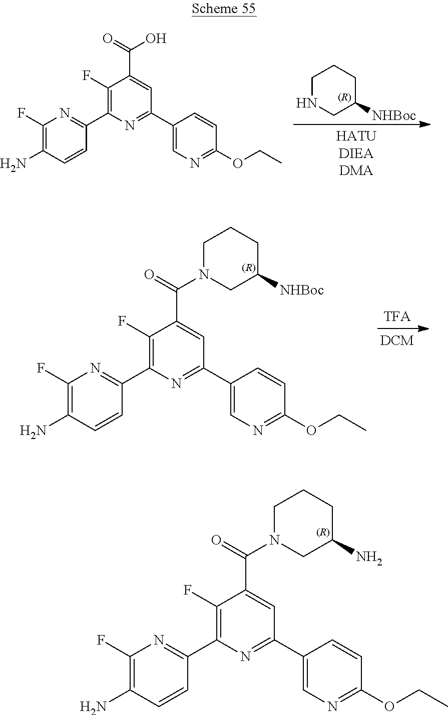

Step 1: To a solution of 5-amino-6″-ethoxy-3′,6-difluoro-[2,2′:6′,3″-terpyridine]-4′-carboxylic acid (see, Example 52, Step 4) (1 equiv. 80 mg, 0.22 mmol) and tert-butyl (R)-piperidin-3-ylcarbamate (1.5 equiv. 65 mg, 0.32 mmol) in DMA (5 mL) was added HATU (1.5 equiv. 122 mg, 0.32 mmol) at 0° C., followed by dropwise addition of DIEA (2 equiv. 55 mg, 0.43 mmol). The mixture was then stirred at room temperature for 2 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The product was used in the next step directly without further purification. LC purity: 86.2% (UV at 254 nm); Mass calculated for C₂₈H₃₂F₂N₆O₄[M+1]⁺ 555.2, found 555.2; Retention time: 0.832 min.

Step 2: To a solution of tert-butyl (R)-(1-(5-amino-6″-ethoxy-3′,6-difluoro-[2,2′:6′,3″-terpyridine]-4′-carbonyl)piperidin-3-yl)carbamate (1 equiv. 80 mg, 0.144 mmol) in DCM (3 mL) was added TFA (1 mL) dropwise at 0° C. and the resulting solution was stirred at room temperature for 2 h under nitrogen. The yellow solution was bubbled with nitrogen to remove DCM and the residue was dissolved into MeCN, purified by Prep-HPLC with following condition: Column: XBridge Prep OBD C18 Column, 30*150 mm, 5 μm; Mobile Phase A: Water (10nmol/LNH₄HCO₃), Mobile Phase B: ACN; Flow rate: 60 mL/min; Gradient (B %): 27% B to 42% B in 8 min; Wavelength: 254 nm/220 nm; RT1(min): 5.7). Pure fractions were evaporated to afford (R)-(5-amino-6″-ethoxy-3′,6-difluoro-[2,2′:6′,3″-terpyridin]-4′-yl)(3-aminopiperidin-1-yl)methanone (33 mg, 0.07 mmol, 50% yield) was a white solid.

Example 60: LC purity: 99.3% (UV at 254 nm); Mass calculated for C₂₃H₂₄F₂N₆O₂ [M+1]⁺ 455.2, found 455.2. Retention time: 1.282 min. ¹H NMR (400 MHz, DMSO) δ 8.98-8.92 (m, 1H), 8.46 (s, 1H), 8.06-7.96 (m, 2H), 7.31-7.28 (m, 1H), 6.93 (dd, J=8.7, 2.0 Hz, 1H), 5.98 (s, 2H), 4.39-4.37 (m, 3H), 3.43 (d, J=13.4 Hz, 1H), 3.07-2.97 (m, 1H), 2.79 (s, 1H), 2.65 (dd, J=9.0, 4.7 Hz, 1H), 1.88 (d, J=12.2 Hz, 2H), 1.46 (s, 2H), 1.36 (t, J=7.0 Hz, 3H), 1.26 (d, J=11.8 Hz, 1H).

### Example 61. (R)-(6-(5-amino-6-fluoropyrazin-2-yl)-6′-ethoxy-5-fluoro-[2,3′-bipyridin]-4-yl)(3-aminopiperidin-1-yl)methanone

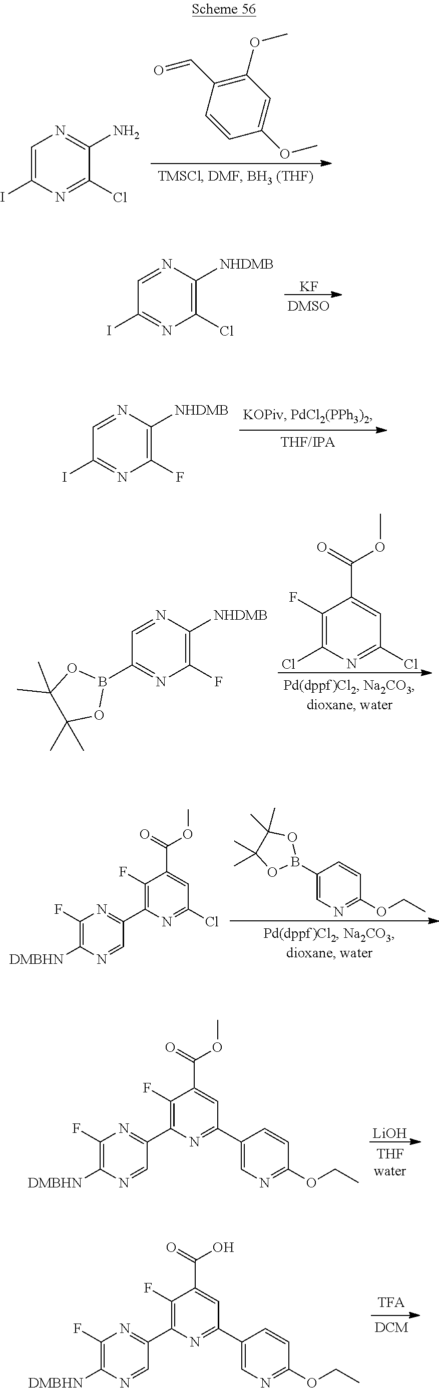

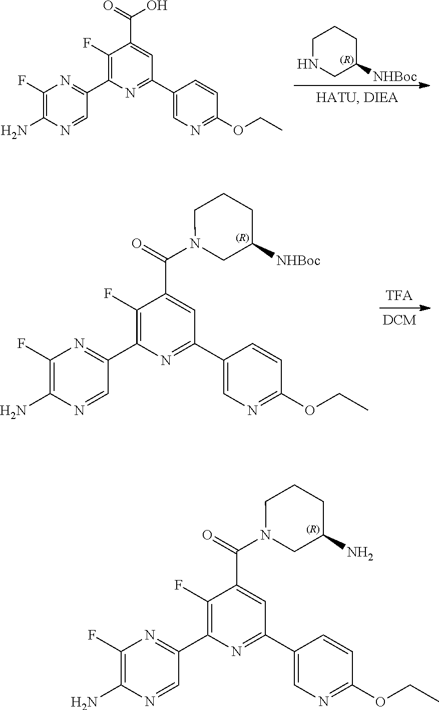

Step 1: To a solution of 3-chloro-5-iodopyrazin-2-amine (1.00 equiv. 50 g, 195.77 mmol) and 2,4-dimethoxybenzaldehyde (1.30 equiv. 42.3 g, 254.51 mmol) in DMF (500 mL) was added TMSCl (3.00 equiv. 63.8 g, 587.26 mmol) at 0° C. in one portion and the reaction was stirred at 60° C. for 2 h under N₂ atmosphere. Upon completion, the yellow mixture was cooled to −5° C. and BH₃ (2.04 equiv. 400 mmol, 400 mL, 1 M in THF) was added dropwise at this temperature (Caution: H2 gas was released during the addition. Please handle with care). After addition (~20 min), the yellow solution was warmed and stirred at ambient temperature for 2 h. The solution was concentrated in vacuo and the crude product was purified by column chromatography on silica gel (PE in EtOAc=5%) to afford 3-chloro-N-(2,4-dimethoxybenzyl)-5-iodopyrazin-2-amine (64 g, 157.79 mmol, 80.6% yield) as a white solid. LC purity: 95% (UV at 254 nm); Mass calculated for C₁₃H₁₃CIN₃O₂[M+1]⁺, 406.0, found 405.9; Retention time: 0.965 min. ¹H NMR (400 MHz, DMSO-d₆) δ 8.19 (s, 1H), 7.49 (t, J=6.0 Hz, 1H), 6.97-6.95 (d, J=8.4 Hz, 1H), 6.55-6.45 (d, J=2.4 Hz, 1H), 6.43-6.41 (d, J=8.4 Hz, 1H), 4.44-4.42 (d, J=6 Hz, 1H), 3.80 (s, 3H), 3.74 (s, 3H).

Step 2: To a solution of 3-chloro-N-(2,4-dimethoxybenzyl)-5-iodopyrazin-2-amine (1.00 equiv. 50 g, 123.27 mmol), 18-crown-6 (2.00 equiv. 65.2 g, 246.67 mmol) and KF (3.00 equiv. 21.5 g, 370.05 mmol) in DMSO (500 mL) was added DIEA (2.99 equiv. 47.7 g, 369.08 mmol) at ambient temperature and the reaction was stirred at 120° C. for 8 h under N₂ atmosphere. Upon completion, the reaction mixture was diluted with water and extracted with EtOAc. The organic layers were washed with brine over 4 times, dried over anhydrous sodium sulfate, filtered and concentrated in vacuo. The residue was purified by column chromatography on silica gel (PE in EtOAc=10%) to afford N-(2,4-dimethoxybenzyl)-3-fluoro-5-iodopyrazin-2-amine (33.6 g, 86.33 mmol, 70.0% yield) as a white solid. LC purity: 90% (UV at 254 nm); Mass calculated for C₁₃H₁₃FIN₃O₂[M+1]⁺, 390.0, found 390.0; Retention time: 0.938 min.

Step 3: To a stirred solution of N-(2,4-dimethoxybenzyl)-3-fluoro-5-iodopyrazin-2-amine (1.00 equiv. 5 g, 12.85 mmol) and bis(pinacolato)diboron (1.50 equiv. 4.9 g, 19.30 mmol) in THF (36 mL)/IPA (12 mL) were added KOPiv (1.26 equiv. 2.3 g, 16.17 mmol) and PdCl₂(PPh₃)₂(0.05 equiv. 451 mg, 0.64 mmol) under N₂ atmosphere. The reaction was stirred at 40° C. for 20 h. The resulting mixture was evaporated carefully at ambient temperature. The residue was diluted with dichloromethane, filtered to remove undissolved salts and the filtrate was evaporated to give a semi-solid crude (The crude material is stable in both semi-solid form and in dioxane solution at ambient temperature) which was used in the next step without purification. LC purity: 83% (UV at 254 nm); Mass calculated for C₁₉H₂₅BFN₃O₄[M+1]⁺, 390.2, found 390.3; Retention time: 1.948 & 0.691 min.

Step 4: To a mixture of methyl 2,6-dichloro-3-fluoroisonicotinate (1.00 equiv. 800 mg, 3.57 mmol), N-(2,4-dimethoxybenzyl)-3-fluoro-5-(4,4,5,5-tetramethyl-1,3,2-dioxaborolan-2-yl)pyrazin-2-amine (0.80 equiv. 1113 mg, 2.86 mmol) and Na₂CO₃ (2.00 equiv. 757 mg, 7.14 mmol) in 1,4-dioxane (10 mL) and water (1 mL) was added Pd(dtbpf)Cl₂ (0.10 equiv. 232 mg, 0.357 mmol). The reaction was stirred at 40° C. for 2 h under N₂ atmosphere. The reaction was then diluted with water and extracted with ethyl acetate. The combined organic layers were washed with brine, dried over sodium sulfate, filtered, and concentrated under reduced pressure. The residue was purified by column chromatography on silica gel with petroleum ether/ethyl acetate (3/2) to give methyl 6-chloro-2-(5-((2,4-dimethoxybenzyl)amino)-6-fluoropyrazin-2-yl)-3-fluoroisonicotinate (480 mg, 1.06 mmol, 30.0% yield) as a yellow solid. LC purity: 83% (UV at 254 nm); Mass calculated for C₂₀H₁₇ClF₂N₄O₄ [M+1]⁺ 451.1, found 451.1; Retention time: 0.766 min.

Step 5: To a mixture of methyl 6-chloro-2-(5-((2,4-dimethoxybenzyl)amino)-6-fluoropyrazin-2-yl)-3-fluoroisonicotinate (1.00 equiv. 500 mg, 1.11 mmol), 2-ethoxy-5-(4,4,5,5-tetramethyl-1,3,2-dioxaborolan-2-yl)pyridine (1.20 equiv. 334 mg, 1.34 mmol) and Na₂CO₃ (2.00 equiv. 235 mg, 2.22 mmol) in 1,4-dioxane (10 mL) and water (1 mL) was added Pd(dppf)Cl₂ (0.12 equiv. 97 mg, 0.13 mmol). The reaction was stirred at 80° C. for 2 h under N₂ atmosphere. The reaction was then diluted with water and extracted with ethyl acetate. The combined organic layers were washed with brine, dried over sodium sulfate, filtered, and concentrated under reduced pressure. The residue was purified by column chromatography on silica gel with petroleum ether/ethyl acetate (1/1) to give methyl 6-(5-((2,4-dimethoxybenzyl)amino)-6-fluoropyrazin-2-yl)-6′-ethoxy-5-fluoro-[2,3′-bipyridine]-4-carboxylate (400 mg, 0.74 mmol, 66.3% yield) as a yellow solid. LC purity: 80% (UV at 254 nm); Mass calculated for Chemical Formula: C₂₇H₂₅F₂N₅O₅[M+1]⁺, 538.2, found 538.2; Retention time: 0.994 min.

Step 6: To a solution of methyl 6-(5-((2,4-dimethoxybenzyl)amino)-6-fluoropyrazin-2-yl)-6′-ethoxy-5-fluoro-[2,3′-bipyridine]-4-carboxylate (1.00 equiv. 400 mg, 0.74 mmol) in THF (3 mL) and water (1 mL) was added LiGH (10.04 equiv. 178 mg, 7.43 mmol). The reaction was stirred for 1 h at ambient temperature. The mixture was evaporated and the residue was added DMSO, filtered and purified via phase flash chromatography (C18 silica gel) using a gradient of 20% to 50% ACN in water (with 10 mmol/L NH₄HCO₃.) to afford 6-(5-((2,4-dimethoxybenzyl)amino)-6-fluoropyrazin-2-yl)-6′-ethoxy-5-fluoro-[2,3′-bipyridine]-4-carboxylic acid (310 mg, 0.59 mmol, 79.8% yield) as a yellow solid. LC purity: 90% (UV at 254 nm); Mass calculated for Chemical Formula: C₂₆H₂₃F₂N₅O₅[M+1]⁺, 524.2, found 524.2; Retention time: 0.530 min.

Step 7: To a solution of 6-(5-((2,4-dimethoxybenzyl)amino)-6-fluoropyrazin-2-yl)-6′-ethoxy-5-fluoro-[2,3′-bipyridine]-4-carboxylic acid (1 equiv. 310 mg, 0.59 mmol) in DCM (3 mL) and added TFA (1 mL) dropwise at 0° C. and the resulting solution was stirred at room temperature for 2h. Concentrated to afford crude product which was used directly in the next step without further purification. Mass calculated for Chemical Formula: C₁₇H₁₃F₂N₅03 [M+1]⁺, 374.1, found 374.1; Retention time: 0.426 min.

Step 8: To a solution of 6-(5-amino-6-fluoropyrazin-2-yl)-6′-ethoxy-5-fluoro-[2,3′-bipyridine]-4-carboxylic acid (1 equiv. 60 mg, 0.16 mmol) and tert-butyl (R)-piperidin-3-ylcarbamate (1.5 equiv. 48 mg, 0.24 mmol) in DMA (5 mL) were added HATU (1.5 equiv. 92 mg, 0.24 mmol) and DIEA (2 equiv. 41 mg, 0.32 mmol). The mixture was stirred at room temperature overnight. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The product was used in the next step directly without further purification. LC purity: 85.6% (UV at 254 nm); Mass calculated for C₂₇H₃₁F₂N₇O₄[M+1]⁺ 556.2, found 556.2; Retention time: 0.843 min.

Step 9: To a solution of tert-butyl (R)-(1-(6-(5-amino-6-fluoropyrazin-2-yl)-6′-ethoxy-5-fluoro-[2,3′-bipyridine]-4-carbonyl)piperidin-3-yl)carbamate (1 equiv. 60 mg, 0.11 mmol) in DCM (3 mL) was added TFA (1 mL) dropwise at 0° C. and the resulting solution was stirred at room temperature for 2 h The yellow solution was bubbled with nitrogen to remove DCM and the residue was dissolved into MeCN, purified by Prep-HPLC (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 24% B to 39% B in 8 min; Wavelength: 254 nm/220 nm; RT1(min): 6.38). Pure fractions were evaporated to afford (R)-(6-(5-amino-6-fluoropyrazin-2-yl)-6′-ethoxy-5-fluoro-[2,3′-bipyridin]-4-yl)(3-aminopiperidin-1-yl)methanone (33 mg, 0.07 mmol, 67% yield) was a white solid.

Example 61: LC purity: 99.3% (UV at 254 nm); Mass calculated for C₂₂H₂₃F₂N₇O₂ [M+1]⁺ 456.2, found 456.2. Retention time: 1.282 min. ¹H NMR (400 MHz, DMSO) δ 8.96 (s, 1H), 8.75 (dd, J=4.7, 2.7 Hz, 1H), 8.48 (dd, J=8.9, 2.6 Hz, 1H), 8.03 (d, J=3.6 Hz, 1H), 7.40 (s, 2H), 6.94 (dd, J=8.7, 1.9 Hz, 1H), 4.39-4.37 (m, 2H), 3.31-2.93 (m, 3H), 2.68-2.63 (m, 3H), 1.87-1.78 (m, 2H), 1.56-1.53 (m, 2H), 1.36 (t, J=7.0 Hz, 3H), 1.34-1.32 (m, 1H).

### Example 62. (S)-(5-amino-6″-ethoxy-3′,6-difluoro-[2,2′:6′,3″-terpyridin]-4′-yl)(3-aminopiperidin-1-yl)methanone

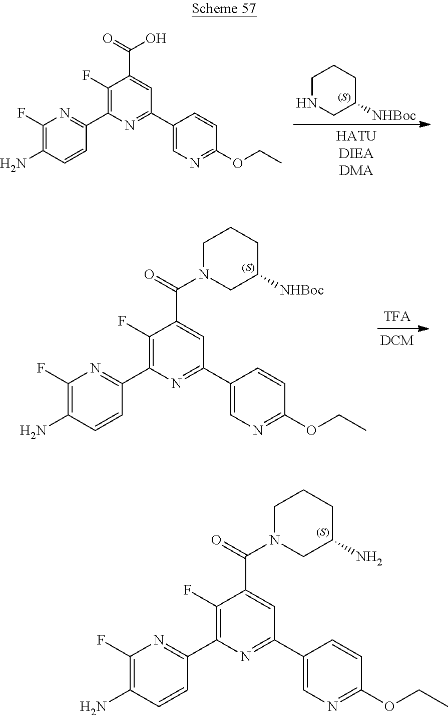

Step 1: To a solution of 5-amino-6″-ethoxy-3′,6-difluoro-[2,2′: 6′,3″-terpyridine]-4′-carboxylic acid (1.0 equiv. 80 mg, 0.21 mmol) and tert-butyl (S)-piperidin-3-ylcarbamate (1.2 equiv. 52 mg, 0.26 mmol) in DMA (5 mL) were added DIEA (3.0 equiv. 83 mg, 0.6 mmol) and HATU (1.5 equiv. 122 mg, 0.32 mmol). The resulting mixture was stirred at room temperature for 1 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure to afford tert-butyl (S)-(1-(5-amino-6″-ethoxy-3′,6-difluoro-[2,2′:6′,3″-terpyridine]-4′-carbonyl)piperidin-3-yl)carbamate (60 mg, 0.108 mmol, 50% yield) as a light yellow solid, which was used directly in the next step without further purification. LC purity: 80% (UV at 254 nm); Mass calculated for C₂₈H₃₂F₂N₆O₄[M+1]⁺ 555.2, found 555.2; Retention time: 0.812 min.

Step 2: To a stirred solution of tert-butyl (S)-(1-(5-amino-6″-ethoxy-3′,6-difluoro-[2,2′:6′,3″-terpyridine]-4′-carbonyl)piperidin-3-yl)carbamate (1.0 equiv. 60 mg, 0.108 mmol) in DCM (3 mL) was added TFA (1 mL). The mixture was stirred at room temperature for 1 h. The reaction mixture was diluted with water and extracted with EtOAc (2×50 mL). The combined organic layers were washed with brine (2×50 mL), dried over anhydrous sodium sulfate, filtered and concentrated. The residue was purified by prep-HPLC (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 25% B to 40% B in 8 min; Wavelength: 254 nm/220 nm; RT1(min): 7.03). The product was concentrated under reduced pressure and lyophilized to afford (S)-(5-amino-6″-ethoxy-3′,6-difluoro-[2,2′:6′,3″-terpyridin]-4′-yl)(3-aminopiperidin-1-yl)methanone (23 mg, 0.05 mmol, 47% yield) as an off-white solid.

Example 62: LC purity: 97.5% (UV at 254 nm); Mass calculated for C₂₃H₂₄F₂N₆O₂ [M+1]⁺ 455.2, found 455.2; Retention time: 1.271 min. ¹H NMR (400 MHz, DMSO) δ 8.98-8.93 (m, 1H), 8.47 (dd, J=8.8, 2.6 Hz, 1H), 8.05-7.95 (m, 2H), 7.33-7.25 (m, 1H), 6.93 (dd, J=8.7, 2.0 Hz, 1H), 5.98 (s, 2H), 4.44-4.32 (m, 3H), 3.43 (s, 1H), 3.02 (t, J=12.3 Hz, 1H), 2.86-2.60 (m, 2H), 1.99-1.54 (m, 4H), 1.47 (s, 1H), 1.36 (t, J=7.0 Hz, 3H), 1.32-1.22 (m, 1H).

### Example 63. (R)-(6-(5-amino-6-fluoropyrazin-2-yl)-6′-ethoxy-5,5′-difluoro-[2,3′-bipyridin]-4-yl)(3-aminopiperidin-1-yl)methanone

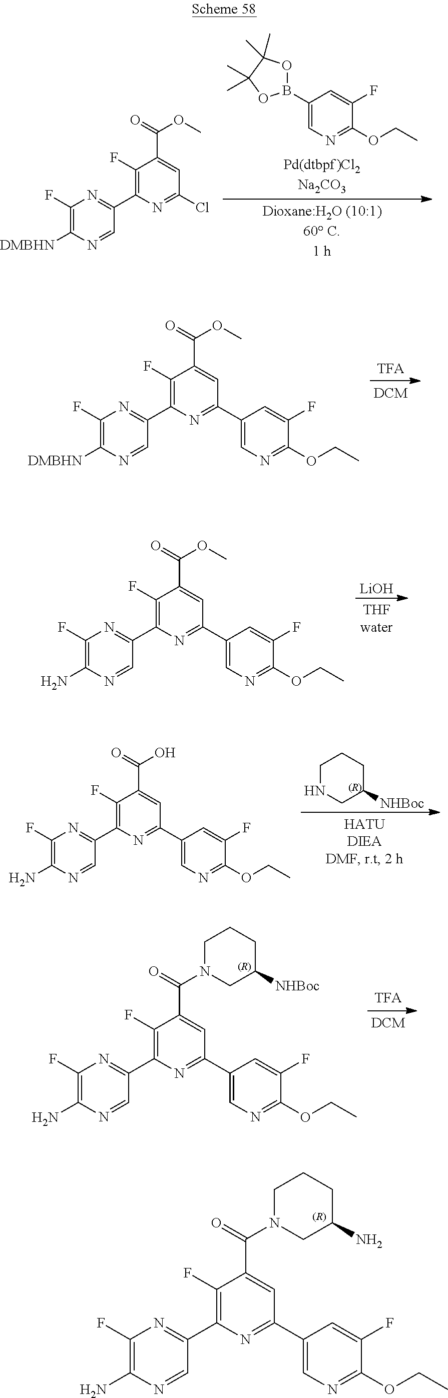

Step 1: To a solution of methyl 6-chloro-2-(5-((2,4-dimethoxybenzyl)amino)-6-fluoropyrazin-2-yl)-3-fluoroisonicotinate (1 equiv. 200 mg, 0.44 mmol) and 2-ethoxy-3-fluoro-5-(4,4,5,5-tetramethyl-1,3,2-dioxaborolan-2-yl)pyridine (1.5 equiv. 178 mg, 0.66 mmol) in 1,4-dioxane (5 mL) and water (0.5 mL) were treated with Pd(dtbpf)Cl₂ (0.1 equiv. 29 mg, 0.04 mmol) and Na₂CO₃ (2 equiv. 94 mg, 0.88 mmol). The resulting mixture was stirred at 60° C. for 4 h under nitrogen atmosphere. The resulting mixture was diluted with water and extracted with EtOAc (2×100 mL). The combined organic layers were washed with saturated aqueous potassium fluoride (1×30 mL), dried over anhydrous Na₂SO₄, filtered, and concentrated under reduced pressure. The residue was purified by reversed-phase flash chromatography with the following conditions: column, C18 silica gel; mobile phase, MeCN in water (0.1% FA), 0% to 80% gradient in 18 min; detector, UV 254 nm. The resulting mixture was concentrated under vacuum. This resulted in methyl 6-(5-((2,4-dimethoxybenzyl)amino)-6-fluoropyrazin-2-yl)-6′-ethoxy-5,5′-difluoro-[2,3′-bipyridine]-4-carboxylate (200 mg, 0.36 mmol, 81% yield) as a yellow solid. Mass calculated for C₂₇H₂₄F₃N₅O₅[M+H]⁺ 556.2, found 556.2. Retention time: 1.309 min.

Step 2: To a solution of methyl 6-(5-((2,4-dimethoxybenzyl)amino)-6-fluoropyrazin-2-yl)-6′-ethoxy-5,5′-difluoro-[2,3′-bipyridine]-4-carboxylate (1 equiv. 200 mg, 0.36 mmol) in DCM (2 mL) was added TFA (2 mL). The yellow solution was bubbled with nitrogen to remove DCM and the residue was dissolved in MeCN, purified by reversed-phase flash chromatography with the following conditions: column, C18 silica gel; mobile phase, MeCN in water (0.1% FA), 0% to 80% gradient in 18 min; detector, UV 254 nm. The pure fractions were concentrated under vacuum to afford methyl 6-(5-amino-6-fluoropyrazin-2-yl)-6′-ethoxy-5,5′-difluoro-[2,3′-bipyridine]-4-carboxylate (100 mg, 0.24 mmol, 68% yield) was a yellow solid. LC purity: 90.4% (UV at 254 nm); Mass calculated for C₁₈H₁₄F₃N₅O₃[M+1]⁺ 406.1, found 406.1. Retention time: 1.032 min.

Step 3: To a solution of methyl 6-(5-amino-6-fluoropyrazin-2-yl)-6′-ethoxy-5,5′-difluoro-[2,3′-bipyridine]-4-carboxylate (1 equiv. 100 mg, 0.24 mmol) in THF (1 mL) and water (1 mL) was treated with LiGH (3 equiv. 30 mg, 0.74 mmol). The resulting mixture was stirred at room temperature for 4 h, then acidified to pH 5-6 using 1 N HCl. The precipitated solid was collected by filtration, washed with water (2 mL), and dried to afford crude product 6-(5-amino-6-fluoropyrazin-2-yl)-6′-ethoxy-5,5′-difluoro-[2,3′-bipyridine]-4-carboxylic acid (60 mg, 0. 15 mmol, 62% yield) as a yellow solid. LC purity: 85% (UV at 254 nm); Mass calculated for C₁₇H₁₂F₃N₅O₃[M+1]⁺, 392.1, found 392.1. Retention time: 0.565 min.

Step 4: To a solution of 6-(5-amino-6-fluoropyrazin-2-yl)-6′-ethoxy-5,5′-difluoro-[2,3′-bipyridine]-4-carboxylic acid (1 equiv. 60 mg, 0.15 mmol) and tert-butyl (R)-piperidin-3-ylcarbamate (1.5 equiv. 46 mg, 0.23 mmol) in DMA (5 mL) were added HATU (1.5 equiv. 87 mg, 0.23 mmol) and DIEA (2 equiv. 40 mg, 0.3 mmol). The mixture was stirred at room temperature for 2 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The product was used in the next step directly without further purification. LC purity: 90% (UV at 254 nm); Mass calculated for C₂₇H₃₀F₃N₇O₄[M+1]⁺, 574.2, found 574.2; Retention time: 1.170 min.

Step 5: To a solution of tert-butyl (R)-(1-(6-(5-amino-6-fluoropyrazin-2-yl)-6′-ethoxy-5,5′-difluoro-[2,3′-bipyridine]-4-carbonyl)piperidin-3-yl)carbamate (1 equiv. 60 mg, 0.11 mmol) in DCM (3 mL) was added TFA (1 mL). The resulting solution was stirred at room temperature for 2 h under nitrogen. The yellow solution was bubbled with nitrogen to remove DCM and the residue was dissolved in MeCN, and purified by Prep-HPLC (Column: YMC-Actus-Triart C18 150*30.0 mml. D.S-5 μm; Mobile Phase A: Water (10 mmol/LNH₄HCO₃), Mobile Phase B: ACN; Flow rate: 60 mL/min; Gradient (B %): 32% B to 44% B in 8 min; Wavelength: 254 nm/220 nm; RT1(min): 7.52). Pure fractions were evaporated to afford (R)-(6-(5-amino-6-fluoropyrazin-2-yl)-6′-ethoxy-5,5′-difluoro-[2,3′-bipyridin]-4-yl)(3-aminopiperidin-1-yl)methanone (12 mg, 0.025 mmol, 24% yield) as a white solid.

Example 63: LC purity: 98.8% (UV at 254 nm); Mass calculated for C₂₂H₂₂F₃N₇O₂ [M+1]⁺ 474.2, found 474.2. Retention time: 1.244 min. H NMR (400 MHz, DMSO) δ 8.80 (s, 2H), 8.45 (d, J=11.4 Hz, 1H), 8.09 (s, 1H), 7.41 (s, 2H), 4.60-4.31 (m, 2H), 2.90-3.13 (m, 2H), 2.68 (s, 3H), 2.32-1.10 (m, 9H).

### Example 64. (R)-(3-amino-3-methylpiperidin-1-yl)(5-amino-6″-ethoxy-3′,5″,6-trifluoro-[2,2′:6′,3″-terpyridin]-4′-yl)methanone

### Example 65. (S)-(3-amino-3-methylpiperidin-1-yl)(5-amino-6″-ethoxy-3′,5″,6-trifluoro-[2,2′:6′,3″-terpyridin]-4′-yl)methanone

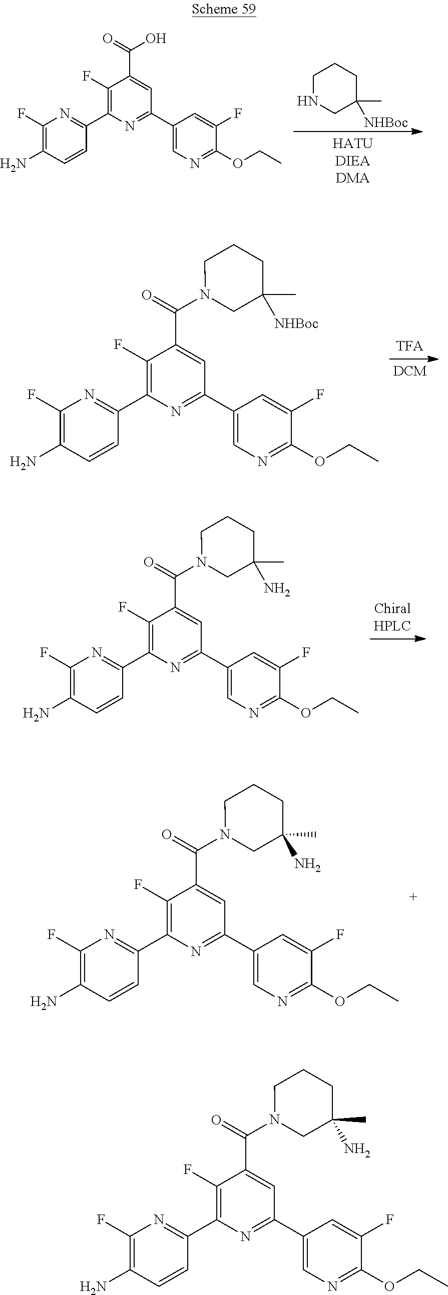 [SMILES: CCOc1ncc(-c2cc(C(=O)O)c(F)c(-c3ccc(N)c(F)n3)n2)cc1F ; CC1(NC(=O)OC(C)(C)C)CCCNC1 ; CCOc1ncc(-c2cc(C(=O)N3CCCC(C)(NC(=O)OC(C)(C)C)C3)c(F)c(-c3ccc(N)c(F)n3)n2)cc1F ; CCOc1ncc(-c2cc(C(=O)N3CCCC(C)(N)C3)c(F)c(-c3ccc(N)c(F)n3)n2)cc1F ; CCOc1ncc(-c2cc(C(=O)N3CCC[C@@](C)(N)C3)c(F)c(-c3ccc(N)c(F)n3)n2)cc1F ; CCOc1ncc(-c2cc(C(=O)N3CCC[C@](C)(N)C3)c(F)c(-c3ccc(N)c(F)n3)n2)cc1F]

Step 1: To a solution of 5-amino-6″-ethoxy-3′,5″,6-trifluoro-[2,2′:6′,3″-terpyridine]-4′-carboxylic acid (1.0 equiv. 150 mg, 0.38 mmol) and tert-butyl (3-methylpiperidin-3-yl)carbamate (1.2 equiv. 99 mg, 0.46 mmol) in DMA (5 mL) were added DIEA (4.0 equiv. 198 mg, 1.5 mmol) and HATU (2.0 equiv. 292 mg, 0.77 mmol). The mixture was stirred at room temperature for overnight. The resulting mixture was quenched with water and extracted with EtOAc (2×100 mL). The combined organic layers were washed with brine (1×30 mL), dried over anhydrous Na₂SO₄. After filtration, the filtrate was concentrated under reduced pressure. The residue was purified via reversed phase flash chromatography using a gradient of 70% to 100% ACN in water (with 10 mmol/L NH₄HCO₃) to afford tert-butyl (1-(5-amino-6″-ethoxy-3′,5″,6-trifluoro-[2,2′:6′,3″-terpyridine]-4′-carbonyl)-3-methylpiperidin-3-yl)carbamate (120 mg, 0.2 mmol, 53% yield) as a yellow solid. LC purity: 76.3% (UV at 254 nm); Mass calculated for C₂₉H₃₃F₃N₆O₄ [M+1]⁺, 587.3, found 587.3; Retention time: 0.982 min.

Step 2: To a solution of tert-butyl (1-(5-amino-6″-ethoxy-3′,5″,6-trifluoro-[2,2′:6′,3″-terpyridine]-4′-carbonyl)-3-methylpiperidin-3-yl)carbamate (1.0 equiv. 120 mg, 0.2 mmol) in DCM (5 mL) was added TFA (1 mL) and the mixture was stirred at room temperature for 2 h. The resulting mixture was concentrated under reduced pressure. The residue was purified by prep-HPLC with following condition: Column: XBridge Prep Shield RP OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 32% B to 47% B in 8 min; Wavelength: 254 nm/220 nm; RT1(min): 7.78. The pure fractions were concentrated and lyophilized to afford (3-amino-3-methylpiperidin-1-yl)(5-amino-6″-ethoxy-3′,5″,6-trifluoro-[2,2′:6′,3″-terpyridin]-4′-yl)methanone (50 mg, 0.1 mmol, 50% yield) as a white solid. LC purity: 97.2% (UV at 254 nm); Mass calculated for C₂₋₄H₂₅F₃N₆O₂ [M+1]⁺, 487.2, found 487.2; Retention time: 0.980 min.

Step 3: The racemate (3-amino-3-methylpiperidin-1-yl)(5-amino-6″-ethoxy-3′,5″,6-trifluoro-[2,2′:6′,3″-terpyridin]-4′-yl)methanone (50 mg, 0.1 mmol) was purified by prep-chiral-HPLC with following conditions: Column: CHIRALPAK IF-3; Mobile Phase A: Hex (0.1% DEA): (MeOH: DCM=1: 1)=70: 30; Flow rate: 1 mL/min mL/min. The pure fractions of first peak (RT1 (min): 2.516, assigned as the R isomer) and second peak (RT2 (min): 3.341, assigned as the S isomer) were concentrated and the residue was re-dissolved in CH₃CN and H₂O, and then was lyophilized to afford the title compound, RT1 (23 mg, 0.046 mmol, 46% yield) and RT2 (22 mg, 0.044 mmol, 44% yield), both obtained as a white solid (stereochemistry arbitrarily assigned).

Example 64: RT1: LC purity: 99.8% (UV at 254 nm); Mass calculated for C₂₋₄H₂₅F₃N₆O₂[M+1]⁺, 487.2, found 487.2; Retention time: 0.980 min. ¹H NMR (400 MHz, DMSO) δ 8.87-8.73 (m, 1H), 8.50-8.32 (m, 1H), 8.13-7.96 (m, 2H), 7.30 (dd, J=11.2, 8.1 Hz, 1H), 5.99 (d, J=4.2 Hz, 2H), 4.58-4.41 (m, 2H), 3.95-3.57 (m, 1H), 3.49 (s, 1H), 3.22 (d, J=21.3 Hz, 1H), 3.13-2.94 (m, 1H), 1.62 (s, 2H), 1.51 (d, J=20.3 Hz, 3H), 1.43-1.36 (m, 3H), 1.09 (s, 1H), 0.91 (s, 1H).

Example 65: RT2: LC purity: 99.8% (UV at 254 nm); Mass calculated for C₂₋₄H₂₅F₃N₆O₂[M+1]⁺, 487.2, found 487.2; Retention time: 0.987 min. H NMR (400 MHz, DMSO) δ 8.78 (d, J=23.6 Hz, 1H), 8.52-8.30 (m, 1H), 8.15-7.94 (m, 2H), 7.31 (dd, J=11.1, 8.1 Hz, 1H), 5.99 (d, J=4.1 Hz, 2H), 4.56-4.39 (m, 2H), 3.99-3.58 (m, 1H), 3.51 (d, J=12.2 Hz, 1H), 3.23 (d, J=24.8 Hz, 1H), 3.14-2.94 (m, 1H), 1.69 (s, 2H), 1.51 (d, J=20.9 Hz, 3H), 1.43-1.36 (m, 3H), 1.10 (s, 1H), 0.91 (s, 1H).

### Example 66. (5-amino-6″-ethoxy-3′,6-difluoro-[2,2′:6′,3″-terpyridin]-4′-yl)(4-aminopiperidin-1-yl)methanone

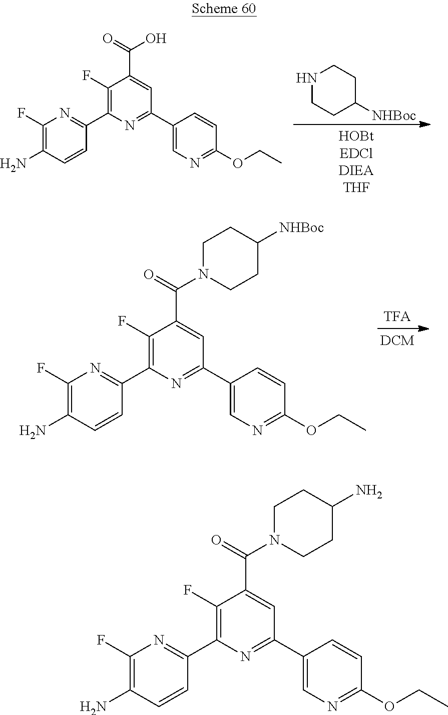 [SMILES: CCOc1ccc(-c2cc(C(=O)O)c(F)c(-c3ccc(N)c(F)n3)n2)cn1 ; CC(C)(C)OC(=O)NC1CCNCC1 ; CCOc1ccc(-c2cc(C(=O)N3CCC(NC(=O)OC(C)(C)C)CC3)c(F)c(-c3ccc(N)c(F)n3)n2)cn1 ; CCOc1ccc(-c2cc(C(=O)N3CCC(N)CC3)c(F)c(-c3ccc(N)c(F)n3)n2)cn1]

Step 1: To a solution of 5-amino-6″-ethoxy-3′,6-difluoro-[2,2′:6′,3″-terpyridine]-4′-carboxylic acid (1.0 equiv. 70 mg, 0.19 mmol), tert-butyl piperidin-4-ylcarbamate (1.2 equiv. 45 mg, 0.22 mmol) and HOBT (2.0 equiv. 51 mg, 0.38 mmol) in THF (3 mL) were added EDCI (2.0 equiv. 72 mg, 0.38 mmol) and DIEA (2.0 equiv. 49 mg, 0.38 mmol). The mixture was stirred at room temperature for 1 h. The resulting mixture was diluted with water and extracted with EtOAc. The organic layers were washed, dried and concentrated to afford crude tert-butyl (1-(5-amino-6″-ethoxy-3′,6-difluoro-[2,2′:6′,3″-terpyridine]-4′-carbonyl)piperidin-4-yl)carbamate (70 mg, 0.126 mmol, 66.4% yield) as a black oil. LC purity: 77.4% (UV at 254 nm); Mass calculated for C₂₈H₃₂F₂N₆O₄[M+1]⁺ 555.2, found 555.2; Retention time: 0.843 min.

Step 2: To a solution of tert-butyl (1-(5-amino-6″-ethoxy-3′,6-difluoro-[2,2′:6′,3″-terpyridine]-4′-carbonyl)piperidin-4-yl)carbamate (1.0 equiv. 60 mg, 0.11 mmol) in DCM (2.5 mL) was added TFA (1.0 equiv. 0.5 mL) at room temperature. The reaction was stirred at room temperature for 1 h. The resulting mixture was treated with 2 mL NH₄OH at 0° C. and concentrated. The residue was purified by prep-HPLC with following condition: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 21% B to 41% B in 8 min; Wavelength: 254 nm/220 nm nm; RT1(min): 7.23. The pure fractions were concentrated and lyophilized to afford title compound (24 mg, 0.05 mmol, 48% yield) as an off-white solid.

Example 66: LC purity: 99.0% (UV at 254 nm); Mass calculated for C₂₃H₂₄F₂N₆O₂ [M+1]⁺, 455.2, found 455.2; Retention time: 1.260 min. ¹H NMR (400 MHz, DMSO) δ 8.84 (d, J=2.5 Hz, 1H), 8.39 (dd, J=8.8, 2.6 Hz, 1H), 7.95 (d, J=8.1 Hz, 1H), 7.85 (s, 1H), 7.32 (dd, J=11.1, 8.2 Hz, 1H), 6.92 (d, J=8.7 Hz, 1H), 4.40 (s, 1H), 4.33-4.31 (m, 2H), 3.43 (d, J=13.5 Hz, 1H), 3.11-3.09 (m, 1H), 2.92 (s, 2H), 1.88 (s, 1H), 1.75 (s, 1H), 1.32 (t, J=7.0 Hz, 3H), 1.26 (s, 2H).

### Example 67. (5-amino-6″-ethoxy-3′,5″,6-trifluoro-[2,2′:6′,3″-terpyridin]-4′-yl)(4-aminopiperidin-1-yl)methanone

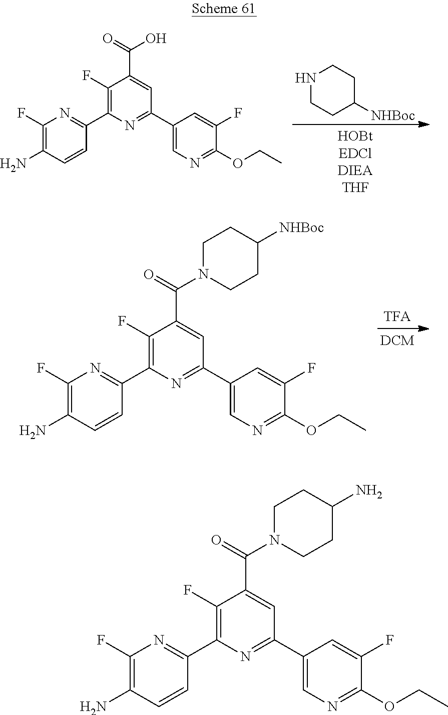 [SMILES: CCOc1ncc(-c2cc(C(=O)O)c(F)c(-c3ccc(N)c(F)n3)n2)cc1F ; CC(C)(C)OC(=O)NC1CCNCC1 ; CCOc1ncc(-c2cc(C(=O)N3CCC(NC(=O)OC(C)(C)C)CC3)c(F)c(-c3ccc(N)c(F)n3)n2)cc1F ; CCOc1ncc(-c2cc(C(=O)N3CCC(N)CC3)c(F)c(-c3ccc(N)c(F)n3)n2)cc1F]

Step 1: To a solution of 5-amino-6″-ethoxy-3′,5″,6-trifluoro-[2,2′:6′,3″-terpyridine]-4′-carboxylic acid (1.0 equiv. 70 mg, 0.18 mmol), tert-butyl piperidin-4-ylcarbamate (1.2 equiv. 43 mg, 0.22 mmol) and HOBt (2.0 equiv. 48 mg, 0.36 mmol) in THF (3 mL) were added EDCI (2.0 equiv. 69 mg, 0.36 mmol) and DIEA (2.0 equiv. 46 mg, 0.36 mmol) at room temperature. The reaction was stirred at room temperature for 1 h. The resulting mixture was diluted with water and extracted with EtOAc. The combined organic layers were washed with brine (1×30 mL), dried over anhydrous Na₂SO₄, and concentrated to afford crude tert-butyl (1-(5-amino-6″-ethoxy-3′, 5″,6-trifluoro-[2,2′:6′,3″-terpyridine]-4′-carbonyl)piperidin-4-yl)carbamate (70 mg, 0.12 mmol, 67.9% yield) as a black oil. LC purity: 85.5% (UV at 254 nm); Mass calculated for C₂₈H₃₁F₃N₆O₄[M+1]⁺ 573.2, found 573.2; Retention time: 1.063 min.

Step 2: To a stirred solution of tert-butyl (1-(5-amino-6″-ethoxy-3′,5″,6-trifluoro-[2,2′:6′,3″-terpyridine]-4′-carbonyl)piperidin-4-yl)carbamate (1.0 equiv. 60 mg, 0.1 mmol) in DCM (2.5 mL) was added TFA (1.0 equiv. 0.5 mL, 0.1 mmol), and the resulting mixture was stirred at room temperature for 1 h. The resulting mixture was treated with 2 mL NH₄OH at 0° C. and concentrated. The residue was purified by prep-HPLC with following condition: Column: XBridge Prep OBD C18 Column, 30*150 mm, 5 μm; Mobile Phase A: Water (10 mmol/L NH₄HCO₃+0.05% NH₃H2O), Mobile Phase B: ACN; Flow rate: 60 mL/min; Gradient (B %): 25% B to 45% B in 8 min; Wavelength: 254 nm/220 nm; RT1(min): 7.35. The pure fractions were concentrated and lyophilized to afford title compound (41 mg, 0.087 mmol, 81% yield) as an off-white solid.

Example 67: LC purity: 98.8% (UV at 254 nm); Mass calculated for C₂₃H₂₃F₃N₆O₂ [M+1]⁺, 473.2, found 473.2; Retention time: 1.253 min. ¹H NMR (400 MHz, DMSO) δ 8.61 (s, 1H), 8.26 (d, J=11.5 Hz, 1H), 7.92 (d, J=8.1 Hz, 1H), 7.83 (s, 1H), 7.31 (dd, J=11.1, 8.1 Hz, 1H), 4.41 (m, 3H), 3.43 (d, J=13.6 Hz, 1H), 3.12-3.09 (m, 1H), 2.93 (d, J=13.0 Hz, 2H), 1.91 (s, 1H), 1.77 (s, 1H), 1.33 (t, J=7.0 Hz, 3H), 1.28 (s, 2H).

### Example 68. (4-amino-4-methylpiperidin-1-yl)(5-amino-6″-ethoxy-3′,6-difluoro-[2,2′:6′,3″-terpyridin]-4′-yl)methanone

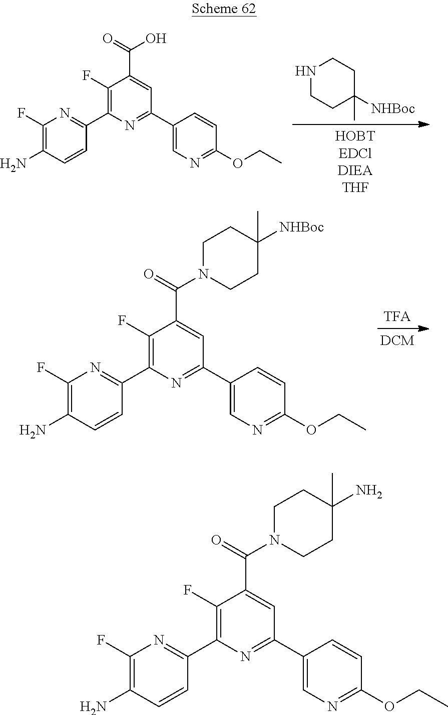 [SMILES: CCOc1ccc(-c2cc(C(=O)O)c(F)c(-c3ccc(N)c(F)n3)n2)cn1 ; CC1(NC(=O)OC(C)(C)C)CCNCC1 ; CCOc1ccc(-c2cc(C(=O)N3CCC(C)(NC(=O)OC(C)(C)C)CC3)c(F)c(-c3ccc(N)c(F)n3)n2)cn1 ; CCOc1ccc(-c2cc(C(=O)N3CCC(C)(N)CC3)c(F)c(-c3ccc(N)c(F)n3)n2)cn1]

Step 1: To a solution of 5-amino-6″-ethoxy-3′,6-difluoro-[2,2′:6′,3″-terpyridine]-4′-carboxylic acid (1.0 equiv. 70 mg, 0.19 mmol), tert-butyl (4-methylpiperidin-4-yl)carbamate (1.2 equiv. 48 mg, 0.23 mmol) and HOBT (2.0 equiv. 51 mg, 0.38 mmol) in THF (3 mL) were added EDCI (2.0 equiv. 72 mg, 0.38 mmol) and DIEA (2.0 equiv. 49 mg, 0.38 mmol) at room temperature. The reaction was stirred at room temperature for 1 h. The resulting mixture was diluted with water and extracted with EtOAc. The combined organic layers were washed with brine (1×30 mL), dried over anhydrous Na₂SO₄, filtered and concentrated to afford crude tert-butyl (1-(5-amino-6″-ethoxy-3′,6-difluoro-[2,2′:6′,3″-terpyridine]-4′-carbonyl)-4-methylpiperidin-4-yl)carbamate (70 mg, 0.12 mmol, 54% yield) as a black oil. LC purity: 82.6% (UV at 254 nm); Mass calculated for C₂₉H₃₄F₂N₆O₄[M+1]⁺ 569.3, found 569.1; Retention time: 0.872 min.

Step 2: To a solution of tert-butyl (1-(5-amino-6″-ethoxy-3′,6-difluoro-[2,2′:6′,3″-terpyridine]-4′-carbonyl)-4-methylpiperidin-4-yl)carbamate (1.0 equiv. 60 mg, 0.1 mmol) in DCM (2.5 mL) was added TFA (1.0 equiv. 0.5 mL), and the reaction mixture was stirred at room temperature for 1 h. The resulting mixture was treated with 2 mL NH₄0H at 0° C. and concentrated. The residue was purified by prep-HPLC with following condition: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 24% B to 44% B in 8 min; Wavelength: 254 nm/220 nm; RT1(min): 7.28. The pure fractions were concentrated and lyophilized to afford title compound (42 mg, 0.09 mmol, 83% yield) as a white solid.

Example 68: LC purity: 97.9% (UV at 254 nm); Mass calculated for C₂₋₄H₂₆F₂N₆O₂ [M+1]⁺, 469.2, found 469.2; Retention time: 0.728 min. ¹H NMR (400 MHz, DMSO) δ 8.95 (d, J=2.5 Hz, 1H), 8.47 (dd, J=8.7, 2.6 Hz, 1H), 8.01 (dd, J=8.0, 1.5 Hz, 1H), 7.96 (s, 1H), 7.30 (dd, J=11.2, 8.1 Hz, 1H), 6.93 (d, J=8.7 Hz, 1H), 5.98 (s, 2H), 4.39 (m, 2H), 3.97 (m, 1H), 3.54-3.38 (m, 2H), 3.21 (d, J=13.8 Hz, 1H), 1.58-1.47 (m, 4H), 1.36 (m, 5H), 1.10 (s, 3H).

### Example 69. (4-amino-4-methylpiperidin-1-yl)(5-amino-6″-ethoxy-3′,5″,6-trifluoro-[2,2′:6′,3″-terpyridin]-4′-yl)methanone

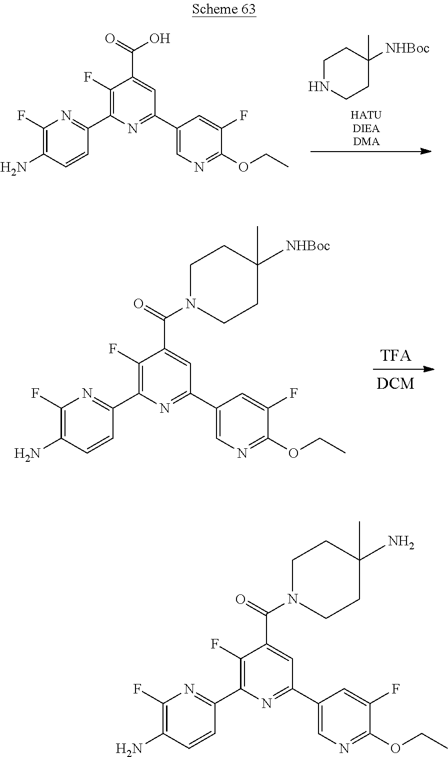 [SMILES: CCOc1ncc(-c2cc(C(=O)O)c(F)c(-c3ccc(N)c(F)n3)n2)cc1F ; CC1(NC(=O)OC(C)(C)C)CCNCC1 ; CCOc1ncc(-c2cc(C(=O)N3CCC(C)(NC(=O)OC(C)(C)C)CC3)c(F)c(-c3ccc(N)c(F)n3)n2)cc1F ; CCOc1ncc(-c2cc(C(=O)N3CCC(C)(N)CC3)c(F)c(-c3ccc(N)c(F)n3)n2)cc1F]

Step 1: To a solution of 5-amino-6″-ethoxy-3′,5″,6-trifluoro-[2,2′:6′,3″-terpyridine]-4′-carboxylic acid (1.0 equiv. 100 mg, 0.26 mmol) and tert-butyl (4-methylpiperidin-4-yl)carbamate (1.2 equiv. 66 mg, 0.31 mmol) in THF (5 ml) were added HOBT (1.5 equiv. 52 mg, 0.39 mmol), DIEA (4.0 equiv. 132 mg, 1.04 mmol) and EDCI (2.0 equiv. 98 mg, 0.52 mmol). The mixture was stirred at room temperature for overnight. The resulting mixture was quenched with water and extracted with EtOAc (2×100 mL). The combined organic layers were washed with brine (1×30 mL), dried over anhydrous Na₂SO₄. After filtration, the filtrate was concentrated under reduced pressure. The residue was purified via reversed phase flash chromatography using a gradient of 70% to 100% ACN in water (with 10 mmol/L NH₄HCO₃) to afford tert-butyl (1-(5-amino-6″-ethoxy-3′,5″,6-trifluoro-[2,2′:6′,3″-terpyridine]-4′-carbonyl)-4-methylpiperidin-4-yl)carbamate (130 mg, 0.22 mmol, 86% yield) as a yellow solid. LC purity:72.0% (UV at 254 nm); Mass calculated for C₂₉H₃₃F₃N₆O₄ [M+1]⁺, 587.3, found 587.3; Retention time: 1.104 min.

Step 2: To a stirred solution of tert-butyl (1-(5-amino-6″-ethoxy-3′,5″,6-trifluoro-[2,2′:6′,3″-terpyridine]-4′-carbonyl)-4-methylpiperidin-4-yl)carbamate (1.0 equiv. 70 mg, 0.1 mmol) in DCM (3 mL) was added TFA (1 mL). The mixture was stirred at room temperature for 1 h. The solid was filtered out. The filtrate was purified by Prep-HPLC with following condition: UV 254 nm/220 nm X-Bridge Prep OBD C18 Column, 30*150 mm, 5 m Water (10 mmol/L NH₄HCO₃+0.05% NH₃H₂O) ACN 60 mL/min 33% B to 53% B in 8 min 7.59. The pure fractions were concentrated and lyophilized to afford title compound (22 mg, 0.0453 mmol, 37.95% yield) as an off-white solid.

Example 69: LC purity: 99.5% (UV at 254 nm); Mass calculated for C₂₋₄H₂₅F₃N₆O₂ [M+1]⁺, 487.2, found 487.2; Retention time: 1.412 min. ¹H NMR (400 MHz, DMSO) δ 8.80 (d, J=1.9 Hz, 1H), 8.42 (dd, J=11.9, 2.1 Hz, 1H), 8.07-8.02 (m, 2H), 7.30 (dd, J=11.1, 8.1 Hz, 1H), 5.99 (s, 2H), 4.54-4.44 (m, 2H), 3.96 (s, 1H), 3.51-3.40 (m, 2H), 3.21 (s, 1H), 1.40 (t, J=7.1 Hz, 7H), 1.10 (s, 3H).

### Example 70. (R)-(5-amino-6″-ethoxy-3′,4,6-trifluoro-[2,2′:6′,3″-terpyridin]-4′-yl)(3-aminopiperidin-1-yl)methanone

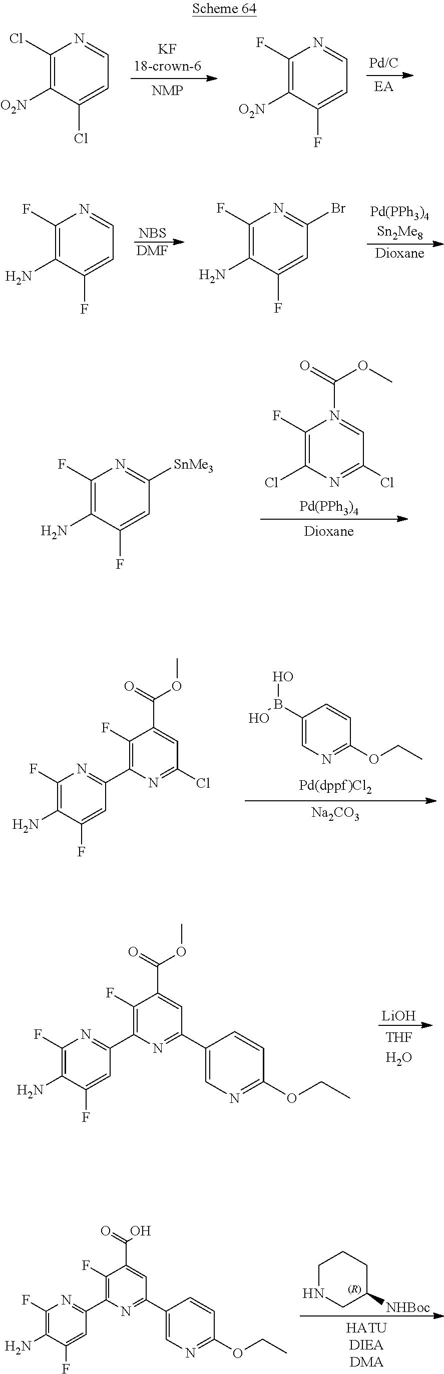

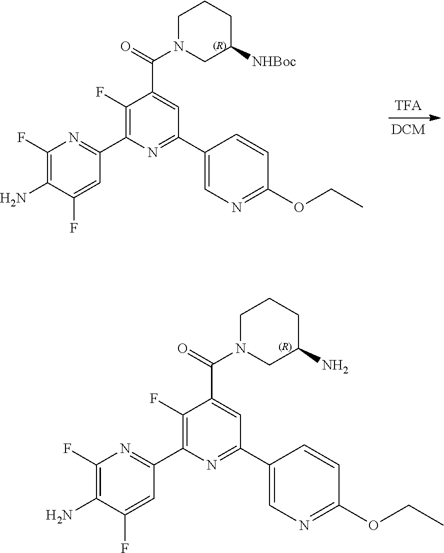

Step 1: To a solution of 2,4-dichloro-3-nitropyridine (1.0 equiv. 20.0 g, 104 mmol) in NMP (100 mL) were added KF (3.0 equiv. 18.0 g, 311 mmol) and 18-crown-6 (2.0 equiv. 54.8 g, 207 mmol). The mixture was stirred at 100° C. for 36 hours. The reaction mixture was cooled to room temperature and then partitioned between water and MTBE. The separated organic layer was washed with brine, dried over Na₂SO₄, filtered and concentrated in vacuo. The residue was purified by column chromatography on silica gel (Hexane: EtOAc=5:1) to afford 2,4-difluoro-3-nitropyridine (10.00 g, 62.5 mmol, 60.28% yield) as a pale-yellow oil. LC purity: 68.0% (UV at 254 nm); Mass calculated for C₅H2F₂N₂O₂ [M+1]⁺, 161.1, found 161.1; Retention time: 0.823 min.

Step 2: To a solution of 2,4-difluoro-3-nitropyridine (1.0 equiv. 6.00 g, 37.5 mmol) in EtOAc (40 mL) was added Pd/C (3.0 g). The suspension was purged with nitrogen for 5 minutes. Then the nitrogen atmosphere was replaced by hydrogen balloon (1 atm) and purged for 5 minutes. The reaction was stirred at room temperature for 2 hours. Reaction progress was monitored by TLC. Upon completion, the reaction was purged back with nitrogen. The suspension was then filtered over Celite and the filtrate cake was washed with EtOAc (30 mL). The combined filtrates were concentrated under reduced pressure to afford 2,4-difluoropyridin-3-amine (3.90 g, 30.0 mmol, 80% yield) as a white solid. LC purity: 89.3% (UV at 254 nm); Mass calculated for C₅H4F₂N₂[M+1]⁺, 131.1, found 131.1; Retention time: 0.838 min.

Step 3: To a stirred solution of 2,4-difluoropyridin-3-amine (1.0 equiv. 1.9 g, 14.6 mmol) in DMF (20 mL) was added NBS (1.2 equiv. 3.12 g, 17.5 mmol). The mixture was stirred at room temperature for 2 h. The resulting mixture was diluted with water and extracted with EtOAc (2×100 mL). The combined organic layers were washed with brine (1×30 mL), dried over anhydrous Na₂SO₄, filtered, concentrated. The residue was purified by column chromatography on silica gel (petroleum ether/EtOAc=5:1) to afford 6-bromo-2,4-difluoropyridin-3-amine (2.30 g, 11.0 mmol, 75% yield) as a pale-yellow oil. LC purity: 85.0% (UV at 254 nm); Mass calculated for C₅H₃BrF₂N₂[M+1]⁺, 209.1, found 209.1; Retention time: 0.843 min.

Step 4: To a stirred solution of 6-bromo-2,4-difluoropyridin-3-amine (1.0 equiv. 2.3 g, 11.0 mmol) in 1,4-dioxane (50 mL) were added Sn₂Me₆ (2.0 equiv. 7.2 g, 22.0 mmol) and Pd(PPh₃)₄(0.1 equiv. 1.2 g, 1.1 mmol). The resulting mixture was stirred over night at 100° C. under a nitrogen atmosphere. The resulting mixture was diluted with water and extracted with EtOAc (2×100 mL). The combined organic layers were washed with saturated aqueous potassium fluoride (1×30 mL), dried over anhydrous Na₂SO₄, filtered, and concentrated under reduced pressure to afford crude 2,4-difluoro-6-(trimethylstannyl)pyridin-3-amine (2.9 g, 9.9 mmol, 89% yield) as a light-yellow solid, which was used directly in the next step without further purification. LC purity: 76.2% (UV at 254 nm); Mass calculated for C₈H₁₂F₂N₂Sn [M+1]⁺, 295.1, found 295.1; Retention time: 0.743 min.

Step 5: To a solution of 2,4-difluoro-6-(trimethylstannyl)pyridin-3-amine (1.0 equiv. 500 mg, 1.7 mmol) and methyl 2,6-dichloro-3-fluoroisonicotinate (1.0 equiv. 380 mg, 1.7 mmol) in 1,4-dioxane (10 mL) was added Pd(PPh₃)₄(0.1 equiv. 118 mg, 0.17 mmol). The resulting mixture was stirred at 100° C. under a nitrogen atmosphere overnight. The resulting mixture was diluted with water and extracted with EtOAc (2×100 mL). The combined organic layers were washed with saturated aqueous potassium fluoride (1×30 mL), dried over anhydrous Na₂SO₄, filtered, and concentrated under reduced pressure. The residue was purified by reversed-phase flash chromatography, eluting with a gradient of 50% to 70% ACN in water (with 10 mmol/L NH₄HCO₃) to afford methyl 5′-amino-6-chloro-3,4′,6′-trifluoro-[2,2′-bipyridine]-4-carboxylate (220 mg, 0.69 mmol, 41% yield) as a yellow solid. LC purity: 63.1% (UV at 254 nm); Mass calculated for C₁₂H₇ClF₃N₃O₂ [M+1]⁺, 318.1, found 318.1; Retention time: 0.935 min.

Step 6: To a solution of methyl 5′-amino-6-chloro-3,4′,6′-trifluoro-[2,2′-bipyridine]-4-carboxylate (1.0 equiv. 80 mg, 0.25 mmol) and (6-ethoxypyridin-3-yl)boronic acid (1.5 equiv. 63 mg, 0.38 mmol) in 1,4-dioxane (5 mL) and water (0.5 mL) were added Na₂CO₃ (3.0 equiv. 79 mg, 0.75 mmol) and Pd(dppf)Cl₂ (0.1 equiv. 18 mg, 0.025 mmol). The mixture was stirred at 80° C. for 2 h under nitrogen atmosphere. The resulting mixture was diluted with water and extracted with EtOAc (2×100 mL). The combined organic layers were washed with brine (1×30 mL), dried over anhydrous Na₂SO₄. After filtration, the filtrate was concentrated under reduced pressure. The residue was purified via reversed phase flash chromatography (C18 silica gel) using a gradient of 50% to 70% ACN in water (with 10 mmol/L NH₄HCO₃) to afford methyl 5-amino-6″-ethoxy-3′,4,6-trifluoro-[2,2′:6′,3″-terpyridine]-4′-carboxylate (60 mg, 0.15 mmol, 58% yield) as a yellow solid. LC purity: 67.3% (UV at 254 nm); Mass calculated for C₁₉H₁₅F₃N₄O₃[M+1]⁺, 405.1, found, 405.1; Retention time: 0.992 min.

Step 7: To a stirred solution of methyl 5-amino-6″-ethoxy-3′,4,6-trifluoro-[2,2′:6′,3″-terpyridine]-4′-carboxylate (1.0 equiv. 60 mg, 0.15 mmol) in THF (3 mL) and Water (1 mL) was added LiGH (3.0 equiv. 12 mg, 0.45 mmol). The resulting mixture was stirred at room temperature for 2 h, then acidified to pH 5-6 using 1 N HCl. The precipitated solid was collected by filtration, washed with water (2 mL), and dried to afford crude product 5-amino-6″-ethoxy-3′,4,6-trifluoro-[2,2′:6′,3″-terpyridine]-4′-carboxylic acid (50 mg, 0.13 mmol, 86% yield) as a light-yellow solid, which was used directly in the next step without further purification. LC purity: 87.1% (UV at 254 nm); Mass calculated for C₁₈H₁₃F₃N₄O₃ [M+1]⁺, 391.1, found 391.1; Retention time: 0.732 min.

Step 8: To a solution of 5-amino-6″-ethoxy-3′,4,6-trifluoro-[2,2′:6′,3″-terpyridine]-4′-carboxylic acid (1.0 equiv. 60 mg, 0.2 mmol) and tert-butyl (R)-piperidin-3-ylcarbamate (1.5 equiv. 46 mg, 0.2 mmol) in DMA (3 mL) were added DIEA (3.0 equiv. 59 mg, 0.5 mmol) and HATU (1.5 equiv. 90 mg, 0.2 mmol). The mixture was stirred at room temperature for 1 h. The resulting mixture was quenched with water and extracted with EtOAc (2×100 mL). The combined organic layers were washed with brine (1×100 mL), dried over anhydrous Na₂SO₄. After filtration, the filtrate was concentrated under reduced pressure to afford tert-butyl (R)-(1-(5-amino-6″-ethoxy-3′,4,6-trifluoro-[2,2′:6′,3″-terpyridine]-4′-carbonyl)piperidin-3-yl)carbamate (60 mg, 0.1 mmol, 68% yield) as a light yellow solid, which was used directly in the next step without further purification. LC purity: 80.0% (UV at 254 nm); Mass calculated for C₂₈H₃₁F₃N₆O₄[M+1]⁺ 573.2, found 573.1; Retention time: 0.865 min.

Step 9: To a stirred solution of tert-butyl (R)-(1-(5-amino-6″-ethoxy-3′,4,6-trifluoro-[2,2′:6′,3″-terpyridine]-4′-carbonyl)piperidin-3-yl)carbamate (1.0 equiv. 60 mg, 0.1 mmol) in DCM (3 mL) was added TFA (1 mL). The mixture was stirred at room temperature for 1 h. The resulting mixture was concentrated under reduced pressure. The residue was purified by prep-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient (B %): 30% B to 55% B in 8 min; Wavelength: 254 nm/220 nm; RT1(min): 7.58). The product was concentrated under reduced pressure and lyophilized to afford the title compound (30 mg, 0.064 mmol, 61% yield) as a white solid.

Example 70: LC purity: 99.8% (UV at 254 nm); Mass calculated for C₂₃H₂₃F₃N₆O₂ [M+1]⁺ 473.2, found 473.2; Retention time: 1.184 min. ¹H NMR (400 MHz, DMSO) δ 9.00-8.93 (m, 1H), 8.50 (dd, J=8.7, 2.6 Hz, 1H), 8.08-8.00 (m, 2H), 6.93 (dd, J=8.7, 2.0 Hz, 1H), 6.07 (s, 2H), 4.44-4.31 (m, 3H), 3.11-2.96 (m, 1H), 2.91-2.52 (m, 3H), 2.00-1.53 (m, 3H), 1.50-1.15 (m, 6H).

### Example 71. (4-amino-4-methylpiperidin-1-yl)(5-amino-6″-ethoxy-3′,4,6-trifluoro-[2,2′:6′,3″-terpyridin]-4′-yl)methanone

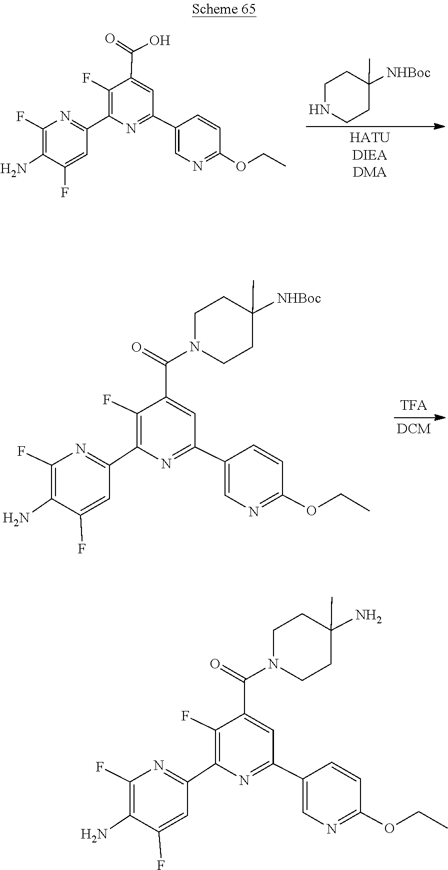 [SMILES: CCOc1ccc(-c2cc(C(=O)O)c(F)c(-c3cc(F)c(N)c(F)n3)n2)cn1 ; CC1(NC(=O)OC(C)(C)C)CCNCC1 ; CCOc1ccc(-c2cc(C(=O)N3CCC(C)(NC(=O)OC(C)(C)C)CC3)c(F)c(-c3cc(F)c(N)c(F)n3)n2)cn1 ; CCOc1ccc(-c2cc(C(=O)N3CCC(C)(N)CC3)c(F)c(-c3cc(F)c(N)c(F)n3)n2)cn1]

Step 1: To a solution of 5-amino-6″-ethoxy-3′,4,6-trifluoro-[2,2′:6′,3″-terpyridine]-4′-carboxylic acid (see, Example 70, Step 7) (1.0 equiv. 60 mg, 0.15 mmol) and tert-butyl (4-methylpiperidin-4-yl)carbamate (1.3 equiv. 43 mg, 0.2 mmol) in DMA (3 mL) were added DIEA (3.0 equiv. 59 mg, 0.4 mmol) and HATU (1.5 equiv. 90 mg, 0.23 mmol). The mixture was stirred at room temperature for 1 h. The resulting mixture was quenched with water and extracted with EtOAc (2×100 mL). The combined organic layers were washed with brine (3×100 mL), dried over anhydrous Na₂SO₄. After filtration, the filtrate was concentrated under reduced pressure to afford tert-butyl (1-(5-amino-6″-ethoxy-3′,4,6-trifluoro-[2,2′:6′,3″-terpyridine]-4′-carbonyl)-4-methylpiperidin-4-yl)carbamate (60 mg, 0.1 mmol, 66% yield) as a light yellow solid which was used directly in the next step without further purification. LC purity: 82.0% (UV at 254 nm); Mass calculated for C₂₉H₃₃F₃N₆O₄[M+1]⁺ 587.3, found 587.3; Retention time: 1.120 min.

Step 2: To a stirred solution of tert-butyl (1-(5-amino-6″-ethoxy-3′,4,6-trifluoro-[2,2′:6′,3″-terpyridine]-4′-carbonyl)-4-methylpiperidin-4-yl)carbamate (1.0 equiv. 60 mg, 0.1 mmol) in DCM (3 mL) was added TFA (1 mL). The mixture was stirred at room temperature for 1 h. The resulting mixture was concentrated under reduced pressure. The residue was purified by prep-HPLC (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient (B %): 30% B to 50% B in 8 min; Wavelength: 254 nm/220 nm; RT1(min): 7.28). The product was concentrated under reduced pressure and lyophilized to afford the title compound (27 mg, 0.05 mmol, 54% yield) as a white solid.

Example 71: LC purity: 99.5% (UV at 254 nm); Mass calculated for C₂₋₄H₂₅F₃N₆O₂ [M+1]⁺ 487.2, found 487.3; Retention time: 1.167 min. ¹H NMR (400 MHz, DMSO) δ 8.97 (d, J=2.5 Hz, 1H), 8.50 (dd, J=8.7, 2.5 Hz, 1H), 8.04 (d, J=10.9 Hz, 2H), 6.93 (d, J=8.7 Hz, 1H), 6.07 (s, 2H), 4.44-4.34 (m, 2H), 3.95 (s, 1H), 3.60-3.34 (m, 2H), 3.26-3.11 (m, 1H), 1.72-1.18 (m, 9H), 1.10 (s, 3H).

### Example 72. (S)-(5-amino-6″-ethoxy-3′,4,6-trifluoro-[2,2′:6′,3″-terpyridin]-4′-yl)(3-aminopiperidin-1-yl)methanone

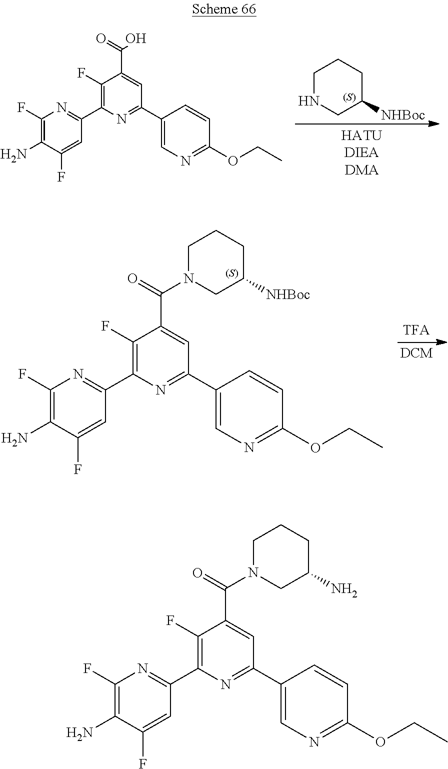

Step 1: To a solution of 5-amino-6″-ethoxy-3′,4,6-trifluoro-[2,2′:6′,3″-terpyridine]-4′-carboxylic acid (see, Example 70, Step 7) (1.0 equiv. 70 mg, 0.18 mmol) and tert-butyl (S)-piperidin-3-ylcarbamate (1.2 equiv. 43 mg, 0.22 mmol) in DMA (5 mL) were added DIEA (5.0 equiv. 116 mg, 0.9 mmol) and HATU (2.0 equiv. 136 mg, 0.36 mmol). The mixture was stirred at 25° C. for 1 h. The resulting mixture was quenched with water and extracted with EtOAc (2×100 mL). The combined organic layers were washed with brine (3×100 mL), dried over anhydrous Na₂SO₄. After filtration, the filtrate was concentrated under reduced pressure to afford tert-butyl (S)-(1-(5-amino-6″-ethoxy-3′,4,6-trifluoro-[2,2′:6′,3″-terpyridine]-4′-carbonyl)piperidin-3-yl)carbamate (50 mg, 0.087 mmol, 49% yield) as a light yellow solid, which was used directly in the next step without further purification. LC purity: 90.0% (UV at 254 nm); Mass calculated for C₂₈H₃₁F₃N₆O₄[M+1]⁺ 573.2, found 573.3; Retention time: 0.933 min.

Step 2: To a solution of tert-butyl (S)-(1-(5-amino-6″-ethoxy-3′,4,6-trifluoro-[2,2′:6′,3″-terpyridine]-4′-carbonyl)piperidin-3-yl)carbamate (1.0 equiv. 50 mg, 0.1 mmol) in DCM (3 mL) was added dropwise TFA (1 mL). The mixture was stirred at room temperature for 1 h. The resulting solution was concentrated under reduced pressure. The residue was purified by Prep-HPLC (Column: XBridge Prep OBD C18 Column, 30*150 mm, 5 m; Mobile Phase A: Water (10 mmol/LNH4HCO3), Mobile Phase B: ACN; Flow rate: 60 mL/min mL/min; Gradient (B %): 30% B to 55% B in 8 min; Wavelength: 254 nm/220 nm; RT1(min): 7.58). The product was concentrated under reduced pressure and lyophilized to the title compound (28 mg, 0.06 mmol, 66.81% yield) as a white solid.

Example 72: LC purity: 99.9% (UV at 254 nm); Mass calculated for C₂₃H₂₃F₃N₆O₂ [M+1]⁺ 472.2, found 472.3; Retention time: 1.186 min. ¹H NMR (400 MHz, DMSO) δ 9.00-8.94 (m, 1H), 8.51 (dd, J=8.5, 2.7 Hz, 1H), 8.09-8.01 (m, 2H), 6.93 (dd, J=8.7, 2.0 Hz, 1H), 6.08 (s, 2H), 4.44-4.32 (m, 3H), 3.42 (d, J=14.9 Hz, 1H), 3.11-2.96 (m, 1H), 2.82 (d, J=11.6 Hz, 1H), 2.69-2.59 (m, 1H), 1.80 (dd, J=60.1, 22.9 Hz, 4H), 1.49 (d, J=20.1 Hz, 1H), 1.40-1.33 (m, 3H), 1.32-1.20 (m, 1H).

### Example 73. (5-amino-6″-ethoxy-3′,5″,6-trifluoro-[2,2′:6′,3″-terpyridin]-4′-yl)(1-amino-3-azabicyclo[3.1.1]heptan-3-yl)methanone

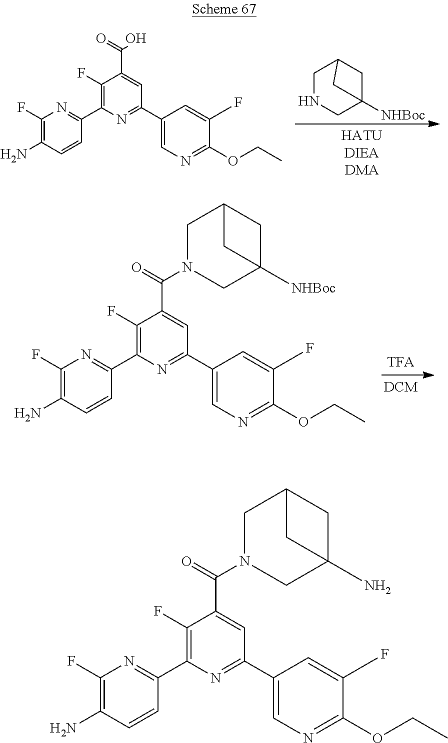

Step 1: To a stirred mixture of 5-amino-6″-ethoxy-3′,5″,6-trifluoro-[2,2′:6′,3″-terpyridine]-4′-carboxylic acid (1.0 equiv. 100 mg, 0.3 mmol) and tert-butyl (3-azabicyclo[3.1.1]heptan-1-yl)carbamate (1.5 equiv. 82 mg, 0.4 mmol) in DMA (5 mL) were added HATU (1.5 equiv. 146 mg, 0.4 mmol) and DIEA (2.0 equiv. 66 mg, 0.5 mmol) at room temperature. The reaction was stirred at room temperature for 1 h under a Nitrogen atmosphere. The resulting mixture was extracted with EtOAc. The organic layers were washed with sat. brine, dried and then afford tert-butyl (3-(5-amino-6″-ethoxy-3′,5″,6-trifluoro-[2,2′:6′,3″-terpyridine]-4′-carbonyl)-3-azabicyclo[3.1.1]heptan-1-yl)carbamate (99 mg, 0.2 mmol, 66.2% yield) as a crude yellow solid. LC purity: 56.1% (UV at 254 nm); Mass calculated for C₂₉H₃₁F₃N₆O₄[M+1]⁺ 585.2, found 585.3; Retention time: 0.855 min.

Step 2: To a stirred mixture of tert-butyl (3-(5-amino-6″-ethoxy-3′,5″,6-trifluoro-[2,2′:6′,3″-terpyridine]-4′-carbonyl)-3-azabicyclo[3.1.1]heptan-1-yl)carbamate (1.0 equiv. 60 mg, 0.1 mmol) in DCM (3 mL) was added TFA (1 mL), and the resulting mixture was stirred at room temperature for 1 h. The resulting mixture was purified with following condition: Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min; Gradient (B %): 28% B to 48% B in 8.5 min; Wavelength: 254 nm/220 nm nm; RT1(min): 8.22. The pure fractions were concentrated and lyophilized to afford title compound (22.1 mg, 0.0457 mmol, 44.6% yield) as a white solid.

Example 73: LC purity: 99.8% (UV at 254 nm); Mass calculated for C₂₋₄H₂₃F₃N₆O₂ [M+1]⁺ 485.2, found 485.2; Retention time: 1.241 min. ¹H NMR (400 MHz, DMSO) δ 8.80 (dd, J=5.3, 2.0 Hz, 1H), 8.45-8.38 (m, 1H), 8.17-7.87 (m, 2H), 7.33-7.25 (m, 1H), 5.99 (d, J=3.4 Hz, 2H), 4.52-4.43 (m, 2H), 3.56 (d, J=33.2 Hz, 2H), 3.34 (s, 1H), 3.26 (s, 1H), 2.42-2.16 (m, 1H), 2.05 (s, 2H), 1.92-1.83 (m, 2H), 1.57 (d, J=6.5 Hz, 2H), 1.44-1.33 (m, 3H).

### Example 74. (5-amino-6″-ethoxy-3′,5″,6-trifluoro-[2,2′:6′,3″-terpyridin]-4′-yl)(4-amino-2-azabicyclo[2.2.2]octan-2-yl)methanone

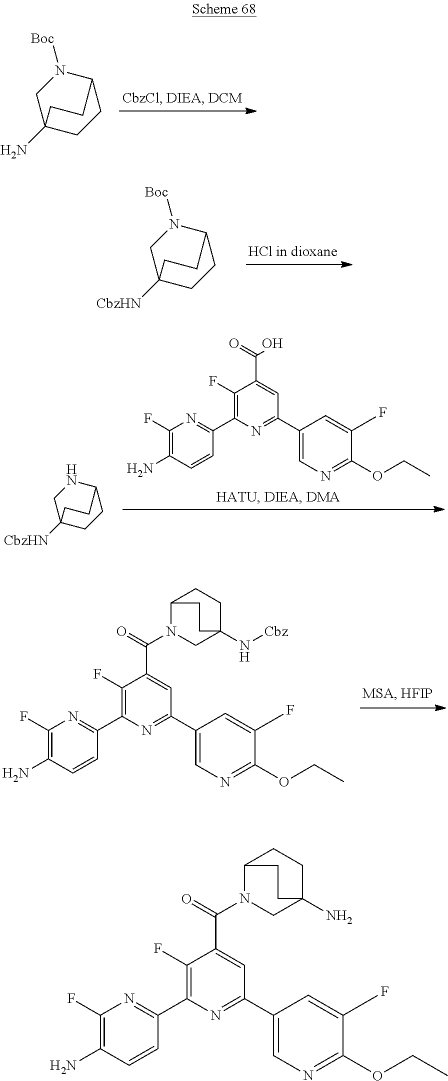 [SMILES: CC(C)(C)OC(=O)N1CC2(N)CCC1CC2 ; CC(C)(C)OC(=O)N1CC2(NC(=O)OCc3ccccc3)CCC1CC2 ; CCOc1ncc(-c2cc(C(=O)O)c(F)c(-c3ccc(N)c(F)n3)n2)cc1F ; O=C(NC12CCC(CC1)NC2)OCc1ccccc1 ; CCOc1ncc(-c2cc(C(=O)N3CC4(NC(=O)OCc5ccccc5)CCC3CC4)c(F)c(-c3ccc(N)c(F)n3)n2)cc1F ; CCOc1ncc(-c2cc(C(=O)N3CC4(N)CCC3CC4)c(F)c(-c3ccc(N)c(F)n3)n2)cc1F]

Step 1: To a solution of tert-butyl 4-amino-2-azabicyclo[2.2.2]octane-2-carboxylate (1 equiv. 300 mg, 1.3 mmol), CbzCl (1.5 equiv. 339.5 mg, 2 mmol) in DCM (10 mL) was added DIEA (2 equiv. 342 mg, 2.7 mmol) dropwise at 0° cand the resulting solution was stirred at room temperature for 2 h under nitrogen. The yellow solution was bubbled with nitrogen to remove DCM and the residue was dissolved in MeCN, purified by reversed-phase flash chromatography with the following conditions: column, C18 silica gel; mobile phase, MeCN in water (0.1% FA), 0% to 80% gradient in 18 min; detector, UV 254 nm. The resulting mixture was concentrated under vacuum. This resulted in tert-butyl 4-(((benzyloxy)carbonyl)amino)-2-azabicyclo[2.2.2]octane-2-carboxylate (200 mg, 0.55 mmol, 41.8% yield) as a yellow solid. LC purity: 90.4% (UV at 254 nm); Mass calculated for C₂₀H₂₈N₂O₄ [M+1]⁺ 361.1, found 361.1. Retention time: 1.032 min.

Step 2: To a solution of tert-butyl 4-(((benzyloxy)carbonyl)amino)-2-azabicyclo[2.2.2]octane-2-carboxylate (1 equiv. 200 mg, 0.55 mmol) in 1,4-dioxane (5 mL) was added HCl in 1,4-dioxane (1 M, 5 mL) dropwise at 0° C. and the resulting solution was stirred at room temperature for 2 h under nitrogen. The resulting mixture was concentrated under reduced pressure. The crude product was used in the next step directly without further purification.

Step 3: To a solution of 5-amino-6″-ethoxy-3′,5″,6-trifluoro-[2,2′:6′,3″-terpyridine]-4′-carboxylic acid (1 equiv. 100 mg, 0.26 mmol) and benzyl (2-azabicyclo[2.2.2]octan-4-yl)carbamate (1 equiv. 67 mg, 0.26 mmol) in DMA (5 mL) was added HATU (1.5 equiv. 146 mg, 0.38 mmol) and DIEA (1.5 equiv. 50 mg, 0.38 mmol) at room temperature. The reaction was stirred at room temperature for 1 h under nitrogen atmosphere. The resulting mixture was extracted with EtOAc (2×10 mL). The combined organic layers were washed with saturated aqueous NaCl solution (6×5 mL), dried over anhydrous Na₂SO₄. After filtration, the filtrate was concentrated under reduced pressure. The crude product was used in the next step directly without further purification.

Step 4: To a solution of benzyl (2-(5-amino-6″-ethoxy-3′,5″,6-trifluoro-[2,2′:6′,3″-terpyridine]-4′-carbonyl)-2-azabicyclo[2.2.2]octan-4-yl)carbamate (1 equiv. 70 mg, 0.11 mmol) in HFIP (5 mL) was added MSA (1 mL) dropwise at 0° C., and the resulting solution was stirred at room temperature for 2 h under nitrogen atmosphere. The yellow solution was bubbled with nitrogen to remove DCM and the residue was dissolved into MeCN, purified by Prep-HPLC (Column: XBridge Prep OBD C18 Column, 30*150 mm, 5 μm; Mobile Phase A: Water (10 mmol/L NH₄HCO₃+0.05% NH₃H₂O), Mobile Phase B: ACN; Flow rate: 60 mL/min mL/min; Gradient (B %): 31% B to 51% B in 8 min; Wavelength: 254 nm/220 nm nm; RT1(min): 6.85). Pure fractions were evaporated to afford the title compound (18 mg, 0.036 mmol, 32% yield) as a white solid.

Example 74: LC purity: 99.8% (UV at 254 nm); Mass calculated for C₂₅H₂₅F₃N₆O₂ [M+1]⁺ 499.5, found 499.5 [M+1]. Retention time: 1.227 min. H NMR (400 MHz, DMSO) δ 8.81 (dd, J=8.5, 2.0 Hz, 1H), 8.43-8.41 (m, 1H), 8.10-8.01 (m, 2H), 7.31-7.28 (m, 1H), 6.00 (d, J=4.5 Hz, 2H), 4.52-4.42 (m, 2H), 3.38 (s, 1H), 3.29 (s, 2H), 1.81 (d, J=24.6 Hz, 4H), 1.55 (t, J=8.1 Hz, 4H), 1.40-1.37 (m, 3H).

### Example 75. (5-amino-6″-ethoxy-3′,5″,6-trifluoro-[2,2′:6′,3″-terpyridin]-4′-yl)((3aS,7aR)-1-methyloctahydro-5H-pyrrolo[3,2-c]pyridin-5-yl)methanone

### Example 76. (5-amino-6″-ethoxy-3′,5″,6-trifluoro-[2,2′:6′,3″-terpyridin]-4′-yl)((3aR,7aS)-1-methyloctahydro-5H-pyrrolo[3,2-c]pyridin-5-yl)methanone

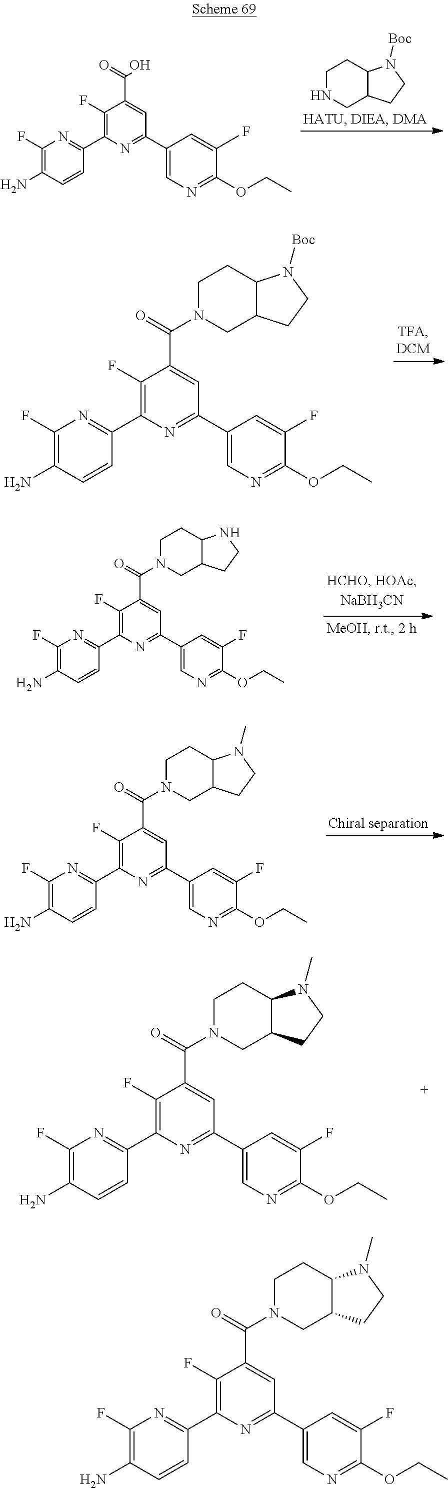

Step 1: To a solution of 5-amino-6″-ethoxy-3′, 5″, 6-trifluoro-[2,2′: 6′, 3″-terpyridine]-4′-carboxylic acid (1.0 equiv. 400 mg, 1.0 mmol) in DMA (3 mL) were added DIEA (3.0 equiv. 1.2 g, 3.1 mmol) and HATU (1.5 equiv. 570 mg, 1.5 mmol). The mixture was stirred at room temperature for 2 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The product was used in the next step directly without further purification. LC purity: 85.1% (UV at 254 nm); Mass calculated for C₃₀H₃₃F₃N₆O₄[M+1]⁺ 599.3, found 599.3; Retention time: 0.976 min.

Step 2: To a stirred mixture of tert-butyl 5-(5-amino-6″-ethoxy-3′,5″,6-trifluoro-[2,2′:6′,3″-terpyridine]-4′-carbonyl)octahydro-1H-pyrrolo[3,2-c]pyridine-1-carboxylate (1.0 equiv. 589 mg, 1.0 mmol) in DCM (9 mL) was added TFA (3 mL), and the resulting mixture was stirred at room temperature for 1 h. The mixture was evaporated and the residue was dissolved in DMSO, filtered, and the filtrate was purified by reversed-phase flash chromatography (C18 silica gel) with a gradient of 20-50% acetonitrile in water containing 10 mmol/L ammonium bicarbonate to afford (5-amino-6″-ethoxy-3′,5″,6-trifluoro-[2,2′:6′,3″-terpyridin]-4′-yl)(octahydro-5H-pyrrolo[3,2-c]pyridin-5-yl)methanone (315 mg, 0.6 mmol, 61.8% yield) as a yellow solid. LC purity: 99% (UV at 254 nm); Mass calculated for Chemical Formula: C₂₅H₂₅F₃N₆O₂[M+1]⁺, 499.2, found 499.2; Retention time: 0.674 min.

Step 3: To a stirred solution of (5-amino-6″-ethoxy-3′,5″,6-trifluoro-[2,2′:6′,3″-terpyridin]-4′-yl)(octahydro-5H-pyrrolo[3,2-c]pyridin-5-yl)methanone (1.00 eq, 315 mg, 0.6 mmol) and 40% formaldehyde (1.00 eq, 47 mg, 0.6 mmol) in MeOH (15 mL) was added Acetic acid (5.00 eq, 192 mg, 3.2 mmol) at room temperature. The resulting mixture was stirred at room temperature for 20 min. This was followed by the addition of a solution of NaBH₃CN (4.00 eq, 156 mg, 2.5 mmol) in MeOH (5 mL). The resulting mixture was stirred at room temperature for 2 h. The mixture was diluted with water and extracted with EtOAc. The combined extracts were washed with brine, dried over anhydrous sodium sulfate, filtered and concentrated. The residue was dissolved in DMSO, filtered, and purified by Prep-HPLC (Column: X-Bridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 30% B to 55% B in 8 min; Wavelength: 254 nm/220 nm; RT1(min): 6.58) to afford the title compound (5-amino-6″-ethoxy-3′, 5″,6-trifluoro-[2,2′:6′,3″-terpyridin]-4′-yl)(1-methyloctahydro-5H-pyrrolo[3,2-c]pyridin-5-yl)methanone (150 mg, 0.3 mmol, 43.01% yield) as a white solid. LC purity: 97.9% (UV at 254 nm); Mass calculated for Chemical Formula: C₂₆H₂₇F₃N₆O₂[M+1]⁺, 513.2, found 513.2; Retention time: 0.603 min.

Step 4: The racemate (5-amino-6″-ethoxy-3′, 5″,6-trifluoro-[2,2′:6′,3″-terpyridin]-4′-yl)(1-methyloctahydro-5H-pyrrolo[3,2-c]pyridin-5-yl)methanone (100 mg, 0.19 mmol) was purified by prep-chiral-HPLC with following conditions: Column: CHIRAL ART Amylose-C NEO, 5 μm, 20 mm 250 mm; Mobile Phase A: IPA, Mobile Phase B: Hex (with 0.5% NH₃ (2 M in MeOH)); Flow rate: 20 mL/min; Gradient (B %): 80% B; Wavelength: 220/254 nm; RT1(min): 34.65; RT2(min): 46.69; Sample Solvent: EtOH: DCM=1: 1; Injection Volume: 0.5 mL; Number Of Runs: 12; Single injection run time(min): 52. The pure fractions of the first peak (RT1 (min): 34.65) and the second peak (RT2 (min): 46.69) were concentrated. Each residue was re-dissolved in CH₃CN and H₂O, then lyophilized to afford the title compounds Example 75 (44 mg, 0.09 mmol, 45.1% yield) and Example 76 (41 mg, 0.08 mmol, 42.1% yield), both obtained as white solids.

Example 75: LC purity: 98.8% (UV at 254 nm); Mass calculated for C₂₆H₂₇F₃N₆O₂ [M+1]⁺, 513.2, found 513.2; Retention time: 1.248 min. ¹H NMR (400 MHz, DMSO) δ 8.80 (s, 1H), 8.43-8.40 (m, 1H), 8.06-8.04 (m, 2H), 7.33-7.27 (dd, J=11.2, 8.1 Hz, 1H), 6.00 (s, 2H), 4.55-4.43 (m, 2H), 4.08-3.78 (m, 1H), 3.30-3.24 (m, 1H), 3.23-3.13 (m, 1H), 3.12-3.05 (m, 1H), 3.04-2.90 (m, 1H), 2.48-2.03 (m, 6H), 2.01-1.59 (m, 3H), 1.39 (t, J=7.1 Hz, 3H), 1.24 (s, 1H).

Example 76: LC purity: 99.1% (UV at 254 nm); Mass calculated for C₂₆H₂₇F₃N₆O₂ [M+1]⁺, 513.2, found 513.2; Retention time: 1.246 min. ¹H NMR (400 MHz, DMSO) δ 8.80 (s, 1H), 8.42 (d, J=11.8 Hz, 1H), 8.09-8.01 (m, 2H), 7.30 (dd, J=11.2, 8.1 Hz, 1H), 5.99 (s, 2H), 4.51-4.42 (m, 2H), 4.06-3.75 (m, 1H), 3.31-3.12 (m, 2H), 3.10-2.88 (m, 2H), 2.41-2.18 (m, 5H), 2.17-2.05 (m, 1H), 1.90-1.60 (m, 3H), 1.39 (t, J=7.0 Hz, 3H), 1.23 (s, 1H).

### Example 77. (5-amino-6″-ethoxy-3′,5″,6-trifluoro-[2,2′:6′,3″-terpyridin]-4′-yl)((3aR,7aR)-1-methyloctahydro-6H-pyrrolo[2,3-c]pyridin-6-yl)methanone

Example 78. (5-amino-6″-ethoxy-3′,5″,6-trifluoro-[2,2′:6′,3″-terpyridin]-4′-yl)((3aS,7aS)-1-methyloctahydro-6H-pyrrolo[2,3-c]pyridin-6-yl)methanone

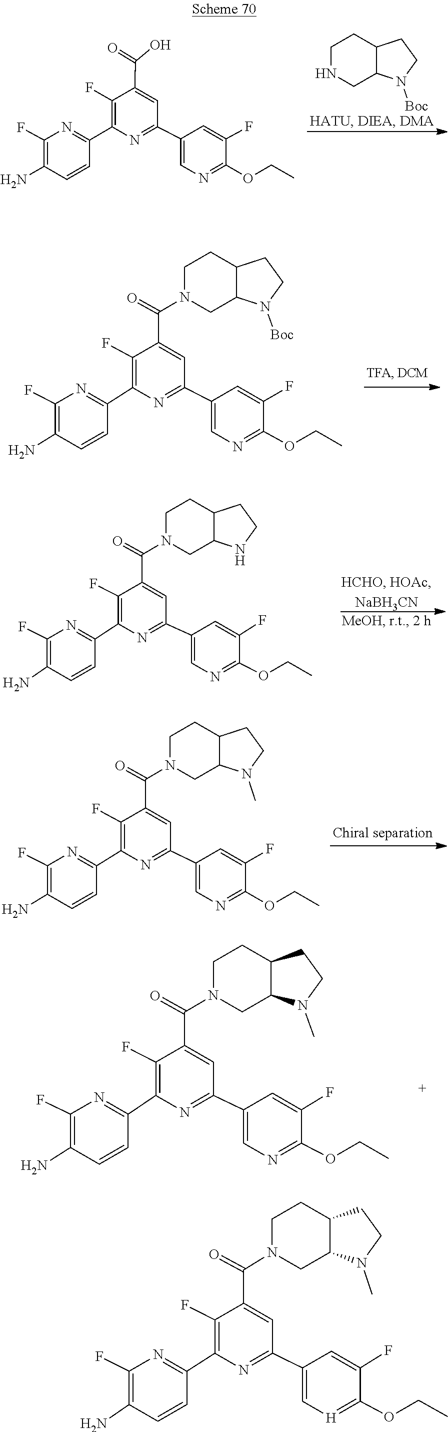

Step 1: To a solution of 5-amino-6″-ethoxy-3′,5″,6-trifluoro-[2,2′:6′,3″-terpyridine]-4′-carboxylic acid (1.0 equiv. 400 mg, 1.0 mmol) and tert-butyl octahydro-1H-pyrrolo[2,3-c]pyridine-1-carboxylate(1.5eq, 347 mg, 1.5 mmol) in DMA (5 mL) were added DIEA (3.0 equiv. 1.2 g, 3.1 mmol) and HATU (1.5 equiv. 570 mg, 1.5 mmol). The mixture was stirred at room temperature for 2 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The product was used in the next step directly without further purification. LC purity: 81.2% (UV at 254 nm); Mass calculated for C₃₀H₃₃F₃N₆O₄[M+1]⁺ 599.3, found 599.3; Retention time: 1.078 min.

Step 2: To a stirred mixture of tert-butyl 6-(5-amino-6″-ethoxy-3′,5″,6-trifluoro-[2,2′:6′,3″-terpyridine]-4′-carbonyl)octahydro-1H-pyrrolo[2,3-c]pyridine-1-carboxylate (1.0 equiv. 420 mg, 0.7 mmol) in DCM (9 mL) was added TFA (3 mL), and the resulting mixture was stirred at room temperature for 1 h. The mixture was evaporated and the residue was dissolved in DMSO, filtered, and the filtrate was purified by reversed-phase flash chromatography (C18 silica gel) with a gradient of 20-50% ACN in water (with 10 mmol/L NH₄HCO₃.) to afford (5-amino-6″-ethoxy-3′,5″,6-trifluoro-[2,2′: 6′,3″-terpyridin]-4′-yl)(octahydro-6H-pyrrolo[2,3-c]pyridin-6-yl)methanone (260 mg, 0.6 mmol, 74.4% yield) as a yellow solid. LC purity: 97% (UV at 254 nm); Mass calculated for Chemical Formula: C₂₅H₂₅F₃N₆O₂[M+1]⁺, 499.2, found 499.2; Retention time: 0.689 min.

Step 3: To a stirred solution of (5-amino-6″-ethoxy-3′,5″,6-trifluoro-[2,2′:6′,3″-terpyridin]-4′-yl)(octahydro-6H-pyrrolo[2,3-c]pyridin-6-yl)methanone (1.00 eq, 255 mg, 0.5 mmol) and 40% formaldehyde (1.00 eq, 38 mg, 0.5 mmol) in MeOH (10 mL) was added Acetic acid (5.00 eq, 150 mg, 2.5 mmol) at room temperature. The resulting mixture was stirred at room temperature for 20 min. This was followed by the addition of a solution of NaBH₃CN (4.00 eq, 124 mg, 2.0 mmol) in MeOH (5 mL) at room temperature. The resulting mixture was stirred at room temperature for 2 h. The mixture was diluted with water and extracted with EtOAc. The combined extracts were washed with brine, dried over anhydrous sodium sulfate, filtered and concentrated. The residue was dissolved in DMSO, filtered and purified via Prep-HPLC (Column: X-Bridge Prep OBD C18 Column, 30*150 mm, 5 μm; Mobile Phase A: Water(10 mmol/L NH₄HCO₃), Mobile Phase B: ACN; Flow rate: 60 mL/min mL/min; Gradient (B %): 30% B to 55% B in 8 min; Wavelength: 254 nm/220 nm nm; RT1(min): 6.58) to afford title compound (5-amino-6″-ethoxy-3′,5″,6-trifluoro-[2,2′:6′,3″-terpyridin]-4′-yl)(1-methyloctahydro-6H-pyrrolo[2,3-c]pyridin-6-yl)methanone (74 mg, 0.1 mmol, 28.85% yield) as a white solid. LC purity: 98.2% (UV at 254 nm); Mass calculated for Chemical Formula: C₂₆H₂₇F₃N₆O₂[M+1]⁺, 513.2, found 513.2; Retention time: 1.015 min.

Step 4: The racemate (5-amino-6″-ethoxy-3′,5″,6-trifluoro-[2,2′:6′,3″-terpyridin]-4′-yl)(1-methyloctahydro-6H-pyrrolo[2,3-c]pyridin-6-yl)methanone (74 mg, 0.1 mmol) was purified by prep-chiral-HPLC with following conditions: Column: CHIRALPAK IH, 5 μm, 20 mm 250 mm; Mobile Phase A: EtOH: DCM=1: 1, Mobile Phase B: Hex (with 0.5% NH₃ (2 M in MeOH)); Flow rate: 20 mL/min; Gradient (B %): 85% B; Wavelength: 220/254 nm; RT1(min): 17.52; RT2(min): 23.72; Sample Solvent: EtOH: DCM=1: 1; Injection Volume: 0.5 mL; Number Of Runs: 8; Single injection run time(min): 27. The pure fractions of the first peak (RT1 (min): 17.52) and second peak (RT2 (min): 23.72) were concentrated. Each residue was re-dissolved in acetonitrile and water, then lyophilized to afford the title compounds Example 77 (21 mg, 0.04 mmol, 28.4% yield) and Example 78 (21 mg, 0.04 mmol, 28.4% yield), both obtained as white solids.

Example 77: LC purity: 9.8% (UV at 254 nm); Mass calculated for C₂₆H₂₇F₃N₆O₂ [M+1]⁺, 513.2, found 513.2; Retention time: 1.337 min. ¹H NMR (400 MHz, DMSO) δ 8.79 (t, J=2.2 Hz, 1H), 8.48-8.37 (m, 1H), 8.09-7.88 (m, 2H), 7.30 (dd, J=11.1, 8.1 Hz, 1H), 5.99 (d, J=3.2 Hz, 2H), 4.53-4.42 (m, 2H), 4.32-3.85 (m, 1H), 3.41-3.31 (m, 2H), 3.23-2.95 (m, 2H), 2.38-2.29 (m, 2H), 2.30 (s, 2H), 1.96-1.80 (m, 2H), 1.82-1.47 (m, 3H), 1.39 (t, J=7.0 Hz, 4H).

Example 78: LC purity: 99.9% (UV at 254 nm); Mass calculated for C₂₆H₂₇F₃N₆O₂ [M+1]⁺, 513.2, found 513.2; Retention time: 1.333 min. ¹H NMR (400 MHz, DMSO) δ 8.78 (t, J=2.2 Hz, 1H), 8.48-8.36 (m, 1H), 8.09-8.02 (m, 1H), 7.94 (d, J=16.8 Hz, 1H), 7.30 (dd, J=11.2, 8.1 Hz, 1H), 5.99 (d, J=3.3 Hz, 2H), 4.52-4.42 (m, 2H), 4.33-3.84 (m, 1H), 3.42-3.33 (m, 2H), 3.25-2.97 (m, 2H), 2.25-2.05 (m, 4H), 1.89-1.76 (m, 3H), 1.57-1.52 (m, 2H), 1.44-1.23 (m, 4H).

### Example 79. (5-amino-6″-ethoxy-3′,6-difluoro-[2,2′:6′,3″-terpyridin]-4′-yl)(4-amino-2-azabicyclo[2.2.2]octan-2-yl)methanone

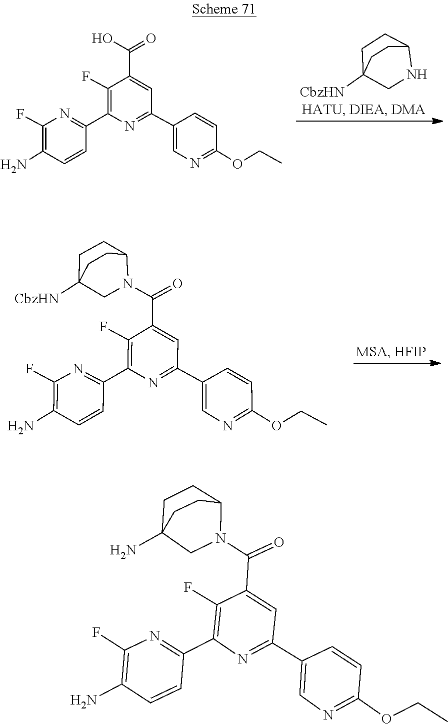 [SMILES: CCOc1ccc(-c2cc(C(=O)O)c(F)c(-c3ccc(N)c(F)n3)n2)cn1 ; O=C(NC12CCC(CC1)NC2)OCc1ccccc1 ; CCOc1ccc(-c2cc(C(=O)N3CC4(NC(=O)OCc5ccccc5)CCC3CC4)c(F)c(-c3ccc(N)c(F)n3)n2)cn1 ; CCOc1ccc(-c2cc(C(=O)N3CC4(N)CCC3CC4)c(F)c(-c3ccc(N)c(F)n3)n2)cn1]

Step 1: To a solution of 5-amino-6″-ethoxy-3′,6-difluoro-[2,2′:6′,3″-terpyridine]-4′-carboxylic acid (1.0 eq, 100 mg, 0.27 mmol) and benzyl (2-azabicyclo[2.2.2]octan-4-yl)carbamate (1.0 eq, 70 mg, 0.27 mmol) in DMA (5 mL) were added HATU (2.0 eq, 205 mg, 0.54 mmol) and DIEA (3.0 eq, 104 mg, 0.81 mmol). The reaction was stirred at room temperature for 1 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in benzyl (2-(5-amino-6″-ethoxy-3′,6-difluoro-[2,2′:6′,3″-terpyridine]-4′-carbonyl)-2-azabicyclo[2.2.2]octan-4-yl)carbamate (100 mg, 0.16 mmol, 57.0% yield) as a yellow solid. LC purity: 94.2% (UV at 254 nm); Mass calculated for C₃₃H₃₂F₂N₆O₄[M+1]⁺ 615.2, found 615.2. Retention time:1.083 min.

Step 2: To a solution of benzyl (2-(5-amino-6″-ethoxy-3′,6-difluoro-[2,2′:6′,3″-terpyridine]-4′-carbonyl)-2-azabicyclo[2.2.2]octan-4-yl)carbamate (1.0 eq, 70 mg, 0.11 mmol) in HFIP (5 mL) was added MSA (1 mL) dropwise at 0° C., and the resulting solution was stirred at room temperature for 2 h. The resulting mixture was concentrated under reduced pressure. The residue was purified with following condition: Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient (B %): 26% B to 50% B in 8 min; Wave Length: 254 nm/220 nm; RT1(min): 7.11. The pure fractions were concentrated and lyophilized to afford title compound (22.61 mg, 0.04 mmol, 41.2% yield) as a white solid.

### Example 79: LC purity: 99.9% (UV at 254 nm); Mass calculated for C₂₅H₂₆F₂N₆O₂

[M+1]⁺ 481.2, found 481.2; Retention time: 1.113 min ¹H NMR (400 MHz, DMSO+D₂O) δ 8.87 (dd, J=9.0, 2.5 Hz, 1H), 8.51-8.33 (m, 1H), 8.01-7.94 (m, 1H), 7.93-7.86 (m, 1H), 7.31 (dd, J=11.2, 8.0 Hz, 1H), 6.92 (dd, J=8.7, 4.9 Hz, 1H), 4.42-4.29 (m, 2H), 3.43-2.97 (m, 3H), 1.79 (s, 2H), 1.63-1.55 (m, 6H), 1.36-1.28 (m, 3H).

### Example 80. (6-(5-amino-6-fluoropyrazin-2-yl)-6′-ethoxy-5-fluoro-[2,3′-bipyridin]-4-yl)(4-amino-2-azabicyclo[2.2.2]octan-2-yl)methanone

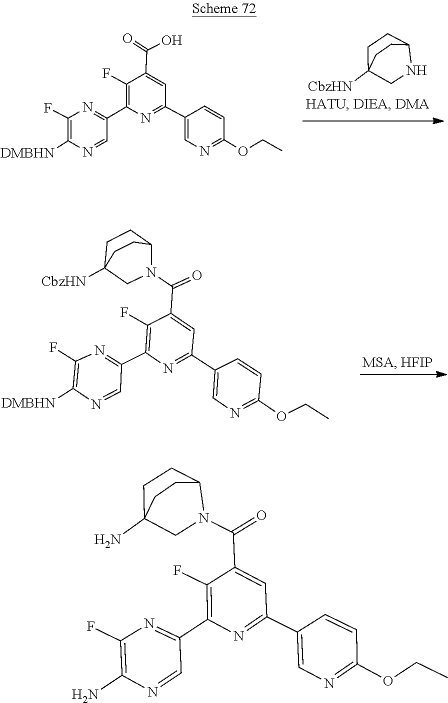

Step 1: To a solution of 6-(5-((2,4-dimethoxybenzyl)amino)-6-fluoropyrazin-2-yl)-6′-ethoxy-5-fluoro-[2,3′-bipyridine]-4-carboxylic acid (1.0 eq, 130 mg, 0.26 mmol) and benzyl (2-azabicyclo[2.2.2]octan-4-yl)carbamate (1.0 eq., 67 mg, 0.26 mmol) in DMA (5 mL) were added HATU (2.0 eq, 198 mg, 0.52 mmol) and DIEA (3.0 eq, 100 mg, 0.78 mmol). The reaction was stirred at room temperature for 1 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in benzyl (2-(6-(5-((2,4-dimethoxybenzyl)amino)-6-fluoropyrazin-2-yl)-6′-ethoxy-5-fluoro-[2,3′-bipyridine]-4-carbonyl)-2-azabicyclo[2.2.2]octan-4-yl)carbamate (100 mg, 0.13 mmol, 44.9% yield) as a yellow solid. LC purity: 85.4% (UV at 254 nm); Mass calculated for C₄₁H₄₁F₂N₇O₆[M+1]⁺ 766.3, found 766.3. Retention time:1.045 min.

Step 2: To a solution of benzyl (2-(6-(5-((2,4-dimethoxybenzyl)amino)-6-fluoropyrazin-2-yl)-6′-ethoxy-5-fluoro-[2,3′-bipyridine]-4-carbonyl)-2-azabicyclo[2.2.2]octan-4-yl)carbamate (1.0 eq, 70 mg, 0.11 mmol) in HFIP (5 mL) was added MSA (1 mL) dropwise at 0° C., and the resulting solution was stirred at room temperature for 2 h. The resulting mixture was concentrated under reduced pressure. The residue was purified with following condition: Column: XBridge Prep OBD C18 Column, 30*150 mm, 5 μm; Mobile Phase A: Water(10 mmol/LNH₄HCO₃), Mobile Phase B: ACN; Flow rate: 60 mL/min mL/min; Gradient (B %): 25% B to 40% B in 8 min; Wave Length: 254 nm/220 nm; RT1(min): 7.25. The pure fractions were concentrated and lyophilized to afford title compound (16.28 mg, 0.03 mmol, 34.5% yield) as a white solid.

Example 80: LC purity: 97.3% % (UV at 254 nm); Mass calculated for C₂₋₄H₂₅F₂N₇O₂[M+1]⁺ 482.2, found 482.1; Retention time: 1.042. ¹H NMR (400 MHz, DMSO+D₂O) δ 8.91-8.86 (m, 1H), 8.69 (dd, J=10.5, 4.6 Hz, 1H), 8.49-8.38 (m, 1H), 7.95 (dd, J=8.2, 3.7 Hz, 1H), 6.92 (dd, J=8.8, 5.0 Hz, 1H), 4.34 (dd, J=8.4, 5.7 Hz, 2H), 3.34 (d, J=34.6 Hz, 2H), 3.09 (d, J=52.8 Hz, 1H), 1.79 (s, 2H), 1.63-1.56 (m, 6H), 1.35-1.27 (m, 3H).

### Example 81. (5-amino-6″-ethoxy-3′,6-difluoro-[2,2′:6′,3″-terpyridin]-4′-yl)((3aS,7aR)-1-methyloctahydro-5H-pyrrolo[3,2-c]pyridin-5-yl)methanone

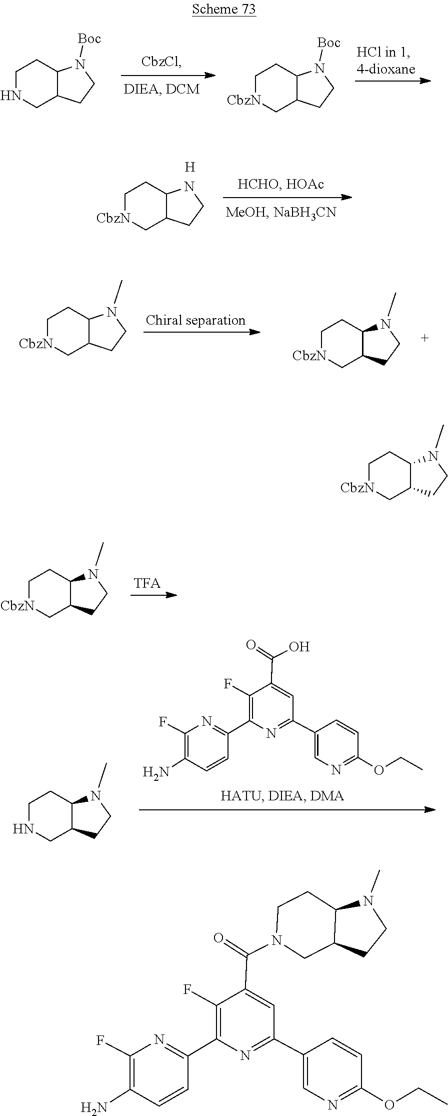

Step 1: To a stirred solution of tert-butyl octahydro-1H-pyrrolo[3,2-c]pyridine-1-carboxylate (1.0 eq, 780 mg, 3.4 mmol) and CbzCl (1.2 eq, 0.6 mL, 4.1 mmol) in DCM (10 mL) was added TEA (2.0 eq, 696 mg, 6.9 mmol). The resulting solution was stirred at room temperature for 2 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The residue was purified by reversed-phase flash chromatography (C18 silica gel) using a gradient of 0% to 100% ACN in water (with 0.1% FA) to afford 5-benzyl 1-(tert-butyl) hexahydro-1H-pyrrolo[3,2-c]pyridine-1,5(4H)-dicarboxylate (1000 mg, 2.2 mmol, 64.2% yield) as a yellow oil. LC purity: 79.8% (UV at 254 nm); Mass calculated for C₂₀H₂₈N₂O₄[M+1-56]⁺ 305.2, found 305.2; Retention time: 0.886 min.

Step 2: To a solution of 5-benzyl 1-(tert-butyl) hexahydro-1H-pyrrolo[3,2-c]pyridine-1,5(4H)-dicarboxylate (1.0 eq, 1000 mg, 3.8 mmol) in 1,4-dioxane (5 mL) was added HCl in 1,4-dioxane (1 M, 5 mL). The resulting solution was stirred at room temperature for 2 h. The resulting mixture was concentrated under reduced pressure. The crude product was purified by reversed-phase flash chromatography (C18 silica gel) using a gradient of 0% to 100% ACN in water (with 0.1% FA) to afford benzyl octahydro-5H-pyrrolo[3,2-c]pyridine-5-carboxylate (700 mg, 2.7 mmol, 97.9% yield) as a yellow oil. LC purity: 72.9% (UV at 254 nm); Mass calculated for C₁₅H₂₀N₂O₂[M+1]⁺ 261.2, found 261.2; Retention time: 0.700 min.

Step 3: To a solution of benzyl octahydro-5H-pyrrolo[3,2-c]pyridine-5-carboxylate (1.0 eq, 690 mg, 2.6 mmol) and HCHO (1.1 eq, 219 mg, 2.9 mmol) in MeOH (5 mL) was added HOAc (0.25 mL). The resulting mixture was stirred at room temperature for 20 min. This was followed by the addition of a solution of NaBH₃CN (3.0 eq, 501 mg, 7.9 mmol) in MeOH (2 mL). The resulting mixture was stirred at room temperature for 1 h. The resulting mixture was stirred at room temperature for 2 h. The mixture was diluted with water and extracted with EtOAc. The combined extracts were washed with brine, dried over anhydrous sodium sulfate, filtered and concentrated. The residue was dissolved in DMSO, filtered and purified via Prep-HPLC (Column: XBridge Prep OBD C18 Column, 30*150 mm, 5 m; Mobile Phase A: Water(10 mmol/LNH₄HCO₃), Mobile Phase B: ACN; Flow rate: 60 mL/min; Gradient (B %): 30% B to 48% B in 8 min; Wave Length: 254 nm/220n; RT1(min): 7.89). The pure fractions were concentrated to afford benzyl 1-methyloctahydro-5H-pyrrolo[3,2-c]pyridine-5-carboxylate (390 mg, 1.42 mmol, 53.6% yield) as a colorless oil. LC purity: 95% (UV at 254 nm); Mass calculated for C₁₆H₂₂N₂O₂[M+1]⁺ 275.2, found 275.2; Retention time: 0.847 min.

Step 4: The racemate benzyl 1-methyloctahydro-5H-pyrrolo[3,2-c]pyridine-5-carboxylate (100 mg, 0.36 mmol) was purified by prep-chiral-HPLC with following conditions: Column: CHIRALPAK IG, 5 μm, 20 mm 250 mm; Mobile Phase A: EtOH: DCM=1: 1, Mobile Phase B: Hex (with 0.5% NH₃ (2 M in MeOH)); Flow rate: 20 mL/min; Gradient (B %): 85% B; Wave Length: 220/254 nm; RT1(min): 7.76; RT2(min): 9.92; Sample Solvent: EtOH: DCM=1: 1; Injection Volume: 0.3 mL; Number Of Runs: 10; Single injection run time(min): 10. The pure fractions of the first peak (RT1 (min): 3.551, and second peak (RT2 (min): 4.484) were concentrated. Each residue was re-dissolved in acetonitrile and water, then lyophilized to afford benzyl (3aS,7aR)-1-methyloctahydro-5H-pyrrolo[3,2-c]pyridine-5-carboxylate (40 mg, 0.15 mmol, 41.7% yield), first peak, LC purity: 98% (UV at 254 nm); Mass calculated for C₁₆H₂₂N₂O₂ [M+1]⁺, 275.2, found 275.2; Retention time: 0.847 min; and benzyl (3aR,7aS)-1-methyloctahydro-5H-pyrrolo[3,2-c]pyridine-5-carboxylate (40 mg, 0.15 mmol, 41.7% yield), second peak, both obtained as white solids, LC purity: 98% (UV at 254 nm); Mass calculated for C₁₆H₂₂N₂O₂ [M+1]⁺, 275.2, found 275.2; Retention time: 0.847 min.

Step 5: To a solution of benzyl (3aS,7aR)-1-methyloctahydro-5H-pyrrolo[3,2-c]pyridine-5-carboxylate (1.0 eq, 40 mg, 0.14 mmol) in DCM (0.5 mL) was added TFA (3 mL). The resulting solution was stirred at 50° C. for 12 h. The resulting mixture was concentrated under reduced pressure. The crude product was used in the next step directly without further purification. LC purity: N/A (UV at 254 nm); Mass calculated for C₈H₁₆N₂ [M+1]⁺, 141.1, found 141.1; Retention time: 0.104 min.

Step 6: To a solution of (3aS,7aR)-1-methyloctahydro-1H-pyrrolo[3,2-c]pyridine (1.0 eq, 40 mg, 0.14 mmol) and 5-amino-6″-ethoxy-3′,6-difluoro-[2,2′:6′,3″-terpyridine]-4′-carboxylic acid (1.0 eq, 106 mg, 0.14 mmol) in DMA (5 mL) were added HATU (1.5 eq, 163 mg, 0.21 mmol) and DIEA (1.5 eq, 55 mg, 0.21 mmol). The reaction was stirred at room temperature for 1 h. After filtration, the filtrate was purified by Prep-HPLC under the following conditions: Column: XBridge Prep OBD C18 Column, 30*150 mm, 5 μm; Mobile Phase A: Water (10 mmol/LNH₄HCO₃), Mobile Phase B: ACN; Flow rate: 60 mL/min; Gradient (B %): 40% B to 55% B in 8 min; Wavelength: 254 nm/220 nm; RT1(min): 7.99. The pure fractions were concentrated and lyophilized to afford title compound (24.74 mg, 0.05 mmol, 17.5% yield) as a white solid.

Example 81: LC purity: 99.8% (UV at 254 nm); Mass calculated for C₂₆H₂₈F₂N₆O₂ [M+1]⁺ 495.2, found 495.2. Retention time: 1.073 min. ¹H NMR (400 MHz, DMSO+D₂O) δ 8.91 (t, J=2.3 Hz, 1H), 8.44 (dd, J=8.6, 2.6 Hz, 1H), 7.99 (dd, J=8.1, 1.6 Hz, 1H), 7.94 (s, 1H), 7.31 (dd, J=11.1, 8.1 Hz, 1H), 6.92 (d, J=8.7 Hz, 1H), 4.41-4.32 (m, 2H), 3.87-3.80 (m, 1H), 3.27 (d, J=12.3 Hz, 2H), 3.20-2.89 (m, 2H), 2.37-2.29 (m, 1H), 2.19 (d, J=18.3 Hz, 4H), 2.11-2.02 (m, 1H), 1.82-1.69 (d, J=55.3 Hz, 3H), 1.34 (t, J=7.1 Hz, 3H), 1.28-1.13 (m, 1H).

### Example 82. (5-amino-6″-ethoxy-3′,6-difluoro-[2,2′:6′,3″-terpyridin]-4′-yl)((3aR,7aS)-1-methyloctahydro-5H-pyrrolo[3,2-c]pyridin-5-yl)methanone

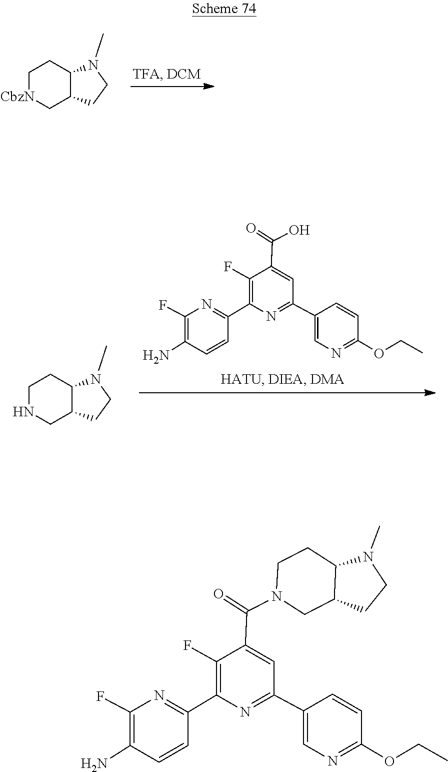

Step 1: To a solution of benzyl (3aR,7aS)-1-methyloctahydro-5H-pyrrolo[3,2-c]pyridine-5-carboxylate (1.0 eq, 40 mg, 0.1 mmol) in DCM (1 mL) was added TFA (3 mL). The mixture was stirred at 50° C. for 12 h. The resulting mixture was concentrated under reduced pressure to afford crude product (3aR,7aS)-1-methyloctahydro-1H-pyrrolo[3,2-c]pyridine (40 mg) which was used in the next step directly without further purification. LC purity: N/A (UV at 254 nm); Mass calculated for C₈H₁₆N₂[M+1]⁺, 141.1, found 141.1; Retention time: 0.104 min.

Step 2: To a solution of (3aR,7aS)-1-methyloctahydro-1H-pyrrolo[3,2-c]pyridine (1.0 eq, 35 mg, 0.2 mmol) and 5-amino-6″-ethoxy-3′,6-difluoro-[2,2′:6′,3″-terpyridine]-4′-carboxylic acid (1.0 eq, 93 mg, 0.2 mmol) in DMA (2 mL) were added HATU (1.5 eq, 142 mg, 0.3 mmol) and DIEA (5.0 eq, 161 mg, 1.2 mmol). The mixture was stirred at room temperature for 2 h. After filtration, the filtrate was purified by Prep-HPLC under the following conditions: Column: XBridge Prep OBD C18 Column, 30*150 mm, 5 μm; Mobile Phase A: Water (10 mmol/LNH₄HCO₃), Mobile Phase B: ACN; Flow rate: 60 mL/min; Gradient (B %): 40% B to 55% B in 8 min; Wavelength: 254 nm/220 nm; RT1(min): 7.99. The pure fractions were concentrated and lyophilized to afford title compound (24.45 mg, 0.04 mmol, 19% yield) as a white solid.

Example 82: LC purity: 99.8% (UV at 254 nm); Mass calculated for C₂₆H₂₈F₂N₆O₂ [M+1]⁺ 495.2, found 495.2. Retention time: 1.081 min. ¹H NMR (400 MHz, DMSO) δ 8.95 (s, 1H), 8.47 (dd, J=8.7, 2.5 Hz, 1H), 8.04-7.96 (m, 2H), 7.30 (dd, J=11.2, 8.1 Hz, 1H), 6.93 (d, J=8.7 Hz, 1H), 5.98 (s, 2H), 4.44-4.34 (m, 2H), 4.03-3.84 (m, 1H), 3.10-2.92 (m, 4H), 2.23 (s, 2H), 2.21-2.02 (m, 3H), 2.13-2.02 (m, 1H), 1.83-1.69 (m, 3H), 1.36-1.30 (m, 4H).

### Example 83. (6-(5-amino-6-fluoropyrazin-2-yl)-6′-ethoxy-5,5′-difluoro-[2,3′-bipyridin]-4-yl)(4-amino-2-azabicyclo[2.2.2]octan-2-yl)methanone

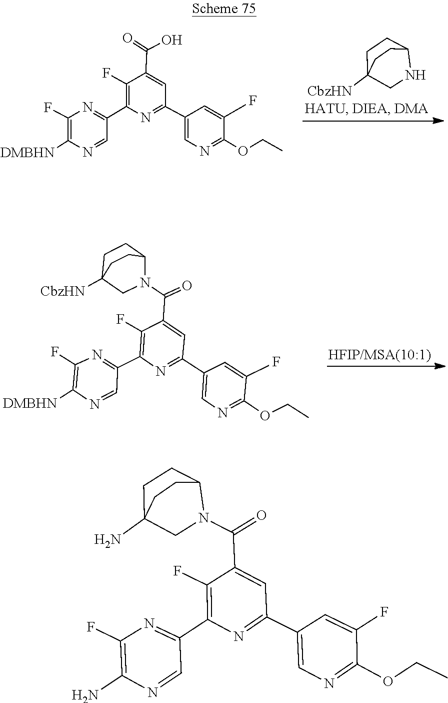

Step 1: To a solution of 6-(5-((2,4-dimethoxybenzyl)amino)-6-fluoropyrazin-2-yl)-6′-ethoxy-5,5′-difluoro-[2,3′-bipyridine]-4-carboxylic acid (1.0 equiv. 100 mg, 0.18 mmol) and benzyl (2-azabicyclo[2.2.2]octan-4-yl)carbamate (1.5 eq, 72 mg, 0.28 mmol) in DMA (5 mL) were added DIEA (3.0 equiv. 70 mg, 0.54 mmol) and HATU (1.5 equiv. 103 mg, 0.28 mmol). The mixture was stirred at room temperature for 2 h. The resulting mixture was diluted with water and extracted with EtOAc (2×100 mL). The combined organic layers were washed with brine (1×100 mL), dried over anhydrous Na₂SO₄. After filtration, the filtrate was concentrated under reduced pressure. The residue was purified by reversed phase flash chromatography using a gradient of 60% to 80% ACN in water (0.1% FA) to afford benzyl (2-(6-(5-((2,4-dimethoxybenzyl)amino)-6-fluoropyrazin-2-yl)-6′-ethoxy-5,5′-difluoro-[2,3′-bipyridine]-4-carbonyl)-2-azabicyclo[2.2.2]octan-4-yl)carbamate (85 mg, 0.11 mmol, 60% yield) as a yellow solid. LC purity: 95.6% (UV at 254 nm); Mass calculated for C₄₁H₄₀F₃N₇O₆[M+1]⁺ 784.4, found 784.4; Retention time: 1.280 min.

Step 2: To a stirred mixture of benzyl (2-(6-(5-((2,4-dimethoxybenzyl)amino)-6-fluoropyrazin-2-yl)-6′-ethoxy-5,5′-difluoro-[2,3′-bipyridine]-4-carbonyl)-2-azabicyclo[2.2.2]octan-4-yl)carbamate (1.0 equiv. 81 mg, 0.11 mmol) in HFIP (5 mL) was added MSA (0.5 mL), and the resulting mixture was stirred at room temperature for 1 h. The mixture was evaporated and the residue was purified by Prep-HPLC with following condition: Column: XBridge Prep OBD C18 Column, 30*150 mm, 5 μm; Mobile Phase A: Water(10 mmol/LNH₄HCO₃), Mobile Phase B: ACN; Flow rate: 60 mL/min; Gradient (B %): 30% B to 50% B in 8 min; Wave Length: 254 nm/220 nm; RT1(min): 7.54. The pure fractions were concentrated and lyophilized to afford title compound (23.37 mg, 0.047 mmol, 44.9% yield) as a white solid.

### Example 83: LC purity: 99.8% (UV at 254 nm); Mass calculated for Chemical

Formula: C₂₋₄H₂₄F₃N₇O₂[M+1]⁺, 500.2, found 500.2; Retention time: 1.198 min. ¹H NMR (400 MHz, DMSO) δ 8.84-8.74 (m, 2H), 8.49-8.39 (m, 1H), 8.09 (dd, J=5.9, 3.5 Hz, 1H), 7.40 (d, J=5.3 Hz, 2H), 4.54-4.40 (m, 2H), 3.39 (s, 1H), 3.29 (s, 1H), 3.01 (s, 1H), 1.84 (s, 2H), 1.75-1.44 (m, 8H), 1.44-1.34 (m, 3H).

### Example 84. Biological Data

THP-1 DUAL cell reporter assay for ISRE (interferon stimulated response element) and NFkB: THP-1 DUAL cells (InVivoGen #thpd-nfis) were kept in culture in culture medium (RPMI-1640+10% heat-inactivated fetal bovine serum+1% Penicillin Streptomycin+1X GlutaMax+5 mM HEPES+100 g/mL Normocin+100 g/mL Zeocin+10 g/mL Blasticidin). On the day of the assay, cells were seeded in 384-well plates (Corning #3570) at 25,000 cells/well in 45 μl assay medium (RPMI-1640+10% heat-inactivated fetal bovine serum+100 Penicillin Streptomycin+1X GutaMax+5 mM DEPES+100 μg/mL Normocin). Compound stocks were 3-fold serially diluted using TECAN EVO200. From the diluted stock plate, 50 nL were dispensed into the plate containing cells with an Echo655 instrument. Cells were incubated at 37° C. 5% CO2 for 18 h. The next day, 5 μl of 50 μg/mL R848 (InVivoGen #tlrl-r848-1) were added to each well where the final concentration of R848 is 5 ag/mL. Cells were incubated at 37° C. 500 CO2 for 24 h. For QUANTI-Luc (ISRE) detection, plates were spun down at 600 g for 3 min and 15 μl of supernatant transferred to a new white opaque 384-well plate (Corning #3570). QUANTI-Luc assay solution (InVivoGen #rep-qlc2) was added at 40 μl per well and measured on a plate reader (Ensight, PerkinElmer). For QUANTI-Blue (NFkB) detection, plates were spun down at 600 g for 3 min and 5 μl of supernatant transferred to a new bottom clear 384-well plate (Corning #3764). QUANTI-Blue assay solution (InVivoGen #rep-qbS2) was added at 45 l per well and incubated for 15 min at 37° C. Signal was measured on a plate reader (Ensight, PerkinElmer).

TABLE 3
THP-1 DUAL cell reporter assay for ISRE and NFkB.

| Example | ISRE | NFKB |
|---|---|---|
| 1 | | D |
| 2 | A | D |
| 3 | A | — |
| 4 | A | D |
| 5 | A | D |
| 6 | A | — |
| 7 | A | — |
| 8 | A | D |
| 9 | A | D |
| 10 | A | — |
| 11 | A | D |
| 12 | A | — |
| 13 | A | D |
| 14 | A | — |
| 15 | A | D |
| 16 | A | C |
| 17 | A | D |
| 18 | A | D |
| 19 | A | D |
| 20 | A | D |
| 21 | A | — |
| 22 | A | — |
| 23 | A | — |
| 24 | A | — |
| 25 | A | — |
| 26 | A | — |
| 27 | A | D |
| 28 | A | — |
| 29 | A | — |
| 30 | A | D |
| 31 | A | D |
| 32 | A | D |
| 33 | A | — |
| 34 | A | D |
| 35 | A | — |
| 36 | A | — |
| 37 | A | D |
| 38 | A | — |
| 39 | A | — |
| 40 | A | D |
| 41 | A | — |
| 42 | A | D |
| 43 | A | D |
| 44 | A | D |
| 45 | A | — |
| 46 | A | — |
| 47 | A | — |
| 48 | A | — |
| 49 | A | — |
| 50 | A | — |
| 51 | A | — |
| 52 | A | — |
| 53 | A | — |
| 54 | A | — |
| 55 | A | — |
| 56 | A | — |
| 57 | A | — |
| 58 | A | — |
| 59 | A | — |
| 60 | A | — |
| 61 | A | — |
| 62 | A | — |
| 63 | A | — |
| 64 | A | — |
| 65 | A | — |
| 66 | A | — |
| 67 | A | — |
| 68 | A | — |
| 69 | A | — |
| 70 | A | — |
| 71 | A | — |
| 72 | A | — |
| 73 | A | — |
| 74 | A | — |
| 75 | B | — |
| 76 | A | — |
| 77 | A | — |
| 78 | A | — |
| 79 | — | — |
| 80 | — | — |
| 81 | — | — |
| 82 | — | — |
| 83 | — | — |

A ≤ 100 nM, 100 nM <B ≤ 1000 nM, 1000 nM < C ≤ 10000 nM, D > 10000 nM

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula I: 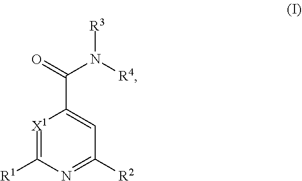
or pharmaceutically acceptable salt thereof,
wherein
R¹ is a C₃-C₈ cycloalkyl, heterocyclyl, C₆-C₁₀ aryl, or heteroaryl, which are each substituted by 0, 1, 2, or 3 R^{1a};
R² is a C₃-C₈ cycloalkyl, heterocyclyl, C₆-C₁₀ aryl, or heteroaryl, which are each substituted by 0, 1, 2, or 3 R^{2a};
X is C—X^{1A} or N;
X^{1a} is H, C₁-C₆ alkyl, C₂-C₆ alkenyl, C₂-C₆ alkynyl, C₁-C₆ alkoxy, C₁-C₆ aminoalkyl, C₁-C₆ sulfonylalkyl, C₁-C₆ haloalkyl, C₁-C₆ haloalkoxy, halogen, or —CN;
R³ is —(C₀-C₃ alkylene)(C₃-C₈ cycloalkyl), —(C₀-C₃ alkylene)(heterocyclyl), —(C₀-C₃ alkylene)(C₆-C₁₀ aryl), or —(C₀-C₃ alkylene)(heteroaryl), which are each substituted by 0, 1, 2, or 3 R^{3a};
R^{3a} is C₁-C₆ alkyl, C₂-C₆ alkenyl, C₂-C₆ alkynyl, C₁-C₆ alkoxy, C₁-C₆ haloalkyl, C₁-C₆ haloalkoxy, C₃-C₆ cycloalkyl, heterocyclyl, C₆-C₁₀ aryl, heteroaryl, halogen, —OR^{3b}, —NR^{3c}R^{3d}, —CN, —C(O)R^{3b}, —C(O)NR^{3c}R^{3d}, —OC(O)NR^{3c}R^{3d}, —N(R^{3b})C(O)NR^{3c}R^{3d}, or —S(O)₂NR^{3c}R^{3d};
R⁴ is C₁-C₆ alkyl, C₂-C₆ alkenyl, C₂-C₆ alkynyl, or C₁-C₆ haloalkyl; or
alternatively, R³ and R⁴ together with the nitrogen to which they are attached form a heterocyclyl, which is substituted by 0, 1, 2, or 3 R^{3a};
each R^{1a} and R^{2a} is independently C₁-C₆ alkyl, C₂-C₆ alkenyl, C₂-C₆ alkynyl, C₁-C₆ alkoxy, C₁-C₆ haloalkyl, C₁-C₆ haloalkoxy, C₃-C₆ cycloalkyl, heterocyclyl, C₆-C₁₀ aryl, heteroaryl, halogen, —OR^{1b}, —NR^{1c}R^{1d}, —CN, —C(O)R^{1b}, —C(O)NR^{1c}R^{1d}, —OC(O)NR^{1c}R^{1d}, —N(R^{1b})C(O)NR^{1c}R^{1d} or —S(O)₂NR^{1c}R^{1d}; and
each R^{1b}, R^{1c}, R^{1d}, R^{3b}, R^{3c}, and R^{3d} is independently H, C₁-C₆ alkyl, or C₁-C₆ haloalkyl, or
alternatively, R^{3c} and R^{3d} together with the nitrogen to which they are attached form a heterocyclyl.

2. The compound or pharmaceutically acceptable salt thereof of claim 1, wherein X¹ is C—F.

3. The compound or pharmaceutically acceptable salt thereof of claim 1, wherein X¹ is C—H.

4. The compound or pharmaceutically acceptable salt thereof of claim 1, wherein R¹ is heterocyclyl or heteroaryl, which are each substituted by 0, 1, 2, or 3 R^{1a}.

5. The compound or pharmaceutically acceptable salt thereof of claim 1, wherein R¹ is a 4- to 7-membered heterocyclyl or 5- to 6-membered heteroaryl, which are each substituted by 0, 1, 2, or 3 R^{1a}.

6. The compound or pharmaceutically acceptable salt thereof of claim 1, wherein R¹ is a 5- to 6-membered heteroaryl, which is substituted by 0, 1, 2, or 3 R^{1a}.

7. The compound or pharmaceutically acceptable salt thereof of claim 6, wherein R¹ is a pyrazolyl, pyridyl, or pyrimidyl, which are each substituted by 0, 1, 2, or 3 R^{1a}.

8. The compound or pharmaceutically acceptable salt thereof of claim 1, wherein
each R^{1a} is independently C₁-C₆ alkyl, C₁-C₆ alkoxy, C₁-C₆ haloalkyl, C₁-C₆ haloalkoxy, halogen, —OR^{1b}, —NR^{1c}R^{1d}, or —CN; and
each R^{1b}, R^{1c}, and R^{id} is independently H and C₁-C₆ alkyl.

9. The compound or pharmaceutically acceptable salt thereof of claim 1, wherein R¹ is

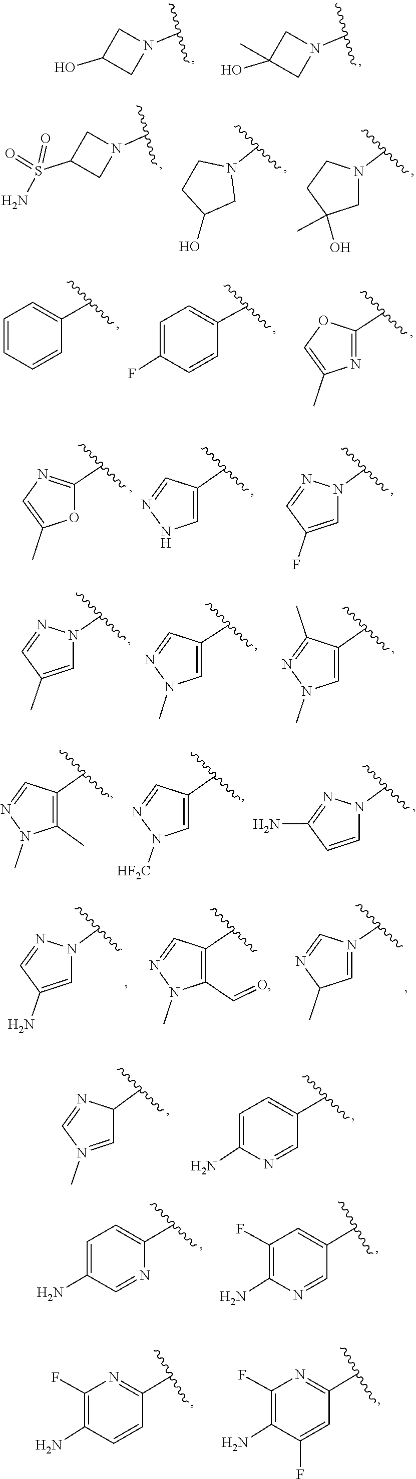

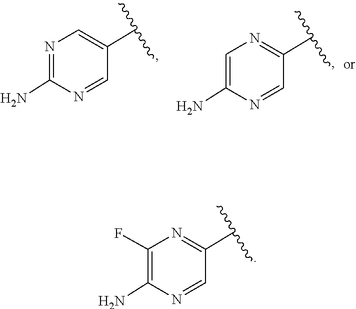 [SMILES: *c1cnc(N)nc1 ; *c1cnc(N)cn1 ; *c1cnc(N)c(F)n1]

10. The compound or pharmaceutically acceptable salt thereof of claim 1, wherein R² is phenyl or pyridyl, which are each substituted by 0, 1, 2, or 3 R^{2a}.

11. The compound or pharmaceutically acceptable salt thereof of claim 1, wherein each R^{2a} is independently C₁-C₆ alkyl, C₁-C₆ alkoxy, C₁-C₆ haloalkyl, C₁-C₆ haloalkoxy, C₃-C₆ cycloalkyl, halogen, —OR^{1b}, —NR^{1c}R^{1d}, or —CN.

12. The compound or pharmaceutically acceptable salt thereof of claim 1, wherein R² is

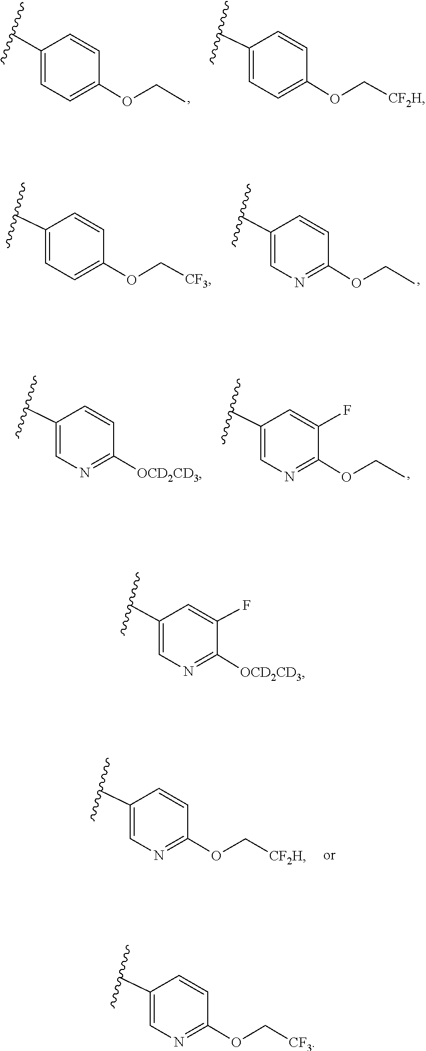

13. The compound or pharmaceutically acceptable salt thereof of claim 1, wherein R³ is heterocyclyl, substituted by 0, 1, 2, or 3 R^{3a}.

14. The compound or pharmaceutically acceptable salt thereof of claim 1, wherein R⁴ is C₁-C₆ alkyl.

15. The compound or pharmaceutically acceptable salt thereof of claim 1, wherein R³ and R⁴ together with the nitrogen to which they are attached form a heterocyclyl, which is substituted by 0, 1, 2, or 3 R^{3a}.

16. The compound or pharmaceutically acceptable salt thereof of claim 1, wherein, unless otherwise indicated,
the heteroaryl in each instance is 5- to 10-membered ring having 1, 2, 3, or 4 heteroatoms selected from N, O, and S; and
the heterocyclyl in each instance is 3- to 12-membered ring having 1, 2, 3, or 4 heteroatoms selected from N, O, and S.

17. The compound or pharmaceutically acceptable salt thereof of claim 15, wherein R³ and R⁴ together with the nitrogen to which they are attached form

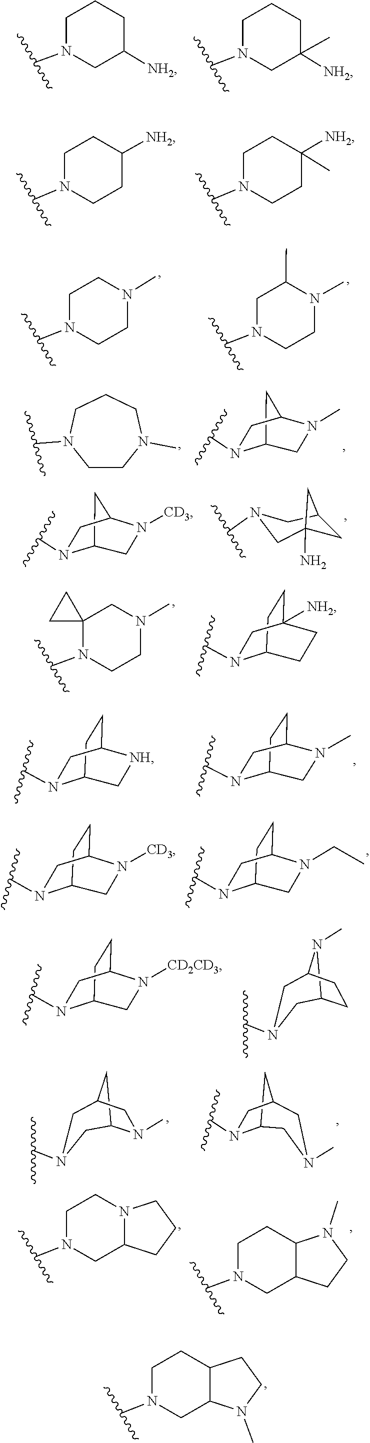

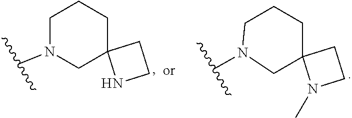 [SMILES: *N1CCCC2(CCN2)C1 ; *N1CCCC2(CCN2C)C1]

18. The compound or pharmaceutically acceptable salt thereof of claim 1, wherein the compound is any one shown in Table 1.

19. A pharmaceutical composition comprising a compound or pharmaceutically acceptable salt thereof of claim 1, and a pharmaceutically acceptable excipient.

20-21. (canceled)

22. A method of treating an autoimmune disorder or an inflammatory condition in a subject in need thereof, comprising administering to the subject a therapeutically effective amount of a compound or pharmaceutically acceptable salt thereof of claim 1.

23-24. (canceled)
